SEQUENCE LISTING (203 sequences)

SEQ ID NO: 1 - DNA, 639 bp, synthetic construct
  source 1..639: mol_type = other DNA
agccgcagcgaaagcaaaaaaaaccgcggcggccgcgaagaaattctggaacagtgggtgggcgcgcgcaaaaaactggaagaactggaacgcgatctgcgcaaaattaaaaaaaaaattaaaaaactggaagaagaaaacccgtggctgggcaacattaaaggcattctgggcaaaaaagatcgcgaaggcgaaggcgcgccgccggcgaaacgcgcgcgcgcggatcagatggaagtggatagcggcccgcgcaaacgcccgtttcgcggcgaatttaccgataaagaacgccgcgatcatcgccgccgcaaagcgctggaaaacaaacgcaaacagctgagcagcggcggcaaaagcctgagcaaagaagaagaagaagaactgcgcaaactgaccgaagaagatgaacgccgcgaacgccgcgtggcgggcccgcgcgtgggcggcgtgaacccgctggaaggcggcacccgcggcgcgccgggcggcggctttgtgccgagcatgcagggcgtgccggaaagcccgtttgcgcgcaccggcgaaggcctggatgtgcgcggcaaccagggctttccgtgggatattctgtttccggcggatccgccgtttagcccgcagagctgccgcccgcag

SEQ ID NO: 2 - DNA, 639 bp, synthetic construct
  source 1..639: mol_type = other DNA
agccgcagcgaaagcaaaaaaaaccgcggcggccgcgaagaagtgctggaacagtgggtgaacggccgcaaaaaactggaagaactggaacgcgaactgcgccgcgcgcgcaaaaaaattaaaaaactggaagatgataacccgtggctgggcaacgtgaaaggcattctgggcaaaaaagataaagatggcgaaggcgcgccgccggcgaaacgcgcgcgcaccgatcagatggaaattgatagcggcccgcgcaaacgcccgctgcgcggcggctttaccgatcgcgaacgccaggatcatcgccgccgcaaagcgctgaaaaacaaaaaaaaacagctgagcgcgggcggcaaaagcctgagcaaagaagaagaagaagaactgaaacgcctgacccgcgaagatgaagaacgcaaaaaagaagaacatggcccgagccgcctgggcgtgaacccgagcgaaggcggcccgcgcggcgcgccgggcggcggctttgtgccgagcatgcagggcattccggaaagccgctttacccgcaccggcgaaggcctggatgtgcgcggcagccgcggctttccgcaggatattctgtttccgagcgatccgccgtttagcccgcagagctgccgcccgcag

SEQ ID NO: 3 - DNA, 639 bp, synthetic construct
  source 1..639: mol_type = other DNA
agccagagcgaaacccgccgcggccgccgcggcacccgcgaagaaaccctggaaaaatggattaccgcgcgcaaaaaagcggaagaactggaaaaagatctgcgcaaaacccgcaaaaccattaaaaaactggaagaagaaaacccgtggctgggcaacattgtgggcattattcgcaaaggcaaagatggcgaaggcgcgccgccggcgaaacgcccgcgcaccgatcagatggaagtggatagcggcccgggcaaacgcccgcataaaagcggctttaccgataaagaacgcgaagatcatcgccgccgcaaagcgctggaaaacaaaaaaaaacagctgagcgcgggcggcaaaattctgagcaaagaagaagaagaagaactgcgccgcctgaccgatgaagatgaagaacgcaaacgccgcgtggcgggcccgcgcgtgggcgatgtgaacccgagccgcggcggcccgcgcggcgcgccgggcggcggctttgtgccgcagatggcgggcgtgccggaaagcccgtttagccgcaccggcgaaggcctggatattcgcggcacccagggctttccgtgggtgagcccgagcccgccgcagcagcgcctgccgctgctggaatgcaccccgcag

SEQ ID NO: 4 - DNA, 639 bp, synthetic construct
  source 1..639: mol_type = other DNA
agccagagcgaaagcaaaaaaaaccgccgcggcggccgcgaagatattctggaaaaatggattaccacccgccgcaaagcggaagaactggaaaaagatctgcgcaaagcgcgcaaaaccattaaaaaactggaagatgaaaacccgtggctgggcaacattattggcattattcgcaaaggcaaagatggcgaaggcgcgccgccggcgaaacgcccgcgcaccgatcagatggaaattgatagcggcaccggcaaacgcccgcataaaagcggctttaccgataaagaacgcgaagatcatcgccgccgcaaagcgctggaaaacaaaaaaaaacagctgagcagcggcggcaaaaacctgagccgcgaagaagaagaagaactgggccgcctgaccgtggaagatgaagaacgccgccgccgcgtggcgggcccgcgcaccggcgatgtgaacctgagcggcggcggcccgcgcggcgcgccgggcggcggctttgtgccgcgcatggaaggcgtgccggaaagcccgtttacccgcaccggcgaaggcctggatattcgcggcaaccagggctttccgtgggtgcgcccgagcccgccgcagcagcgcctgccgctgctggaatgcaccccgcag

SEQ ID NO: 5 - protein, 213 aa, synthetic construct
SRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSCRPQ

SEQ ID NO: 6 - protein, 213 aa, synthetic construct
SRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSCRPQ

SEQ ID NO: 7 - protein, 213 aa, synthetic construct
SQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLECTPQ

SEQ ID NO: 8 - protein, 213 aa, synthetic construct
SQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLECTPQ

SEQ ID NO: 9 - DNA, 141 bp, synthetic construct
  source 1..141: mol_type = other DNA
ggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggc

SEQ ID NO: 10 - DNA, 141 bp, synthetic construct
  source 1..141: mol_type = other DNA
ggccagaacctgagcaccagcaacccgctgggcttttttccggatcatcagctggatccggcgtttcgcgcgaacaccgcgaacccggattgggattttaacccgaacaaagatacctggccggatgcgaacaaagtgggc

SEQ ID NO: 11 - protein, 47 aa, synthetic construct
GTNLSTSNPLGFFPDHQLDPAFRANSANPDWDFNPNKDTWPDANKVG

SEQ ID NO: 12 - protein, 47 aa, synthetic construct
GQNLSTSNPLGFFPDHQLDPAFRANTANPDWDFNPNKDTWPDANKVG

SEQ ID NO: 13 - DNA, 66 bp, synthetic construct
  source 1..66: mol_type = other DNA
ggaagcggagctactaacttcagcctgctgaagcaggctggagacgtggaggagaaccctggacct

SEQ ID NO: 14 - protein, 22 aa, synthetic construct
GSGATNFSLLKQAGDVEENPGP

SEQ ID NO: 15 - DNA, 3198 bp, synthetic construct
  source 1..3198: mol_type = other DNA
atggccagcagaagtgaatcaaaaaagaatcggggagggcgggaagaaatcctggaacagtgggtcggagcacggaagaaactggaagaactggagagggacctgcgcaagatcaagaagaagatcaagaagctggaggaggagaacccctggctgggcaatatcaagggcatcctgggcaagaaggatcgggagggagagggagcaccacctgcaaagagggccagagccgaccagatggaggtggatagcggaccaaggaagcgccctttcagaggagagtttaccgacaaggagcggagagatcacaggcgccggaaggccctggagaacaagaggaagcagctgagctccggcggcaagtccctgtctaaggaggaggaggaggagctgcgcaagctgacagaggaggacgagagaagggagaggagggtggcaggacctagggtgggaggcgtgaacccactggagggaggaaccagaggagcacctggaggaggattcgtgccatccatgcagggagtgcccgagtctccttttgcccggacaggcgagggcctggatgtgagaggcaatcagggcttcccctgggacatcctgtttcctgccgatccacccttctctcctcagagctgccggccacagagcagatccgagtctaagaagaacaggggaggaagagaggaggtgctggagcagtgggtgaatggccggaagaagctggaggagctggagcgggagctgagaagggccagaaagaagatcaagaagctggaagacgataatccttggctgggcaatgtgaaaggcatcctgggcaagaaggacaaggatggagagggagcacctccagcaaagagggcaagaaccgaccagatggagatcgattctggaccaaggaagcgccccctgagaggaggcttcacagaccgggagagacaggatcaccgccggagaaaggccctgaagaacaagaagaagcagctgtccgccggaggcaagagcctgtccaaagaagaggaagaggagctgaagaggctgacccgcgaggacgaggagaggaagaaggaggagcacggaccaagcaggctgggagtgaatccttccgagggaggacctaggggagcaccaggaggaggcttcgtgccatctatgcagggcatccccgagagccggtttaccagaacaggagagggcctggacgtgaggggctcccgcggctttcctcaggacatcctgttcccatctgatccccctttttccccccagtcttgtaggcctcagggcaccaacctgtctacaagcaatccactgggcttctttcccgaccaccagctggatcctgccttccgcgccaacagcgccaatcccgactgggacttcaacccaaataaggacacctggccagatgccaacaaggtcggcggccagaacctgtccacatctaatcctctgggcttctttccagaccaccagctggatccagccttccgggccaacacagctaaccctgactgggacttcaaccccaataaggatacttggcccgacgccaacaaggtcggcggaagcggagctactaacttcagcctgctgaagcaggctggagacgtggaggagaaccctggacctatgagccagtccgagacaaggaggggccggagaggaaccagggaggagacactggagaagtggatcacagcccgcaagaaggccgaggagctggagaaggacctgcggaagaccagaaagacaatcaagaagctggaagaagagaacccatggctgggcaatatcgtgggcatcatcagaaagggcaaggacggcgagggagcaccaccagcaaagaggcccaggactgatcagatggaagtcgatagcggaccaggcaagcggcctcacaagtccggcttcacagacaaggagagagaggaccataggcgccggaaggccctggaaaacaagaagaagcaattatccgccggcggcaagatcctgtccaaagaggaagaagaggagctgagaaggctgaccgacgaggatgaggagaggaaaagaagggtggcaggaccaagggtgggcgacgtgaatcccagcaggggaggaccaagaggcgcccctggcggcggcttcgtgccacagatggcaggagtgccagagagccccttttccaggacaggagagggcctggatatcagaggcacccagggctttccttgggtgtctccaagccctccacagcagcggctgccactgctggagtgcacccctcagtcccagtctgagagcaagaagaacagaaggggcggcagagaggacatcctggagaagtggatcaccacacgcagaaaagctgaagaactggaaaaggacctgaggaaggcccgcaaaacaatcaagaagctggaggatgaaaatccatggctgggaaacatcatcggcatcatcaggaagggcaaggacggggaaggcgcaccacctgcaaagcggcctagaacagatcagatggaaatcgattctggcaccggcaagaggccacacaagagcggcttcaccgacaaggagcgcgaggatcacagaaggcgcaaggccctggagaacaagaagaagcaattaagcagcggcggcaagaatctgtccagagaagaagaggaggagctgggccgcctgaccgtggaggacgaggagcggagaaggcgcgtggcaggaccacgcacaggcgatgtgaacctgtccggaggaggaccaaggggagcacctggaggcggcttcgtgcctagaatggagggagtgcctgagtcccccttcacccgcaccggagagggcctggacatcagaggcaatcagggattcccatgggtgaggcccagcccaccacagcagcgcctgccactgctggagtgtaccccccagggcacaaacctgtccacctctaatcccctgggcttctttcctgatcatcagctggacccagccttcagggccaactccgccaatccagattgggacttcaacccgaataaggatacttggccagatgcaaacaaggtcggaggacagaacctgagcacatccaaccctctgggcttctttcctgaccatcagctggatcccgcctttcgcgccaataccgccaaccctgattgggacttcaaccctaataaggatacttggcctgatgctaataaggtcgggtga

SEQ ID NO: 16 - DNA, 3198 bp, synthetic construct
  source 1..3198: mol_type = other DNA
atggccggcactaacctgtctacatcaaaccctctgggatttttccccgatcatcagctggaccccgcatttcgcgctaactctgctaaccctgactgggatttcaaccctaataaggacacatggccagatgccaacaaggtcggcggccagaacctgtccacctctaatcccctgggcttctttcctgaccaccagctggatcctgccttcagggccaacaccgccaatcccgactgggacttcaacccaaataaggatacctggcctgacgctaacaaggtcggcagccggtccgagtctaagaagaataggggaggaagggaggagatcctggagcagtgggtgggcgccagaaagaagctggaggagctggagcgggacctgagaaagatcaagaagaagatcaagaagctggaggaggagaacccctggctgggcaatatcaagggcatcctgggcaagaaggatcgggagggagagggagcaccacctgcaaagagggccagagccgaccagatggaggtggattccggccctaggaagcgcccattcagaggcgagtttacagacaaggagcggagagatcacaggcgccggaaggccctggagaacaagaggaagcagctgagctccggcggcaagagcctgtccaaggaggaggaggaggagctgcgcaagctgaccgaggaggacgagagaagggagaggagggtggcaggacctagggtgggaggcgtgaacccactggagggaggaacaagaggagcacccggaggaggcttcgtgccttctatgcagggcgtgcctgagagcccatttgccaggaccggagagggcctggacgtgagaggcaatcagggcttcccatgggacatcctgtttcccgccgatccacccttcagcccacagtcctgcaggccccagtctcgcagcgagtccaagaagaacagaggcggaagggaggaggtgctggagcagtgggtgaatggcaggaagaagctggaagaactggagagggagctgagaagggcccgcaagaagatcaagaagctggaagacgataatccttggctgggcaatgtgaaaggcatcctgggcaagaaggacaaggatggagagggagcacctccagcaaagagggcaagaacagaccagatggagatcgattccggaccaaggaagcgccctctgaggggaggcttcaccgaccgggagagacaggatcaccgccggagaaaggccctgaagaacaagaagaagcagctgagcgccggcggcaagtctctgagtaaagaagaagaggaggagctgaagcggctgacaagagaggacgaggagaggaagaaggaggagcacggaccatccaggctgggagtgaatccttctgagggaggaccaaggggcgcccctggcggaggcttcgtgcctagcatgcagggcatcccagagtccaggtttaccaggacaggcgaaggcctggacgtgcggggctctagaggctttccccaggacatcctgttccctagcgatccccctttttctcctcagagctgtagaccacagggaagcggagctactaacttcagcctgctgaagcaggctggagacgtggaggagaaccctggacctatgggcaccaacctgtccacatctaaccctctgggcttctttccagatcatcagctggacccagccttcagggccaacagcgccaatccagactgggacttcaaccccaataaggacacatggcctgacgcaaacaaggtcggaggacagaacctgagcacctccaatccactgggcttctttcccgaccaccagctggatccagccttccgcgccaacactgctaaccctgattgggacttcaaccctaataaggatacatggcctgatgccaataaggtcggctctcagagcgagacaaggaggggccggagaggaaccagggaggagacactggagaagtggatcaccgcccgcaagaaggccgaggagctggagaaggacctgaggaagacccgcaagacaatcaagaagctggaagaagagaacccatggctgggcaatatcgtgggcatcatcagaaagggcaaggacggcgagggagcaccaccagcaaagaggccccgcacagatcagatggaagtggattccggacctggcaagcggccacacaagtctggcttcaccgacaaggagagagaggaccataggcgccggaaggccctggaaaacaagaagaagcaattatctgccggcggcaagatcctgagtaaagaagaggaagaggagctgagaaggctgaccgacgaggatgaggagaggaagcgccgggtggccggcccacgcgtgggcgacgtgaatccctccaggggaggaccaagaggagcacctggaggcggcttcgtgccccagatggccggcgtgcccgagtcccctttttctcggaccggcgagggcctggatatcagaggcacacagggctttccatgggtgtccccctctcctccacagcagaggctgccactgctggagtgcacaccccagagccagagcgaatctaagaagaacagaaggggaggccgcgaggacatcctggaaaaatggatcaccacacgcagaaaagctgaagaactggaaaaggacctgcggaaggccagaaagaccatcaagaagctggaggatgaaaatccatggctgggaaacatcatcggcatcatccggaagggcaaggacggggaaggcgcaccacctgcaaagcggcctagaaccgatcagatggaaatcgatagcggcacaggcaagaggccacacaagtccggcttcaccgataaagagcgcgaggatcacagaaggcgcaaggccctggagaacaagaagaagcaattaagcagcggcggcaagaatctgtccagagaagaggaggaagagctgggccgcctgacagtggaggacgaggagcggagaaggcgcgtggcaggacccagaaccggcgatgtgaacctgtccggaggaggacctaggggagcaccaggaggcggcttcgtgcctagaatggagggcgtgccagagtctccctttacccggacaggcgagggcctggacatcagaggcaatcagggctttccctgggtccgcccctccccccctcagcagagactgccactgctggaatgcacaccacagtga

SEQ ID NO: 17 - DNA, 3762 bp, synthetic construct
  source 1..3762: mol_type = other DNA
atggccggcaccaatctgtctacctcaaatcccctgggcttcttccccgatcatcagctggaccctgccttccgagcaaattccgctaatcctgattgggatttcaacccaaataaggacacatggccagatgccaacaaggtcggcggccagaacctgtccacctctaatcctctgggcttctttccagaccaccagctggatcccgccttcagggccaacacagccaatcccgactgggacttcaaccctaataaggacacctggcctgacgccaacaaggtcggcagcaggtccgagtctaagaagaataggggaggaagggaggagatcctggagcagtgggtgggagcacgcaagaagctggaggagctggagcgggacctgagaaagatcaagaagaagatcaagaagctggaggaggagaacccctggctgggcaatatcaagggcatcctgggcaagaaggatcgggagggagagggagcaccacctgcaaagagggccagagccgaccagatggaggtggattccggaccaaggaagcgccctttcagaggagagtttacagacaaggagcggagagatcacaggcgccggaaggccctggagaacaagcggaagcagctgagctccggcggcaagagcctgtccaaggaggaggaggaggagctgagaaagctgaccgaggaggacgagagaagggagaggagggtggccggccccagggtgggcggcgtgaaccctctggagggaggaacaaggggagcaccaggaggaggcttcgtgccttccatgcagggcgtgcccgagtctccttttgccaggaccggagagggcctggacgtgcgcggcaatcagggcttcccatgggacatcctgtttcccgccgatccacccttctctccccagagctgcaggcctcagtctcgcagcgagtccaagaagaacagaggcggaagggaggaggtgctggagcagtgggtgaatggcaggaagaagctggaagaactggagagggagctgagaagggcccgcaagaagatcaagaagctggaagacgataatccttggctgggcaatgtgaaaggcatcctgggcaagaaggacaaggatggagagggagcacctccagcaaagagggcaagaacagaccagatggagatcgattctggaccaaggaagcgccccctgaggggaggcttcaccgaccgggagagacaggatcaccgccggagaaaggccctgaagaacaagaagaagcagctgagcgccggcggcaagtctctgagtaaagaagaagaggaggagctgaagcggctgaccagagaggacgaggagcggaagaaggaggagcacggcccaagcagactgggagtgaatccatccgagggaggacctagaggcgcccctggcggcggcttcgtgccttctatgcagggcatcccagagagcaggtttaccaggacaggcgaaggcctggacgtgcggggctccagaggctttccccaggacatcctgttcccttctgatccccctttttccccacagtcttgtaggccccagggcaccaacctgtccacatctaacccactgggcttctttcctgatcaccagctggatccagccttccgcgccaactccgccaatccagactgggacttcaaccccaataaggacacatggcctgatgctaacaaggtcggaggccagaacctgagcacctccaatcccctgggcttctttcctgaccaccagctggatcctgccttccgcgccaacacagctaaccctgattgggacttcaacccaaataaggatacctggcctgatgcaaacaaggtcggaggaagcggagctactaacttcagcctgctgaagcaggctggagacgtggaggagaaccctggacctatgggcaccaacctgtctacaagcaatccactgggcttctttcccgaccatcagctggacccagccttcagggccaacagcgccaaccctgactgggacttcaacccaaataaggacacgtggcctgatgccaacaaggtcggaggacaaaacctgtccacctctaaccccctgggcttctttcccgatcatcaattagacccagccttccgcgctaacactgctaaccctgactgggacttcaacccgaataaggatacttggcctgatgccaataaggtcggcagccagtccgagacaaggaggggccggagaggaaccagggaggagacactggagaagtggatcaccgccagaaagaaggccgaggagctggagaaggacctgaggaagacccgcaagacaatcaagaagctggaagaagagaacccttggctgggcaatatcgtgggcatcatcagaaagggcaaggacggcgagggagcaccaccagccaagaggccacgcacagatcagatggaagtggatagcggaccaggcaagaggcctcacaagtccggcttcaccgacaaggagagggaggaccataggcgccggaaggccctggaaaacaagaagaagcaattatccgccggcggcaagatcctgtctaaagaagaggaagaagagctgagaaggctgaccgacgaggatgaggagaggaagaggagggtggcaggacctagagtgggcgacgtgaatccatccaggggaggaccaagaggagcaccaggaggcggcttcgtgccacagatggcaggagtgccagagagccccttttccaggacaggagagggcctggatatcaggggaacccagggctttccttgggtgtctccaagccctccacagcagcggctgccactgctggagtgcacaccccagtcccagtctgagagcaagaagaacagaaggggcggcagagaggacatcctggaaaaatggatcaccacacgcagaaaagctgaagaactggaaaaggacctgcggaaggccagaaagaccatcaagaagctggaggatgaaaatccatggctgggaaatatcatcggcatcatccggaagggcaaggacggggaaggcgcaccacctgcaaagcggcccaggaccgatcagatggaaatcgattctggaaccggcaagcggcctcacaagagtggcttcaccgataaggagagagaggatcacagaaggcgcaaggccctggagaacaagaagaagcaattaagcagcggcggcaagaatctgtccagagaagaggaagaggagctgggcagactgacagtggaggacgaggagcggagaaggcgcgtggcaggaccaagaaccggcgatgtgaacctgtccggaggaggaccaaggggagcacctgggggaggcttcgtgccaaggatggagggagtgcctgagtcccccttcaccagaaccggcgaaggcctggacatcaggggcaatcagggattcccatgggtgcggccctccccaccccagcagagactgcctctgctggagtgtaccccacagggcactaacctgtccacctctaacccgttaggcttctttcctgaccatcaattagatcccgccttccgggccaacagcgccaatcctgattgggacttcaacccgaataaggacacctggcccgacgcaaacaaggtcggagggcaaaacctgagcacctccaaccctttaggcttctttccagatcatcagctggatccagcctttagagccaataccgccaaccctgactgggatttcaaccctaacaaagatacctggcccgacgctaacaaagtgggatga

SEQ ID NO: 18 - DNA, 3900 bp, synthetic construct
  source 1..3900: mol_type = other DNA
atggccagtcggagcgaatcaaagaaaaataggggagggcgggaagaaatcctggagcagtgggtcggagcacgaaagaaactggaagaactggagagggacctgcgcaagatcaagaagaagatcaagaagctggaggaggagaacccctggctgggcaatatcaagggcatcctgggcaagaaggatcgggagggagagggagcaccacctgcaaagagggccagagccgaccagatggaggtggatagcggccctaggaagcgcccattcagaggcgagtttacagacaaggagcggagagatcacaggcgccggaaggccctggagaacaagcggaagcagctgagctccggcggcaagtccctgtctaaggaggaggaggaggagctgagaaagctgaccgaggaggacgagagaagggagaggagggtggcaggacctagggtgggaggcgtgaacccactggagggaggaacaaggggagcacctggaggaggattcgtgccatccatgcagggagtgcctgagtctccatttgccaggaccggagagggcctggatgtgcgcggaaatcagggcttcccctgggacatcctgtttcctgccgatccacccttctccccacagtcttgcaggccacagggaaccaacctgagcacatccaatcctctgggcttctttccagaccaccagctggatcctgccttcagagccaactccgccaatccagactgggacttcaaccccaataaggacacatggcctgatgccaacaaggtcggcggccagaacctgtctaccagcaatcccctgggcttctttcctgaccaccagctggatccagccttccgggccaacactgctaaccctgattgggacttcaaccctaataaggatacctggccagacgccaacaaggtcggcggaagcggagctactaacttcagcctgctgaagcaggctggagacgtggaggagaaccctggacctatgtccaggtctgagagcaagaagaataggggaggaagagaggaggtgctggagcagtgggtgaacggccgcaagaagctggaggagctggagagggagctgagaagggcccgcaagaagatcaagaagctggaagacgataatccttggctgggcaatgtgaaaggcatcctgggcaagaaggacaaggatggagagggagcacctccagcaaagagggcaagaacagaccagatggagatcgattctggaccaaggaagcgccctctgaggggaggcttcaccgaccgggagagacaggatcaccgccggagaaaggccctgaagaacaagaagaagcagctgtccgccggcggcaagtccctgagcaaagaagaggaagaggagctgaagaggctgacccgcgaggacgaggagcggaagaaggaggagcacggaccaagcagactgggagtgaatccttccgagggaggaccaagaggagcacccggaggaggcttcgtgccatctatgcagggcatccccgagagccggtttaccagaacaggagagggcctggacgtgaggggctcccgcggctttcctcaggacatcctgttcccatctgatcccccttttagcccacagtcctgtaggccccagggcactaacctgagcacatccaacccactgggcttctttcctgatcatcagctggacccagccttccgcgccaacagcgccaaccctgactgggacttcaacccaaataaggacacatggccagatgctaacaaggtcggaggacaaaacctgtctaccagcaaccctctgggcttctttcccgatcatcagctggaccccgccttcagggccaacacagccaatcccgactgggacttcaacccgaataaggacacctggccagatgcaaacaaggtcggaggaagcggagctactaacttcagcctgctgaagcaggctggagacgtggaggagaaccctggacctatgagccagtctgagacaaggaggggccggagaggaaccagggaggagacactggagaagtggatcaccgccagaaagaaggccgaggagctggagaaggacctgcggaagaccagaaagacaatcaagaagctggaagaagagaacccatggctgggcaatatcgtgggcatcatccgcaagggcaaggacggcgagggagcaccaccagcaaagaggccccgcacagatcagatggaagtggatagcggccctggcaagaggccacacaagtccggcttcaccgacaaggagagggaggaccataggcgccggaaggccctggaaaacaagaagaagcaattatccgccggcggcaagatcctgtccaaagaggaagaagaggagctgagaaggctgaccgacgaggatgaggagaggaaaagaagggtggcaggaccaagagtgggcgacgtgaatcccagcagaggcggaccaagaggagcacctggaggcggcttcgtgccccagatggccggcgtgcccgagtctccttttagcagaactggagagggcctggatatcaggggaacacagggctttccatgggtgagcccatcccctccacagcagaggctgccactgctggagtgcacccctcagggaaccaacctgtctaccagcaacccgctgggcttctttcccgaccatcagctggaccctgccttccgcgccaactccgccaaccctgattgggacttcaacccgaataaggatacctggcccgacgctaacaaggtcggaggccagaacctgtccacctctaaccccttaggcttctttcccgatcaccagctggatcccgccttcagagccaacactgctaaccccgattgggacttcaacccgaataaggacacgtggccagacgctaacaaggtcgggggaagcggagctactaacttcagcctgctgaagcaggctggagacgtggaggagaaccctggacctatgtcgcagtccgagtctaagaagaatagaaggggcggccgggaggatatcctggaaaaatggatcaccacacgcagaaaagctgaagaactggaaaaggacctgaggaaggcccgcaagaccatcaagaagctggaggatgaaaatccatggctgggaaacatcatcggcatcatcagaaagggcaaggacggggaaggcgccccacctgcaaagcggcctagaaccgatcagatggaaatcgattctggcacaggcaagcggccacacaagagtggcttcaccgataaggagagagaggatcacagaaggcgcaaggccctggagaacaagaagaagcaattaagcagcggcggcaagaatctgtccagagaagaagaggaggagctgggcagactgacagtggaggacgaggagcggagaaggcgcgtggcaggaccaaggaccggcgatgtgaacctgagcggaggaggacctaggggagcaccaggaggcggcttcgtgcctaggatggagggagtgccagagtccccctttaccaggactggcgagggcctggacatcaggggaaatcagggattcccatgggtgcggcctagcccaccacagcagagactgccactgctggagtgtacaccccagggcacaaacctgagcacatccaatccgctgggcttctttccagatcatcaattagatccagccttcagggccaactccgccaatccggattgggacttcaacccgaataaggacacttggcccgacgcaaacaaggtcggagggcaaaacctgtctaccagcaatccacttggcttctttcctgaccatcagctggatcccgcctttcgcgccaataccgccaatcctgactgggacttcaatcctaacaaagacacctggcccgacgcaaacaaagtgggatga

SEQ ID NO: 19 - DNA, 2634 bp, synthetic construct
  source 1..2634: mol_type = other DNA
atggcctcacggtcagagtcaaagaaaaataggggggggcgggaagaaatcctggaacagtgggtcggagcacggaaaaaactggaagagctggagagggacctgcgcaagatcaagaagaagatcaagaagctggaggaggagaacccctggctgggcaatatcaagggcatcctgggcaagaaggatcgggagggagagggagcaccacctgcaaagagggccagagccgaccagatggaggtggatagcggccctaggaagcgcccattcagaggcgagtttaccgacaaggagcggagagatcacaggcgccggaaggccctggagaacaagcggaagcagctgagctccggcggcaagtccctgtctaaggaggaggaggaggagctgagaaagctgacagaggaggacgagagaagggagcgccgggtggccggcccaagggtgggcggcgtgaaccccctggagggaggaaccaggggagcacctggaggaggcttcgtgccatctatgcagggcgtgcctgagagcccatttgccaggacaggagagggcctggatgtgcgcggcaatcagggcttcccctgggacatcctgtttcctgccgatccacccttcagcccacagtcctgcaggcctcagagcagatccgagtctaagaagaacaggggaggaagagaggaggtgctggagcagtgggtgaatggccggaagaagctggaggagctggagcgggagctgagaagggccagaaagaagatcaagaagctggaagacgataatccttggctgggcaatgtgaaaggcatcctgggcaagaaggacaaggatggagagggagcacctccagcaaagagggcaagaaccgaccagatggagatcgatagcggaccaaggaagcgccctctgagaggaggcttcacagaccgggagagacaggatcaccgccggagaaaggccctgaagaacaagaagaagcagctgtccgccggaggcaagagcctgtccaaagaagaggaagaggagctgaagaggctgacccgcgaggacgaggagcggaagaaggaggagcacggcccttccagactgggcgtgaatccatctgagggaggaccaaggggagcaccaggcggcggcttcgtgccaagcatgcagggcatccccgagtcccggtttaccagaacaggagagggcctggacgtgaggggctctcgcggctttcctcaggacatcctgttcccaagcgatccccctttttctccacagagctgtcgcccccagggaagcggagctactaacttcagcctgctgaagcaggctggagacgtggaggagaaccctggacctatgtctcagagcgagacaaggaggggccggagaggaaccagggaggagacactggagaagtggatcacagccagaaagaaggccgaggagctggagaaggacctgcggaagaccagaaagacaatcaagaagctggaagaagaaaatccatggctgggaaatatcgtgggcatcatcaggaagggcaaggacggcgagggagcaccaccagccaagaggcctcgcactgatcagatggaggtggattccggccctggcaagaggccacacaagtctggcttcacagacaaggagagggaggaccataggcgccggaaggccctggaaaacaagaagaagcaattatctgccggcggcaagatcctgagcaaagaggaagaggaggagctgagaaggctgaccgacgaggatgaggagaggaagaggagggtggcaggaccaagagtgggcgacgtgaatcctagcagaggcggaccaagaggcgccccaggcgggggcttcgtgccacagatggcaggagtgccagagtcccctttttctaggaccggagagggcctggatatcaggggaacacagggctttccatgggtgtccccatctcctccacagcagaggctgccactgctggagtgcacccctcagagccagtccgagtctaagaagaatagaaggggcggccgcgaggacatcctggagaagtggatcaccacacgcagaaaagctgaagaactggaaaaggacctgaggaaggcccgcaaaacaatcaagaagctggaggatgagaacccttggctgggcaatatcatcggaattatcaggaagggcaaggatggcgaaggcgccccacctgcaaagcggccaaggactgatcagatggaaatcgatagcggaacaggcaagcggccccacaagtccggcttcaccgacaaggagagagaggatcacagaaggcgcaaggccctggagaacaagaagaagcaattaagcagcggcggcaagaatctgtccagagaagaagaggaggagctgggcagactgaccgtggaggacgaggagcggagaaggcgcgtggcaggacctcgcacaggcgatgtgaacctgtccggaggaggacctaggggagcaccaggaggcggcttcgtgccacgcatggagggcgtgccagagtctccctttacccgcaccggagagggcctggacatcaggggcaatcagggctttccctgggtccgcccctccccccctcagcagagactgcccctgctggaatgcacaccacagtga

SEQ ID NO: 20 - DNA, 2772 bp, synthetic construct
  source 1..2772: mol_type = other DNA
atggcctcacggtcagagtcaaagaagaacagaggcggaagagaagaaatcctggagcagtgggtcggagcacggaaaaagctggaagaactggagagggacctgcgcaagatcaagaagaagatcaagaagctggaggaggagaacccctggctgggcaatatcaagggcatcctgggcaagaaggatagggagggagagggagcaccacctgcaaagagggccagagccgaccagatggaggtggatagcggaccaaggaagcgccccttccgcggagagtttaccgacaaggagcggagagatcacaggcgccggaaggccctggagaacaagaggaagcagctgagctccggcggcaagtccctgtctaaggaggaggaggaggagctgcgcaagctgacagaggaggacgagagaagggagaggagggtggcaggaccaagggtgggaggagtgaatcctctggagggaggaaccagaggagcaccaggaggaggcttcgtgccaagcatgcagggagtgccagagtccccctttgccaggacaggagagggcctggacgtgagaggcaaccagggcttcccttgggacatcctgtttccagccgatccacccttcagccctcagtcctgcaggccacagggaagcggagctactaacttcagcctgctgaagcaggctggagacgtggaggagaaccctggacctatgagccggtccgagtctaagaagaataggggaggaagagaggaggtgctggagcagtgggtgaacggcagaaagaagctggaggagctggagagggagctgagaagggcccgcaagaagatcaagaagctggaagacgataatccttggctgggcaatgtgaaaggcatcctgggcaagaaggacaaggatggagagggagcacctccagcaaagagggcaagaaccgaccagatggagatcgatagcggacctaggaagcgcccactgaggggaggctttacagaccgggagagacaggatcaccgccggagaaaggccctgaagaacaagaagaagcagctgtccgccggaggcaagagcctgtccaaagaagaggaagaggagctgaagaggctgacccgcgaggacgaggagaggaagaaggaggagcacggaccatctaggctgggagtgaatcccagcgagggaggaccaaggggagcacctggaggaggcttcgtgccctccatgcagggcatccctgagtctcggtttaccagaaccggcgagggcctggacgtgaggggcagccgcggctttccacaggacatcctgttcccctccgatccccctttttctccccagagctgtcgccctcaaggaagcggagctactaacttcagcctgctgaagcaggctggagacgtggaggagaaccctggacctatgtctcagagcgagacaaggaggggccggagaggaaccagggaggagacactggagaagtggatcacagcccgcaagaaggccgaggagctggagaaggacctgcggaagaccagaaagacaatcaagaagctggaagaagagaacccttggctgggcaatatcgtgggcatcatcaggaagggcaaggacggcgagggagcaccaccagccaagaggccacgcactgatcagatggaggtggattctggaccaggcaagcggccccacaagagcggcttcacagacaaggagagagaggaccataggcgccggaaggccctggaaaacaagaagaagcaattaagcgccggcggcaagatcctgtccaaagaggaagaggaggagctgagaaggctgaccgacgaggatgaggagaggaaaagaagggtggcaggacctagggtgggcgacgtgaatccaagcaggggaggacctagaggagcaccaggaggcggcttcgtgccacagatggcaggagtgcctgagtccccattttctcggaccggcgagggcctggatatcagaggcacacagggcttcccctgggtgtccccttctcctccacagcagcggctgcctctgctggagtgcacccctcagggaagcggagctactaacttcagcctgctgaagcaggctggagacgtggaggagaaccctggacctatgtcgcagagcgaatctaagaagaatagaaggggcggcagagaggatatcctggagaagtggatcaccacacgcagaaaagctgaagaactggaaaaggacctgaggaaggcccgcaagaccatcaagaagctggaggatgaaaatccatggctgggaaatatcatcggcatcatccggaagggcaaggacggggaaggcgccccacctgcaaagcggcccaggactgatcagatggaaatcgattccggcacaggcaagaggcctcacaagtctggcttcacagataaagagcgcgaggatcacagaaggcgcaaggccctggagaacaagaagaagcaattatctagcggcggcaagaatctgtccagagaagaagaggaggagctgggccgcctgaccgtggaggacgaggagcggagaaggcgcgtggcaggaccaagaacaggcgatgtgaacctgtctggaggcggcccaaggggcgcccccggcggaggcttcgtgccaagaatggaaggcgtgccagagtccccttttacccggacaggggaaggcctggacattagaggcaatcagggctttccctgggtgcgaccaagcccccctcagcagcgactgcctctgctggagtgtacccctcagtga

SEQ ID NO: 21 - DNA, 1569 bp, synthetic construct
  source 1..1569: mol_type = other DNA
atggcctcacggtctgagtcaaagaagaatcgggggggaagagaagaaatcctggaacagtgggtcggcgcacggaaaaaactggaagaactggagcgggacctgagaaagatcaagaagaagatcaagaagctggaggaagagaacccctggctgggcaatatcaagggcatcctgggcaagaaggatcgggagggcgagggagcaccacctgcaaagagggcaagggcagaccagatggaggtggattccggacctaggaagcggcccttccggggagagtttaccgacaaggagcggagagatcacaggcgccggaaggccctggagaacaagcggaagcagctgagctccggcggcaagtctctgagcaaggaggaggaggaggagctgagaaagctgacagaggaggacgagagaagggagcgccgggtggccggcccaagggtgggcggcgtgaaccccctggagggaggaaccaggggagcaccaggaggaggcttcgtgccttctatgcagggcgtgccagagagcccctttgccaggacaggagagggcctggatgtgcgcggcaatcagggcttcccatgggacatcctgtttcccgccgatccacccttctcccctcagtcttgcaggccacagtcccgctctgagagcaagaagaacaggggaggaagggaggaggtgctggagcagtgggtgaatggcaggaagaagctggaggagctggagcgggagctgagaagggccagaaagaagatcaagaagctggaagacgataatccttggctgggcaatgtgaaaggcatcctgggcaagaaggacaaggatggagagggagcacctccagcaaagagggcacgcaccgaccagatggagatcgattccggaccaaggaagcggcccctgaggggaggcttcacagacagggagcgccaggatcaccgccggagaaaggccctgaagaacaagaagaagcagctgtctgccggcggcaagtccctgtctaaagaagaggaggaggagctgaagcggctgaccagagaggacgaggagcggaagaaggaggagcacggcccttccagactgggcgtgaatccatctgagggaggaccaagaggcgcccctggcggaggcttcgtgcctagcatgcagggcatcccagagtccaggtttaccagaaccggagagggcctggacgtgcggggctctagaggctttccccaggacatcctgttccctagcgatcccccttttagcccccagtcctgtaggcctcagggcaccaacctgagcacatccaatccactgggcttctttccagaccaccagctggatccagccttccgcgccaacagcgccaatccagactgggacttcaaccccaataaggacacctggcctgatgccaacaaggtcggcggccagaacctgtctacaagcaatcctctgggcttctttcctgatcaccagctggatcctgcctttcgggccaatacagccaaccctgactgggacttcaatcctaacaaagacacttggcccgatgctaataaggtcggctga

SEQ ID NO: 22 - DNA, 1569 bp, synthetic construct
  source 1..1569: mol_type = other DNA
atggccagtcagagcgagacccgcagaggacggagaggaacacgagaagagacactggagaaatggattacagcacggaagaaggcagaagagctggagaaggacctgaggaagacccgcaagacaatcaagaagctggaggaggagaacccctggctgggcaatatcgtgggcatcatcaggaagggcaaggatggagagggagcaccacctgccaagaggcctcgcacagaccagatggaggtggatagcggaccaggcaagcggcctcacaagtccggcttcaccgacaaggagagagaggatcaccggagaaggaaggccctggagaacaagaagaagcagctgtccgccggcggcaagatcctgtctaaggaggaggaggaggagctgcgccggctgacagacgaggatgaggagaggaagagaagggtggcaggaccaagggtgggcgacgtgaatccttctaggggaggaccaaggggagcaccaggaggaggcttcgtgcctcagatggccggcgtgccagagtctccctttagccggacaggcgagggcctggatatcagaggcacccagggctttccttgggtgtctccaagcccaccacagcagcggctgccactgctggagtgcacaccccagtcccagtctgagagcaagaagaacaggaggggaggaagagaggacatcctggagaagtggatcaccacaagaaggaaggccgaggagctggagaaggacctgcggaaggccagaaagaccatcaagaagctggaggatgaaaatccttggctgggaaatatcatcggaattattagaaaaggcaaggacggagagggagcacctccagcaaagcggccaagaacagaccagatggagatcgattctggaaccggcaagaggccccacaagagtggcttcaccgataaggagcgcgaggatcaccgccggagaaaggccctggaaaacaagaagaagcaattaagctccggcggcaagaatctgagcagagaagaagaggaggagctgggccgcctgacagtggaggacgaggagaggcgccggagagtggcaggacctagaaccggcgatgtgaacctgtccggaggcggcccaaggggagcacctggaggcggcttcgtgccacgcatggagggcgtgcctgagtctcccttcaccaggacaggagagggcctggacatcagaggcaatcagggattcccatgggtgcggcccagcccacctcagcagagactgcctctgctggagtgtaccccacagggcacaaacctgtccacctctaatcctctgggcttctttccagaccaccagctggatccagccttcagggccaactccgccaaccctgactgggacttcaaccctaataaggacacatggccagatgccaacaaggtcggcggccagaacctgagcacctccaatcccctgggcttctttcctgaccaccagctggatcccgcctttcgcgccaataccgccaatcccgactgggacttcaatccaaataaggacacctggcccgatgctaacaaagtgggatga

SEQ ID NO: 23 - DNA, 1287 bp, synthetic construct
  source 1..1287: mol_type = other DNA
atggccagtcggagcgaatcaaagaaaaatagagggggaagagaagaaatcctggagcagtgggtcggggcacggaaaaaactggaagaactggagcgggacctgagaaagatcaagaagaagatcaagaagctggaggaagagaacccctggctgggcaatatcaagggcatcctgggcaagaaggatagggagggcgagggagcaccacctgcaaagagggcaagggcagaccagatggaggtggattccggaccaaggaagcggcccttccggggagagtttaccgacaaggagcggagagatcacaggcgccggaaggccctggagaacaagcggaagcagctgagctccggcggcaagtctctgagcaaggaggaggaggaggagctgagaaagctgacagaggaggacgagagaagggagaggagggtggcaggacctagggtgggaggcgtgaacccactggagggaggaaccaggggagcacctggaggaggctttgtgccatctatgcagggagtgccagagagccctttcgccaggacaggagagggcctggatgtgcgcggcaatcagggcttcccctgggacatcctgtttcctgccgatccacccttcagcccacagtcctgcaggccacagtcccgctctgagagcaagaagaacaggggaggaagggaggaggtgctggagcagtgggtgaatggccggaagaagctggaggagctggagcgggagctgagaagggccagaaagaagatcaagaagctggaagacgataatccttggctgggcaatgtgaaaggcatcctgggcaagaaggacaaggatggagagggagcacctccagcaaagagggcacgcaccgaccagatggagatcgattctggacctaggaagcggcccctgagaggaggctttacagacagggagcgccaggatcaccgccggagaaaggccctgaagaacaagaagaagcagctgagcgccggcggcaagtccctgtctaaagaagaggaggaggagctgaagcggctgaccagagaggacgaggagcggaagaaggaggagcacggaccatccagactgggagtgaatccttctgagggaggaccaagaggcgccccaggcggcggctttgtgccaagcatgcagggcatccccgagtccaggttcaccagaaccggcgaaggcctggatgtgcggggcagcagaggcttcccccaggatattctgtttccctccgacccccccttcagtccccagtcttgccgacctcagtga

SEQ ID NO: 24 - DNA, 1287 bp, synthetic construct
  source 1..1287: mol_type = other DNA
atggcctcacagagcgaaacacggcgggggcggaggggaactagagaggaaacactggaaaaatggattacagcacggaaaaaggcagaggaactggagaaggacctgaggaagacccgcaagacaatcaagaagctggaggaggagaacccatggctgggcaatatcgtgggcatcatccggaagggcaaggatggagagggagcaccacctgcaaagaggccccgcaccgaccagatggaggtggattctggccctggcaagaggccacacaagagcggcttcacagacaaggagcgcgaggatcaccggagaaggaaggccctggagaacaagaagaagcagctgagcgccggcggcaagatcctgtccaaggaggaggaggaggagctgcgccggctgaccgacgaggatgaggagcggaagagaagggtggcaggaccaagagtgggcgacgtgaatccctctaggggaggaccaaggggagcacctggaggaggcttcgtgcctcagatggcaggagtgccagagtcccctttttctaggaccggagagggcctggatatcaggggaacacagggctttccatgggtgtctccaagcccaccacagcagaggctgccactgctggagtgcacccctcagtcccagtctgagagcaagaagaacaggaggggaggaagggaggacatcctggagaagtggatcaccacaagaaggaaggccgaggagctggagaaggacctgcggaaggccagaaaaacaatcaagaagctggaagatgagaacccctggctgggcaatatcatcggcatcatcagaaaaggcaaggacggcgagggagcacctccagcaaagcggcctagaaccgaccagatggagatcgattccggcacaggcaagcggccacacaagtctggcttcaccgacaaggagagagaggatcaccgccggagaaaggccctggaaaacaagaagaagcaattaagctccggcggcaagaatctgagcagagaagaagaggaggagctgggcagactgaccgtggaggacgaggagaggcgccggagagtggcaggacccagaacaggcgatgtgaacctgagcggaggaggacctaggggagcaccaggaggcggcttcgtgcctagaatggagggcgtgccagagtccccctttaccaggacaggagagggcctggacatcaggggcaatcagggctttccctgggtccgcccttcaccaccacagcagagactgcccctgctggaatgcacaccacagtga

SEQ ID NO: 25 - protein, 1065 aa, synthetic construct
MASRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSCRPQSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSCRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGSGATNFSLLKQAGDVEENPGPMSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLECTPQSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLECTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 26 - protein, 1065 aa, synthetic construct
MAGTNLSTSNPLGFFPDHQLDPAFRANSANPDWDFNPNKDTWPDANKVGGQNLSTSNPLGFFPDHQLDPAFRANTANPDWDFNPNKDTWPDANKVGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSCRPQSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSCRPQGSGATNFSLLKQAGDVEENPGPMGTNLSTSNPLGFFPDHQLDPAFRANSANPDWDFNPNKDTWPDANKVGGQNLSTSNPLGFFPDHQLDPAFRANTANPDWDFNPNKDTWPDANKVGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLECTPQSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLECTPQ

SEQ ID NO: 27 - protein, 1253 aa, synthetic construct
MAGTNLSTSNPLGFFPDHQLDPAFRANSANPDWDFNPNKDTWPDANKVGGQNLSTSNPLGFFPDHQLDPAFRANTANPDWDFNPNKDTWPDANKVGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSCRPQSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSCRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGSGATNFSLLKQAGDVEENPGPMGTNLSTSNPLGFFPDHQLDPAFRANSANPDWDFNPNKDTWPDANKVGGQNLSTSNPLGFFPDHQLDPAFRANTANPDWDFNPNKDTWPDANKVGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLECTPQSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLECTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 28 - protein, 1299 aa, synthetic construct
MASRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSCRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGSGATNFSLLKQAGDVEENPGPMSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSCRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGSGATNFSLLKQAGDVEENPGPMSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLECTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGSGATNFSLLKQAGDVEENPGPMSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLECTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 29 - protein, 877 aa, synthetic construct
MASRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSCRPQSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSCRPQGSGATNFSLLKQAGDVEENPGPMSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLECTPQSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLECTPQ

SEQ ID NO: 30 - protein, 923 aa, synthetic construct
MASRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSCRPQGSGATNFSLLKQAGDVEENPGPMSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSCRPQGSGATNFSLLKQAGDVEENPGPMSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLECTPQGSGATNFSLLKQAGDVEENPGPMSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLECTPQ

SEQ ID NO: 31 - protein, 522 aa, synthetic construct
MASRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSCRPQSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSCRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 32 - protein, 522 aa, synthetic construct
MASQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLECTPQSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLECTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 33 - protein, 428 aa, synthetic construct
MASRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSCRPQSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSCRPQ

SEQ ID NO: 34 - protein, 428 aa, synthetic construct
MASQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLECTPQSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLECTPQ

SEQ ID NO: 35 - DNA, 3434 bp, synthetic construct
  source 1..3434: mol_type = other DNA
aagcttgcaccatggccggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggcagccgcagcgaaagcaaaaaaaaccgcggcggccgcgaagaaattctggaacagtgggtgggcgcgcgcaaaaaactggaagaactggaacgcgatctgcgcaaaattaaaaaaaaaattaaaaaactggaagaagaaaacccgtggctgggcaacattaaaggcattctgggcaaaaaagatcgcgaaggcgaaggcgcgccgccggcgaaacgcgcgcgcgcggatcagatggaagtggatagcggcccgcgcaaacgcccgtttcgcggcgaatttaccgataaagaacgccgcgatcatcgccgccgcaaagcgctggaaaacaaacgcaaacagctgagcagcggcggcaaaagcctgagcaaagaagaagaagaagaactgcgcaaactgaccgaagaagatgaacgccgcgaacgccgcgtggcgggcccgcgcgtgggcggcgtgaacccgctggaaggcggcacccgcggcgcgccgggcggcggctttgtgccgagcatgcagggcgtgccggaaagcccgtttgcgcgcaccggcgaaggcctggatgtgcgcggcaaccagggctttccgtgggatattctgtttccggcggatccgccgtttagcccgcagagctgccgcccgcagagccgcagcgaaagcaaaaaaaaccgcggcggccgcgaagaagtgctggaacagtgggtgaacggccgcaaaaaactggaagaactggaacgcgaactgcgccgcgcgcgcaaaaaaattaaaaaactggaagatgataacccgtggctgggcaacgtgaaaggcattctgggcaaaaaagataaagatggcgaaggcgcgccgccggcgaaacgcgcgcgcaccgatcagatggaaattgatagcggcccgcgcaaacgcccgctgcgcggcggctttaccgatcgcgaacgccaggatcatcgccgccgcaaagcgctgaaaaacaaaaaaaaacagctgagcgcgggcggcaaaagcctgagcaaagaagaagaagaagaactgaaacgcctgacccgcgaagatgaagaacgcaaaaaagaagaacatggcccgagccgcctgggcgtgaacccgagcgaaggcggcccgcgcggcgcgccgggcggcggctttgtgccgagcatgcagggcattccggaaagccgctttacccgcaccggcgaaggcctggatgtgcgcggcagccgcggctttccgcaggatattctgtttccgagcgatccgccgtttagcccgcagagctgccgcccgcagggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggcagccagagcgaaacccgccgcggccgccgcggcacccgcgaagaaaccctggaaaaatggattaccgcgcgcaaaaaagcggaagaactggaaaaagatctgcgcaaaacccgcaaaaccattaaaaaactggaagaagaaaacccgtggctgggcaacattgtgggcattattcgcaaaggcaaagatggcgaaggcgcgccgccggcgaaacgcccgcgcaccgatcagatggaagtggatagcggcccgggcaaacgcccgcataaaagcggctttaccgataaagaacgcgaagatcatcgccgccgcaaagcgctggaaaacaaaaaaaaacagctgagcgcgggcggcaaaattctgagcaaagaagaagaagaagaactgcgccgcctgaccgatgaagatgaagaacgcaaacgccgcgtggcgggcccgcgcgtgggcgatgtgaacccgagccgcggcggcccgcgcggcgcgccgggcggcggctttgtgccgcagatggcgggcgtgccggaaagcccgtttagccgcaccggcgaaggcctggatattcgcggcacccagggctttccgtgggtgagcccgagcccgccgcagcagcgcctgccgctgctggaatgcaccccgcagagccagagcgaaagcaaaaaaaaccgccgcggcggccgcgaagatattctggaaaaatggattaccacccgccgcaaagcggaagaactggaaaaagatctgcgcaaagcgcgcaaaaccattaaaaaactggaagatgaaaacccgtggctgggcaacattattggcattattcgcaaaggcaaagatggcgaaggcgcgccgccggcgaaacgcccgcgcaccgatcagatggaaattgatagcggcaccggcaaacgcccgcataaaagcggctttaccgataaagaacgcgaagatcatcgccgccgcaaagcgctggaaaacaaaaaaaaacagctgagcagcggcggcaaaaacctgagccgcgaagaagaagaagaactgggccgcctgaccgtggaagatgaagaacgccgccgccgcgtggcgggcccgcgcaccggcgatgtgaacctgagcggcggcggcccgcgcggcgcgccgggcggcggctttgtgccgcgcatggaaggcgtgccggaaagcccgtttacccgcaccggcgaaggcctggatattcgcggcaaccagggctttccgtgggtgcgcccgagcccgccgcagcagcgcctgccgctgctggaatgcaccccgcagggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggctgatgagaattccgt

SEQ ID NO: 36 - DNA, 3431 bp, synthetic construct
  source 1..3431: mol_type = other DNA
aagcttgcaccatggcaggcaccaatctgtcaacctctaaccctctgggctttttccccgaccatcagctggaccccgcattccgagcaaactccgctaaccctgactgggatttcaacccaaataaggacacctggcccgatgccaataaggtcggcggccagaacctgtccacatctaatccactgggcttctttcccgaccaccagctggatcctgccttccgggccaacacagccaatcctgactgggacttcaaccctaataaggatacctggcccgatgccaacaaggtcggcagcaggtccgagtctaagaagaataggggaggaagggaggagatcctggagcagtgggtgggagcacgcaagaagctggaggagctggagcgggacctgagaaagatcaagaagaagatcaagaagctggaggaggagaacccctggctgggcaatatcaagggcatcctgggcaagaaggatcgggagggagagggagcaccacctgcaaagagggccagagccgaccagatggaggtggattccggccctaggaagcgcccattcagaggcgagtttaccgacaaggagcggagagatcacaggcgccggaaggccctggagaacaagcggaagcagctgagctccggcggcaagagcctgtccaaggaggaggaggaggagctgagaaagctgacagaggaggacgagagaagggagaggagggtggcaggacctagggtgggaggcgtgaacccactggagggaggaaccaggggagcacctggaggaggattcgtgccatccatgcagggagtgcctgagtctccatttgccaggacaggagagggcctggacgtgcgcggaaatcagggcttcccctgggacatcctgtttcctgccgatccacccttctcccctcagtcttgcaggccacagtctcgcagcgagtccaagaagaacagaggcggaagggaggaggtgctggagcagtgggtgaatggccggaagaagctggaagaactggagagggagctgagaagggcccgcaagaagatcaagaagctggaagacgataatccttggctgggcaatgtgaaaggcatcctgggcaagaaggacaaggatggagagggagcacctccagcaaagagggcaagaaccgaccagatggagatcgattctggaccaaggaagcgccctctgagaggaggcttcacagaccgggagagacaggatcaccgccggagaaaggccctgaagaacaagaagaagcagctgagcgccggcggcaagtctctgagtaaagaagaagaggaggagctgaagcggctgaccagagaggacgaggagcggaagaaggaggagcacggaccaagcagactgggagtgaatccttccgagggaggaccaagaggagcacccggaggaggcttcgtgccatctatgcagggcatccccgagagcaggtttaccagaaccggcgaaggcctggacgtgcggggctccagaggctttcctcaggacatcctgttcccatctgatcccccttttagcccccagtcctgtaggcctcagggcaccaacctgtccacatctaaccctctgggcttctttcctgatcatcagctggacccagccttccgcgccaacagcgccaatcccgattgggacttcaacccaaataaggatacctggccagacgctaacaaggtcggaggacagaacctgagcacatccaatcctctgggcttctttccagaccaccagctggatccagccttcagggctaatacagccaatcccgactgggacttcaacccaaataaggacacgtggccagacgcaaacaaggtcggctctcagagcgagacaaggaggggccggagaggaaccagggaggagacactggagaagtggatcaccgccagaaagaaggccgaggagctggagaaggacctgaggaagacccgcaagacaatcaagaagctggaagaagagaacccatggctgggcaatatcgtgggcatcatccgcaagggcaaggacggcgagggagcaccaccagcaaagaggcccaggactgatcagatggaggtggatagcggccctggcaagaggccacacaagtccggcttcacagacaaggagagggaggaccataggcgccggaaggccctggaaaacaagaagaagcaattatccgccggcggcaagatcctgtctaaagaagaggaagaagagctgagaaggctgaccgacgaggatgaggagaggaaaagaagggtggcaggaccaagagtgggcgacgtgaatccctccagaggcggaccaagaggagcacctggaggcggcttcgtgccccagatggccggcgtgcccgagtctccttttagcagaaccggagagggcctggatatcaggggaacccagggctttccatgggtgtccccatctcctccacagcagcggctgccactgctggagtgcacccctcagagccagagcgaatctaagaagaacagaaggggcggcagagaggacatcctggaaaaatggatcaccacacgcagaaaagctgaagaactggaaaaggacctgcggaaggccagaaaaacaatcaagaagctggaggatgaaaatccatggctgggaaacatcatcggcatcatcagaaagggcaaggacggggaaggcgcaccacctgcaaagcggcctagaacagatcagatggaaatcgattctggaaccggcaagcggccacacaagagcggcttcaccgacaaggagagagaggatcacagaaggcgcaaggccctggagaacaagaagaagcaattaagcagcggcggcaagaatctgtccagagaagaggaagaggagctgggcagactgaccgtggaggacgaggagcggagaaggcgcgtggcaggacccaggacaggcgatgtgaacctgagcggaggaggacctaggggagcaccaggaggcggcttcgtgcctaggatggagggagtgccagagtccccctttaccagaaccggcgagggcctggacatcaggggaaatcagggattcccatgggtgcggccttccccaccacagcagagactgccactgctggagtgtaccccccagggcacaaacctgagcacctccaatcccctgggcttctttcctgaccatcagctggaccctgccttcagggccaacagcgccaacccagattgggacttcaaccctaataaggacacctggccagatgcaaacaaggtcggcggccaaaacctgtctacaagcaaccccctgggcttctttccagatcaccagctggatcccgcctttcgcgccaataccgccaaccctgattgggatttcaaccctaacaaagacacttggcctgacgctaataaggtcggctgatgagaattc

SEQ ID NO: 37 - protein, 1136 aa, synthetic construct
MAGTNLSTSNPLGFFPDHQLDPAFRANSANPDWDFNPNKDTWPDANKVGGQNLSTSNPLGFFPDHQLDPAFRANTANPDWDFNPNKDTWPDANKVGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSCRPQSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSCRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLECTPQSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLECTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 38 - protein, 8 aa, synthetic construct
SIINFEKL

SEQ ID NO: 39 - protein, 17 aa, synthetic construct
ISQAVHAAHAEINEAGR

SEQ ID NO: 40 - protein, 9 aa, synthetic construct
KLEDDNPWL

SEQ ID NO: 41 - protein, 9 aa, synthetic construct
KLEEENPWL

SEQ ID NO: 42 - protein, 9 aa, synthetic construct
FPWDILFPA

SEQ ID NO: 43 - DNA, 639 bp, synthetic construct
  source 1..639: mol_type = other DNA
agccgtagcgaaagcaagaaaaatcgtggtggtcgtgaggaaattctggaacagtgggttggtgcacgtaagaaactggaggaactggaacgtgatctgcgtaaaattaagaaaaaaattaagaaactggaggaagaaaatccgtggctgggtaatattaaaggtattctgggtaagaaagatcgtgaaggtgaaggtgcaccgccggcaaaacgtgcacgtgcagatcagatggaagttgatagcggtccgcgtaaacgtccgtttcgtggtgaatttaccgataaagaacgtcgtgatcatcgccgtcgtaaagcactggaaaataaacgtaaacagctgagcagcggtggtaaaagcctgagcaaagaggaagaagaggaactgcgtaaactgaccgaggaagatgaacgtcgtgaacgtcgtgttgcaggtccgcgtgttggtggtgttaatccgctggaaggtggcacccgtggtgcaccgggtggtggttttgttccgagtatgcagggtgttccggaaagcccgtttgcacgtaccggtgaaggtctggatgttcgtggtaatcagggttttccgtgggatattctgtttccggcagatccgccgtttagcccgcagagcagtcgtccgcag

SEQ ID NO: 44 - DNA, 639 bp, synthetic construct
  source 1..639: mol_type = other DNA
agccgcagcgaaagcaaaaagaatcgcggcggccgcgaagaagtgctggaacagtgggtgaatggccgcaaaaagctggaagaactggaacgcgaactgcgccgcgcgcgcaaaaagattaaaaagctggaagatgataatccgtggctgggcaatgtgaaaggcattctgggcaaaaaggataaagatggcgaaggcgcgccgccggcgaaacgcgcgcgcaccgatcagatggaaattgatagcggcccgcgcaaacgcccgctgcgcggcggctttaccgatcgcgaacgccaggatcatcgtcgccgcaaagcgctgaaaaataaaaagaaacagctgagcgcgggcggcaaaagcctgagcaaagaagaagaggaagaactgaaacgcctgacccgcgaagatgaagagcgcaaaaaggaagagcatggcccgagccgcctgggcgtgaatccgagcgaaggcggcccgcgcggcgcgccgggcggtggctttgtgccgagtatgcagggcatcccggaaagccgctttacccgcaccggcgaaggcctggatgtgcgcggcagccgcggctttccgcaggatattctgtttccgagcgatccgccgtttagcccgcagagcagccgcccgcag

SEQ ID NO: 45 - DNA, 639 bp, synthetic construct
  source 1..639: mol_type = other DNA
agccagagcgaaacccgccgtggccgccgtggcacccgcgaggaaaccctggaaaaatggattaccgcgcgcaagaaagcggaggaactggaaaaagatctgcgcaaaacccgcaaaaccattaagaaactggaggaagaaaatccgtggctgggcaatattgtgggcattattcgcaagggcaaagatggcgaaggcgcgccgccggcgaaacgcccgcgcaccgatcagatggaagtggatagcggtccgggcaaacgcccgcataaaagcggctttaccgataaagagcgcgaagatcatcgccgccgtaaagcgctggaaaataagaaaaaacagctgagcgcgggcggtaaaattctgagcaaagaggaagaggaagaactgcgccgtctgaccgatgaagatgaggaacgcaaacgccgtgtggcgggtccgcgcgtgggcgatgtgaatccgagccgcggcggcccgcgcggcgcgccgggcggtggctttgtgccgcagatggcgggcgtgccggaaagcccgtttagccgcaccggcgaaggcctggatattcgtggcacccagggctttccgtgggtgagcccgagcccgccgcagcagcgcctgccgctgctggaaagcaccccgcag

SEQ ID NO: 46 - DNA, 639 bp, synthetic construct
  source 1..639: mol_type = other DNA
agccagagcgaaagcaaaaaaaatcgccgcggcggccgcgaagatattctggagaagtggattaccacccgccgcaaagcggaagagctggaaaaggatctgcgcaaagcgcgcaagaccattaaaaagctggaagatgagaatccgtggctgggcaatattattggcattattcgcaaaggcaaagatggcgaaggcgcaccgccggcgaaacgcccgcgcaccgatcagatggaaattgatagcggcaccggcaaacgtccgcataaaagcggctttaccgataaagaacgcgaagatcatcgtcgtcgcaaagcgctggaaaataaaaagaagcagctgagcagcggtggcaaaaatctgagccgcgaagaggaagaggaactgggccgcctgaccgtggaagatgaagagcgccgtcgccgtgtggcgggtccgcgcaccggcgatgtgaatctgagcggcggtggcccgcgtggtgcaccgggtggtggttttgtgccgcgcatggaaggcgtgccggagagcccgtttacccgtaccggcgaaggcctggatattcgtggcaatcagggttttccgtgggtgcgtccgagcccgccgcagcaacgtctgccgctcctggagagcaccccgcag

SEQ ID NO: 47 - protein, 213 aa, synthetic construct
SRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQ

SEQ ID NO: 48 - protein, 213 aa, synthetic construct
SRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSSRPQ

SEQ ID NO: 49 - protein, 213 aa, synthetic construct
SQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQ

SEQ ID NO: 50 - protein, 213 aa, synthetic construct
SQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLESTPQ

SEQ ID NO: 51 - DNA, 141 bp, synthetic construct
  source 1..141: mol_type = other DNA
ggcaccaatctgagcaccagcaatccgctgggcttttttccggatcatcagctcgatccggcgtttcgcgcgaatagcgcgaatccggattgggattttaatccgaataaagatacctggccggatgcgaataaagtgggc

SEQ ID NO: 52 - DNA, 141 bp, synthetic construct
  source 1..141: mol_type = other DNA
ggcaccaatctgagcaccagcaatccgctgggcttttttccggatcaccagctcgatccggcgtttcgcgcgaatagcgcgaatccggattgggattttaatccgaataaagatacctggccggatgcgaataaagtgggc

SEQ ID NO: 53 - DNA, 141 bp, synthetic construct
  source 1..141: mol_type = other DNA
ggtcaaaacctcagtacgagtaacccactcggtttcttcccagaccaccaactcgacccagccttccgtgccaacactgccaacccggactgggacttcaacccgaacaaggacacttggccggacgccaacaaggttggt

SEQ ID NO: 54 - skipped

SEQ ID NO: 55 - protein, 14 aa, synthetic construct
GSAPVPYPDPLEPR

SEQ ID NO: 56 - protein, 12 aa, synthetic construct
GSEQKLISEEDL

SEQ ID NO: 57 - protein, 10 aa, synthetic construct
GSDYKDDDDK

SEQ ID NO: 58 - protein, 10 aa, synthetic construct
GSWSHPQFEK

SEQ ID NO: 59 - protein, 8 aa, synthetic construct
GSHHHHHH

SEQ ID NO: 60 - DNA, 1596 bp, synthetic construct
  source 1..1596: mol_type = other DNA
atgagccgtagcgaaagcaagaaaaatcgtggtggtcgtgaggaaattctggaacagtgggttggtgcacgtaagaaactggaggaactggaacgtgatctgcgtaaaattaagaaaaaaattaagaaactggaggaagaaaatccgtggctgggtaatattaaaggtattctgggtaagaaagatcgtgaaggtgaaggtgcaccgccggcaaaacgtgcacgtgcagatcagatggaagttgatagcggtccgcgtaaacgtccgtttcgtggtgaatttaccgataaagaacgtcgtgatcatcgccgtcgtaaagcactggaaaataaacgtaaacagctgagcagcggtggtaaaagcctgagcaaagaggaagaagaggaactgcgtaaactgaccgaggaagatgaacgtcgtgaacgtcgtgttgcaggtccgcgtgttggtggtgttaatccgctggaaggtggcacccgtggtgcaccgggtggtggttttgttccgagtatgcagggtgttccggaaagcccgtttgcacgtaccggtgaaggtctggatgttcgtggtaatcagggttttccgtgggatattctgtttccggcagatccgccgtttagcccgcagagcagtcgtccgcagggtggtggcagccgcagcgaaagcaaaaagaatcgcggcggccgcgaagaagtgctggaacagtgggtgaatggccgcaaaaagctggaagaactggaacgcgaactgcgccgcgcgcgcaaaaagattaaaaagctggaagatgataatccgtggctgggcaatgtgaaaggcattctgggcaaaaaggataaagatggcgaaggcgcgccgccggcgaaacgcgcgcgcaccgatcagatggaaattgatagcggcccgcgcaaacgcccgctgcgcggcggctttaccgatcgcgaacgccaggatcatcgtcgccgcaaagcgctgaaaaataaaaagaaacagctgagcgcgggcggcaaaagcctgagcaaagaagaagaggaagaactgaaacgcctgacccgcgaagatgaagagcgcaaaaaggaagagcatggcccgagccgcctgggcgtgaatccgagcgaaggcggcccgcgcggcgcgccgggcggtggctttgtgccgagtatgcagggcatcccggaaagccgctttacccgcaccggcgaaggcctggatgtgcgcggcagccgcggctttccgcaggatattctgtttccgagcgatccgccgtttagcccgcagagcagccgcccgcagggcaccaatctgagcaccagcaatccgctgggcttttttccggatcatcagctcgatccggcgtttcgcgcgaatagcgcgaatccggattgggattttaatccgaataaagatacctggccggatgcgaataaagtgggcggtcaaaacctcagtacgagtaacccactcggtttcttcccagaccaccaactcgacccagccttccgtgccaacactgccaacccggactgggacttcaacccgaacaaggacacttggccggacgccaacaaggttggtggatcccaccaccaccaccaccac

SEQ ID NO: 61 - DNA, 1587 bp, synthetic construct
  source 1..1587: mol_type = other DNA
atgagccgtagcgaaagcaagaaaaatcgtggtggtcgtgaggaaattctggaacagtgggttggtgcacgtaagaaactggaggaactggaacgtgatctgcgtaaaattaagaaaaaaattaagaaactggaggaagaaaatccgtggctgggtaatattaaaggtattctgggtaagaaagatcgtgaaggtgaaggtgcaccgccggcaaaacgtgcacgtgcagatcagatggaagttgatagcggtccgcgtaaacgtccgtttcgtggtgaatttaccgataaagaacgtcgtgatcatcgccgtcgtaaagcactggaaaataaacgtaaacagctgagcagcggtggtaaaagcctgagcaaagaggaagaagaggaactgcgtaaactgaccgaggaagatgaacgtcgtgaacgtcgtgttgcaggtccgcgtgttggtggtgttaatccgctggaaggtggcacccgtggtgcaccgggtggtggttttgttccgagtatgcagggtgttccggaaagcccgtttgcacgtaccggtgaaggtctggatgttcgtggtaatcagggttttccgtgggatattctgtttccggcagatccgccgtttagcccgcagagcagtcgtccgcagagccgcagcgaaagcaaaaagaatcgcggcggccgcgaagaagtgctggaacagtgggtgaatggccgcaaaaagctggaagaactggaacgcgaactgcgccgcgcgcgcaaaaagattaaaaagctggaagatgataatccgtggctgggcaatgtgaaaggcattctgggcaaaaaggataaagatggcgaaggcgcgccgccggcgaaacgcgcgcgcaccgatcagatggaaattgatagcggcccgcgcaaacgcccgctgcgcggcggctttaccgatcgcgaacgccaggatcatcgtcgccgcaaagcgctgaaaaataaaaagaaacagctgagcgcgggcggcaaaagcctgagcaaagaagaagaggaagaactgaaacgcctgacccgcgaagatgaagagcgcaaaaaggaagagcatggcccgagccgcctgggcgtgaatccgagcgaaggcggcccgcgcggcgcgccgggcggtggctttgtgccgagtatgcagggcatcccggaaagccgctttacccgcaccggcgaaggcctggatgtgcgcggcagccgcggctttccgcaggatattctgtttccgagcgatccgccgtttagcccgcagagcagccgcccgcagggcaccaatctgagcaccagcaatccgctgggcttttttccggatcatcagctcgatccggcgtttcgcgcgaatagcgcgaatccggattgggattttaatccgaataaagatacctggccggatgcgaataaagtgggcggtcaaaacctcagtacgagtaacccactcggtttcttcccagaccaccaactcgacccagccttccgtgccaacactgccaacccggactgggacttcaacccgaacaaggacacttggccggacgccaacaaggttggtggatcccaccaccaccaccaccac

SEQ ID NO: 62 - DNA, 1596 bp, synthetic construct
  source 1..1596: mol_type = other DNA
atgagccagagcgaaacccgccgtggccgccgtggcacccgcgaggaaaccctggaaaaatggattaccgcgcgcaagaaagcggaggaactggaaaaagatctgcgcaaaacccgcaaaaccattaagaaactggaggaagaaaatccgtggctgggcaatattgtgggcattattcgcaagggcaaagatggcgaaggcgcgccgccggcgaaacgcccgcgcaccgatcagatggaagtggatagcggtccgggcaaacgcccgcataaaagcggctttaccgataaagagcgcgaagatcatcgccgccgtaaagcgctggaaaataagaaaaaacagctgagcgcgggcggtaaaattctgagcaaagaggaagaggaagaactgcgccgtctgaccgatgaagatgaggaacgcaaacgccgtgtggcgggtccgcgcgtgggcgatgtgaatccgagccgcggcggcccgcgcggcgcgccgggcggtggctttgtgccgcagatggcgggcgtgccggaaagcccgtttagccgcaccggcgaaggcctggatattcgtggcacccagggctttccgtgggtgagcccgagcccgccgcagcagcgcctgccgctgctggaaagcaccccgcagggtggtggcagccagagcgaaagcaaaaaaaatcgccgcggcggccgcgaagatattctggagaagtggattaccacccgccgcaaagcggaagagctggaaaaggatctgcgcaaagcgcgcaagaccattaaaaagctggaagatgagaatccgtggctgggcaatattattggcattattcgcaaaggcaaagatggcgaaggcgcaccgccggcgaaacgcccgcgcaccgatcagatggaaattgatagcggcaccggcaaacgtccgcataaaagcggctttaccgataaagaacgcgaagatcatcgtcgtcgcaaagcgctggaaaataaaaagaagcagctgagcagcggtggcaaaaatctgagccgcgaagaggaagaggaactgggccgcctgaccgtggaagatgaagagcgccgtcgccgtgtggcgggtccgcgcaccggcgatgtgaatctgagcggcggtggcccgcgtggtgcaccgggtggtggttttgtgccgcgcatggaaggcgtgccggagagcccgtttacccgtaccggcgaaggcctggatattcgtggcaatcagggttttccgtgggtgcgtccgagcccgccgcagcaacgtctgccgctcctggagagcaccccgcagggcaccaatctgagcaccagcaatccgctgggcttttttccggatcaccagctcgatccggcgtttcgcgcgaatagcgcgaatccggattgggattttaatccgaataaagatacctggccggatgcgaataaagtgggcggtcaaaacctcagtacgagtaacccactcggtttcttcccagaccaccaactcgacccagccttccgtgccaacactgccaacccggactgggacttcaacccgaacaaggacacttggccggacgccaacaaggttggtggatcccaccaccaccaccaccac

SEQ ID NO: 63 - DNA, 3465 bp, synthetic construct
  source 1..3465: mol_type = other DNA
atgggcaccaatctgagcaccagcaatccgctgggcttttttccggatcatcagctcgatccggcgtttcgcgcgaatagcgcgaatccggattgggattttaatccgaataaagatacctggccggatgcgaataaagtgggcggtcaaaacctcagtacgagtaacccactcggtttcttcccagaccaccaactcgacccagccttccgtgccaacactgccaacccggactgggacttcaacccgaacaaggacacttggccggacgccaacaaggttggtggtggtggcagccgtagcgaaagcaagaaaaatcgtggtggtcgtgaggaaattctggaacagtgggttggtgcacgtaagaaactggaggaactggaacgtgatctgcgtaaaattaagaaaaaaattaagaaactggaggaagaaaatccgtggctgggtaatattaaaggtattctgggtaagaaagatcgtgaaggtgaaggtgcaccgccggcaaaacgtgcacgtgcagatcagatggaagttgatagcggtccgcgtaaacgtccgtttcgtggtgaatttaccgataaagaacgtcgtgatcatcgccgtcgtaaagcactggaaaataaacgtaaacagctgagcagcggtggtaaaagcctgagcaaagaggaagaagaggaactgcgtaaactgaccgaggaagatgaacgtcgtgaacgtcgtgttgcaggtccgcgtgttggtggtgttaatccgctggaaggtggcacccgtggtgcaccgggtggtggttttgttccgagtatgcagggtgttccggaaagcccgtttgcacgtaccggtgaaggtctggatgttcgtggtaatcagggttttccgtgggatattctgtttccggcagatccgccgtttagcccgcagagcagtcgtccgcagggtggtggcagccgcagcgaaagcaaaaagaatcgcggcggccgcgaagaagtgctggaacagtgggtgaatggccgcaaaaagctggaagaactggaacgcgaactgcgccgcgcgcgcaaaaagattaaaaagctggaagatgataatccgtggctgggcaatgtgaaaggcattctgggcaaaaaggataaagatggcgaaggcgcgccgccggcgaaacgcgcgcgcaccgatcagatggaaattgatagcggcccgcgcaaacgcccgctgcgcggcggctttaccgatcgcgaacgccaggatcatcgtcgccgcaaagcgctgaaaaataaaaagaaacagctgagcgcgggcggcaaaagcctgagcaaagaagaagaggaagaactgaaacgcctgacccgcgaagatgaagagcgcaaaaaggaagagcatggcccgagccgcctgggcgtgaatccgagcgaaggcggcccgcgcggcgcgccgggcggtggctttgtgccgagtatgcagggcatcccggaaagccgctttacccgcaccggcgaaggcctggatgtgcgcggcagccgcggctttccgcaggatattctgtttccgagcgatccgccgtttagcccgcagagcagccgcccgcagggcaccaatctgagcaccagcaatccgctgggcttttttccggatcatcagctcgatccggcgtttcgcgcgaatagcgcgaatccggattgggattttaatccgaataaagatacctggccggatgcgaataaagtgggcggtcaaaacctcagtacgagtaacccactcggtttcttcccagaccaccaactcgacccagccttccgtgccaacactgccaacccggactgggacttcaacccgaacaaggacacttggccggacgccaacaaggttggtggtggtggcagccagagcgaaacccgccgtggccgccgtggcacccgcgaggaaaccctggaaaaatggattaccgcgcgcaagaaagcggaggaactggaaaaagatctgcgcaaaacccgcaaaaccattaagaaactggaggaagaaaatccgtggctgggcaatattgtgggcattattcgcaagggcaaagatggcgaaggcgcgccgccggcgaaacgcccgcgcaccgatcagatggaagtggatagcggtccgggcaaacgcccgcataaaagcggctttaccgataaagagcgcgaagatcatcgccgccgtaaagcgctggaaaataagaaaaaacagctgagcgcgggcggtaaaattctgagcaaagaggaagaggaagaactgcgccgtctgaccgatgaagatgaggaacgcaaacgccgtgtggcgggtccgcgcgtgggcgatgtgaatccgagccgcggcggcccgcgcggcgcgccgggcggtggctttgtgccgcagatggcgggcgtgccggaaagcccgtttagccgcaccggcgaaggcctggatattcgtggcacccagggctttccgtgggtgagcccgagcccgccgcagcagcgcctgccgctgctggaaagcaccccgcagggtggtggcagccagagcgaaagcaaaaaaaatcgccgcggcggccgcgaagatattctggagaagtggattaccacccgccgcaaagcggaagagctggaaaaggatctgcgcaaagcgcgcaagaccattaaaaagctggaagatgagaatccgtggctgggcaatattattggcatcattcgcaaaggcaaagatggcgaaggcgcaccgccggcgaaacgcccgcgcaccgatcagatggaaattgatagcggcaccggcaaacgtccgcataaaagcggctttaccgataaagaacgcgaagatcatcgtcgtcgcaaagcgctggaaaataaaaagaagcagctgagcagcggtggcaaaaatctgagccgcgaagaggaagaggaactgggccgcctgaccgtggaagatgaagagcgccgtcgccgtgtggcgggtccgcgcaccggcgatgtgaatctgagcggcggtggcccgcgtggtgcaccgggtggtggttttgtgccgcgcatggaaggcgtgccggagagcccgtttacccgtaccggcgaaggcctggatattcgtggcaatcagggttttccgtgggtgcgtccgagcccgccgcagcaacgtctgccgctcctggagagcaccccgcagggcaccaatctgagcaccagcaatccgctgggcttttttccggatcatcagctcgatccggcgtttcgcgcgaatagcgcgaatccggattgggattttaatccgaataaagatacctggccggatgcgaataaagtgggcggtcaaaacctcagtacgagtaacccactcggtttcttcccagaccaccaactcgacccagccttccgtgccaacactgccaacccggactgggacttcaacccgaacaaggacacttggccggacgccaacaaggttggtggatcccaccaccaccaccaccac

SEQ ID NO: 64 - DNA, 3438 bp, synthetic construct
  source 1..3438: mol_type = other DNA
atgggcaccaatctgagcaccagcaatccgctgggcttttttccggatcatcagctcgatccggcgtttcgcgcgaatagcgcgaatccggattgggattttaatccgaataaagatacctggccggatgcgaataaagtgggcggtcaaaacctcagtacgagtaacccactcggtttcttcccagaccaccaactcgacccagccttccgtgccaacactgccaacccggactgggacttcaacccgaacaaggacacttggccggacgccaacaaggttggtagccgtagcgaaagcaagaaaaatcgtggtggtcgtgaggaaattctggaacagtgggttggtgcacgtaagaaactggaggaactggaacgtgatctgcgtaaaattaagaaaaaaattaagaaactggaggaagaaaatccgtggctgggtaatattaaaggtattctgggtaagaaagatcgtgaaggtgaaggtgcaccgccggcaaaacgtgcacgtgcagatcagatggaagttgatagcggtccgcgtaaacgtccgtttcgtggtgaatttaccgataaagaacgtcgtgatcatcgccgtcgtaaagcactggaaaataaacgtaaacagctgagcagcggtggtaaaagcctgagcaaagaggaagaagaggaactgcgtaaactgaccgaggaagatgaacgtcgtgaacgtcgtgttgcaggtccgcgtgttggtggtgttaatccgctggaaggtggcacccgtggtgcaccgggtggtggttttgttccgagtatgcagggtgttccggaaagcccgtttgcacgtaccggtgaaggtctggatgttcgtggtaatcagggttttccgtgggatattctgtttccggcagatccgccgtttagcccgcagagcagtcgtccgcagagccgcagcgaaagcaaaaagaatcgcggcggccgcgaagaagtgctggaacagtgggtgaatggccgcaaaaagctggaagaactggaacgcgaactgcgccgcgcgcgcaaaaagattaaaaagctggaagatgataatccgtggctgggcaatgtgaaaggcattctgggcaaaaaggataaagatggcgaaggcgcgccgccggcgaaacgcgcgcgcaccgatcagatggaaattgatagcggcccgcgcaaacgcccgctgcgcggcggctttaccgatcgcgaacgccaggatcatcgtcgccgcaaagcgctgaaaaataaaaagaaacagctgagcgcgggcggcaaaagcctgagcaaagaagaagaggaagaactgaaacgcctgacccgcgaagatgaagagcgcaaaaaggaagagcatggcccgagccgcctgggcgtgaatccgagcgaaggcggcccgcgcggcgcgccgggcggtggctttgtgccgagtatgcagggcatcccggaaagccgctttacccgcaccggcgaaggcctggatgtgcgcggcagccgcggctttccgcaggatattctgtttccgagcgatccgccgtttagcccgcagagcagccgcccgcagggcaccaatctgagcaccagcaatccgctgggcttttttccggatcatcagctcgatccggcgtttcgcgcgaatagcgcgaatccggattgggattttaatccgaataaagatacctggccggatgcgaataaagtgggcggtcaaaacctcagtacgagtaacccactcggtttcttcccagaccaccaactcgacccagccttccgtgccaacactgccaacccggactgggacttcaacccgaacaaggacacttggccggacgccaacaaggttggtagccagagcgaaacccgccgtggccgccgtggcacccgcgaggaaaccctggaaaaatggattaccgcgcgcaagaaagcggaggaactggaaaaagatctgcgcaaaacccgcaaaaccattaagaaactggaggaagaaaatccgtggctgggcaatattgtgggcattattcgcaagggcaaagatggcgaaggcgcgccgccggcgaaacgcccgcgcaccgatcagatggaagtggatagcggtccgggcaaacgcccgcataaaagcggctttaccgataaagagcgcgaagatcatcgccgccgtaaagcgctggaaaataagaaaaaacagctgagcgcgggcggtaaaattctgagcaaagaggaagaggaagaactgcgccgtctgaccgatgaagatgaggaacgcaaacgccgtgtggcgggtccgcgcgtgggcgatgtgaatccgagccgcggcggcccgcgcggcgcgccgggcggtggctttgtgccgcagatggcgggcgtgccggaaagcccgtttagccgcaccggcgaaggcctggatattcgtggcacccagggctttccgtgggtgagcccgagcccgccgcagcagcgcctgccgctgctggaaagcaccccgcagggtggtggcagccagagcgaaagcaaaaaaaatcgccgcggcggccgcgaagatattctggagaagtggattaccacccgccgcaaagcggaagagctggaaaaggatctgcgcaaagcgcgcaagaccattaaaaagctggaagatgagaatccgtggctgggcaatattattggcatcattcgcaaaggcaaagatggcgaaggcgcaccgccggcgaaacgcccgcgcaccgatcagatggaaattgatagcggcaccggcaaacgtccgcataaaagcggctttaccgataaagaacgcgaagatcatcgtcgtcgcaaagcgctggaaaataaaaagaagcagctgagcagcggtggcaaaaatctgagccgcgaagaggaagaggaactgggccgcctgaccgtggaagatgaagagcgccgtcgccgtgtggcgggtccgcgcaccggcgatgtgaatctgagcggcggtggcccgcgtggtgcaccgggtggtggttttgtgccgcgcatggaaggcgtgccggagagcccgtttacccgtaccggcgaaggcctggatattcgtggcaatcagggttttccgtgggtgcgtccgagcccgccgcagcaacgtctgccgctcctggagagcaccccgcagggcaccaatctgagcaccagcaatccgctgggcttttttccggatcatcagctcgatccggcgtttcgcgcgaatagcgcgaatccggattgggattttaatccgaataaagatacctggccggatgcgaataaagtgggcggtcaaaacctcagtacgagtaacccactcggtttcttcccagaccaccaactcgacccagccttccgtgccaacactgccaacccggactgggacttcaacccgaacaaggacacttggccggacgccaacaaggttggtggatcccaccaccaccaccaccac

SEQ ID NO: 65 - DNA, 2892 bp, synthetic construct
  source 1..2892: mol_type = other DNA
atgagccgtagcgaaagcaagaaaaatcgtggtggtcgtgaggaaattctggaacagtgggttggtgcacgtaagaaactggaggaactggaacgtgatctgcgtaaaattaagaaaaaaattaagaaactggaggaagaaaatccgtggctgggtaatattaaaggtattctgggtaagaaagatcgtgaaggtgaaggtgcaccgccggcaaaacgtgcacgtgcagatcagatggaagttgatagcggtccgcgtaaacgtccgtttcgtggtgaatttaccgataaagaacgtcgtgatcatcgccgtcgtaaagcactggaaaataaacgtaaacagctgagcagcggtggtaaaagcctgagcaaagaggaagaagaggaactgcgtaaactgaccgaggaagatgaacgtcgtgaacgtcgtgttgcaggtccgcgtgttggtggtgttaatccgctggaaggtggcacccgtggtgcaccgggtggtggttttgttccgagtatgcagggtgttccggaaagcccgtttgcacgtaccggtgaaggtctggatgttcgtggtaatcagggttttccgtgggatattctgtttccggcagatccgccgtttagcccgcagagcagtcgtccgcagggtggtggcagccgcagcgaaagcaaaaagaatcgcggcggccgcgaagaagtgctggaacagtgggtgaatggccgcaaaaagctggaagaactggaacgcgaactgcgccgcgcgcgcaaaaagattaaaaagctggaagatgataatccgtggctgggcaatgtgaaaggcattctgggcaaaaaggataaagatggcgaaggcgcgccgccggcgaaacgcgcgcgcaccgatcagatggaaattgatagcggcccgcgcaaacgcccgctgcgcggcggctttaccgatcgcgaacgccaggatcatcgtcgccgcaaagcgctgaaaaataaaaagaaacagctgagcgcgggcggcaaaagcctgagcaaagaagaagaggaagaactgaaacgcctgacccgcgaagatgaagagcgcaaaaaggaagagcatggcccgagccgcctgggcgtgaatccgagcgaaggcggcccgcgcggcgcgccgggcggtggctttgtgccgagtatgcagggcatcccggaaagccgctttacccgcaccggcgaaggcctggatgtgcgcggcagccgcggctttccgcaggatattctgtttccgagcgatccgccgtttagcccgcagagcagccgcccgcagggcaccaatctgagcaccagcaatccgctgggcttttttccggatcatcagctcgatccggcgtttcgcgcgaatagcgcgaatccggattgggattttaatccgaataaagatacctggccggatgcgaataaagtgggcggtcaaaacctcagtacgagtaacccactcggtttcttcccagaccaccaactcgacccagccttccgtgccaacactgccaacccggactgggacttcaacccgaacaaggacacttggccggacgccaacaaggttggtggtggtggcagccagagcgaaacccgccgtggccgccgtggcacccgcgaggaaaccctggaaaaatggattaccgcgcgcaagaaagcggaggaactggaaaaagatctgcgcaaaacccgcaaaaccattaagaaactggaggaagaaaatccgtggctgggcaatattgtgggcattattcgcaagggcaaagatggcgaaggcgcgccgccggcgaaacgcccgcgcaccgatcagatggaagtggatagcggtccgggcaaacgcccgcataaaagcggctttaccgataaagagcgcgaagatcatcgccgccgtaaagcgctggaaaataagaaaaaacagctgagcgcgggcggtaaaattctgagcaaagaggaagaggaagaactgcgccgtctgaccgatgaagatgaggaacgcaaacgccgtgtggcgggtccgcgcgtgggcgatgtgaatccgagccgcggcggcccgcgcggcgcgccgggcggtggctttgtgccgcagatggcgggcgtgccggaaagcccgtttagccgcaccggcgaaggcctggatattcgtggcacccagggctttccgtgggtgagcccgagcccgccgcagcagcgcctgccgctgctggaaagcaccccgcagggtggtggcagccagagcgaaagcaaaaaaaatcgccgcggcggccgcgaagatattctggagaagtggattaccacccgccgcaaagcggaagagctggaaaaggatctgcgcaaagcgcgcaagaccattaaaaagctggaagatgagaatccgtggctgggcaatattattggcatcattcgcaaaggcaaagatggcgaaggcgcaccgccggcgaaacgcccgcgcaccgatcagatggaaattgatagcggcaccggcaaacgtccgcataaaagcggctttaccgataaagaacgcgaagatcatcgtcgtcgcaaagcgctggaaaataaaaagaagcagctgagcagcggtggcaaaaatctgagccgcgaagaggaagaggaactgggccgcctgaccgtggaagatgaagagcgccgtcgccgtgtggcgggtccgcgcaccggcgatgtgaatctgagcggcggtggcccgcgtggtgcaccgggtggtggttttgtgccgcgcatggaaggcgtgccggagagcccgtttacccgtaccggcgaaggcctggatattcgtggcaatcagggttttccgtgggtgcgtccgagcccgccgcagcaacgtctgccgctcctggagagcaccccgcagggatcccaccaccaccaccaccac

SEQ ID NO: 66 - DNA, 2169 bp, synthetic construct
  source 1..2169: mol_type = other DNA
atgggcaccaatctgagcaccagcaatccgctgggcttttttccggatcatcagctcgatccggcgtttcgcgcgaatagcgcgaatccggattgggattttaatccgaataaagatacctggccggatgcgaataaagtgggcggtcaaaacctcagtacgagtaacccactcggtttcttcccagaccaccaactcgacccagccttccgtgccaacactgccaacccggactgggacttcaacccgaacaaggacacttggccggacgccaacaaggttggtggtggtggcagccgtagcgaaagcaagaaaaatcgtggtggtcgtgaggaaattctggaacagtgggttggtgcacgtaagaaactggaggaactggaacgtgatctgcgtaaaattaagaaaaaaattaagaaactggaggaagaaaatccgtggctgggtaatattaaaggtattctgggtaagaaagatcgtgaaggtgaaggtgcaccgccggcaaaacgtgcacgtgcagatcagatggaagttgatagcggtccgcgtaaacgtccgtttcgtggtgaatttaccgataaagaacgtcgtgatcatcgccgtcgtaaagcactggaaaataaacgtaaacagctgagcagcggtggtaaaagcctgagcaaagaggaagaagaggaactgcgtaaactgaccgaggaagatgaacgtcgtgaacgtcgtgttgcaggtccgcgtgttggtggtgttaatccgctggaaggtggcacccgtggtgcaccgggtggtggttttgttccgagtatgcagggtgttccggaaagcccgtttgcacgtaccggtgaaggtctggatgttcgtggtaatcagggttttccgtgggatattctgtttccggcagatccgccgtttagcccgcagagcagtcgtccgcagggcaccaatctgagcaccagcaatccgctgggcttttttccggatcatcagctcgatccggcgtttcgcgcgaatagcgcgaatccggattgggattttaatccgaataaagatacctggccggatgcgaataaagtgggcggtcaaaacctcagtacgagtaacccactcggtttcttcccagaccaccaactcgacccagccttccgtgccaacactgccaacccggactgggacttcaacccgaacaaggacacttggccggacgccaacaaggttggtggtggtggcagccagagcgaaacccgccgtggccgccgtggcacccgcgaggaaaccctggaaaaatggattaccgcgcgcaagaaagcggaggaactggaaaaagatctgcgcaaaacccgcaaaaccattaagaaactggaggaagaaaatccgtggctgggcaatattgtgggcattattcgcaagggcaaagatggcgaaggcgcgccgccggcgaaacgcccgcgcaccgatcagatggaagtggatagcggtccgggcaaacgcccgcataaaagcggctttaccgataaagagcgcgaagatcatcgccgccgtaaagcgctggaaaataagaaaaaacagctgagcgcgggcggtaaaattctgagcaaagaggaagaggaagaactgcgccgtctgaccgatgaagatgaggaacgcaaacgccgtgtggcgggtccgcgcgtgggcgatgtgaatccgagccgcggcggcccgcgcggcgcgccgggcggtggctttgtgccgcagatggcgggcgtgccggaaagcccgtttagccgcaccggcgaaggcctggatattcgtggcacccagggctttccgtgggtgagcccgagcccgccgcagcagcgcctgccgctgctggaaagcaccccgcagggcaccaatctgagcaccagcaatccgctgggcttttttccggatcatcagctcgatccggcgtttcgcgcgaatagcgcgaatccggattgggattttaatccgaataaagatacctggccggatgcgaataaagtgggcggtcaaaacctcagtacgagtaacccactcggtttcttcccagaccaccaactcgacccagccttccgtgccaacactgccaacccggactgggacttcaacccgaacaaggacacttggccggacgccaacaaggttggtggatcccaccaccaccaccaccac

SEQ ID NO: 67 - DNA, 1878 bp, synthetic construct
  source 1..1878: mol_type = other DNA
atgagccgtagcgaaagcaagaaaaatcgtggtggtcgtgaggaaattctggaacagtgggttggtgcacgtaagaaactggaggaactggaacgtgatctgcgtaaaattaagaaaaaaattaagaaactggaggaagaaaatccgtggctgggtaatattaaaggtattctgggtaagaaagatcgtgaaggtgaaggtgcaccgccggcaaaacgtgcacgtgcagatcagatggaagttgatagcggtccgcgtaaacgtccgtttcgtggtgaatttaccgataaagaacgtcgtgatcatcgccgtcgtaaagcactggaaaataaacgtaaacagctgagcagcggtggtaaaagcctgagcaaagaggaagaagaggaactgcgtaaactgaccgaggaagatgaacgtcgtgaacgtcgtgttgcaggtccgcgtgttggtggtgttaatccgctggaaggtggcacccgtggtgcaccgggtggtggttttgttccgagtatgcagggtgttccggaaagcccgtttgcacgtaccggtgaaggtctggatgttcgtggtaatcagggttttccgtgggatattctgtttccggcagatccgccgtttagcccgcagagcagtcgtccgcagggcaccaatctgagcaccagcaatccgctgggcttttttccggatcatcagctcgatccggcgtttcgcgcgaatagcgcgaatccggattgggattttaatccgaataaagatacctggccggatgcgaataaagtgggcggtcaaaacctcagtacgagtaacccactcggtttcttcccagaccaccaactcgacccagccttccgtgccaacactgccaacccggactgggacttcaacccgaacaaggacacttggccggacgccaacaaggttggtggtggtggcagccagagcgaaacccgccgtggccgccgtggcacccgcgaggaaaccctggaaaaatggattaccgcgcgcaagaaagcggaggaactggaaaaagatctgcgcaaaacccgcaaaaccattaagaaactggaggaagaaaatccgtggctgggcaatattgtgggcattattcgcaagggcaaagatggcgaaggcgcgccgccggcgaaacgcccgcgcaccgatcagatggaagtggatagcggtccgggcaaacgcccgcataaaagcggctttaccgataaagagcgcgaagatcatcgccgccgtaaagcgctggaaaataagaaaaaacagctgagcgcgggcggtaaaattctgagcaaagaggaagaggaagaactgcgccgtctgaccgatgaagatgaggaacgcaaacgccgtgtggcgggtccgcgcgtgggcgatgtgaatccgagccgcggcggcccgcgcggcgcgccgggcggtggctttgtgccgcagatggcgggcgtgccggaaagcccgtttagccgcaccggcgaaggcctggatattcgtggcacccagggctttccgtgggtgagcccgagcccgccgcagcagcgcctgccgctgctggaaagcaccccgcagggcaccaatctgagcaccagcaatccgctgggcttttttccggatcatcagctcgatccggcgtttcgcgcgaatagcgcgaatccggattgggattttaatccgaataaagatacctggccggatgcgaataaagtgggcggtcaaaacctcagtacgagtaacccactcggtttcttcccagaccaccaactcgacccagccttccgtgccaacactgccaacccggactgggacttcaacccgaacaaggacacttggccggacgccaacaaggttggtggatcccaccaccaccaccaccac

SEQ ID NO: 68 - DNA, 1887 bp, synthetic construct
  source 1..1887: mol_type = other DNA
atgggcaccaatctgagcaccagcaatccgctgggcttttttccggatcatcagctcgatccggcgtttcgcgcgaatagcgcgaatccggattgggattttaatccgaataaagatacctggccggatgcgaataaagtgggcggtcaaaacctcagtacgagtaacccactcggtttcttcccagaccaccaactcgacccagccttccgtgccaacactgccaacccggactgggacttcaacccgaacaaggacacttggccggacgccaacaaggttggtggtggtggcagccgtagcgaaagcaagaaaaatcgtggtggtcgtgaggaaattctggaacagtgggttggtgcacgtaagaaactggaggaactggaacgtgatctgcgtaaaattaagaaaaaaattaagaaactggaggaagaaaatccgtggctgggtaatattaaaggtattctgggtaagaaagatcgtgaaggtgaaggtgcaccgccggcaaaacgtgcacgtgcagatcagatggaagttgatagcggtccgcgtaaacgtccgtttcgtggtgaatttaccgataaagaacgtcgtgatcatcgccgtcgtaaagcactggaaaataaacgtaaacagctgagcagcggtggtaaaagcctgagcaaagaggaagaagaggaactgcgtaaactgaccgaggaagatgaacgtcgtgaacgtcgtgttgcaggtccgcgtgttggtggtgttaatccgctggaaggtggcacccgtggtgcaccgggtggtggttttgttccgagtatgcagggtgttccggaaagcccgtttgcacgtaccggtgaaggtctggatgttcgtggtaatcagggttttccgtgggatattctgtttccggcagatccgccgtttagcccgcagagcagtcgtccgcagggcaccaatctgagcaccagcaatccgctgggcttttttccggatcatcagctcgatccggcgtttcgcgcgaatagcgcgaatccggattgggattttaatccgaataaagatacctggccggatgcgaataaagtgggcggtcaaaacctcagtacgagtaacccactcggtttcttcccagaccaccaactcgacccagccttccgtgccaacactgccaacccggactgggacttcaacccgaacaaggacacttggccggacgccaacaaggttggtggtggtggcagccagagcgaaacccgccgtggccgccgtggcacccgcgaggaaaccctggaaaaatggattaccgcgcgcaagaaagcggaggaactggaaaaagatctgcgcaaaacccgcaaaaccattaagaaactggaggaagaaaatccgtggctgggcaatattgtgggcattattcgcaagggcaaagatggcgaaggcgcgccgccggcgaaacgcccgcgcaccgatcagatggaagtggatagcggtccgggcaaacgcccgcataaaagcggctttaccgataaagagcgcgaagatcatcgccgccgtaaagcgctggaaaataagaaaaaacagctgagcgcgggcggtaaaattctgagcaaagaggaagaggaagaactgcgccgtctgaccgatgaagatgaggaacgcaaacgccgtgtggcgggtccgcgcgtgggcgatgtgaatccgagccgcggcggcccgcgcggcgcgccgggcggtggctttgtgccgcagatggcgggcgtgccggaaagcccgtttagccgcaccggcgaaggcctggatattcgtggcacccagggctttccgtgggtgagcccgagcccgccgcagcagcgcctgccgctgctggaaagcaccccgcagggatcccaccaccaccaccaccac

SEQ ID NO: 69 - DNA, 1596 bp, synthetic construct
  source 1..1596: mol_type = other DNA
atgagccgtagcgaaagcaagaaaaatcgtggtggtcgtgaggaaattctggaacagtgggttggtgcacgtaagaaactggaggaactggaacgtgatctgcgtaaaattaagaaaaaaattaagaaactggaggaagaaaatccgtggctgggtaatattaaaggtattctgggtaagaaagatcgtgaaggtgaaggtgcaccgccggcaaaacgtgcacgtgcagatcagatggaagttgatagcggtccgcgtaaacgtccgtttcgtggtgaatttaccgataaagaacgtcgtgatcatcgccgtcgtaaagcactggaaaataaacgtaaacagctgagcagcggtggtaaaagcctgagcaaagaggaagaagaggaactgcgtaaactgaccgaggaagatgaacgtcgtgaacgtcgtgttgcaggtccgcgtgttggtggtgttaatccgctggaaggtggcacccgtggtgcaccgggtggtggttttgttccgagtatgcagggtgttccggaaagcccgtttgcacgtaccggtgaaggtctggatgttcgtggtaatcagggttttccgtgggatattctgtttccggcagatccgccgtttagcccgcagagcagtcgtccgcagggcaccaatctgagcaccagcaatccgctgggcttttttccggatcatcagctcgatccggcgtttcgcgcgaatagcgcgaatccggattgggattttaatccgaataaagatacctggccggatgcgaataaagtgggcggtcaaaacctcagtacgagtaacccactcggtttcttcccagaccaccaactcgacccagccttccgtgccaacactgccaacccggactgggacttcaacccgaacaaggacacttggccggacgccaacaaggttggtggtggtggcagccagagcgaaacccgccgtggccgccgtggcacccgcgaggaaaccctggaaaaatggattaccgcgcgcaagaaagcggaggaactggaaaaagatctgcgcaaaacccgcaaaaccattaagaaactggaggaagaaaatccgtggctgggcaatattgtgggcattattcgcaagggcaaagatggcgaaggcgcgccgccggcgaaacgcccgcgcaccgatcagatggaagtggatagcggtccgggcaaacgcccgcataaaagcggctttaccgataaagagcgcgaagatcatcgccgccgtaaagcgctggaaaataagaaaaaacagctgagcgcgggcggtaaaattctgagcaaagaggaagaggaagaactgcgccgtctgaccgatgaagatgaggaacgcaaacgccgtgtggcgggtccgcgcgtgggcgatgtgaatccgagccgcggcggcccgcgcggcgcgccgggcggtggctttgtgccgcagatggcgggcgtgccggaaagcccgtttagccgcaccggcgaaggcctggatattcgtggcacccagggctttccgtgggtgagcccgagcccgccgcagcagcgcctgccgctgctggaaagcaccccgcagggatcccaccaccaccaccaccac

SEQ ID NO: 70 - DNA, 1746 bp, synthetic construct
  source 1..1746: mol_type = other DNA
atgggcaccaatctgagcaccagcaatccgctgggcttttttccggatcatcagctcgatccggcgtttcgcgcgaatagcgcgaatccggattgggattttaatccgaataaagatacctggccggatgcgaataaagtgggcggtggtggcagccgtagcgaaagcaagaaaaatcgtggtggtcgtgaggaaattctggaacagtgggttggtgcacgtaagaaactggaggaactggaacgtgatctgcgtaaaattaagaaaaaaattaagaaactggaggaagaaaatccgtggctgggtaatattaaaggtattctgggtaagaaagatcgtgaaggtgaaggtgcaccgccggcaaaacgtgcacgtgcagatcagatggaagttgatagcggtccgcgtaaacgtccgtttcgtggtgaatttaccgataaagaacgtcgtgatcatcgccgtcgtaaagcactggaaaataaacgtaaacagctgagcagcggtggtaaaagcctgagcaaagaggaagaagaggaactgcgtaaactgaccgaggaagatgaacgtcgtgaacgtcgtgttgcaggtccgcgtgttggtggtgttaatccgctggaaggtggcacccgtggtgcaccgggtggtggttttgttccgagtatgcagggtgttccggaaagcccgtttgcacgtaccggtgaaggtctggatgttcgtggtaatcagggttttccgtgggatattctgtttccggcagatccgccgtttagcccgcagagcagtcgtccgcagggtcaaaacctcagtacgagtaacccactcggtttcttcccagaccaccaactcgacccagccttccgtgccaacactgccaacccggactgggacttcaacccgaacaaggacacttggccggacgccaacaaggttggtggtggtggcagccagagcgaaacccgccgtggccgccgtggcacccgcgaggaaaccctggaaaaatggattaccgcgcgcaagaaagcggaggaactggaaaaagatctgcgcaaaacccgcaaaaccattaagaaactggaggaagaaaatccgtggctgggcaatattgtgggcattattcgcaagggcaaagatggcgaaggcgcgccgccggcgaaacgcccgcgcaccgatcagatggaagtggatagcggtccgggcaaacgcccgcataaaagcggctttaccgataaagagcgcgaagatcatcgccgccgtaaagcgctggaaaataagaaaaaacagctgagcgcgggcggtaaaattctgagcaaagaggaagaggaagaactgcgccgtctgaccgatgaagatgaggaacgcaaacgccgtgtggcgggtccgcgcgtgggcgatgtgaatccgagccgcggcggcccgcgcggcgcgccgggcggtggctttgtgccgcagatggcgggcgtgccggaaagcccgtttagccgcaccggcgaaggcctggatattcgtggcacccagggctttccgtgggtgagcccgagcccgccgcagcagcgcctgccgctgctggaaagcaccccgcagggcaccaatctgagcaccagcaatccgctgggcttttttccggatcatcagctcgatccggcgtttcgcgcgaatagcgcgaatccggattgggattttaatccgaataaagatacctggccggatgcgaataaagtgggcggatcccaccaccaccaccaccac

SEQ ID NO: 71 - DNA, 1596 bp, synthetic construct
  source 1..1596: mol_type = other DNA
atgagccgtagcgaaagcaagaaaaatcgtggtggtcgtgaggaaattctggaacagtgggttggtgcacgtaagaaactggaggaactggaacgtgatctgcgtaaaattaagaaaaaaattaagaaactggaggaagaaaatccgtggctgggtaatattaaaggtattctgggtaagaaagatcgtgaaggtgaaggtgcaccgccggcaaaacgtgcacgtgcagatcagatggaagttgatagcggtccgcgtaaacgtccgtttcgtggtgaatttaccgataaagaacgtcgtgatcatcgccgtcgtaaagcactggaaaataaacgtaaacagctgagcagcggtggtaaaagcctgagcaaagaggaagaagaggaactgcgtaaactgaccgaggaagatgaacgtcgtgaacgtcgtgttgcaggtccgcgtgttggtggtgttaatccgctggaaggtggcacccgtggtgcaccgggtggtggttttgttccgagtatgcagggtgttccggaaagcccgtttgcacgtaccggtgaaggtctggatgttcgtggtaatcagggttttccgtgggatattctgtttccggcagatccgccgtttagcccgcagagcagtcgtccgcagggtggtggcagccagagcgaaacccgccgtggccgccgtggcacccgcgaggaaaccctggaaaaatggattaccgcgcgcaagaaagcggaggaactggaaaaagatctgcgcaaaacccgcaaaaccattaagaaactggaggaagaaaatccgtggctgggcaatattgtgggcattattcgcaagggcaaagatggcgaaggcgcgccgccggcgaaacgcccgcgcaccgatcagatggaagtggatagcggtccgggcaaacgcccgcataaaagcggctttaccgataaagagcgcgaagatcatcgccgccgtaaagcgctggaaaataagaaaaaacagctgagcgcgggcggtaaaattctgagcaaagaggaagaggaagaactgcgccgtctgaccgatgaagatgaggaacgcaaacgccgtgtggcgggtccgcgcgtgggcgatgtgaatccgagccgcggcggcccgcgcggcgcgccgggcggtggctttgtgccgcagatggcgggcgtgccggaaagcccgtttagccgcaccggcgaaggcctggatattcgtggcacccagggctttccgtgggtgagcccgagcccgccgcagcagcgcctgccgctgctggaaagcaccccgcagggcaccaatctgagcaccagcaatccgctgggcttttttccggatcatcagctcgatccggcgtttcgcgcgaatagcgcgaatccggattgggattttaatccgaataaagatacctggccggatgcgaataaagtgggcggtcaaaacctcagtacgagtaacccactcggtttcttcccagaccaccaactcgacccagccttccgtgccaacactgccaacccggactgggacttcaacccgaacaaggacacttggccggacgccaacaaggttggtggatcccaccaccaccaccaccac

SEQ ID NO: 72 - protein, 532 aa, synthetic construct
MSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGGGSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSSRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGSHHHHHH

SEQ ID NO: 73 - protein, 529 aa, synthetic construct
MSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSSRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGSHHHHHH

SEQ ID NO: 74 - protein, 532 aa, synthetic construct
MSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGGGSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLESTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGSHHHHHH

SEQ ID NO: 75 - protein, 1155 aa, synthetic construct
MGTNLSTSNPLGFFPDHQLDPAFRANSANPDWDFNPNKDTWPDANKVGGQNLSTSNPLGFFPDHQLDPAFRANTANPDWDFNPNKDTWPDANKVGGGGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGGGSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSSRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGGGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGGGSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLESTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGSHHHHHH

SEQ ID NO: 76 - protein, 1146 aa, synthetic construct
MGTNLSTSNPLGFFPDHQLDPAFRANSANPDWDFNPNKDTWPDANKVGGQNLSTSNPLGFFPDHQLDPAFRANTANPDWDFNPNKDTWPDANKVGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSSRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGGGSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLESTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGSHHHHHH

SEQ ID NO: 77 - protein, 1146 aa, synthetic construct; identical to SEQ ID NO: 76

SEQ ID NO: 78 - protein, 723 aa, synthetic construct
MGTNLSTSNPLGFFPDHQLDPAFRANSANPDWDFNPNKDTWPDANKVGGQNLSTSNPLGFFPDHQLDPAFRANTANPDWDFNPNKDTWPDANKVGGGGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGGGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGSHHHHHH

SEQ ID NO: 79 - protein, 626 aa, synthetic construct
MSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGGGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGSHHHHHH

SEQ ID NO: 80 - protein, 629 aa, synthetic construct
MGTNLSTSNPLGFFPDHQLDPAFRANSANPDWDFNPNKDTWPDANKVGGQNLSTSNPLGFFPDHQLDPAFRANTANPDWDFNPNKDTWPDANKVGGGGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGGGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGSHHHHHH

SEQ ID NO: 81 - protein, 532 aa, synthetic construct
MSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGGGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGSHHHHHH

SEQ ID NO: 82 - protein, 582 aa, synthetic construct
MGTNLSTSNPLGFFPDHQLDPAFRANSANPDWDFNPNKDTWPDANKVGGGGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGQNLSTSNPLGFFPDHQLDPAFRANTANPDWDFNPNKDTWPDANKVGGGGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGTNLSTSNPLGFFPDHQLDPAFRANSANPDWDFNPNKDTWPDANKVGGSHHHHHH

SEQ ID NO: 83 - protein, 532 aa, synthetic construct
MSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGGGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGSHHHHHH

SEQ ID NO: 84 - protein, 524 aa, synthetic construct
MSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGGGSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSSRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 85 - protein, 521 aa, synthetic construct
MSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSSRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 86 - protein, 524 aa, synthetic construct
MSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGGGSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLESTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 87 - protein, 1147 aa, synthetic construct
MGTNLSTSNPLGFFPDHQLDPAFRANSANPDWDFNPNKDTWPDANKVGGQNLSTSNPLGFFPDHQLDPAFRANTANPDWDFNPNKDTWPDANKVGGGGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGGGSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSSRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGGGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGGGSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLESTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 88 - protein, 1138 aa, synthetic construct
MGTNLSTSNPLGFFPDHQLDPAFRANSANPDWDFNPNKDTWPDANKVGGQNLSTSNPLGFFPDHQLDPAFRANTANPDWDFNPNKDTWPDANKVGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSSRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGGGSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLESTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 89 - protein, 1138 aa, synthetic construct; identical to SEQ ID NO: 88

SEQ ID NO: 90 - protein, 715 aa, synthetic construct
MGTNLSTSNPLGFFPDHQLDPAFRANSANPDWDFNPNKDTWPDANKVGGQNLSTSNPLGFFPDHQLDPAFRANTANPDWDFNPNKDTWPDANKVGGGGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGGGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 91 - protein, 618 aa, synthetic construct
MSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGGGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 92 - protein, 621 aa, synthetic construct
MGTNLSTSNPLGFFPDHQLDPAFRANSANPDWDFNPNKDTWPDANKVGGQNLSTSNPLGFFPDHQLDPAFRANTANPDWDFNPNKDTWPDANKVGGGGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGGGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQ

SEQ ID NO: 93 - protein, 524 aa, synthetic construct
MSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGGGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQ

SEQ ID NO: 94 - protein, 574 aa, synthetic construct
MGTNLSTSNPLGFFPDHQLDPAFRANSANPDWDFNPNKDTWPDANKVGGGGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGQNLSTSNPLGFFPDHQLDPAFRANTANPDWDFNPNKDTWPDANKVGGGGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGTNLSTSNPLGFFPDHQLDPAFRANSANPDWDFNPNKDTWPDANKVG

SEQ ID NO: 95 - protein, 524 aa, synthetic construct
MSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGGGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 96 - DNA, 3000 bp, synthetic construct
  source 1..3000: mol_type = other DNA
atgagccgtagcgaaagcaagaaaaatcgtggtggtcgtgaggaaattctggaacagtgggttggtgcacgtaagaaactggaggaactggaacgtgatctgcgtaaaattaagaaaaaaattaagaaactggaggaagaaaatccgtggctgggtaatattaaaggtattctgggtaagaaagatcgtgaaggtgaaggtgcaccgccggcaaaacgtgcacgtgcagatcagatggaagttgatagcggtccgcgtaaacgtccgtttcgtggtgaatttaccgataaagaacgtcgtgatcatcgccgtcgtaaagcactggaaaataaacgtaaacagctgagcagcggtggtaaaagcctgagcaaagaggaagaagaggaactgcgtaaactgaccgaggaagatgaacgtcgtgaacgtcgtgttgcaggtccgcgtgttggtggtgttaatccgctggaaggtggcacccgtggtgcaccgggtggtggttttgttccgagtatgcagggtgttccggaaagcccgtttgcacgtaccggtgaaggtctggatgttcgtggtaatcagggttttccgtgggatattctgtttccggcagatccgccgtttagcccgcagagcagtcgtccgcagggtggtggcagccgcagcgaaagcaaaaagaatcgcggcggccgcgaagaagtgctggaacagtgggtgaatggccgcaaaaagctggaagaactggaacgcgaactgcgccgcgcgcgcaaaaagattaaaaagctggaagatgataatccgtggctgggcaatgtgaaaggcattctgggcaaaaaggataaagatggcgaaggcgcgccgccggcgaaacgcgcgcgcaccgatcagatggaaattgatagcggcccgcgcaaacgcccgctgcgcggcggctttaccgatcgcgaacgccaggatcatcgtcgccgcaaagcgctgaaaaataaaaagaaacagctgagcgcgggcggcaaaagcctgagcaaagaagaagaggaagaactgaaacgcctgacccgcgaagatgaagagcgcaaaaaggaagagcatggcccgagccgcctgggcgtgaatccgagcgaaggcggcccgcgcggcgcgccgggcggtggctttgtgccgagtatgcagggcatcccggaaagccgctttacccgcaccggcgaaggcctggatgtgcgcggcagccgcggctttccgcaggatattctgtttccgagcgatccgccgtttagcccgcagagcagccgcccgcagggcaccaatctgagcaccagcaatccgctgggcttttttccggatcatcagctcgatccggcgtttcgcgcgaatagcgcgaatccggattgggattttaatccgaataaagatacctggccggatgcgaataaagtgggcggctccgcgccggtgccgtacccggacccgctggaaccgcgcggctcccaccaccaccaccaccactaggaggtaattatgagccagagcgaaacccgccgtggccgccgtggcacccgcgaggaaaccctggaaaaatggattaccgcgcgcaagaaagcggaggaactggaaaaagatctgcgcaaaacccgcaaaaccattaagaaactggaggaagaaaatccgtggctgggcaatattgtgggcattattcgcaagggcaaagatggcgaaggcgcgccgccggcgaaacgcccgcgcaccgatcagatggaagtggatagcggtccgggcaaacgcccgcataaaagcggctttaccgataaagagcgcgaagatcatcgccgccgtaaagcgctggaaaataagaaaaaacagctgagcgcgggcggtaaaattctgagcaaagaggaagaggaagaactgcgccgtctgaccgatgaagatgaggaacgcaaacgccgtgtggcgggtccgcgcgtgggcgatgtgaatccgagccgcggcggcccgcgcggcgcgccgggcggtggctttgtgccgcagatggcgggcgtgccggaaagcccgtttagccgcaccggcgaaggcctggatattcgtggcacccagggctttccgtgggtgagcccgagcccgccgcagcagcgcctgccgctgctggaaagcaccccgcagggtggtggcagccagagcgaaagcaaaaaaaatcgccgcggcggccgcgaagatattctggagaagtggattaccacccgccgcaaagcggaagagctggaaaaggatctgcgcaaagcgcgcaagaccattaaaaagctggaagatgagaatccgtggctgggcaatattattggcatcattcgcaaaggcaaagatggcgaaggcgcaccgccggcgaaacgcccgcgcaccgatcagatggaaattgatagcggcaccggcaaacgtccgcataaaagcggctttaccgataaagaacgcgaagatcatcgtcgtcgcaaagcgctggaaaataaaaagaagcagctgagcagcggtggcaaaaatctgagccgcgaagaggaagaggaactgggccgcctgaccgtggaagatgaagagcgccgtcgccgtgtggcgggtccgcgcaccggcgatgtgaatctgagcggcggtggcccgcgtggtgcaccgggtggtggttttgtgccgcgcatggaaggcgtgccggagagcccgtttacccgtaccggcgaaggcctggatattcgtggcaatcagggttttccgtgggtgcgtccgagcccgccgcagcaacgtctgccgctcctggagagcaccccgcagggtcaaaacctcagtacgagtaacccactcggtttcttcccagaccaccaactcgacccagccttccgtgccaacactgccaacccggactgggacttcaacccgaacaaggacacttggccggacgccaacaaggttggtggctccgaacagaaactgattagcgaagaagatctgggatcccaccaccaccaccaccac

SEQ ID NO: 97 - DNA, 1704 bp, synthetic construct
  source 1..1704: mol_type = other DNA
atgagccgtagcgaaagcaagaaaaatcgtggtggtcgtgaggaaattctggaacagtgggttggtgcacgtaagaaactggaggaactggaacgtgatctgcgtaaaattaagaaaaaaattaagaaactggaggaagaaaatccgtggctgggtaatattaaaggtattctgggtaagaaagatcgtgaaggtgaaggtgcaccgccggcaaaacgtgcacgtgcagatcagatggaagttgatagcggtccgcgtaaacgtccgtttcgtggtgaatttaccgataaagaacgtcgtgatcatcgccgtcgtaaagcactggaaaataaacgtaaacagctgagcagcggtggtaaaagcctgagcaaagaggaagaagaggaactgcgtaaactgaccgaggaagatgaacgtcgtgaacgtcgtgttgcaggtccgcgtgttggtggtgttaatccgctggaaggtggcacccgtggtgcaccgggtggtggttttgttccgagtatgcagggtgttccggaaagcccgtttgcacgtaccggtgaaggtctggatgttcgtggtaatcagggttttccgtgggatattctgtttccggcagatccgccgtttagcccgcagagcagtcgtccgcagggcaccaatctgagcaccagcaatccgctgggcttttttccggatcatcagctcgatccggcgtttcgcgcgaatagcgcgaatccggattgggattttaatccgaataaagatacctggccggatgcgaataaagtgggcggctccgcgccggtgccgtacccggacccgctggaaccgcgcggctcccaccaccaccaccaccactaggaggtaattatgagccagagcgaaacccgccgtggccgccgtggcacccgcgaggaaaccctggaaaaatggattaccgcgcgcaagaaagcggaggaactggaaaaagatctgcgcaaaacccgcaaaaccattaagaaactggaggaagaaaatccgtggctgggcaatattgtgggcattattcgcaagggcaaagatggcgaaggcgcgccgccggcgaaacgcccgcgcaccgatcagatggaagtggatagcggtccgggcaaacgcccgcataaaagcggctttaccgataaagagcgcgaagatcatcgccgccgtaaagcgctggaaaataagaaaaaacagctgagcgcgggcggtaaaattctgagcaaagaggaagaggaagaactgcgccgtctgaccgatgaagatgaggaacgcaaacgccgtgtggcgggtccgcgcgtgggcgatgtgaatccgagccgcggcggcccgcgcggcgcgccgggcggtggctttgtgccgcagatggcgggcgtgccggaaagcccgtttagccgcaccggcgaaggcctggatattcgtggcacccagggctttccgtgggtgagcccgagcccgccgcagcagcgcctgccgctgctggaaagcaccccgcagggtcaaaacctcagtacgagtaacccactcggtttcttcccagaccaccaactcgacccagccttccgtgccaacactgccaacccggactgggacttcaacccgaacaaggacacttggccggacgccaacaaggttggtggctccgaacagaaactgattagcgaagaagatctgggatcccaccaccaccaccaccac

SEQ ID NO: 98 - DNA, 1986 bp, synthetic construct
  source 1..1986: mol_type = other DNA
atgagccgtagcgaaagcaagaaaaatcgtggtggtcgtgaggaaattctggaacagtgggttggtgcacgtaagaaactggaggaactggaacgtgatctgcgtaaaattaagaaaaaaattaagaaactggaggaagaaaatccgtggctgggtaatattaaaggtattctgggtaagaaagatcgtgaaggtgaaggtgcaccgccggcaaaacgtgcacgtgcagatcagatggaagttgatagcggtccgcgtaaacgtccgtttcgtggtgaatttaccgataaagaacgtcgtgatcatcgccgtcgtaaagcactggaaaataaacgtaaacagctgagcagcggtggtaaaagcctgagcaaagaggaagaagaggaactgcgtaaactgaccgaggaagatgaacgtcgtgaacgtcgtgttgcaggtccgcgtgttggtggtgttaatccgctggaaggtggcacccgtggtgcaccgggtggtggttttgttccgagtatgcagggtgttccggaaagcccgtttgcacgtaccggtgaaggtctggatgttcgtggtaatcagggttttccgtgggatattctgtttccggcagatccgccgtttagcccgcagagcagtcgtccgcagggcaccaatctgagcaccagcaatccgctgggcttttttccggatcatcagctcgatccggcgtttcgcgcgaatagcgcgaatccggattgggattttaatccgaataaagatacctggccggatgcgaataaagtgggcggtcaaaacctcagtacgagtaacccactcggtttcttcccagaccaccaactcgacccagccttccgtgccaacactgccaacccggactgggacttcaacccgaacaaggacacttggccggacgccaacaaggttggtggctccgcgccggtgccgtacccggacccgctggaaccgcgcggctcccaccaccaccaccaccactaggaggtaattatgagccagagcgaaacccgccgtggccgccgtggcacccgcgaggaaaccctggaaaaatggattaccgcgcgcaagaaagcggaggaactggaaaaagatctgcgcaaaacccgcaaaaccattaagaaactggaggaagaaaatccgtggctgggcaatattgtgggcattattcgcaagggcaaagatggcgaaggcgcgccgccggcgaaacgcccgcgcaccgatcagatggaagtggatagcggtccgggcaaacgcccgcataaaagcggctttaccgataaagagcgcgaagatcatcgccgccgtaaagcgctggaaaataagaaaaaacagctgagcgcgggcggtaaaattctgagcaaagaggaagaggaagaactgcgccgtctgaccgatgaagatgaggaacgcaaacgccgtgtggcgggtccgcgcgtgggcgatgtgaatccgagccgcggcggcccgcgcggcgcgccgggcggtggctttgtgccgcagatggcgggcgtgccggaaagcccgtttagccgcaccggcgaaggcctggatattcgtggcacccagggctttccgtgggtgagcccgagcccgccgcagcagcgcctgccgctgctggaaagcaccccgcagggcaccaatctgagcaccagcaatccgctgggcttttttccggatcatcagctcgatccggcgtttcgcgcgaatagcgcgaatccggattgggattttaatccgaataaagatacctggccggatgcgaataaagtgggcggtcaaaacctcagtacgagtaacccactcggtttcttcccagaccaccaactcgacccagccttccgtgccaacactgccaacccggactgggacttcaacccgaacaaggacacttggccggacgccaacaaggttggtggctccgaacagaaactgattagcgaagaagatctgggatcccaccaccaccaccaccac

SEQ ID NO: 99 - DNA, 1686 bp, synthetic construct
  source 1..1686: mol_type = other DNA
atgagccgcagcgaaagcaaaaagaatcgcggcggccgcgaagaagtgctggaacagtgggtgaatggccgcaaaaagctggaagaactggaacgcgaactgcgccgcgcgcgcaaaaagattaaaaagctggaagatgataatccgtggctgggcaatgtgaaaggcattctgggcaaaaaggataaagatggcgaaggcgcgccgccggcgaaacgcgcgcgcaccgatcagatggaaattgatagcggcccgcgcaaacgcccgctgcgcggcggctttaccgatcgcgaacgccaggatcatcgtcgccgcaaagcgctgaaaaataaaaagaaacagctgagcgcgggcggcaaaagcctgagcaaagaagaagaggaagaactgaaacgcctgacccgcgaagatgaagagcgcaaaaaggaagagcatggcccgagccgcctgggcgtgaatccgagcgaaggcggcccgcgcggcgcgccgggcggtggctttgtgccgagtatgcagggcatcccggaaagccgctttacccgcaccggcgaaggcctggatgtgcgcggcagccgcggctttccgcaggatattctgtttccgagcgatccgccgtttagcccgcagagcagccgcccgcagggcaccaatctgagcaccagcaatccgctgggcttttttccggatcatcagctcgatccggcgtttcgcgcgaatagcgcgaatccggattgggattttaatccgaataaagatacctggccggatgcgaataaagtgggcggctccgattataaagatgatgatgataaaggctcccaccaccaccaccaccactaggaggtaattatgagccagagcgaaagcaaaaaaaatcgccgcggcggccgcgaagatattctggagaagtggattaccacccgccgcaaagcggaagagctggaaaaggatctgcgcaaagcgcgcaagaccattaaaaagctggaagatgagaatccgtggctgggcaatattattggcatcattcgcaaaggcaaagatggcgaaggcgcaccgccggcgaaacgcccgcgcaccgatcagatggaaattgatagcggcaccggcaaacgtccgcataaaagcggctttaccgataaagaacgcgaagatcatcgtcgtcgcaaagcgctggaaaataaaaagaagcagctgagcagcggtggcaaaaatctgagccgcgaagaggaagaggaactgggccgcctgaccgtggaagatgaagagcgccgtcgccgtgtggcgggtccgcgcaccggcgatgtgaatctgagcggcggtggcccgcgtggtgcaccgggtggtggttttgtgccgcgcatggaaggcgtgccggagagcccgtttacccgtaccggcgaaggcctggatattcgtggcaatcagggttttccgtgggtgcgtccgagcccgccgcagcaacgtctgccgctcctggagagcaccccgcagggtcaaaacctcagtacgagtaacccactcggtttcttcccagaccaccaactcgacccagccttccgtgccaacactgccaacccggactgggacttcaacccgaacaaggacacttggccggacgccaacaaggttggtggctcctggagccatccgcagtttgaaaaaggatcccaccaccaccaccaccac

SEQ ID NO: 100 - DNA, 3402 bp, synthetic construct
  source 1..3402: mol_type = other DNA
atgagccgtagcgaaagcaagaaaaatcgtggtggtcgtgaggaaattctggaacagtgggttggtgcacgtaagaaactggaggaactggaacgtgatctgcgtaaaattaagaaaaaaattaagaaactggaggaagaaaatccgtggctgggtaatattaaaggtattctgggtaagaaagatcgtgaaggtgaaggtgcaccgccggcaaaacgtgcacgtgcagatcagatggaagttgatagcggtccgcgtaaacgtccgtttcgtggtgaatttaccgataaagaacgtcgtgatcatcgccgtcgtaaagcactggaaaataaacgtaaacagctgagcagcggtggtaaaagcctgagcaaagaggaagaagaggaactgcgtaaactgaccgaggaagatgaacgtcgtgaacgtcgtgttgcaggtccgcgtgttggtggtgttaatccgctggaaggtggcacccgtggtgcaccgggtggtggttttgttccgagtatgcagggtgttccggaaagcccgtttgcacgtaccggtgaaggtctggatgttcgtggtaatcagggttttccgtgggatattctgtttccggcagatccgccgtttagcccgcagagcagtcgtccgcagggcaccaatctgagcaccagcaatccgctgggcttttttccggatcatcagctcgatccggcgtttcgcgcgaatagcgcgaatccggattgggattttaatccgaataaagatacctggccggatgcgaataaagtgggcggctccgcgccggtgccgtacccggacccgctggaaccgcgcggctcccaccaccaccaccaccactaggaggtaattatgagccagagcgaaacccgccgtggccgccgtggcacccgcgaggaaaccctggaaaaatggattaccgcgcgcaagaaagcggaggaactggaaaaagatctgcgcaaaacccgcaaaaccattaagaaactggaggaagaaaatccgtggctgggcaatattgtgggcattattcgcaagggcaaagatggcgaaggcgcgccgccggcgaaacgcccgcgcaccgatcagatggaagtggatagcggtccgggcaaacgcccgcataaaagcggctttaccgataaagagcgcgaagatcatcgccgccgtaaagcgctggaaaataagaaaaaacagctgagcgcgggcggtaaaattctgagcaaagaggaagaggaagaactgcgccgtctgaccgatgaagatgaggaacgcaaacgccgtgtggcgggtccgcgcgtgggcgatgtgaatccgagccgcggcggcccgcgcggcgcgccgggcggtggctttgtgccgcagatggcgggcgtgccggaaagcccgtttagccgcaccggcgaaggcctggatattcgtggcacccagggctttccgtgggtgagcccgagcccgccgcagcagcgcctgccgctgctggaaagcaccccgcagggtcaaaacctcagtacgagtaacccactcggtttcttcccagaccaccaactcgacccagccttccgtgccaacactgccaacccggactgggacttcaacccgaacaaggacacttggccggacgccaacaaggttggtggctccgaacagaaactgattagcgaagaagatctgggctcccaccaccaccaccaccactaggaggtaattatgagccgcagcgaaagcaaaaagaatcgcggcggccgcgaagaagtgctggaacagtgggtgaatggccgcaaaaagctggaagaactggaacgcgaactgcgccgcgcgcgcaaaaagattaaaaagctggaagatgataatccgtggctgggcaatgtgaaaggcattctgggcaaaaaggataaagatggcgaaggcgcgccgccggcgaaacgcgcgcgcaccgatcagatggaaattgatagcggcccgcgcaaacgcccgctgcgcggcggctttaccgatcgcgaacgccaggatcatcgtcgccgcaaagcgctgaaaaataaaaagaaacagctgagcgcgggcggcaaaagcctgagcaaagaagaagaggaagaactgaaacgcctgacccgcgaagatgaagagcgcaaaaaggaagagcatggcccgagccgcctgggcgtgaatccgagcgaaggcggcccgcgcggcgcgccgggcggtggctttgtgccgagtatgcagggcatcccggaaagccgctttacccgcaccggcgaaggcctggatgtgcgcggcagccgcggctttccgcaggatattctgtttccgagcgatccgccgtttagcccgcagagcagccgcccgcagggcaccaatctgagcaccagcaatccgctgggcttttttccggatcatcagctcgatccggcgtttcgcgcgaatagcgcgaatccggattgggattttaatccgaataaagatacctggccggatgcgaataaagtgggcggctccgattataaagatgatgatgataaaggctcccaccaccaccaccaccactaggaggtaattatgagccagagcgaaagcaaaaaaaatcgccgcggcggccgcgaagatattctggagaagtggattaccacccgccgcaaagcggaagagctggaaaaggatctgcgcaaagcgcgcaagaccattaaaaagctggaagatgagaatccgtggctgggcaatattattggcatcattcgcaaaggcaaagatggcgaaggcgcaccgccggcgaaacgcccgcgcaccgatcagatggaaattgatagcggcaccggcaaacgtccgcataaaagcggctttaccgataaagaacgcgaagatcatcgtcgtcgcaaagcgctggaaaataaaaagaagcagctgagcagcggtggcaaaaatctgagccgcgaagaggaagaggaactgggccgcctgaccgtggaagatgaagagcgccgtcgccgtgtggcgggtccgcgcaccggcgatgtgaatctgagcggcggtggcccgcgtggtgcaccgggtggtggttttgtgccgcgcatggaaggcgtgccggagagcccgtttacccgtaccggcgaaggcctggatattcgtggcaatcagggttttccgtgggtgcgtccgagcccgccgcagcaacgtctgccgctcctggagagcaccccgcagggtcaaaacctcagtacgagtaacccactcggtttcttcccagaccaccaactcgacccagccttccgtgccaacactgccaacccggactgggacttcaacccgaacaaggacacttggccggacgccaacaaggttggtggatcctggagccatccgcagtttgaaaaaggctcccaccaccaccaccaccac

SEQ ID NO: 101 - DNA, 3489 bp, synthetic construct
  source 1..3489: mol_type = other DNA
atgagccgtagcgaaagcaagaaaaatcgtggtggtcgtgaggaaattctggaacagtgggttggtgcacgtaagaaactggaggaactggaacgtgatctgcgtaaaattaagaaaaaaattaagaaactggaggaagaaaatccgtggctgggtaatattaaaggtattctgggtaagaaagatcgtgaaggtgaaggtgcaccgccggcaaaacgtgcacgtgcagatcagatggaagttgatagcggtccgcgtaaacgtccgtttcgtggtgaatttaccgataaagaacgtcgtgatcatcgccgtcgtaaagcactggaaaataaacgtaaacagctgagcagcggtggtaaaagcctgagcaaagaggaagaagaggaactgcgtaaactgaccgaggaagatgaacgtcgtgaacgtcgtgttgcaggtccgcgtgttggtggtgttaatccgctggaaggtggcacccgtggtgcaccgggtggtggttttgttccgagtatgcagggtgttccggaaagcccgtttgcacgtaccggtgaaggtctggatgttcgtggtaatcagggttttccgtgggatattctgtttccggcagatccgccgtttagcccgcagagcagtcgtccgcagggcaccaatctgagcaccagcaatccgctgggcttttttccggatcatcagctcgatccggcgtttcgcgcgaatagcgcgaatccggattgggattttaatccgaataaagatacctggccggatgcgaataaagtgggcggctccgcgccggtgccgtacccggacccgctggaaccgcgcggctcccaccaccaccaccaccactaggaggtaattatgagccagagcgaaacccgccgtggccgccgtggcacccgcgaggaaaccctggaaaaatggattaccgcgcgcaagaaagcggaggaactggaaaaagatctgcgcaaaacccgcaaaaccattaagaaactggaggaagaaaatccgtggctgggcaatattgtgggcattattcgcaagggcaaagatggcgaaggcgcgccgccggcgaaacgcccgcgcaccgatcagatggaagtggatagcggtccgggcaaacgcccgcataaaagcggctttaccgataaagagcgcgaagatcatcgccgccgtaaagcgctggaaaataagaaaaaacagctgagcgcgggcggtaaaattctgagcaaagaggaagaggaagaactgcgccgtctgaccgatgaagatgaggaacgcaaacgccgtgtggcgggtccgcgcgtgggcgatgtgaatccgagccgcggcggcccgcgcggcgcgccgggcggtggctttgtgccgcagatggcgggcgtgccggaaagcccgtttagccgcaccggcgaaggcctggatattcgtggcacccagggctttccgtgggtgagcccgagcccgccgcagcagcgcctgccgctgctggaaagcaccccgcagggtcaaaacctcagtacgagtaacccactcggtttcttcccagaccaccaactcgacccagccttccgtgccaacactgccaacccggactgggacttcaacccgaacaaggacacttggccggacgccaacaaggttggtggctccgaacagaaactgattagcgaagaagatctgggctcccaccaccaccaccaccactaacgcgaaattaatacgactcactataggggaattgtgagcggataacaattcccctccagaaataattttgtttaactttaagaaggagatatacatatgagccgcagcgaaagcaaaaagaatcgcggcggccgcgaagaagtgctggaacagtgggtgaatggccgcaaaaagctggaagaactggaacgcgaactgcgccgcgcgcgcaaaaagattaaaaagctggaagatgataatccgtggctgggcaatgtgaaaggcattctgggcaaaaaggataaagatggcgaaggcgcgccgccggcgaaacgcgcgcgcaccgatcagatggaaattgatagcggcccgcgcaaacgcccgctgcgcggcggctttaccgatcgcgaacgccaggatcatcgtcgccgcaaagcgctgaaaaataaaaagaaacagctgagcgcgggcggcaaaagcctgagcaaagaagaagaggaagaactgaaacgcctgacccgcgaagatgaagagcgcaaaaaggaagagcatggcccgagccgcctgggcgtgaatccgagcgaaggcggcccgcgcggcgcgccgggcggtggctttgtgccgagtatgcagggcatcccggaaagccgctttacccgcaccggcgaaggcctggatgtgcgcggcagccgcggctttccgcaggatattctgtttccgagcgatccgccgtttagcccgcagagcagccgcccgcagggcaccaatctgagcaccagcaatccgctgggcttttttccggatcatcagctcgatccggcgtttcgcgcgaatagcgcgaatccggattgggattttaatccgaataaagatacctggccggatgcgaataaagtgggcggctccgattataaagatgatgatgataaaggctcccaccaccaccaccaccactaggaggtaattatgagccagagcgaaagcaaaaaaaatcgccgcggcggccgcgaagatattctggagaagtggattaccacccgccgcaaagcggaagagctggaaaaggatctgcgcaaagcgcgcaagaccattaaaaagctggaagatgagaatccgtggctgggcaatattattggcatcattcgcaaaggcaaagatggcgaaggcgcaccgccggcgaaacgcccgcgcaccgatcagatggaaattgatagcggcaccggcaaacgtccgcataaaagcggctttaccgataaagaacgcgaagatcatcgtcgtcgcaaagcgctggaaaataaaaagaagcagctgagcagcggtggcaaaaatctgagccgcgaagaggaagaggaactgggccgcctgaccgtggaagatgaagagcgccgtcgccgtgtggcgggtccgcgcaccggcgatgtgaatctgagcggcggtggcccgcgtggtgcaccgggtggtggttttgtgccgcgcatggaaggcgtgccggagagcccgtttacccgtaccggcgaaggcctggatattcgtggcaatcagggttttccgtgggtgcgtccgagcccgccgcagcaacgtctgccgctcctggagagcaccccgcagggtcaaaacctcagtacgagtaacccactcggtttcttcccagaccaccaactcgacccagccttccgtgccaacactgccaacccggactgggacttcaacccgaacaaggacacttggccggacgccaacaaggttggtggatcctggagccatccgcagtttgaaaaaggctcccaccaccaccaccaccac

SEQ ID NO: 102 - protein, 499 aa, synthetic construct
MSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGGGSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSSRPQGTNLSTSNPLGFFPDHQLDPAFRANSANPDWDFNPNKDTWPDANKVGGSAPVPYPDPLEPRGSHHHHHH

SEQ ID NO: 103 - protein, 497 aa, synthetic construct
MSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGGGSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLESTPQGQNLSTSNPLGFFPDHQLDPAFRANTANPDWDFNPNKDTWPDANKVGGSEQKLISEEDLGSHHHHHH

SEQ ID NO: 104 - protein, 283 aa, synthetic construct
MSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGTNLSTSNPLGFFPDHQLDPAFRANSANPDWDFNPNKDTWPDANKVGGSAPVPYPDPLEPRGSHHHHHH

SEQ ID NO: 105 - protein, 281 aa, synthetic construct
MSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGQNLSTSNPLGFFPDHQLDPAFRANTANPDWDFNPNKDTWPDANKVGGSEQKLISEEDLGSHHHHHH

SEQ ID NO: 106 - protein, 330 aa, synthetic construct
MSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGSAPVPYPDPLEPRGSHHHHHH

SEQ ID NO: 107 - protein, 328 aa, synthetic construct
MSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGSEQKLISEEDLGSHHHHHH

SEQ ID NO: 108 - protein, 279 aa, synthetic construct
MSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSSRPQGTNLSTSNPLGFFPDHQLDPAFRANSANPDWDFNPNKDTWPDANKVGGSDYKDDDDKGSHHHHHH

SEQ ID NO: 109 - protein, 279 aa, synthetic construct
MSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLESTPQGQNLSTSNPLGFFPDHQLDPAFRANTANPDWDFNPNKDTWPDANKVGGSWSHPQFEKGSHHHHHH

SEQ ID NO: 110 - protein, 283 aa, synthetic construct; identical to SEQ ID NO: 104

SEQ ID NO: 111 - protein, 279 aa, synthetic construct; identical to SEQ ID NO: 108

SEQ ID NO: 112 - protein, 281 aa, synthetic construct; identical to SEQ ID NO: 105

SEQ ID NO: 113 - protein, 279 aa, synthetic construct; identical to SEQ ID NO: 109

SEQ ID NO: 114 - protein, 283 aa, synthetic construct; identical to SEQ ID NO: 104

SEQ ID NO: 115 - protein, 279 aa, synthetic construct; identical to SEQ ID NO: 108

SEQ ID NO: 116 - protein, 281 aa, synthetic construct; identical to SEQ ID NO: 105

SEQ ID NO: 117 - protein, 279 aa, synthetic construct; identical to SEQ ID NO: 109

SEQ ID NO: 118 - protein, 477 aa, synthetic construct
MSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGGGSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSSRPQGTNLSTSNPLGFFPDHQLDPAFRANSANPDWDFNPNKDTWPDANKVG

SEQ ID NO: 119 - protein, 477 aa, synthetic construct
MSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGGGSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLESTPQGQNLSTSNPLGFFPDHQLDPAFRANTANPDWDFNPNKDTWPDANKVG

SEQ ID NO: 120 - protein, 261 aa, synthetic construct
MSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGTNLSTSNPLGFFPDHQLDPAFRANSANPDWDFNPNKDTWPDANKVG

SEQ ID NO: 121 - protein, 261 aa, synthetic construct
MSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGQNLSTSNPLGFFPDHQLDPAFRANTANPDWDFNPNKDTWPDANKVG

SEQ ID NO: 122 - protein, 308 aa, synthetic construct
MSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 123 - protein, 308 aa, synthetic construct
MSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 124 - protein, 261 aa, synthetic construct
MSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSSRPQGTNLSTSNPLGFFPDHQLDPAFRANSANPDWDFNPNKDTWPDANKVG

SEQ ID NO: 125 - protein, 261 aa, synthetic construct
MSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLESTPQGQNLSTSNPLGFFPDHQLDPAFRANTANPDWDFNPNKDTWPDANKVG

SEQ ID NO: 126 - protein, 261 aa, synthetic construct; identical to SEQ ID NO: 120

SEQ ID NO: 127 - protein, 261 aa, synthetic construct; identical to SEQ ID NO: 124

SEQ ID NO: 128 - protein, 261 aa, synthetic construct; identical to SEQ ID NO: 121

SEQ ID NO: 129 - protein, 261 aa, synthetic construct; identical to SEQ ID NO: 125

SEQ ID NO: 130 - protein, 261 aa, synthetic construct; identical to SEQ ID NO: 120

SEQ ID NO: 131 - protein, 261 aa, synthetic construct; identical to SEQ ID NO: 124

SEQ ID NO: 132 - protein, 261 aa, synthetic construct; identical to SEQ ID NO: 121

SEQ ID NO: 133 - protein, 261 aa, synthetic construct; identical to SEQ ID NO: 125

SEQ ID NO: 134 - DNA, 1566 bp, synthetic construct
  source 1..1566: mol_type = other DNA
atggccagccgcagcgaaagcaaaaaaaaccgcggcggccgcgaagaaattctggaacagtgggtgggcgcgcgcaaaaaactggaagaactggaacgcgatctgcgcaaaattaaaaaaaaaattaaaaaactggaagaagaaaacccgtggctgggcaacattaaaggcattctgggcaaaaaagatcgcgaaggcgaaggcgcgccgccggcgaaacgcgcgcgcgcggatcagatggaagtggatagcggcccgcgcaaacgcccgtttcgcggcgaatttaccgataaagaacgccgcgatcatcgccgccgcaaagcgctggaaaacaaacgcaaacagctgagcagcggcggcaaaagcctgagcaaagaagaagaagaagaactgcgcaaactgaccgaagaagatgaacgccgcgaacgccgcgtggcgggcccgcgcgtgggcggcgtgaacccgctggaaggcggcacccgcggcgcgccgggcggcggctttgtgccgagcatgcagggcgtgccggaaagcccgtttgcgcgcaccggcgaaggcctggatgtgcgcggcaaccagggctttccgtgggatattctgtttccggcggatccgccgtttagcccgcagagctgccgcccgcagagccagagcgaaacccgccgcggccgccgcggcacccgcgaagaaaccctggaaaaatggattaccgcgcgcaaaaaagcggaagaactggaaaaagatctgcgcaaaacccgcaaaaccattaaaaaactggaagaagaaaacccgtggctgggcaacattgtgggcattattcgcaaaggcaaagatggcgaaggcgcgccgccggcgaaacgcccgcgcaccgatcagatggaagtggatagcggcccgggcaaacgcccgcataaaagcggctttaccgataaagaacgcgaagatcatcgccgccgcaaagcgctggaaaacaaaaaaaaacagctgagcgcgggcggcaaaattctgagcaaagaagaagaagaagaactgcgccgcctgaccgatgaagatgaagaacgcaaacgccgcgtggcgggcccgcgcgtgggcgatgtgaacccgagccgcggcggcccgcgcggcgcgccgggcggcggctttgtgccgcagatggcgggcgtgccggaaagcccgtttagccgcaccggcgaaggcctggatattcgcggcacccagggctttccgtgggtgagcccgagcccgccgcagcagcgcctgccgctgctggaatgcaccccgcagggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggc

SEQ ID NO: 135 - DNA, 2130 bp, synthetic construct
  source 1..2130: mol_type = other DNA
atggccggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggcagccgcagcgaaagcaaaaaaaaccgcggcggccgcgaagaaattctggaacagtgggtgggcgcgcgcaaaaaactggaagaactggaacgcgatctgcgcaaaattaaaaaaaaaattaaaaaactggaagaagaaaacccgtggctgggcaacattaaaggcattctgggcaaaaaagatcgcgaaggcgaaggcgcgccgccggcgaaacgcgcgcgcgcggatcagatggaagtggatagcggcccgcgcaaacgcccgtttcgcggcgaatttaccgataaagaacgccgcgatcatcgccgccgcaaagcgctggaaaacaaacgcaaacagctgagcagcggcggcaaaagcctgagcaaagaagaagaagaagaactgcgcaaactgaccgaagaagatgaacgccgcgaacgccgcgtggcgggcccgcgcgtgggcggcgtgaacccgctggaaggcggcacccgcggcgcgccgggcggcggctttgtgccgagcatgcagggcgtgccggaaagcccgtttgcgcgcaccggcgaaggcctggatgtgcgcggcaaccagggctttccgtgggatattctgtttccggcggatccgccgtttagcccgcagagctgccgcccgcagggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggcagccagagcgaaacccgccgcggccgccgcggcacccgcgaagaaaccctggaaaaatggattaccgcgcgcaaaaaagcggaagaactggaaaaagatctgcgcaaaacccgcaaaaccattaaaaaactggaagaagaaaacccgtggctgggcaacattgtgggcattattcgcaaaggcaaagatggcgaaggcgcgccgccggcgaaacgcccgcgcaccgatcagatggaagtggatagcggcccgggcaaacgcccgcataaaagcggctttaccgataaagaacgcgaagatcatcgccgccgcaaagcgctggaaaacaaaaaaaaacagctgagcgcgggcggcaaaattctgagcaaagaagaagaagaagaactgcgccgcctgaccgatgaagatgaagaacgcaaacgccgcgtggcgggcccgcgcgtgggcgatgtgaacccgagccgcggcggcccgcgcggcgcgccgggcggcggctttgtgccgcagatggcgggcgtgccggaaagcccgtttagccgcaccggcgaaggcctggatattcgcggcacccagggctttccgtgggtgagcccgagcccgccgcagcagcgcctgccgctgctggaatgcaccccgcagggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggc

SEQ ID NO: 136 - protein, 522 aa, synthetic construct
MASRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSCRPQSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLECTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 137 - protein, 710 aa, synthetic construct
MAGTNLSTSNPLGFFPDHQLDPAFRANSANPDWDFNPNKDTWPDANKVGGQNLSTSNPLGFFPDHQLDPAFRANTANPDWDFNPNKDTWPDANKVGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSCRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLECTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 138 - DNA, 1566 bp, synthetic construct
  source 1..1566: mol_type = other DNA
atggccagccgcagcgaaagcaaaaaaaaccgcggcggccgcgaagaaattctggaacagtgggtgggcgcgcgcaaaaaactggaagaactggaacgcgatctgcgcaaaattaaaaaaaaaattaaaaaactggaagaagaaaacccgtggctgggcaacattaaaggcattctgggcaaaaaagatcgcgaaggcgaaggcgcgccgccggcgaaacgcgcgcgcgcggatcagatggaagtggatagcggcccgcgcaaacgcccgtttcgcggcgaatttaccgataaagaacgccgcgatcatcgccgccgcaaagcgctggaaaacaaacgcaaacagctgagcagcggcggcaaaagcctgagcaaagaagaagaagaagaactgcgcaaactgaccgaagaagatgaacgccgcgaacgccgcgtggcgggcccgcgcgtgggcggcgtgaacccgctggaaggcggcacccgcggcgcgccgggcggcggctttgtgccgagcatgcagggcgtgccggaaagcccgtttgcgcgcaccggcgaaggcctggatgtgcgcggcaaccagggctttccgtgggatattctgtttccggcggatccgccgtttagcccgcagagcagtcgcccgcagagccagagcgaaacccgccgcggccgccgcggcacccgcgaagaaaccctggaaaaatggattaccgcgcgcaaaaaagcggaagaactggaaaaagatctgcgcaaaacccgcaaaaccattaaaaaactggaagaagaaaacccgtggctgggcaacattgtgggcattattcgcaaaggcaaagatggcgaaggcgcgccgccggcgaaacgcccgcgcaccgatcagatggaagtggatagcggcccgggcaaacgcccgcataaaagcggctttaccgataaagaacgcgaagatcatcgccgccgcaaagcgctggaaaacaaaaaaaaacagctgagcgcgggcggcaaaattctgagcaaagaagaagaagaagaactgcgccgcctgaccgatgaagatgaagaacgcaaacgccgcgtggcgggcccgcgcgtgggcgatgtgaacccgagccgcggcggcccgcgcggcgcgccgggcggcggctttgtgccgcagatggcgggcgtgccggaaagcccgtttagccgcaccggcgaaggcctggatattcgcggcacccagggctttccgtgggtgagcccgagcccgccgcagcagcgcctgccgctgctggaaagcaccccgcagggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggc

SEQ ID NO: 139 - DNA, 2130 bp, synthetic construct
  source 1..2130: mol_type = other DNA
atggccggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggcagccgcagcgaaagcaaaaaaaaccgcggcggccgcgaagaaattctggaacagtgggtgggcgcgcgcaaaaaactggaagaactggaacgcgatctgcgcaaaattaaaaaaaaaattaaaaaactggaagaagaaaacccgtggctgggcaacattaaaggcattctgggcaaaaaagatcgcgaaggcgaaggcgcgccgccggcgaaacgcgcgcgcgcggatcagatggaagtggatagcggcccgcgcaaacgcccgtttcgcggcgaatttaccgataaagaacgccgcgatcatcgccgccgcaaagcgctggaaaacaaacgcaaacagctgagcagcggcggcaaaagcctgagcaaagaagaagaagaagaactgcgcaaactgaccgaagaagatgaacgccgcgaacgccgcgtggcgggcccgcgcgtgggcggcgtgaacccgctggaaggcggcacccgcggcgcgccgggcggcggctttgtgccgagcatgcagggcgtgccggaaagcccgtttgcgcgcaccggcgaaggcctggatgtgcgcggcaaccagggctttccgtgggatattctgtttccggcggatccgccgtttagcccgcagagcagtcgcccgcagggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggcagccagagcgaaacccgccgcggccgccgcggcacccgcgaagaaaccctggaaaaatggattaccgcgcgcaaaaaagcggaagaactggaaaaagatctgcgcaaaacccgcaaaaccattaaaaaactggaagaagaaaacccgtggctgggcaacattgtgggcattattcgcaaaggcaaagatggcgaaggcgcgccgccggcgaaacgcccgcgcaccgatcagatggaagtggatagcggcccgggcaaacgcccgcataaaagcggctttaccgataaagaacgcgaagatcatcgccgccgcaaagcgctggaaaacaaaaaaaaacagctgagcgcgggcggcaaaattctgagcaaagaagaagaagaagaactgcgccgcctgaccgatgaagatgaagaacgcaaacgccgcgtggcgggcccgcgcgtgggcgatgtgaacccgagccgcggcggcccgcgcggcgcgccgggcggcggctttgtgccgcagatggcgggcgtgccggaaagcccgtttagccgcaccggcgaaggcctggatattcgcggcacccagggctttccgtgggtgagcccgagcccgccgcagcagcgcctgccgctgctggaaagcaccccgcagggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggc

SEQ ID NO: 140 - protein, 522 aa, synthetic construct
MASRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 141 - protein, 710 aa, synthetic construct
MAGTNLSTSNPLGFFPDHQLDPAFRANSANPDWDFNPNKDTWPDANKVGGQNLSTSNPLGFFPDHQLDPAFRANTANPDWDFNPNKDTWPDANKVGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 142 - DNA, 1575 bp, synthetic construct
  source 1..1575: mol_type = other DNA
atggccagccgcagcgaaagcaaaaaaaaccgcggcggccgcgaagaaattctggaacagtgggtgggcgcgcgcaaaaaactggaagaactggaacgcgatctgcgcaaaattaaaaaaaaaattaaaaaactggaagaagaaaacccgtggctgggcaacattaaaggcattctgggcaaaaaagatcgcgaaggcgaaggcgcgccgccggcgaaacgcgcgcgcgcggatcagatggaagtggatagcggcccgcgcaaacgcccgtttcgcggcgaatttaccgataaagaacgccgcgatcatcgccgccgcaaagcgctggaaaacaaacgcaaacagctgagcagcggcggcaaaagcctgagcaaagaagaagaagaagaactgcgcaaactgaccgaagaagatgaacgccgcgaacgccgcgtggcgggcccgcgcgtgggcggcgtgaacccgctggaaggcggcacccgcggcgcgccgggcggcggctttgtgccgagcatgcagggcgtgccggaaagcccgtttgcgcgcaccggcgaaggcctggatgtgcgcggcaaccagggctttccgtgggatattctgtttccggcggatccgccgtttagcccgcagagcagtcgcccgcagggtggtggcagccagagcgaaacccgccgcggccgccgcggcacccgcgaagaaaccctggaaaaatggattaccgcgcgcaaaaaagcggaagaactggaaaaagatctgcgcaaaacccgcaaaaccattaaaaaactggaagaagaaaacccgtggctgggcaacattgtgggcattattcgcaaaggcaaagatggcgaaggcgcgccgccggcgaaacgcccgcgcaccgatcagatggaagtggatagcggcccgggcaaacgcccgcataaaagcggctttaccgataaagaacgcgaagatcatcgccgccgcaaagcgctggaaaacaaaaaaaaacagctgagcgcgggcggcaaaattctgagcaaagaagaagaagaagaactgcgccgcctgaccgatgaagatgaagaacgcaaacgccgcgtggcgggcccgcgcgtgggcgatgtgaacccgagccgcggcggcccgcgcggcgcgccgggcggcggctttgtgccgcagatggcgggcgtgccggaaagcccgtttagccgcaccggcgaaggcctggatattcgcggcacccagggctttccgtgggtgagcccgagcccgccgcagcagcgcctgccgctgctggaaagcaccccgcagggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggc

SEQ ID NO: 143 - DNA, 1575 bp, synthetic construct
  source 1..1575: mol_type = other DNA
atggccagccgcagcgaaagcaaaaaaaaccgcggcggccgcgaagaaattctggaacagtgggtgggcgcgcgcaaaaaactggaagaactggaacgcgatctgcgcaaaattaaaaaaaaaattaaaaaactggaagaagaaaacccgtggctgggcaacattaaaggcattctgggcaaaaaagatcgcgaaggcgaaggcgcgccgccggcgaaacgcgcgcgcgcggatcagatggaagtggatagcggcccgcgcaaacgcccgtttcgcggcgaatttaccgataaagaacgccgcgatcatcgccgccgcaaagcgctggaaaacaaacgcaaacagctgagcagcggcggcaaaagcctgagcaaagaagaagaagaagaactgcgcaaactgaccgaagaagatgaacgccgcgaacgccgcgtggcgggcccgcgcgtgggcggcgtgaacccgctggaaggcggcacccgcggcgcgccgggcggcggctttgtgccgagcatgcagggcgtgccggaaagcccgtttgcgcgcaccggcgaaggcctggatgtgcgcggcaaccagggctttccgtgggatattctgtttccggcggatccgccgtttagcccgcagagcagtcgcccgcagagccagagcgaaacccgccgcggccgccgcggcacccgcgaagaaaccctggaaaaatggattaccgcgcgcaaaaaagcggaagaactggaaaaagatctgcgcaaaacccgcaaaaccattaaaaaactggaagaagaaaacccgtggctgggcaacattgtgggcattattcgcaaaggcaaagatggcgaaggcgcgccgccggcgaaacgcccgcgcaccgatcagatggaagtggatagcggcccgggcaaacgcccgcataaaagcggctttaccgataaagaacgcgaagatcatcgccgccgcaaagcgctggaaaacaaaaaaaaacagctgagcgcgggcggcaaaattctgagcaaagaagaagaagaagaactgcgccgcctgaccgatgaagatgaagaacgcaaacgccgcgtggcgggcccgcgcgtgggcgatgtgaacccgagccgcggcggcccgcgcggcgcgccgggcggcggctttgtgccgcagatggcgggcgtgccggaaagcccgtttagccgcaccggcgaaggcctggatattcgcggcacccagggctttccgtgggtgagcccgagcccgccgcagcagcgcctgccgctgctggaaagcaccccgcagggtggtggcggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggc

SEQ ID NO: 144 - DNA, 1584 bp, synthetic construct
  source 1..1584: mol_type = other DNA
atggccagccgcagcgaaagcaaaaaaaaccgcggcggccgcgaagaaattctggaacagtgggtgggcgcgcgcaaaaaactggaagaactggaacgcgatctgcgcaaaattaaaaaaaaaattaaaaaactggaagaagaaaacccgtggctgggcaacattaaaggcattctgggcaaaaaagatcgcgaaggcgaaggcgcgccgccggcgaaacgcgcgcgcgcggatcagatggaagtggatagcggcccgcgcaaacgcccgtttcgcggcgaatttaccgataaagaacgccgcgatcatcgccgccgcaaagcgctggaaaacaaacgcaaacagctgagcagcggcggcaaaagcctgagcaaagaagaagaagaagaactgcgcaaactgaccgaagaagatgaacgccgcgaacgccgcgtggcgggcccgcgcgtgggcggcgtgaacccgctggaaggcggcacccgcggcgcgccgggcggcggctttgtgccgagcatgcagggcgtgccggaaagcccgtttgcgcgcaccggcgaaggcctggatgtgcgcggcaaccagggctttccgtgggatattctgtttccggcggatccgccgtttagcccgcagagcagtcgcccgcaggccaccaccagccagagcgaaacccgccgcggccgccgcggcacccgcgaagaaaccctggaaaaatggattaccgcgcgcaaaaaagcggaagaactggaaaaagatctgcgcaaaacccgcaaaaccattaaaaaactggaagaagaaaacccgtggctgggcaacattgtgggcattattcgcaaaggcaaagatggcgaaggcgcgccgccggcgaaacgcccgcgcaccgatcagatggaagtggatagcggcccgggcaaacgcccgcataaaagcggctttaccgataaagaacgcgaagatcatcgccgccgcaaagcgctggaaaacaaaaaaaaacagctgagcgcgggcggcaaaattctgagcaaagaagaagaagaagaactgcgccgcctgaccgatgaagatgaagaacgcaaacgccgcgtggcgggcccgcgcgtgggcgatgtgaacccgagccgcggcggcccgcgcggcgcgccgggcggcggctttgtgccgcagatggcgggcgtgccggaaagcccgtttagccgcaccggcgaaggcctggatattcgcggcacccagggctttccgtgggtgagcccgagcccgccgcagcagcgcctgccgctgctggaaagcaccccgcaggccaccaccggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggc

SEQ ID NO: 145 - protein, 525 aa, synthetic construct
MASRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGGGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 146 - protein, 525 aa, synthetic construct
MASRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGGGGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 147 - protein, 528 aa, synthetic construct
MASRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQATTSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQATTGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 148 - DNA, 2139 bp, synthetic construct
  source 1..2139: mol_type = other DNA
atggccggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggcggtggtggcagccgcagcgaaagcaaaaaaaaccgcggcggccgcgaagaaattctggaacagtgggtgggcgcgcgcaaaaaactggaagaactggaacgcgatctgcgcaaaattaaaaaaaaaattaaaaaactggaagaagaaaacccgtggctgggcaacattaaaggcattctgggcaaaaaagatcgcgaaggcgaaggcgcgccgccggcgaaacgcgcgcgcgcggatcagatggaagtggatagcggcccgcgcaaacgcccgtttcgcggcgaatttaccgataaagaacgccgcgatcatcgccgccgcaaagcgctggaaaacaaacgcaaacagctgagcagcggcggcaaaagcctgagcaaagaagaagaagaagaactgcgcaaactgaccgaagaagatgaacgccgcgaacgccgcgtggcgggcccgcgcgtgggcggcgtgaacccgctggaaggcggcacccgcggcgcgccgggcggcggctttgtgccgagcatgcagggcgtgccggaaagcccgtttgcgcgcaccggcgaaggcctggatgtgcgcggcaaccagggctttccgtgggatattctgtttccggcggatccgccgtttagcccgcagagcagtcgcccgcagggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggcagccagagcgaaacccgccgcggccgccgcggcacccgcgaagaaaccctggaaaaatggattaccgcgcgcaaaaaagcggaagaactggaaaaagatctgcgcaaaacccgcaaaaccattaaaaaactggaagaagaaaacccgtggctgggcaacattgtgggcattattcgcaaaggcaaagatggcgaaggcgcgccgccggcgaaacgcccgcgcaccgatcagatggaagtggatagcggcccgggcaaacgcccgcataaaagcggctttaccgataaagaacgcgaagatcatcgccgccgcaaagcgctggaaaacaaaaaaaaacagctgagcgcgggcggcaaaattctgagcaaagaagaagaagaagaactgcgccgcctgaccgatgaagatgaagaacgcaaacgccgcgtggcgggcccgcgcgtgggcgatgtgaacccgagccgcggcggcccgcgcggcgcgccgggcggcggctttgtgccgcagatggcgggcgtgccggaaagcccgtttagccgcaccggcgaaggcctggatattcgcggcacccagggctttccgtgggtgagcccgagcccgccgcagcagcgcctgccgctgctggaaagcaccccgcagggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggc

SEQ ID NO: 149 - DNA, 2148 bp, synthetic construct
  source 1..2148: mol_type = other DNA
atggccggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggcggtggtggcagccgcagcgaaagcaaaaaaaaccgcggcggccgcgaagaaattctggaacagtgggtgggcgcgcgcaaaaaactggaagaactggaacgcgatctgcgcaaaattaaaaaaaaaattaaaaaactggaagaagaaaacccgtggctgggcaacattaaaggcattctgggcaaaaaagatcgcgaaggcgaaggcgcgccgccggcgaaacgcgcgcgcgcggatcagatggaagtggatagcggcccgcgcaaacgcccgtttcgcggcgaatttaccgataaagaacgccgcgatcatcgccgccgcaaagcgctggaaaacaaacgcaaacagctgagcagcggcggcaaaagcctgagcaaagaagaagaagaagaactgcgcaaactgaccgaagaagatgaacgccgcgaacgccgcgtggcgggcccgcgcgtgggcggcgtgaacccgctggaaggcggcacccgcggcgcgccgggcggcggctttgtgccgagcatgcagggcgtgccggaaagcccgtttgcgcgcaccggcgaaggcctggatgtgcgcggcaaccagggctttccgtgggatattctgtttccggcggatccgccgtttagcccgcagagcagtcgcccgcagggtggtggcggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggcagccagagcgaaacccgccgcggccgccgcggcacccgcgaagaaaccctggaaaaatggattaccgcgcgcaaaaaagcggaagaactggaaaaagatctgcgcaaaacccgcaaaaccattaaaaaactggaagaagaaaacccgtggctgggcaacattgtgggcattattcgcaaaggcaaagatggcgaaggcgcgccgccggcgaaacgcccgcgcaccgatcagatggaagtggatagcggcccgggcaaacgcccgcataaaagcggctttaccgataaagaacgcgaagatcatcgccgccgcaaagcgctggaaaacaaaaaaaaacagctgagcgcgggcggcaaaattctgagcaaagaagaagaagaagaactgcgccgcctgaccgatgaagatgaagaacgcaaacgccgcgtggcgggcccgcgcgtgggcgatgtgaacccgagccgcggcggcccgcgcggcgcgccgggcggcggctttgtgccgcagatggcgggcgtgccggaaagcccgtttagccgcaccggcgaaggcctggatattcgcggcacccagggctttccgtgggtgagcccgagcccgccgcagcagcgcctgccgctgctggaaagcaccccgcagggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggc

SEQ ID NO: 150 - DNA, 2157 bp, synthetic construct
  source 1..2157: mol_type = other DNA
atggccggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggcggtggtggcagccgcagcgaaagcaaaaaaaaccgcggcggccgcgaagaaattctggaacagtgggtgggcgcgcgcaaaaaactggaagaactggaacgcgatctgcgcaaaattaaaaaaaaaattaaaaaactggaagaagaaaacccgtggctgggcaacattaaaggcattctgggcaaaaaagatcgcgaaggcgaaggcgcgccgccggcgaaacgcgcgcgcgcggatcagatggaagtggatagcggcccgcgcaaacgcccgtttcgcggcgaatttaccgataaagaacgccgcgatcatcgccgccgcaaagcgctggaaaacaaacgcaaacagctgagcagcggcggcaaaagcctgagcaaagaagaagaagaagaactgcgcaaactgaccgaagaagatgaacgccgcgaacgccgcgtggcgggcccgcgcgtgggcggcgtgaacccgctggaaggcggcacccgcggcgcgccgggcggcggctttgtgccgagcatgcagggcgtgccggaaagcccgtttgcgcgcaccggcgaaggcctggatgtgcgcggcaaccagggctttccgtgggatattctgtttccggcggatccgccgtttagcccgcagagcagtcgcccgcagggtggtggcggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggcggtggtggcagccagagcgaaacccgccgcggccgccgcggcacccgcgaagaaaccctggaaaaatggattaccgcgcgcaaaaaagcggaagaactggaaaaagatctgcgcaaaacccgcaaaaccattaaaaaactggaagaagaaaacccgtggctgggcaacattgtgggcattattcgcaaaggcaaagatggcgaaggcgcgccgccggcgaaacgcccgcgcaccgatcagatggaagtggatagcggcccgggcaaacgcccgcataaaagcggctttaccgataaagaacgcgaagatcatcgccgccgcaaagcgctggaaaacaaaaaaaaacagctgagcgcgggcggcaaaattctgagcaaagaagaagaagaagaactgcgccgcctgaccgatgaagatgaagaacgcaaacgccgcgtggcgggcccgcgcgtgggcgatgtgaacccgagccgcggcggcccgcgcggcgcgccgggcggcggctttgtgccgcagatggcgggcgtgccggaaagcccgtttagccgcaccggcgaaggcctggatattcgcggcacccagggctttccgtgggtgagcccgagcccgccgcagcagcgcctgccgctgctggaaagcaccccgcagggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggc

SEQ ID NO: 151 - DNA, 2166 bp, synthetic construct
  source 1..2166: mol_type = other DNA
atggccggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggcggtggtggcagccgcagcgaaagcaaaaaaaaccgcggcggccgcgaagaaattctggaacagtgggtgggcgcgcgcaaaaaactggaagaactggaacgcgatctgcgcaaaattaaaaaaaaaattaaaaaactggaagaagaaaacccgtggctgggcaacattaaaggcattctgggcaaaaaagatcgcgaaggcgaaggcgcgccgccggcgaaacgcgcgcgcgcggatcagatggaagtggatagcggcccgcgcaaacgcccgtttcgcggcgaatttaccgataaagaacgccgcgatcatcgccgccgcaaagcgctggaaaacaaacgcaaacagctgagcagcggcggcaaaagcctgagcaaagaagaagaagaagaactgcgcaaactgaccgaagaagatgaacgccgcgaacgccgcgtggcgggcccgcgcgtgggcggcgtgaacccgctggaaggcggcacccgcggcgcgccgggcggcggctttgtgccgagcatgcagggcgtgccggaaagcccgtttgcgcgcaccggcgaaggcctggatgtgcgcggcaaccagggctttccgtgggatattctgtttccggcggatccgccgtttagcccgcagagcagtcgcccgcagggtggtggcggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggcggtggtggcagccagagcgaaacccgccgcggccgccgcggcacccgcgaagaaaccctggaaaaatggattaccgcgcgcaaaaaagcggaagaactggaaaaagatctgcgcaaaacccgcaaaaccattaaaaaactggaagaagaaaacccgtggctgggcaacattgtgggcattattcgcaaaggcaaagatggcgaaggcgcgccgccggcgaaacgcccgcgcaccgatcagatggaagtggatagcggcccgggcaaacgcccgcataaaagcggctttaccgataaagaacgcgaagatcatcgccgccgcaaagcgctggaaaacaaaaaaaaacagctgagcgcgggcggcaaaattctgagcaaagaagaagaagaagaactgcgccgcctgaccgatgaagatgaagaacgcaaacgccgcgtggcgggcccgcgcgtgggcgatgtgaacccgagccgcggcggcccgcgcggcgcgccgggcggcggctttgtgccgcagatggcgggcgtgccggaaagcccgtttagccgcaccggcgaaggcctggatattcgcggcacccagggctttccgtgggtgagcccgagcccgccgcagcagcgcctgccgctgctggaaagcaccccgcagggtggtggcggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggc

SEQ ID NO: 152 - DNA, 2139 bp, synthetic construct
  source 1..2139: mol_type = other DNA
atggccggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggcagccgcagcgaaagcaaaaaaaaccgcggcggccgcgaagaaattctggaacagtgggtgggcgcgcgcaaaaaactggaagaactggaacgcgatctgcgcaaaattaaaaaaaaaattaaaaaactggaagaagaaaacccgtggctgggcaacattaaaggcattctgggcaaaaaagatcgcgaaggcgaaggcgcgccgccggcgaaacgcgcgcgcgcggatcagatggaagtggatagcggcccgcgcaaacgcccgtttcgcggcgaatttaccgataaagaacgccgcgatcatcgccgccgcaaagcgctggaaaacaaacgcaaacagctgagcagcggcggcaaaagcctgagcaaagaagaagaagaagaactgcgcaaactgaccgaagaagatgaacgccgcgaacgccgcgtggcgggcccgcgcgtgggcggcgtgaacccgctggaaggcggcacccgcggcgcgccgggcggcggctttgtgccgagcatgcagggcgtgccggaaagcccgtttgcgcgcaccggcgaaggcctggatgtgcgcggcaaccagggctttccgtgggatattctgtttccggcggatccgccgtttagcccgcagagcagtcgcccgcagggtggtggcggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggcagccagagcgaaacccgccgcggccgccgcggcacccgcgaagaaaccctggaaaaatggattaccgcgcgcaaaaaagcggaagaactggaaaaagatctgcgcaaaacccgcaaaaccattaaaaaactggaagaagaaaacccgtggctgggcaacattgtgggcattattcgcaaaggcaaagatggcgaaggcgcgccgccggcgaaacgcccgcgcaccgatcagatggaagtggatagcggcccgggcaaacgcccgcataaaagcggctttaccgataaagaacgcgaagatcatcgccgccgcaaagcgctggaaaacaaaaaaaaacagctgagcgcgggcggcaaaattctgagcaaagaagaagaagaagaactgcgccgcctgaccgatgaagatgaagaacgcaaacgccgcgtggcgggcccgcgcgtgggcgatgtgaacccgagccgcggcggcccgcgcggcgcgccgggcggcggctttgtgccgcagatggcgggcgtgccggaaagcccgtttagccgcaccggcgaaggcctggatattcgcggcacccagggctttccgtgggtgagcccgagcccgccgcagcagcgcctgccgctgctggaaagcaccccgcagggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggc

SEQ ID NO: 153 - DNA, 2148 bp, synthetic construct
  source 1..2148: mol_type = other DNA
atggccggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggcagccgcagcgaaagcaaaaaaaaccgcggcggccgcgaagaaattctggaacagtgggtgggcgcgcgcaaaaaactggaagaactggaacgcgatctgcgcaaaattaaaaaaaaaattaaaaaactggaagaagaaaacccgtggctgggcaacattaaaggcattctgggcaaaaaagatcgcgaaggcgaaggcgcgccgccggcgaaacgcgcgcgcgcggatcagatggaagtggatagcggcccgcgcaaacgcccgtttcgcggcgaatttaccgataaagaacgccgcgatcatcgccgccgcaaagcgctggaaaacaaacgcaaacagctgagcagcggcggcaaaagcctgagcaaagaagaagaagaagaactgcgcaaactgaccgaagaagatgaacgccgcgaacgccgcgtggcgggcccgcgcgtgggcggcgtgaacccgctggaaggcggcacccgcggcgcgccgggcggcggctttgtgccgagcatgcagggcgtgccggaaagcccgtttgcgcgcaccggcgaaggcctggatgtgcgcggcaaccagggctttccgtgggatattctgtttccggcggatccgccgtttagcccgcagagcagtcgcccgcagggtggtggcggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggcggtggtggcagccagagcgaaacccgccgcggccgccgcggcacccgcgaagaaaccctggaaaaatggattaccgcgcgcaaaaaagcggaagaactggaaaaagatctgcgcaaaacccgcaaaaccattaaaaaactggaagaagaaaacccgtggctgggcaacattgtgggcattattcgcaaaggcaaagatggcgaaggcgcgccgccggcgaaacgcccgcgcaccgatcagatggaagtggatagcggcccgggcaaacgcccgcataaaagcggctttaccgataaagaacgcgaagatcatcgccgccgcaaagcgctggaaaacaaaaaaaaacagctgagcgcgggcggcaaaattctgagcaaagaagaagaagaagaactgcgccgcctgaccgatgaagatgaagaacgcaaacgccgcgtggcgggcccgcgcgtgggcgatgtgaacccgagccgcggcggcccgcgcggcgcgccgggcggcggctttgtgccgcagatggcgggcgtgccggaaagcccgtttagccgcaccggcgaaggcctggatattcgcggcacccagggctttccgtgggtgagcccgagcccgccgcagcagcgcctgccgctgctggaaagcaccccgcagggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggc

SEQ ID NO: 154 - DNA, 2157 bp, synthetic construct
  source 1..2157: mol_type = other DNA
atggccggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggcagccgcagcgaaagcaaaaaaaaccgcggcggccgcgaagaaattctggaacagtgggtgggcgcgcgcaaaaaactggaagaactggaacgcgatctgcgcaaaattaaaaaaaaaattaaaaaactggaagaagaaaacccgtggctgggcaacattaaaggcattctgggcaaaaaagatcgcgaaggcgaaggcgcgccgccggcgaaacgcgcgcgcgcggatcagatggaagtggatagcggcccgcgcaaacgcccgtttcgcggcgaatttaccgataaagaacgccgcgatcatcgccgccgcaaagcgctggaaaacaaacgcaaacagctgagcagcggcggcaaaagcctgagcaaagaagaagaagaagaactgcgcaaactgaccgaagaagatgaacgccgcgaacgccgcgtggcgggcccgcgcgtgggcggcgtgaacccgctggaaggcggcacccgcggcgcgccgggcggcggctttgtgccgagcatgcagggcgtgccggaaagcccgtttgcgcgcaccggcgaaggcctggatgtgcgcggcaaccagggctttccgtgggatattctgtttccggcggatccgccgtttagcccgcagagcagtcgcccgcagggtggtggcggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggcggtggtggcagccagagcgaaacccgccgcggccgccgcggcacccgcgaagaaaccctggaaaaatggattaccgcgcgcaaaaaagcggaagaactggaaaaagatctgcgcaaaacccgcaaaaccattaaaaaactggaagaagaaaacccgtggctgggcaacattgtgggcattattcgcaaaggcaaagatggcgaaggcgcgccgccggcgaaacgcccgcgcaccgatcagatggaagtggatagcggcccgggcaaacgcccgcataaaagcggctttaccgataaagaacgcgaagatcatcgccgccgcaaagcgctggaaaacaaaaaaaaacagctgagcgcgggcggcaaaattctgagcaaagaagaagaagaagaactgcgccgcctgaccgatgaagatgaagaacgcaaacgccgcgtggcgggcccgcgcgtgggcgatgtgaacccgagccgcggcggcccgcgcggcgcgccgggcggcggctttgtgccgcagatggcgggcgtgccggaaagcccgtttagccgcaccggcgaaggcctggatattcgcggcacccagggctttccgtgggtgagcccgagcccgccgcagcagcgcctgccgctgctggaaagcaccccgcagggtggtggcggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggc

SEQ ID NO: 155 - DNA, 2139 bp, synthetic construct
  source 1..2139: mol_type = other DNA
atggccggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggcagccgcagcgaaagcaaaaaaaaccgcggcggccgcgaagaaattctggaacagtgggtgggcgcgcgcaaaaaactggaagaactggaacgcgatctgcgcaaaattaaaaaaaaaattaaaaaactggaagaagaaaacccgtggctgggcaacattaaaggcattctgggcaaaaaagatcgcgaaggcgaaggcgcgccgccggcgaaacgcgcgcgcgcggatcagatggaagtggatagcggcccgcgcaaacgcccgtttcgcggcgaatttaccgataaagaacgccgcgatcatcgccgccgcaaagcgctggaaaacaaacgcaaacagctgagcagcggcggcaaaagcctgagcaaagaagaagaagaagaactgcgcaaactgaccgaagaagatgaacgccgcgaacgccgcgtggcgggcccgcgcgtgggcggcgtgaacccgctggaaggcggcacccgcggcgcgccgggcggcggctttgtgccgagcatgcagggcgtgccggaaagcccgtttgcgcgcaccggcgaaggcctggatgtgcgcggcaaccagggctttccgtgggatattctgtttccggcggatccgccgtttagcccgcagagcagtcgcccgcagggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggcggtggtggcagccagagcgaaacccgccgcggccgccgcggcacccgcgaagaaaccctggaaaaatggattaccgcgcgcaaaaaagcggaagaactggaaaaagatctgcgcaaaacccgcaaaaccattaaaaaactggaagaagaaaacccgtggctgggcaacattgtgggcattattcgcaaaggcaaagatggcgaaggcgcgccgccggcgaaacgcccgcgcaccgatcagatggaagtggatagcggcccgggcaaacgcccgcataaaagcggctttaccgataaagaacgcgaagatcatcgccgccgcaaagcgctggaaaacaaaaaaaaacagctgagcgcgggcggcaaaattctgagcaaagaagaagaagaagaactgcgccgcctgaccgatgaagatgaagaacgcaaacgccgcgtggcgggcccgcgcgtgggcgatgtgaacccgagccgcggcggcccgcgcggcgcgccgggcggcggctttgtgccgcagatggcgggcgtgccggaaagcccgtttagccgcaccggcgaaggcctggatattcgcggcacccagggctttccgtgggtgagcccgagcccgccgcagcagcgcctgccgctgctggaaagcaccccgcagggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggc

SEQ ID NO: 156 - DNA, 2148 bp, synthetic construct
  source 1..2148: mol_type = other DNA
atggccggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggcagccgcagcgaaagcaaaaaaaaccgcggcggccgcgaagaaattctggaacagtgggtgggcgcgcgcaaaaaactggaagaactggaacgcgatctgcgcaaaattaaaaaaaaaattaaaaaactggaagaagaaaacccgtggctgggcaacattaaaggcattctgggcaaaaaagatcgcgaaggcgaaggcgcgccgccggcgaaacgcgcgcgcgcggatcagatggaagtggatagcggcccgcgcaaacgcccgtttcgcggcgaatttaccgataaagaacgccgcgatcatcgccgccgcaaagcgctggaaaacaaacgcaaacagctgagcagcggcggcaaaagcctgagcaaagaagaagaagaagaactgcgcaaactgaccgaagaagatgaacgccgcgaacgccgcgtggcgggcccgcgcgtgggcggcgtgaacccgctggaaggcggcacccgcggcgcgccgggcggcggctttgtgccgagcatgcagggcgtgccggaaagcccgtttgcgcgcaccggcgaaggcctggatgtgcgcggcaaccagggctttccgtgggatattctgtttccggcggatccgccgtttagcccgcagagcagtcgcccgcagggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggcggtggtggcagccagagcgaaacccgccgcggccgccgcggcacccgcgaagaaaccctggaaaaatggattaccgcgcgcaaaaaagcggaagaactggaaaaagatctgcgcaaaacccgcaaaaccattaaaaaactggaagaagaaaacccgtggctgggcaacattgtgggcattattcgcaaaggcaaagatggcgaaggcgcgccgccggcgaaacgcccgcgcaccgatcagatggaagtggatagcggcccgggcaaacgcccgcataaaagcggctttaccgataaagaacgcgaagatcatcgccgccgcaaagcgctggaaaacaaaaaaaaacagctgagcgcgggcggcaaaattctgagcaaagaagaagaagaagaactgcgccgcctgaccgatgaagatgaagaacgcaaacgccgcgtggcgggcccgcgcgtgggcgatgtgaacccgagccgcggcggcccgcgcggcgcgccgggcggcggctttgtgccgcagatggcgggcgtgccggaaagcccgtttagccgcaccggcgaaggcctggatattcgcggcacccagggctttccgtgggtgagcccgagcccgccgcagcagcgcctgccgctgctggaaagcaccccgcagggtggtggcggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggc

SEQ ID NO: 157 - DNA, 2157 bp, synthetic construct
  source 1..2157: mol_type = other DNA
atggccggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggcggtggtggcagccgcagcgaaagcaaaaaaaaccgcggcggccgcgaagaaattctggaacagtgggtgggcgcgcgcaaaaaactggaagaactggaacgcgatctgcgcaaaattaaaaaaaaaattaaaaaactggaagaagaaaacccgtggctgggcaacattaaaggcattctgggcaaaaaagatcgcgaaggcgaaggcgcgccgccggcgaaacgcgcgcgcgcggatcagatggaagtggatagcggcccgcgcaaacgcccgtttcgcggcgaatttaccgataaagaacgccgcgatcatcgccgccgcaaagcgctggaaaacaaacgcaaacagctgagcagcggcggcaaaagcctgagcaaagaagaagaagaagaactgcgcaaactgaccgaagaagatgaacgccgcgaacgccgcgtggcgggcccgcgcgtgggcggcgtgaacccgctggaaggcggcacccgcggcgcgccgggcggcggctttgtgccgagcatgcagggcgtgccggaaagcccgtttgcgcgcaccggcgaaggcctggatgtgcgcggcaaccagggctttccgtgggatattctgtttccggcggatccgccgtttagcccgcagagcagtcgcccgcagggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggcggtggtggcagccagagcgaaacccgccgcggccgccgcggcacccgcgaagaaaccctggaaaaatggattaccgcgcgcaaaaaagcggaagaactggaaaaagatctgcgcaaaacccgcaaaaccattaaaaaactggaagaagaaaacccgtggctgggcaacattgtgggcattattcgcaaaggcaaagatggcgaaggcgcgccgccggcgaaacgcccgcgcaccgatcagatggaagtggatagcggcccgggcaaacgcccgcataaaagcggctttaccgataaagaacgcgaagatcatcgccgccgcaaagcgctggaaaacaaaaaaaaacagctgagcgcgggcggcaaaattctgagcaaagaagaagaagaagaactgcgccgcctgaccgatgaagatgaagaacgcaaacgccgcgtggcgggcccgcgcgtgggcgatgtgaacccgagccgcggcggcccgcgcggcgcgccgggcggcggctttgtgccgcagatggcgggcgtgccggaaagcccgtttagccgcaccggcgaaggcctggatattcgcggcacccagggctttccgtgggtgagcccgagcccgccgcagcagcgcctgccgctgctggaaagcaccccgcagggtggtggcggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggc

SEQ ID NO: 158 - DNA, 2148 bp, synthetic construct
  source 1..2148: mol_type = other DNA
atggccggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggcggtggtggcagccgcagcgaaagcaaaaaaaaccgcggcggccgcgaagaaattctggaacagtgggtgggcgcgcgcaaaaaactggaagaactggaacgcgatctgcgcaaaattaaaaaaaaaattaaaaaactggaagaagaaaacccgtggctgggcaacattaaaggcattctgggcaaaaaagatcgcgaaggcgaaggcgcgccgccggcgaaacgcgcgcgcgcggatcagatggaagtggatagcggcccgcgcaaacgcccgtttcgcggcgaatttaccgataaagaacgccgcgatcatcgccgccgcaaagcgctggaaaacaaacgcaaacagctgagcagcggcggcaaaagcctgagcaaagaagaagaagaagaactgcgcaaactgaccgaagaagatgaacgccgcgaacgccgcgtggcgggcccgcgcgtgggcggcgtgaacccgctggaaggcggcacccgcggcgcgccgggcggcggctttgtgccgagcatgcagggcgtgccggaaagcccgtttgcgcgcaccggcgaaggcctggatgtgcgcggcaaccagggctttccgtgggatattctgtttccggcggatccgccgtttagcccgcagagcagtcgcccgcagggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggcggtggtggcagccagagcgaaacccgccgcggccgccgcggcacccgcgaagaaaccctggaaaaatggattaccgcgcgcaaaaaagcggaagaactggaaaaagatctgcgcaaaacccgcaaaaccattaaaaaactggaagaagaaaacccgtggctgggcaacattgtgggcattattcgcaaaggcaaagatggcgaaggcgcgccgccggcgaaacgcccgcgcaccgatcagatggaagtggatagcggcccgggcaaacgcccgcataaaagcggctttaccgataaagaacgcgaagatcatcgccgccgcaaagcgctggaaaacaaaaaaaaacagctgagcgcgggcggcaaaattctgagcaaagaagaagaagaagaactgcgccgcctgaccgatgaagatgaagaacgcaaacgccgcgtggcgggcccgcgcgtgggcgatgtgaacccgagccgcggcggcccgcgcggcgcgccgggcggcggctttgtgccgcagatggcgggcgtgccggaaagcccgtttagccgcaccggcgaaggcctggatattcgcggcacccagggctttccgtgggtgagcccgagcccgccgcagcagcgcctgccgctgctggaaagcaccccgcagggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggc

SEQ ID NO: 159 - DNA, 2148 bp, synthetic construct
  source 1..2148: mol_type = other DNA
atggccggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggcggtggtggcagccgcagcgaaagcaaaaaaaaccgcggcggccgcgaagaaattctggaacagtgggtgggcgcgcgcaaaaaactggaagaactggaacgcgatctgcgcaaaattaaaaaaaaaattaaaaaactggaagaagaaaacccgtggctgggcaacattaaaggcattctgggcaaaaaagatcgcgaaggcgaaggcgcgccgccggcgaaacgcgcgcgcgcggatcagatggaagtggatagcggcccgcgcaaacgcccgtttcgcggcgaatttaccgataaagaacgccgcgatcatcgccgccgcaaagcgctggaaaacaaacgcaaacagctgagcagcggcggcaaaagcctgagcaaagaagaagaagaagaactgcgcaaactgaccgaagaagatgaacgccgcgaacgccgcgtggcgggcccgcgcgtgggcggcgtgaacccgctggaaggcggcacccgcggcgcgccgggcggcggctttgtgccgagcatgcagggcgtgccggaaagcccgtttgcgcgcaccggcgaaggcctggatgtgcgcggcaaccagggctttccgtgggatattctgtttccggcggatccgccgtttagcccgcagagcagtcgcccgcagggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggcagccagagcgaaacccgccgcggccgccgcggcacccgcgaagaaaccctggaaaaatggattaccgcgcgcaaaaaagcggaagaactggaaaaagatctgcgcaaaacccgcaaaaccattaaaaaactggaagaagaaaacccgtggctgggcaacattgtgggcattattcgcaaaggcaaagatggcgaaggcgcgccgccggcgaaacgcccgcgcaccgatcagatggaagtggatagcggcccgggcaaacgcccgcataaaagcggctttaccgataaagaacgcgaagatcatcgccgccgcaaagcgctggaaaacaaaaaaaaacagctgagcgcgggcggcaaaattctgagcaaagaagaagaagaagaactgcgccgcctgaccgatgaagatgaagaacgcaaacgccgcgtggcgggcccgcgcgtgggcgatgtgaacccgagccgcggcggcccgcgcggcgcgccgggcggcggctttgtgccgcagatggcgggcgtgccggaaagcccgtttagccgcaccggcgaaggcctggatattcgcggcacccagggctttccgtgggtgagcccgagcccgccgcagcagcgcctgccgctgctggaaagcaccccgcagggtggtggcggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggc

SEQ ID NO: 160 - DNA, 2157 bp, synthetic construct
  source 1..2157: mol_type = other DNA
atggccggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggcggtggtggcagccgcagcgaaagcaaaaaaaaccgcggcggccgcgaagaaattctggaacagtgggtgggcgcgcgcaaaaaactggaagaactggaacgcgatctgcgcaaaattaaaaaaaaaattaaaaaactggaagaagaaaacccgtggctgggcaacattaaaggcattctgggcaaaaaagatcgcgaaggcgaaggcgcgccgccggcgaaacgcgcgcgcgcggatcagatggaagtggatagcggcccgcgcaaacgcccgtttcgcggcgaatttaccgataaagaacgccgcgatcatcgccgccgcaaagcgctggaaaacaaacgcaaacagctgagcagcggcggcaaaagcctgagcaaagaagaagaagaagaactgcgcaaactgaccgaagaagatgaacgccgcgaacgccgcgtggcgggcccgcgcgtgggcggcgtgaacccgctggaaggcggcacccgcggcgcgccgggcggcggctttgtgccgagcatgcagggcgtgccggaaagcccgtttgcgcgcaccggcgaaggcctggatgtgcgcggcaaccagggctttccgtgggatattctgtttccggcggatccgccgtttagcccgcagagcagtcgcccgcagggtggtggcggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggcagccagagcgaaacccgccgcggccgccgcggcacccgcgaagaaaccctggaaaaatggattaccgcgcgcaaaaaagcggaagaactggaaaaagatctgcgcaaaacccgcaaaaccattaaaaaactggaagaagaaaacccgtggctgggcaacattgtgggcattattcgcaaaggcaaagatggcgaaggcgcgccgccggcgaaacgcccgcgcaccgatcagatggaagtggatagcggcccgggcaaacgcccgcataaaagcggctttaccgataaagaacgcgaagatcatcgccgccgcaaagcgctggaaaacaaaaaaaaacagctgagcgcgggcggcaaaattctgagcaaagaagaagaagaagaactgcgccgcctgaccgatgaagatgaagaacgcaaacgccgcgtggcgggcccgcgcgtgggcgatgtgaacccgagccgcggcggcccgcgcggcgcgccgggcggcggctttgtgccgcagatggcgggcgtgccggaaagcccgtttagccgcaccggcgaaggcctggatattcgcggcacccagggctttccgtgggtgagcccgagcccgccgcagcagcgcctgccgctgctggaaagcaccccgcagggtggtggcggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggc

SEQ ID NO: 161 - DNA, 2148 bp, synthetic construct
  source 1..2148: mol_type = other DNA
atggccggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggcagccgcagcgaaagcaaaaaaaaccgcggcggccgcgaagaaattctggaacagtgggtgggcgcgcgcaaaaaactggaagaactggaacgcgatctgcgcaaaattaaaaaaaaaattaaaaaactggaagaagaaaacccgtggctgggcaacattaaaggcattctgggcaaaaaagatcgcgaaggcgaaggcgcgccgccggcgaaacgcgcgcgcgcggatcagatggaagtggatagcggcccgcgcaaacgcccgtttcgcggcgaatttaccgataaagaacgccgcgatcatcgccgccgcaaagcgctggaaaacaaacgcaaacagctgagcagcggcggcaaaagcctgagcaaagaagaagaagaagaactgcgcaaactgaccgaagaagatgaacgccgcgaacgccgcgtggcgggcccgcgcgtgggcggcgtgaacccgctggaaggcggcacccgcggcgcgccgggcggcggctttgtgccgagcatgcagggcgtgccggaaagcccgtttgcgcgcaccggcgaaggcctggatgtgcgcggcaaccagggctttccgtgggatattctgtttccggcggatccgccgtttagcccgcagagcagtcgcccgcagggtggtggcggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggcagccagagcgaaacccgccgcggccgccgcggcacccgcgaagaaaccctggaaaaatggattaccgcgcgcaaaaaagcggaagaactggaaaaagatctgcgcaaaacccgcaaaaccattaaaaaactggaagaagaaaacccgtggctgggcaacattgtgggcattattcgcaaaggcaaagatggcgaaggcgcgccgccggcgaaacgcccgcgcaccgatcagatggaagtggatagcggcccgggcaaacgcccgcataaaagcggctttaccgataaagaacgcgaagatcatcgccgccgcaaagcgctggaaaacaaaaaaaaacagctgagcgcgggcggcaaaattctgagcaaagaagaagaagaagaactgcgccgcctgaccgatgaagatgaagaacgcaaacgccgcgtggcgggcccgcgcgtgggcgatgtgaacccgagccgcggcggcccgcgcggcgcgccgggcggcggctttgtgccgcagatggcgggcgtgccggaaagcccgtttagccgcaccggcgaaggcctggatattcgcggcacccagggctttccgtgggtgagcccgagcccgccgcagcagcgcctgccgctgctggaaagcaccccgcagggtggtggcggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggc

SEQ ID NO: 162 - DNA, 2139 bp, synthetic construct
  source 1..2139: mol_type = other DNA
atggccggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggcagccgcagcgaaagcaaaaaaaaccgcggcggccgcgaagaaattctggaacagtgggtgggcgcgcgcaaaaaactggaagaactggaacgcgatctgcgcaaaattaaaaaaaaaattaaaaaactggaagaagaaaacccgtggctgggcaacattaaaggcattctgggcaaaaaagatcgcgaaggcgaaggcgcgccgccggcgaaacgcgcgcgcgcggatcagatggaagtggatagcggcccgcgcaaacgcccgtttcgcggcgaatttaccgataaagaacgccgcgatcatcgccgccgcaaagcgctggaaaacaaacgcaaacagctgagcagcggcggcaaaagcctgagcaaagaagaagaagaagaactgcgcaaactgaccgaagaagatgaacgccgcgaacgccgcgtggcgggcccgcgcgtgggcggcgtgaacccgctggaaggcggcacccgcggcgcgccgggcggcggctttgtgccgagcatgcagggcgtgccggaaagcccgtttgcgcgcaccggcgaaggcctggatgtgcgcggcaaccagggctttccgtgggatattctgtttccggcggatccgccgtttagcccgcagagcagtcgcccgcagggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggcagccagagcgaaacccgccgcggccgccgcggcacccgcgaagaaaccctggaaaaatggattaccgcgcgcaaaaaagcggaagaactggaaaaagatctgcgcaaaacccgcaaaaccattaaaaaactggaagaagaaaacccgtggctgggcaacattgtgggcattattcgcaaaggcaaagatggcgaaggcgcgccgccggcgaaacgcccgcgcaccgatcagatggaagtggatagcggcccgggcaaacgcccgcataaaagcggctttaccgataaagaacgcgaagatcatcgccgccgcaaagcgctggaaaacaaaaaaaaacagctgagcgcgggcggcaaaattctgagcaaagaagaagaagaagaactgcgccgcctgaccgatgaagatgaagaacgcaaacgccgcgtggcgggcccgcgcgtgggcgatgtgaacccgagccgcggcggcccgcgcggcgcgccgggcggcggctttgtgccgcagatggcgggcgtgccggaaagcccgtttagccgcaccggcgaaggcctggatattcgcggcacccagggctttccgtgggtgagcccgagcccgccgcagcagcgcctgccgctgctggaaagcaccccgcagggtggtggcggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggc

SEQ ID NO: 163 - protein, 713 aa, synthetic construct
MAGTNLSTSNPLGFFPDHQLDPAFRANSANPDWDFNPNKDTWPDANKVGGQNLSTSNPLGFFPDHQLDPAFRANTANPDWDFNPNKDTWPDANKVGGGGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 164 - protein, 716 aa, synthetic construct
MAGTNLSTSNPLGFFPDHQLDPAFRANSANPDWDFNPNKDTWPDANKVGGQNLSTSNPLGFFPDHQLDPAFRANTANPDWDFNPNKDTWPDANKVGGGGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGGGGTNLSTSNPLGFFPDHQLDPAFRANSANPDWDFNPNKDTWPDANKVGGQNLSTSNPLGFFPDHQLDPAFRANTANPDWDFNPNKDTWPDANKVGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 165 - protein, 719 aa, synthetic construct
MAGTNLSTSNPLGFFPDHQLDPAFRANSANPDWDFNPNKDTWPDANKVGGQNLSTSNPLGFFPDHQLDPAFRANTANPDWDFNPNKDTWPDANKVGGGGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGGGGTNLSTSNPLGFFPDHQLDPAFRANSANPDWDFNPNKDTWPDANKVGGQNLSTSNPLGFFPDHQLDPAFRANTANPDWDFNPNKDTWPDANKVGGGGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 166 - protein, 722 aa, synthetic construct
MAGTNLSTSNPLGFFPDHQLDPAFRANSANPDWDFNPNKDTWPDANKVGGQNLSTSNPLGFFPDHQLDPAFRANTANPDWDFNPNKDTWPDANKVGGGGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGGGGTNLSTSNPLGFFPDHQLDPAFRANSANPDWDFNPNKDTWPDANKVGGQNLSTSNPLGFFPDHQLDPAFRANTANPDWDFNPNKDTWPDANKVGGGGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGGGGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 167 - protein, 713 aa, synthetic construct
MAGTNLSTSNPLGFFPDHQLDPAFRANSANPDWDFNPNKDTWPDANKVGGQNLSTSNPLGFFPDHQLDPAFRANTANPDWDFNPNKDTWPDANKVGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGGGGTNLSTSNPLGFFPDHQLDPAFRANSANPDWDFNPNKDTWPDANKVGGQNLSTSNPLGFFPDHQLDPAFRANTANPDWDFNPNKDTWPDANKVGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 168 - protein, 716 aa, synthetic construct
MAGTNLSTSNPLGFFPDHQLDPAFRANSANPDWDFNPNKDTWPDANKVGGQNLSTSNPLGFFPDHQLDPAFRANTANPDWDFNPNKDTWPDANKVGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGGGGTNLSTSNPLGFFPDHQLDPAFRANSANPDWDFNPNKDTWPDANKVGGQNLSTSNPLGFFPDHQLDPAFRANTANPDWDFNPNKDTWPDANKVGGGGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 169 - protein, 719 aa, synthetic construct
MAGTNLSTSNPLGFFPDHQLDPAFRANSANPDWDFNPNKDTWPDANKVGGQNLSTSNPLGFFPDHQLDPAFRANTANPDWDFNPNKDTWPDANKVGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGGGGTNLSTSNPLGFFPDHQLDPAFRANSANPDWDFNPNKDTWPDANKVGGQNLSTSNPLGFFPDHQLDPAFRANTANPDWDFNPNKDTWPDANKVGGGGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGGGGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 170 - protein, 713 aa, synthetic construct
MAGTNLSTSNPLGFFPDHQLDPAFRANSANPDWDFNPNKDTWPDANKVGGQNLSTSNPLGFFPDHQLDPAFRANTANPDWDFNPNKDTWPDANKVGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGGGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 171 - protein, 716 aa, synthetic construct
MAGTNLSTSNPLGFFPDHQLDPAFRANSANPDWDFNPNKDTWPDANKVGGQNLSTSNPLGFFPDHQLDPAFRANTANPDWDFNPNKDTWPDANKVGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGGGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGGGGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 172 - protein, 719 aa, synthetic construct
MAGTNLSTSNPLGFFPDHQLDPAFRANSANPDWDFNPNKDTWPDANKVGGQNLSTSNPLGFFPDHQLDPAFRANTANPDWDFNPNKDTWPDANKVGGGGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGGGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGGGGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 173 - protein, 716 aa, synthetic construct
MAGTNLSTSNPLGFFPDHQLDPAFRANSANPDWDFNPNKDTWPDANKVGGQNLSTSNPLGFFPDHQLDPAFRANTANPDWDFNPNKDTWPDANKVGGGGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGGGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 174 - protein, 716 aa, synthetic construct
MAGTNLSTSNPLGFFPDHQLDPAFRANSANPDWDFNPNKDTWPDANKVGGQNLSTSNPLGFFPDHQLDPAFRANTANPDWDFNPNKDTWPDANKVGGGGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGGGGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 175 - protein, 719 aa, synthetic construct
MAGTNLSTSNPLGFFPDHQLDPAFRANSANPDWDFNPNKDTWPDANKVGGQNLSTSNPLGFFPDHQLDPAFRANTANPDWDFNPNKDTWPDANKVGGGGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGGGGTNLSTSNPLGFFPDHQLDPAFRANSANPDWDFNPNKDTWPDANKVGGQNLSTSNPLGFFPDHQLDPAFRANTANPDWDFNPNKDTWPDANKVGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGGGGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 176 - protein, 716 aa, synthetic construct
MAGTNLSTSNPLGFFPDHQLDPAFRANSANPDWDFNPNKDTWPDANKVGGQNLSTSNPLGFFPDHQLDPAFRANTANPDWDFNPNKDTWPDANKVGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGGGGTNLSTSNPLGFFPDHQLDPAFRANSANPDWDFNPNKDTWPDANKVGGQNLSTSNPLGFFPDHQLDPAFRANTANPDWDFNPNKDTWPDANKVGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGGGGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 177 - protein, 713 aa, synthetic construct
MAGTNLSTSNPLGFFPDHQLDPAFRANSANPDWDFNPNKDTWPDANKVGGQNLSTSNPLGFFPDHQLDPAFRANTANPDWDFNPNKDTWPDANKVGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGGGGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 178 - DNA, 3177 bp, synthetic construct
  source 1..3177: mol_type = other DNA
atgagccagagcgaaacccgtcgtggccgtcgtggtacccgtgaagaaaccctggaaaagtggatcaccgcgcgtaagaaagcggaagagctggagaaagacctgcgtaagacccgtaaaaccatcaagaaactggaagaggaaaacccgtggctgggcaacattgtgggtatcattcgtaagggcaaagatggtgagggagccccgccggctaagcgtccgcgtaccgaccagatggaagtggatagcggcccgggcaagcgtccgcacaaaagcggtttcaccgacaaggagcgtgaagaccatcgtcgtcgtaaagcgctggaaaacaagaaaaagcaactgagcgcgggtggcaagattttatctaaagaggaagaggaagagttacgtcgtctgaccgacgaggatgaagagcgtaagcgtcgtgttgcgggcccgcgtgtgggtgatgttaacccgagccgtggtggcccgcgtggcgcgccgggtggcggtttcgttccgcagatggcgggtgttccggaaagcccgtttagccgcaccggcgagggtctggatattcgtggcacgcaaggttttccgtgggttagcccgagcccgccgcagcaacgtctgccgctgctggagagcaccccgcagggtggtggcagccaaagcgagagcaaaaagaaccgtcgtggcggtcgtgaggacatcctggaaaagtggattaccacccgtcgtaaggcggaggaactggaaaaggacctgcgtaaggcgcgtaaaaccatcaaaaagctggaggatgagaatccgtggctgggtaatatcattggtatcattcgtaagggtaaagatggtgagggggctccgccggctaagcgtccgagaaccgaccagatggagattgatagcggcaccggcaagcgtccgcataagagcggctttaccgataaagagcgtgaagaccaccgtcgtcgtaaagcgctggaaaataaaaagaaacagctgagcagcggcggtaaaaacctgagccgtgaagaggaagaggaattaggtcgtctgaccgtggaggacgaagaacgtcgtcgtcgtgtggcgggtccgcgtaccggtgatgttaacctgagcggcggtggccctcgtggcgcgccgggtggcggtttcgtaccgcgtatggagggtgttcctgagagcccgtttacccgcaccggcgaaggtctggacatccgtggcaatcaaggctttccgtgggttcgtccgagcccgccgcagcagcgcctgccgctgctggaaagcaccccgcaggggacaaatctatcaaccagcaacccgctgggctttttcccggaccaccaactggacccggcgttccgtgcgaacagcgcgaacccggactgggattttaacccgaacaaggacacctggccggacgcgaataaagtgggtggccagaatttatctaccagcaacccgctgggtttctttccggaccaccaactggacccggcgtttcgtgcgaacaccgcgaatccggattgggacttcaatccgaataaggacacctggccggatgcgaataaagtgggcggtggtggcagccgtagcgagagcaagaaaaaccgtggtggccgtgaggaaatcctggaacagtgggttggtgcgcgtaagaaactggaggaactggagcgtgacctgcgtaagatcaagaaaaagattaaaaagctggaggaagagaacccgtggctgggtaacatcaaaggcattctgggtaaaaaggatcgtgagggtgaaggtgctccgccggcgaagcgtgcgcgtgcggaccagatggaagttgatagcggcccgcgtaaacgtccgttccgtggtgagtttaccgacaaggaacgtcgtgatcaccgtcgtcgtaaagcgctggagaacaagcgtaaacaactgagcagcggtggcaagagcctgagcaaagaagaggaagaggaactgcgtaagctgaccgaggaagacgaacgtcgtgaacgtcgtgttgcgggtccgcgtgtgggtggcgttaacccgctggagggtggcacccgtggcgcgccgggtggcggtttcgtgccgagcatgcagggtgttccggagagcccgtttgcgcgtaccggcgaaggtctggatgttcgtggcaatcaaggtttcccgtgggacatcctgtttccggcggacccgccgtttagcccgcagagcagccgtccgcaaggtggtggcagccgtagcgaaagcaaaaagaaccgtggcggtcgtgaggaagtgctggagcagtgggttaacggccgtaaaaagctggaagagctggagcgtgaactgcgtcgtgcgcgtaaaaagatcaaaaagctggaagacgataatccgtggctgggcaacgtgaagggtatcctgggtaaaaaggacaaagatggtgagggcgctccgccggcgaaacgcgcgcgtaccgaccaaatggagattgatagcggtccgcgtaaacgtccgctgcgtggcggtttcaccgaccgtgagcgtcaggatcaccgtcgtcgtaaggcgctgaaaaacaaaaagaaacaactgagcgcgggcggtaagagcctgagcaaagaggaagaggaagagctgaaacgtctgacccgtgaagacgaagagcgtaagaaagaagagcatggtccgagccgtctgggtgtgaacccgagcgagggcggtccgcgtggcgcgccgggcggtggcttcgttccgagcatgcagggtatcccggagagccgttttacccgcaccggtgaaggtctggacgttcgtggcagccgtggttttccgcaagacattctgttcccgagcgatccgccgttcagcccgcagagcagccgcccgcaaggtaccaatttaagtaccagcaacccgctgggtttctttccagaccaccagctggacccggcgttccgcgcgaatagcgcgaatccggactgggacttcaatccgaacaaagacacttggcctgacgcgaacaaagtgggtggccagaacttatctaccagcaacccgctgggtttctttcctgaccaccaactggacccggcgtttcgcgcgaacaccgcgaacccggattgggacttcaacccgaataaagacacttggcccgacgcgaacaaagttggtggatcccatcatcatcatcatcattaa

SEQ ID NO: 179 - DNA, 3467 bp, synthetic construct
  source 1..3467: mol_type = other DNA
atggggacaaatctatcaaccagcaacccgctgggctttttcccggaccaccaactggacccggcgttccgtgcgaacagcgcgaacccggactgggattttaacccgaacaaggacacctggccggacgcgaataaagtgggtggccagaatttatctaccagcaacccgctgggtttctttccggaccaccaactggacccggcgtttcgtgcgaacaccgcgaatccggattgggacttcaatccgaataaggacacctggccggatgcgaataaagtgggcggtggtggcagccagagcgaaacccgtcgtggccgtcgtggtacccgtgaagaaaccctggaaaagtggatcaccgcgcgtaagaaagcggaagagctggagaaagacctgcgtaagacccgtaaaaccatcaagaaactggaagaggaaaacccgtggctgggcaacattgtgggtatcattcgtaagggcaaagatggtgagggagccccgccggctaagcgtccgcgtaccgaccagatggaagtggatagcggcccgggcaagcgtccgcacaaaagcggtttcaccgacaaggagcgtgaagaccatcgtcgtcgtaaagcgctggaaaacaagaaaaagcaactgagcgcgggtggcaagattttatctaaagaggaagaggaagagttacgtcgtctgaccgacgaggatgaagagcgtaagcgtcgtgttgcgggcccgcgtgtgggtgatgttaacccgagccgtggtggcccgcgtggcgcgccgggtggcggtttcgttccgcagatggcgggtgttccggaaagcccgtttagccgcaccggcgagggtctggatattcgtggcacgcaaggttttccgtgggttagcccgagcccgccgcagcaacgtctgccgctgctggagagcaccccgcagggtggtggcagccaaagcgagagcaaaaagaaccgtcgtggcggtcgtgaggacatcctggaaaagtggattaccacccgtcgtaaggcggaggaactggaaaaggacctgcgtaaggcgcgtaaaaccatcaaaaagctggaggatgagaatccgtggctgggtaatatcattggtatcattcgtaagggtaaagatggtgagggggctccgccggctaagcgtccgagaaccgaccagatggagattgatagcggcaccggcaagcgtccgcataagagcggctttaccgataaagagcgtgaagaccaccgtcgtcgtaaagcgctggaaaataaaaagaaacagctgagcagcggcggtaaaaacctgagccgtgaagaggaagaggaattaggtcgtctgaccgtggaggacgaagaacgtcgtcgtcgtgtggcgggtccgcgtaccggtgatgttaacctgagcggcggtggccctcgtggcgcgccgggtggcggtttcgtaccgcgtatggagggtgttcctgagagcccgtttacccgcaccggcgaaggtctggacatccgtggcaatcaaggctttccgtgggttcgtccgagcccgccgcagcagcgcctgccgctgctggaaagcaccccgcagggtaccaatttaagcaccagcaacccgctgggtttctttcccgatcatcaactggacccggcgttccgtgcgaattctgctaacccggattgggactttaatcctaataaagatacttggccggacgcgaataaggtgggcggtcagaatctttctaccagcaacccgctgggtttctttccagatcatcagctggacccggcgtttcgtgcgaataccgcgaacccggattgggatttcaacccgaataaagatacctggccggatgcgaacaaagtgggcggtggtggcagccgtagcgagagcaagaaaaaccgtggtggccgtgaggaaatcctggaacagtgggttggtgcgcgtaagaaactggaggaactggagcgtgacctgcgtaagatcaagaaaaagattaaaaagctggaggaagagaacccgtggctgggtaacatcaaaggcattctgggtaaaaaggatcgtgagggtgaaggtgctccgccggcgaagcgtgcgcgtgcggaccagatggaagttgatagcggcccgcgtaaacgtccgttccgtggtgagtttaccgacaaggaacgtcgtgatcaccgtcgtcgtaaagcgctggagaacaagcgtaaacaactgagcagcggtggcaagagcctgagcaaagaagaggaagaggaactgcgtaagctgaccgaggaagacgaacgtcgtgaacgtcgtgttgcgggtccgcgtgtgggtggcgttaacccgctggagggtggcacccgtggcgcgccgggtggcggtttcgtgccgagcatgcagggtgttccggagagcccgtttgcgcgtaccggcgaaggtctggatgttcgtggcaatcaaggtttcccgtgggacatcctgtttccggcggacccgccgtttagcccgcagagcagccgtccgcaaggtggtggcagccgtagcgaaagcaaaaagaaccgtggcggtcgtgaggaagtgctggagcagtgggttaacggccgtaaaaagctggaagagctggagcgtgaactgcgtcgtgcgcgtaaaaagatcaaaaagctggaagacgataatccgtggctgggcaacgtgaagggtatcctgggtaaaaaggacaaagatggtgagggcgctccgccggcgaaacgcgcgcgtaccgaccaaatggagattgatagcggtccgcgtaaacgtccgctgcgtggcggtttcaccgaccgtgagcgtcaggatcaccgtcgtcgtaaggcgctgaaaaacaaaaagaaacaactgagcgcgggcggtaagagcctgagcaaagaggaagaggaagagctgaaacgtctgacccgtgaagacgaagagcgtaagaaagaagagcatggtccgagccgtctgggtgtgaacccgagcgagggcggtccgcgtggcgcgccgggcggtggcttcgttccgagcatgcagggtatcccggagagccgttttacccgcaccggtgaaggtctggacgttcgtggcagccgtggttttccgcaagacattctgttcccgagcgatccgccgttcagcccgcagagcagccgcccgcaaggtaccaatttaagtaccagcaacccgctgggtttctttccagaccaccagctggacccggcgttccgcgcgaatagcgcgaatccggactgggacttcaatccgaacaaagacacttggcctgacgcgaacaaagtgggtggccagaacttatctaccagcaacccgctgggtttctttcctgaccaccaactggacccggcgtttcgcgcgaacaccgcgaacccggattgggacttcaacccgaataaagacacttggcccgacgcgaacaaagttggtggatcccatcatcaccaccaccacta

SEQ ID NO: 180 - DNA, 3444 bp, synthetic construct
  source 1..3444: mol_type = other DNA
atggggacaaatctatcaaccagcaacccgctgggctttttcccggaccaccaactggacccggcgttccgtgcgaacagcgcgaacccggactgggattttaacccgaacaaggacacctggccggacgcgaataaagtgggtggccagaatttatctaccagcaacccgctgggtttctttccggaccaccaactggacccggcgtttcgtgcgaacaccgcgaatccggattgggacttcaatccgaataaggacacctggccggatgcgaataaagtgggcggtggtggcagccagagcgaaacccgtcgtggccgtcgtggtacccgtgaagaaaccctggaaaagtggatcaccgcgcgtaagaaagcggaagagctggagaaagacctgcgtaagacccgtaaaaccatcaagaaactggaagaggaaaacccgtggctgggcaacattgtgggtatcattcgtaagggcaaagatggtgagggagccccgccggctaagcgtccgcgtaccgaccagatggaagtggatagcggcccgggcaagcgtccgcacaaaagcggtttcaccgacaaggagcgtgaagaccatcgtcgtcgtaaagcgctggaaaacaagaaaaagcaactgagcgcgggtggcaagattttatctaaagaggaagaggaagagttacgtcgtctgaccgacgaggatgaagagcgtaagcgtcgtgttgcgggcccgcgtgtgggtgatgttaacccgagccgtggtggcccgcgtggcgcgccgggtggcggtttcgttccgcagatggcgggtgttccggaaagcccgtttagccgcaccggcgagggtctggatattcgtggcacgcaaggttttccgtgggttagcccgagcccgccgcagcaacgtctgccgctgctggagagcaccccgcagggtggtggcagccaaagcgagagcaaaaagaaccgtcgtggcggtcgtgaggacatcctggaaaagtggattaccacccgtcgtaaggcggaggaactggaaaaggacctgcgtaaggcgcgtaaaaccatcaaaaagctggaggatgagaatccgtggctgggtaatatcattggtatcattcgtaagggtaaagatggtgagggggctccgccggctaagcgtccgagaaccgaccagatggagattgatagcggcaccggcaagcgtccgcataagagcggctttaccgataaagagcgtgaagaccaccgtcgtcgtaaagcgctggaaaataaaaagaaacagctgagcagcggcggtaaaaacctgagccgtgaagaggaagaggaattaggtcgtctgaccgtggaggacgaagaacgtcgtcgtcgtgtggcgggtccgcgtaccggtgatgttaacctgagcggcggtggccctcgtggcgcgccgggtggcggtttcgtaccgcgtatggagggtgttcctgagagcccgtttacccgcaccggcgaaggtctggacatccgtggcaatcaaggctttccgtgggttcgtccgagcccgccgcagcagcgcctgccgctgctggaaagcaccccgcagggtaccaatttaagcaccagcaacccgctgggtttctttcccgatcatcaactggacccggcgttccgtgcgaattctgctaacccggattgggactttaatcctaataaagatacttggccggacgcgaataaggtgggcggtcagaatctttctaccagcaacccgctgggtttctttccagatcatcagctggacccggcgtttcgtgcgaataccgcgaacccggattgggatttcaacccgaataaagatacctggccggatgcgaacaaagtgggcggtggtggcagccgtagcgagagcaagaaaaaccgtggtggccgtgaggaaatcctggaacagtgggttggtgcgcgtaagaaactggaggaactggagcgtgacctgcgtaagatcaagaaaaagattaaaaagctggaggaagagaacccgtggctgggtaacatcaaaggcattctgggtaaaaaggatcgtgagggtgaaggtgctccgccggcgaagcgtgcgcgtgcggaccagatggaagttgatagcggcccgcgtaaacgtccgttccgtggtgagtttaccgacaaggaacgtcgtgatcaccgtcgtcgtaaagcgctggagaacaagcgtaaacaactgagcagcggtggcaagagcctgagcaaagaagaggaagaggaactgcgtaagctgaccgaggaagacgaacgtcgtgaacgtcgtgttgcgggtccgcgtgtgggtggcgttaacccgctggagggtggcacccgtggcgcgccgggtggcggtttcgtgccgagcatgcagggtgttccggagagcccgtttgcgcgtaccggcgaaggtctggatgttcgtggcaatcaaggtttcccgtgggacatcctgtttccggcggacccgccgtttagcccgcagagcagccgtccgcaaggtggtggcagccgtagcgaaagcaaaaagaaccgtggcggtcgtgaggaagtgctggagcagtgggttaacggccgtaaaaagctggaagagctggagcgtgaactgcgtcgtgcgcgtaaaaagatcaaaaagctggaagacgataatccgtggctgggcaacgtgaagggtatcctgggtaaaaaggacaaagatggtgagggcgctccgccggcgaaacgcgcgcgtaccgaccaaatggagattgatagcggtccgcgtaaacgtccgctgcgtggcggtttcaccgaccgtgagcgtcaggatcaccgtcgtcgtaaggcgctgaaaaacaaaaagaaacaactgagcgcgggcggtaagagcctgagcaaagaggaagaggaagagctgaaacgtctgacccgtgaagacgaagagcgtaagaaagaagagcatggtccgagccgtctgggtgtgaacccgagcgagggcggtccgcgtggcgcgccgggcggtggcttcgttccgagcatgcagggtatcccggagagccgttttacccgcaccggtgaaggtctggacgttcgtggcagccgtggttttccgcaagacattctgttcccgagcgatccgccgttcagcccgcagagcagccgcccgcaaggtaccaatttaagtaccagcaacccgctgggtttctttccagaccaccagctggacccggcgttccgcgcgaatagcgcgaatccggactgggacttcaatccgaacaaagacacttggcctgacgcgaacaaagtgggtggccagaacttatctaccagcaacccgctgggtttctttcctgaccaccaactggacccggcgtttcgcgcgaacaccgcgaacccggattgggacttcaacccgaataaagacacttggcccgacgcgaacaaagttggttaa

SEQ ID NO: 181 - DNA, 2895 bp, synthetic construct
  source 1..2895: mol_type = other DNA
atgagccagagcgaaacccgtcgtggccgtcgtggtacccgtgaagaaaccctggaaaagtggatcaccgcgcgtaagaaagcggaagagctggagaaagacctgcgtaagacccgtaaaaccatcaagaaactggaagaggaaaacccgtggctgggcaacattgtgggtatcattcgtaagggcaaagatggtgagggagccccgccggctaagcgtccgcgtaccgaccagatggaagtggatagcggcccgggcaagcgtccgcacaaaagcggtttcaccgacaaggagcgtgaagaccatcgtcgtcgtaaagcgctggaaaacaagaaaaagcaactgagcgcgggtggcaagattttatctaaagaggaagaggaagagttacgtcgtctgaccgacgaggatgaagagcgtaagcgtcgtgttgcgggcccgcgtgtgggtgatgttaacccgagccgtggtggcccgcgtggcgcgccgggtggcggtttcgttccgcagatggcgggtgttccggaaagcccgtttagccgcaccggcgagggtctggatattcgtggcacgcaaggttttccgtgggttagcccgagcccgccgcagcaacgtctgccgctgctggagagcaccccgcagggtggtggcagccaaagcgagagcaaaaagaaccgtcgtggcggtcgtgaggacatcctggaaaagtggattaccacccgtcgtaaggcggaggaactggaaaaggacctgcgtaaggcgcgtaaaaccatcaaaaagctggaggatgagaatccgtggctgggtaatatcattggtatcattcgtaagggtaaagatggtgagggggctccgccggctaagcgtccgagaaccgaccagatggagattgatagcggcaccggcaagcgtccgcataagagcggctttaccgataaagagcgtgaagaccaccgtcgtcgtaaagcgctggaaaataaaaagaaacagctgagcagcggcggtaaaaacctgagccgtgaagaggaagaggaattaggtcgtctgaccgtggaggacgaagaacgtcgtcgtcgtgtggcgggtccgcgtaccggtgatgttaacctgagcggcggtggccctcgtggcgcgccgggtggcggtttcgtaccgcgtatggagggtgttcctgagagcccgtttacccgcaccggcgaaggtctggacatccgtggcaatcaaggctttccgtgggttcgtccgagcccgccgcagcagcgcctgccgctgctggaaagcaccccgcagggtggtggcagccgtagcgagagcaagaaaaaccgtggtggccgtgaggaaatcctggaacagtgggttggtgcgcgtaagaaactggaggaactggagcgtgacctgcgtaagatcaagaaaaagattaaaaagctggaggaagagaacccgtggctgggtaacatcaaaggcattctgggtaaaaaggatcgtgagggtgaaggtgctccgccggcgaagcgtgcgcgtgcggaccagatggaagttgatagcggcccgcgtaaacgtccgttccgtggtgagtttaccgacaaggaacgtcgtgatcaccgtcgtcgtaaagcgctggagaacaagcgtaaacaactgagcagcggtggcaagagcctgagcaaagaagaggaagaggaactgcgtaagctgaccgaggaagacgaacgtcgtgaacgtcgtgttgcgggtccgcgtgtgggtggcgttaacccgctggagggtggcacccgtggcgcgccgggtggcggtttcgtgccgagcatgcagggtgttccggagagcccgtttgcgcgtaccggcgaaggtctggatgttcgtggcaatcaaggtttcccgtgggacatcctgtttccggcggacccgccgtttagcccgcagagcagccgtccgcaaggtggtggcagccgtagcgaaagcaaaaagaaccgtggcggtcgtgaggaagtgctggagcagtgggttaacggccgtaaaaagctggaagagctggagcgtgaactgcgtcgtgcgcgtaaaaagatcaaaaagctggaagacgataatccgtggctgggcaacgtgaagggtatcctgggtaaaaaggacaaagatggtgagggcgctccgccggcgaaacgcgcgcgtaccgaccaaatggagattgatagcggtccgcgtaaacgtccgctgcgtggcggtttcaccgaccgtgagcgtcaggatcaccgtcgtcgtaaggcgctgaaaaacaaaaagaaacaactgagcgcgggcggtaagagcctgagcaaagaggaagaggaagagctgaaacgtctgacccgtgaagacgaagagcgtaagaaagaagagcatggtccgagccgtctgggtgtgaacccgagcgagggcggtccgcgtggcgcgccgggcggtggcttcgttccgagcatgcagggtatcccggagagccgttttacccgcaccggtgaaggtctggacgttcgtggcagccgtggttttccgcaagacattctgttcccgagcgatccgccgttcagcccgcagagcagccgcccgcaaggtaccaatttaagtaccagcaacccgctgggtttctttccagaccaccagctggacccggcgttccgcgcgaatagcgcgaatccggactgggacttcaatccgaacaaagacacttggcctgacgcgaacaaagtgggtggccagaacttatctaccagcaacccgctgggtttctttcctgaccaccaactggacccggcgtttcgcgcgaacaccgcgaacccggattgggacttcaacccgaataaagacacttggcccgacgcgaacaaagttggtggatcccatcatcaccaccaccactaa

SEQ ID NO: 182 - DNA, 2895 bp, synthetic construct
  source 1..2895: mol_type = other DNA
atgagccagagcgaaacccgtcgtggtcgtcgtggtacccgtgaagaaaccctggaaaaatggatcaccgctcgtaaaaaagctgaagaactggaaaaagacctgcgtaaaacccgtaaaaccatcaaaaaactggaagaagaaaacccgtggctgggtaacatcgttggtatcatccgtaaaggtaaagacggtgaaggtgctccgccggctaaacgtccgcgtaccgaccagatggaagttgactctggtccgggtaaacgtccgcacaaatctggtttcaccgacaaagaacgtgaagaccaccgtcgtcgtaaagctctggaaaacaaaaaaaaacagctgtctgctggtggtaaaatcctgtctaaagaagaagaagaagaactgcgtcgtctgaccgacgaagacgaagaacgtaaacgtcgtgttgctggtccgcgtgttggtgacgttaacccgtctcgtggtggtccgcgtggtgctcccggcggtggcttcgttccgcagatggctggtgttccggaatctccgttctctcgtaccggtgaaggtctggacatcaggggcacacagggcttcccctgggtatcgccgtctccgccgcagcagcgtctgccgctgctggaatctaccccgcagggtggtggttctcagtctgaatctaaaaaaaaccgtcgtggtggtcgtgaagacatcctggaaaaatggatcaccacccgtcgtaaagctgaagaactggaaaaagacctgcgtaaagctcgtaaaaccatcaaaaaactggaagacgaaaacccgtggctgggtaacatcatcggtatcatccgtaaaggtaaagacggtgaaggtgctccgccggctaaacgtccgcgtaccgaccagatggaaatcgactctggtaccggtaaacgtccgcacaaatctggtttcaccgacaaagaacgtgaagaccaccgtcgtcgtaaagctctggaaaacaaaaaaaaacagctgtcttctggtggtaaaaacctgtctcgtgaagaagaagaagaactgggtcgtctgaccgttgaagacgaagaacgtcgtcgtcgtgttgctggtccgcgtaccggtgacgttaacctgtctggtggtggtccgcgtggtgctcccggcggtggcttcgttccgcgtatggaaggtgttccggaatctccgttcacccgtaccggtgaaggtctggacatccgtggtaatcagggcttcccgtgggttcgtccgtctccgccgcagcagcgtctgccgctgctggaatctaccccgcagggtggtggttctcgttctgaatctaaaaaaaaccgtggtggtcgtgaagaaatcctggaacagtgggttggtgctcgtaaaaaactggaagaactggaacgtgacctgcgtaaaatcaaaaaaaaaatcaaaaaactggaagaagaaaacccgtggctgggtaacatcaaaggtatcctgggtaaaaaagaccgtgaaggtgaaggtgctccgccggctaaacgtgctcgtgctgaccagatggaagttgactctggtccgcgtaaacgtccgttccgtggtgaattcaccgacaaagaacgtcgtgaccaccgtcgtcgtaaagctctggaaaacaaacgtaaacagctgtcttctggtggtaaatctctgtctaaagaagaagaagaagaactgcgtaaactgaccgaagaagacgaacgtcgtgaacgtcgtgttgctggtccgcgtgttggtggtgttaacccgctggaaggtggtacccgtggtgctccgggtggtggtttcgttccgtctatgcagggtgttctggaatctccgttcgctcgtaccggtgaaggtctggacgttcgtggcaatcagggcttcccgtgggacatactgttcccggctgacccgccgttctctccgcagtcttctcgtccgcagggtggtggttctcgttctgaatctaaaaaaaaccgtggtggtcgtgaagaagttctggaacagtgggttaacggtcgtaaaaaactggaagaactggaacgtgaactgcgtcgtgctcgtaaaaaaatcaaaaaactggaagacgacaacccgtggctgggtaacgttaaaggtatcctgggtaaaaaagacaaagacggtgaaggtgctccgccggctaaacgtgctcgtaccgaccagatggaaatcgactctggtccgcgtaaacgtccgttacgtggtggcttcaccgaccgtgagcgtcaggaccaccgtcgtcgtaaagctctgaaaaacaaaaaaaaacagctgtctgctggtggtaaatctctgtctaaagaagaagaagaagaactgaaacgtctgacccgtgaagacgaagaacgtaaaaaagaagaacacggtccgtctcgtctgggtgttaacccgtctgaaggtggtccgcgtggtgctcccggcggtggcttcgttccgtctatgcagggtatcccggaatctcgtttcacccgtaccggtgaaggtctggacgttcgtggttcgaggggcttcccgcaggacatactgttcccgtctgacccgccgttctctccgcagtcttctcgtccgcagggtaccaacctgtctacctctaacccgctgggtttcttcccggaccaccagctcgacccggcgttccgtgctaactcggctaacccggactgggacttcaacccgaacaaagacacctggccggacgctaacaaagttggtggtcagaacctgtctacctctaacccgctgggtttcttcccggaccaccagctcgacccggcgttccgtgctaacactgctaacccggactgggacttcaacccgaacaaagacacctggccggacgctaacaaagttggtggatcccaccaccaccaccaccactaa

SEQ ID NO: 183 - DNA, 2871 bp, synthetic construct
  source 1..2871: mol_type = other DNA
atgagccagagcgaaacccgtcgtggccgtcgtggtacccgtgaagaaaccctggaaaagtggatcaccgcgcgtaagaaagcggaagagctggagaaagacctgcgtaagacccgtaaaaccatcaagaaactggaagaggaaaacccgtggctgggcaacattgtgggtatcattcgtaagggcaaagatggtgagggagccccgccggctaagcgtccgcgtaccgaccagatggaagtggatagcggcccgggcaagcgtccgcacaaaagcggtttcaccgacaaggagcgtgaagaccatcgtcgtcgtaaagcgctggaaaacaagaaaaagcaactgagcgcgggtggcaagattttatctaaagaggaagaggaagagttacgtcgtctgaccgacgaggatgaagagcgtaagcgtcgtgttgcgggcccgcgtgtgggtgatgttaacccgagccgtggtggcccgcgtggcgcgccgggtggcggtttcgttccgcagatggcgggtgttccggaaagcccgtttagccgcaccggcgagggtctggatattcgtggcacgcaaggttttccgtgggttagcccgagcccgccgcagcaacgtctgccgctgctggagagcaccccgcagggtggtggcagccaaagcgagagcaaaaagaaccgtcgtggcggtcgtgaggacatcctggaaaagtggattaccacccgtcgtaaggcggaggaactggaaaaggacctgcgtaaggcgcgtaaaaccatcaaaaagctggaggatgagaatccgtggctgggtaatatcattggtatcattcgtaagggtaaagatggtgagggggctccgccggctaagcgtccgagaaccgaccagatggagattgatagcggcaccggcaagcgtccgcataagagcggctttaccgataaagagcgtgaagaccaccgtcgtcgtaaagcgctggaaaataaaaagaaacagctgagcagcggcggtaaaaacctgagccgtgaagaggaagaggaattaggtcgtctgaccgtggaggacgaagaacgtcgtcgtcgtgtggcgggtccgcgtaccggtgatgttaacctgagcggcggtggccctcgtggcgcgccgggtggcggtttcgtaccgcgtatggagggtgttcctgagagcccgtttacccgcaccggcgaaggtctggacatccgtggcaatcaaggctttccgtgggttcgtccgagcccgccgcagcagcgcctgccgctgctggaaagcaccccgcagggtggtggcagccgtagcgagagcaagaaaaaccgtggtggccgtgaggaaatcctggaacagtgggttggtgcgcgtaagaaactggaggaactggagcgtgacctgcgtaagatcaagaaaaagattaaaaagctggaggaagagaacccgtggctgggtaacatcaaaggcattctgggtaaaaaggatcgtgagggtgaaggtgctccgccggcgaagcgtgcgcgtgcggaccagatggaagttgatagcggcccgcgtaaacgtccgttccgtggtgagtttaccgacaaggaacgtcgtgatcaccgtcgtcgtaaagcgctggagaacaagcgtaaacaactgagcagcggtggcaagagcctgagcaaagaagaggaagaggaactgcgtaagctgaccgaggaagacgaacgtcgtgaacgtcgtgttgcgggtccgcgtgtgggtggcgttaacccgctggagggtggcacccgtggcgcgccgggtggcggtttcgtgccgagcatgcagggtgttccggagagcccgtttgcgcgtaccggcgaaggtctggatgttcgtggcaatcaaggtttcccgtgggacatcctgtttccggcggacccgccgtttagcccgcagagcagccgtccgcaaggtggtggcagccgtagcgaaagcaaaaagaaccgtggcggtcgtgaggaagtgctggagcagtgggttaacggccgtaaaaagctggaagagctggagcgtgaactgcgtcgtgcgcgtaaaaagatcaaaaagctggaagacgataatccgtggctgggcaacgtgaagggtatcctgggtaaaaaggacaaagatggtgagggcgctccgccggcgaaacgcgcgcgtaccgaccaaatggagattgatagcggtccgcgtaaacgtccgctgcgtggcggtttcaccgaccgtgagcgtcaggatcaccgtcgtcgtaaggcgctgaaaaacaaaaagaaacaactgagcgcgggcggtaagagcctgagcaaagaggaagaggaagagctgaaacgtctgacccgtgaagacgaagagcgtaagaaagaagagcatggtccgagccgtctgggtgtgaacccgagcgagggcggtccgcgtggcgcgccgggcggtggcttcgttccgagcatgcagggtatcccggagagccgttttacccgcaccggtgaaggtctggacgttcgtggcagccgtggttttccgcaagacattctgttcccgagcgatccgccgttcagcccgcagagcagccgcccgcaaggtaccaatttaagtaccagcaacccgctgggtttctttccagaccaccagctggacccggcgttccgcgcgaatagcgcgaatccggactgggacttcaatccgaacaaagacacttggcctgacgcgaacaaagtgggtggccagaacttatctaccagcaacccgctgggtttctttcctgaccaccaactggacccggcgtttcgcgcgaacaccgcgaacccggattgggacttcaacccgaataaagacacttggcccgacgcgaacaaagttggttaa

SEQ ID NO: 184 - DNA, 1599 bp, synthetic construct
  source 1..1599: mol_type = other DNA
atgagccagagcgaaacccgtcgtggccgtcgtggtacccgtgaagaaaccctggaaaagtggatcaccgcgcgtaagaaagcggaagagctggagaaagacctgcgtaagacccgtaaaaccatcaagaaactggaagaggaaaacccgtggctgggcaacattgtgggtatcattcgtaagggcaaagatggtgagggagccccgccggctaagcgtccgcgtaccgaccagatggaagtggatagcggcccgggcaagcgtccgcacaaaagcggtttcaccgacaaggagcgtgaagaccatcgtcgtcgtaaagcgctggaaaacaagaaaaagcaactgagcgcgggtggcaagattttatctaaagaggaagaggaagagttacgtcgtctgaccgacgaggatgaagagcgtaagcgtcgtgttgcgggcccgcgtgtgggtgatgttaacccgagccgtggtggcccgcgtggcgcgccgggtggcggtttcgttccgcagatggcgggtgttccggaaagcccgtttagccgcaccggcgagggtctggatattcgtggcacgcaaggttttccgtgggttagcccgagcccgccgcagcaacgtctgccgctgctggagagcaccccgcagggtggtggcagccgtagcgagagcaagaaaaaccgtggtggccgtgaggaaatcctggaacagtgggttggtgcgcgtaagaaactggaggaactggagcgtgacctgcgtaagatcaagaaaaagattaaaaagctggaggaagagaacccgtggctgggtaacatcaaaggcattctgggtaaaaaggatcgtgagggtgaaggtgctccgccggcgaagcgtgcgcgtgcggaccagatggaagttgatagcggcccgcgtaaacgtccgttccgtggtgagtttaccgacaaggaacgtcgtgatcaccgtcgtcgtaaagcgctggagaacaagcgtaaacaactgagcagcggtggcaagagcctgagcaaagaagaggaagaggaactgcgtaagctgaccgaggaagacgaacgtcgtgaacgtcgtgttgcgggtccgcgtgtgggtggcgttaacccgctggagggtggcacccgtggcgcgccgggtggcggtttcgtgccgagcatgcagggtgttccggagagcccgtttgcgcgtaccggcgaaggtctggatgttcgtggcaatcaaggtttcccgtgggacatcctgtttccggcggacccgccgtttagcccgcagagcagccgtccgcaaggtaccaatttaagtaccagcaacccgctgggtttctttccagaccaccagctggacccggcgttccgcgcgaatagcgcgaatccggactgggacttcaatccgaacaaagacacttggcctgacgcgaacaaagtgggtggccagaacttatctaccagcaacccgctgggtttctttcctgaccaccaactggacccggcgtttcgcgcgaacaccgcgaacccggattgggacttcaacccgaataaagacacttggcccgacgcgaacaaagttggtggatcccatcatcaccaccaccactaa

SEQ ID NO: 185 - DNA, 1575 bp, synthetic construct
  source 1..1575: mol_type = other DNA
atgagccagagcgaaacccgtcgtggccgtcgtggtacccgtgaagaaaccctggaaaagtggatcaccgcgcgtaagaaagcggaagagctggagaaagacctgcgtaagacccgtaaaaccatcaagaaactggaagaggaaaacccgtggctgggcaacattgtgggtatcattcgtaagggcaaagatggtgagggagccccgccggctaagcgtccgcgtaccgaccagatggaagtggatagcggcccgggcaagcgtccgcacaaaagcggtttcaccgacaaggagcgtgaagaccatcgtcgtcgtaaagcgctggaaaacaagaaaaagcaactgagcgcgggtggcaagattttatctaaagaggaagaggaagagttacgtcgtctgaccgacgaggatgaagagcgtaagcgtcgtgttgcgggcccgcgtgtgggtgatgttaacccgagccgtggtggcccgcgtggcgcgccgggtggcggtttcgttccgcagatggcgggtgttccggaaagcccgtttagccgcaccggcgagggtctggatattcgtggcacgcaaggttttccgtgggttagcccgagcccgccgcagcaacgtctgccgctgctggagagcaccccgcagggtggtggcagccgtagcgagagcaagaaaaaccgtggtggccgtgaggaaatcctggaacagtgggttggtgcgcgtaagaaactggaggaactggagcgtgacctgcgtaagatcaagaaaaagattaaaaagctggaggaagagaacccgtggctgggtaacatcaaaggcattctgggtaaaaaggatcgtgagggtgaaggtgctccgccggcgaagcgtgcgcgtgcggaccagatggaagttgatagcggcccgcgtaaacgtccgttccgtggtgagtttaccgacaaggaacgtcgtgatcaccgtcgtcgtaaagcgctggagaacaagcgtaaacaactgagcagcggtggcaagagcctgagcaaagaagaggaagaggaactgcgtaagctgaccgaggaagacgaacgtcgtgaacgtcgtgttgcgggtccgcgtgtgggtggcgttaacccgctggagggtggcacccgtggcgcgccgggtggcggtttcgtgccgagcatgcagggtgttccggagagcccgtttgcgcgtaccggcgaaggtctggatgttcgtggcaatcaaggtttcccgtgggacatcctgtttccggcggacccgccgtttagcccgcagagcagccgtccgcaaggtaccaatttaagtaccagcaacccgctgggtttctttccagaccaccagctggacccggcgttccgcgcgaatagcgcgaatccggactgggacttcaatccgaacaaagacacttggcctgacgcgaacaaagtgggtggccagaacttatctaccagcaacccgctgggtttctttcctgaccaccaactggacccggcgtttcgcgcgaacaccgcgaacccggattgggacttcaacccgaataaagacacttggcccgacgcgaacaaagttggttaa

SEQ ID NO: 186 - DNA, 1599 bp, synthetic construct
  source 1..1599: mol_type = other DNA
atgagccagagcgaaacccgtcgtggccgtcgtggtacccgtgaagaaaccctggaaaagtggatcaccgcgcgtaagaaagcggaagagctggagaaagacctgcgtaagacccgtaaaaccatcaagaaactggaagaggaaaacccgtggctgggcaacattgtgggtatcattcgtaagggcaaagatggtgagggagccccgccggctaagcgtccgcgtaccgaccagatggaagtggatagcggcccgggcaagcgtccgcacaaaagcggtttcaccgacaaggagcgtgaagaccatcgtcgtcgtaaagcgctggaaaacaagaaaaagcaactgagcgcgggtggcaagattttatctaaagaggaagaggaagagttacgtcgtctgaccgacgaggatgaagagcgtaagcgtcgtgttgcgggcccgcgtgtgggtgatgttaacccgagccgtggtggcccgcgtggcgcgccgggtggcggtttcgttccgcagatggcgggtgttccggaaagcccgtttagccgcaccggcgagggtctggatattcgtggcacgcaaggttttccgtgggttagcccgagcccgccgcagcaacgtctgccgctgctggagagcaccccgcagggtggtggcagccgtagcgaaagcaaaaagaaccgtggcggtcgtgaggaagtgctggagcagtgggttaacggccgtaaaaagctggaagagctggagcgtgaactgcgtcgtgcgcgtaaaaagatcaaaaagctggaagacgataatccgtggctgggcaacgtgaagggtatcctgggtaaaaaggacaaagatggtgagggcgctccgccggcgaaacgcgcgcgtaccgaccaaatggagattgatagcggtccgcgtaaacgtccgctgcgtggcggtttcaccgaccgtgagcgtcaggatcaccgtcgtcgtaaggcgctgaaaaacaaaaagaaacaactgagcgcgggcggtaagagcctgagcaaagaggaagaggaagagctgaaacgtctgacccgtgaagacgaagagcgtaagaaagaagagcatggtccgagccgtctgggtgtgaacccgagcgagggcggtccgcgtggcgcgccgggcggtggcttcgttccgagcatgcagggtatcccggagagccgttttacccgcaccggtgaaggtctggacgttcgtggcagccgtggttttccgcaagacattctgttcccgagcgatccgccgttcagcccgcagagcagccgcccgcaaggtaccaatttaagtaccagcaacccgctgggtttctttccagaccaccagctggacccggcgttccgcgcgaatagcgcgaatccggactgggacttcaatccgaacaaagacacttggcctgacgcgaacaaagtgggtggccagaacttatctaccagcaacccgctgggtttctttcctgaccaccaactggacccggcgtttcgcgcgaacaccgcgaacccggattgggacttcaacccgaataaagacacttggcccgacgcgaacaaagttggtggatcccatcatcaccaccaccactaa

SEQ ID NO: 187 - protein, 1058 aa, synthetic construct
MSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGGGSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLESTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGGGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGGGSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSSRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGSHHHHHH

SEQ ID NO: 188 - protein, 1155 aa, synthetic construct
MGTNLSTSNPLGFFPDHQLDPAFRANSANPDWDFNPNKDTWPDANKVGGQNLSTSNPLGFFPDHQLDPAFRANTANPDWDFNPNKDTWPDANKVGGGGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGGGSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLESTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGGGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGGGSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSSRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGSHHHHHH

SEQ ID NO: 189 - protein, 964 aa, synthetic construct
MSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGGGSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLESTPQGGGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGGGSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSSRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGSHHHHHH

SEQ ID NO: 190 - protein, 964 aa, synthetic construct
MSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGGGSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLESTPQGGGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVLESPFARTGEGLDVRGNQGFPWDILFPADPPFSPQSSRPQGGGSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSSRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGSHHHHHH

SEQ ID NO: 191 - protein, 532 aa, synthetic construct
MSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGGGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGSHHHHHH

SEQ ID NO: 192 - protein, 532 aa, synthetic construct
MSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGGGSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSSRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGSHHHHHH

SEQ ID NO: 193 - protein, 1147 aa, synthetic construct
MGTNLSTSNPLGFFPDHQLDPAFRANSANPDWDFNPNKDTWPDANKVGGQNLSTSNPLGFFPDHQLDPAFRANTANPDWDFNPNKDTWPDANKVGGGGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGGGSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLESTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGGGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGGGSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSSRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 194 - protein, 956 aa, synthetic construct
MSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGGGSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLESTPQGGGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGGGSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSSRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 195 - protein, 524 aa, synthetic construct
MSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGGGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 196 - protein, 3492 aa, synthetic construct
ATGAGCCAGAGCGAAACCCGTCGTGGCCGTCGTGGTACCCGTGAAGAAACCCTGGAAAAGTGGATCACCGCGCGTAAGAAAGCGGAAGAGCTGGAGAAAGACCTGCGTAAGACCCGTAAAACCATCAAGAAACTGGAAGAGGAAAACCCGTGGCTGGGCAACATTGTGGGTATCATTCGTAAGGGCAAAGATGGTGAGGGAGCCCCGCCGGCTAAGCGTCCGCGTACCGACCAGATGGAAGTGGATAGCGGCCCGGGCAAGCGTCCGCACAAAAGCGGTTTCACCGACAAGGAGCGTGAAGACCATCGTCGTCGTAAAGCGCTGGAAAACAAGAAAAAGCAACTGAGCGCGGGTGGCAAGATTTTATCTAAAGAGGAAGAGGAAGAGTTACGTCGTCTGACCGACGAGGATGAAGAGCGTAAGCGTCGTGTTGCGGGCCCGCGTGTGGGTGATGTTAACCCGAGCCGTGGTGGCCCGCGTGGCGCGCCGGGTGGCGGTTTCGTTCCGCAGATGGCGGGTGTTCCGGAAAGCCCGTTTAGCCGCACCGGCGAGGGTCTGGATATTCGTGGCACGCAAGGTTTTCCGTGGGTTAGCCCGAGCCCGCCGCAGCAACGTCTGCCGCTGCTGGAGAGCACCCCGCAGGGTCAGAATCTTTCTACCAGCAACCCGCTGGGTTTCTTTCCAGATCATCAGCTGGACCCGGCGTTTCGTGCGAATACCGCGAACCCGGATTGGGATTTCAACCCGAATAAAGATACCTGGCCGGATGCGAACAAAGTGGGCGGCTCCGAACAGAAACTGATCTCTGAAGAAGACCTGGGCTCCCATCATCATCATCATCATTAGGAGGTAATTATGAGCCGTAGCGAGAGCAAGAAAAACCGTGGTGGCCGTGAGGAAATCCTGGAACAGTGGGTTGGTGCGCGTAAGAAACTGGAGGAACTGGAGCGTGACCTGCGTAAGATCAAGAAAAAGATTAAAAAGCTGGAGGAAGAGAACCCGTGGCTGGGTAACATCAAAGGCATTCTGGGTAAAAAGGATCGTGAGGGTGAAGGTGCTCCGCCGGCGAAGCGTGCGCGTGCGGACCAGATGGAAGTTGATAGCGGCCCGCGTAAACGTCCGTTCCGTGGTGAGTTTACCGACAAGGAACGTCGTGATCACCGTCGTCGTAAAGCGCTGGAGAACAAGCGTAAACAACTGAGCAGCGGTGGCAAGAGCCTGAGCAAAGAAGAGGAAGAGGAACTGCGTAAGCTGACCGAGGAAGACGAACGTCGTGAACGTCGTGTTGCGGGTCCGCGTGTGGGTGGCGTTAACCCGCTGGAGGGTGGCACCCGTGGCGCGCCGGGTGGCGGTTTCGTGCCGAGTATGCAGGGTGTTCCGGAGAGCCCGTTTGCGCGTACCGGCGAAGGTCTGGATGTTCGTGGCAATCAAGGTTTCCCGTGGGACATCCTGTTTCCGGCGGACCCGCCGTTTAGCCCGCAGAGCAGCCGTCCGCAAGGTACCAATTTAAGCACCAGCAACCCGCTGGGTTTCTTTCCCGATCATCAACTGGACCCGGCGTTCCGTGCGAATTCTGCTAACCCGGATTGGGACTTTAATCCTAATAAAGATACTTGGCCGGACGCGAATAAGGTGGGCGGCTCCGCTCCGGTTCCGTACCCGGACCCGCTGGAACCGCGTGGCTCCCATCACCATCACCATCACTAACGCGAAATTAATACGACTCACTATAGGGGAATTGTGAGCGGATAACAATTCCCCTCCAGAAATAATTTTGTTTAACTTTAAGAAGGAGATATACATATGAGCCGTAGCGAAAGCAAAAAGAACCGTGGCGGTCGTGAGGAAGTGCTGGAGCAGTGGGTTAACGGCCGTAAAAAGCTGGAAGAGCTGGAGCGTGAACTGCGTCGTGCGCGTAAAAAGATCAAAAAGCTGGAAGACGATAATCCGTGGCTGGGCAACGTGAAGGGTATCCTGGGTAAAAAGGACAAAGATGGTGAGGGCGCTCCGCCGGCGAAACGCGCGCGTACCGACCAAATGGAGATTGATAGCGGTCCGCGTAAACGTCCGCTGCGTGGCGGTTTCACCGACCGTGAGCGTCAGGATCACCGTCGTCGTAAGGCGCTGAAAAACAAAAAGAAACAACTGAGCGCGGGCGGTAAGAGCCTGAGCAAAGAGGAAGAGGAAGAGCTGAAACGTCTGACCCGTGAAGACGAAGAGCGTAAGAAAGAAGAGCATGGTCCGAGCCGTCTGGGTGTGAACCCGAGCGAGGGCGGTCCGCGTGGCGCGCCGGGCGGTGGCTTCGTTCCGAGTATGCAGGGTATCCCGGAGAGCCGTTTTACCCGCACCGGTGAAGGTCTGGACGTTCGTGGCAGCCGTGGTTTTCCGCAAGACATTCTGTTCCCGAGCGATCCGCCGTTCAGCCCGCAGAGCAGCCGCCCGCAAGGTACCAATTTAAGTACCAGCAACCCGCTGGGTTTCTTTCCAGACCACCAGCTGGACCCGGCGTTCCGCGCGAATAGCGCGAATCCGGACTGGGACTTCAATCCGAACAAAGACACTTGGCCTGACGCGAACAAAGTGGGTGGCTCCGACTACAAAGACGACGACGACAAAGGCTCCCACCACCACCACCACCACTAGGAGGTAATTATGAGCCAAAGCGAGAGCAAAAAGAACCGTCGTGGCGGTCGTGAGGACATCCTGGAAAAGTGGATTACCACCCGTCGTAAGGCGGAGGAACTGGAAAAGGACCTGCGTAAGGCGCGTAAAACCATCAAAAAGCTGGAGGATGAGAATCCGTGGCTGGGTAATATCATTGGTATCATTCGTAAGGGTAAAGATGGTGAGGGGGCTCCGCCGGCTAAGCGTCCGAGAACCGACCAGATGGAGATTGATAGCGGCACCGGCAAGCGTCCGCATAAGAGCGGCTTTACCGATAAAGAGCGTGAAGACCACCGTCGTCGTAAAGCGCTGGAAAATAAAAAGAAACAGCTGAGCAGCGGCGGTAAAAACCTGAGCCGTGAAGAGGAAGAGGAATTAGGTCGTCTGACCGTGGAGGACGAAGAACGTCGTCGTCGTGTGGCGGGTCCGCGTACCGGTGATGTTAACCTGAGCGGCGGTGGCCCTCGTGGCGCGCCGGGTGGCGGTTTCGTACCGCGTATGGAGGGTGTTCCTGAGAGCCCGTTTACCCGCACCGGCGAAGGTCTGGACATCCGTGGCAATCAAGGCTTTCCGTGGGTTCGTCCGAGCCCGCCGCAGCAGCGCCTGCCGCTGCTGGAAAGCACCCCGCAGGGCCAGAATTTATCTACCAGCAACCCGCTGGGTTTCTTTCCGGACCACCAACTGGACCCGGCGTTTCGTGCGAACACCGCGAATCCGGATTGGGACTTCAATCCGAATAAGGACACCTGGCCGGATGCGAATAAAGTGGGCGGATCCTGGTCTCACCCGCAGTTCGAAAAAGGCTCCCACCACCATCATCACCATTAA

SEQ ID NO: 197 - DNA, 3258 bp, synthetic construct
  source 1..3258: mol_type = other DNA
atgagccagagcgaaacccgtcgtggccgtcgtggtacccgtgaagaaaccctggaaaagtggatcaccgcgcgtaagaaagcggaagagctggagaaagacctgcgtaagacccgtaaaaccatcaagaaactggaagaggaaaacccgtggctgggcaacattgtgggtatcattcgtaagggcaaagatggtgagggagccccgccggctaagcgtccgcgtaccgaccagatggaagtggatagcggcccgggcaagcgtccgcacaaaagcggtttcaccgacaaggagcgtgaagaccatcgtcgtcgtaaagcgctggaaaacaagaaaaagcaactgagcgcgggtggcaagattttatctaaagaggaagaggaagagttacgtcgtctgaccgacgaggatgaagagcgtaagcgtcgtgttgcgggcccgcgtgtgggtgatgttaacccgagccgtggtggcccgcgtggcgcgccgggtggcggtttcgttccgcagatggcgggtgttccggaaagcccgtttagccgcaccggcgagggtctggatattcgtggcacgcaaggttttccgtgggttagcccgagcccgccgcagcaacgtctgccgctgctggagagcaccccgcagggtcagaatctttctaccagcaacccgctgggtttctttccagatcatcagctggacccggcgtttcgtgcgaataccgcgaacccggattgggatttcaacccgaataaagatacctggccggatgcgaacaaagtaggataggaggtaattatgagccgtagcgagagcaagaaaaaccgtggtggccgtgaggaaatcctggaacagtgggttggtgcgcgtaagaaactggaggaactggagcgtgacctgcgtaagatcaagaaaaagattaaaaagctggaggaagagaacccgtggctgggtaacatcaaaggcattctgggtaaaaaggatcgtgagggtgaaggtgctccgccggcgaagcgtgcgcgtgcggaccagatggaagttgatagcggcccgcgtaaacgtccgttccgtggtgagtttaccgacaaggaacgtcgtgatcaccgtcgtcgtaaagcgctggagaacaagcgtaaacaactgagcagcggtggcaagagcctgagcaaagaagaggaagaggaactgcgtaagctgaccgaggaagacgaacgtcgtgaacgtcgtgttgcgggtccgcgtgtgggtggcgttaacccgctggagggtggcacccgtggcgcgccgggtggcggtttcgtgccgagtatgcagggtgttccggagagcccgtttgcgcgtaccggcgaaggtctggatgttcgtggcaatcaaggtttcccgtgggacatcctgtttccggcggacccgccgtttagcccgcagagcagccgtccgcaaggtaccaatttaagcaccagcaacccgctgggtttctttcccgatcatcaactggacccggcgttccgtgcgaattctgctaacccggattgggactttaatcctaataaagatacttggccggacgcgaataaggtgggctaacgcgaaattaatacgactcactataggggaattgtgagcggataacaattcccctccagaaataattttgtttaactttaagaaggagatatacatatgagccgtagcgaaagcaaaaagaaccgtggcggtcgtgaggaagtgctggagcagtgggttaacggccgtaaaaagctggaagagctggagcgtgaactgcgtcgtgcgcgtaaaaagatcaaaaagctggaagacgataatccgtggctgggcaacgtgaagggtatcctgggtaaaaaggacaaagatggtgagggcgctccgccggcgaaacgcgcgcgtaccgaccaaatggagattgatagcggtccgcgtaaacgtccgctgcgtggcggtttcaccgaccgtgagcgtcaggatcaccgtcgtcgtaaggcgctgaaaaacaaaaagaaacaactgagcgcgggcggtaagagcctgagcaaagaggaagaggaagagctgaaacgtctgacccgtgaagacgaagagcgtaagaaagaagagcatggtccgagccgtctgggtgtgaacccgagcgagggcggtccgcgtggcgcgccgggcggtggcttcgttccgagtatgcagggtatcccggagagccgttttacccgcaccggtgaaggtctggacgttcgtggcagccgtggttttccgcaagacattctgttcccgagcgatccgccgttcagcccgcagagcagccgcccgcaaggtaccaatttaagtaccagcaacccgctgggtttctttccagaccaccagctggacccggcgttccgcgcgaatagcgcgaatccggactgggacttcaatccgaacaaagacacttggcctgacgcgaacaaagtaggataggaggtaattatgagccaaagcgagagcaaaaagaaccgtcgtggcggtcgtgaggacatcctggaaaagtggattaccacccgtcgtaaggcggaggaactggaaaaggacctgcgtaaggcgcgtaaaaccatcaaaaagctggaggatgagaatccgtggctgggtaatatcattggtatcattcgtaagggtaaagatggtgagggggctccgccggctaagcgtccgagaaccgaccagatggagattgatagcggcaccggcaagcgtccgcataagagcggctttaccgataaagagcgtgaagaccaccgtcgtcgtaaagcgctggaaaataaaaagaaacagctgagcagcggcggtaaaaacctgagccgtgaagaggaagaggaattaggtcgtctgaccgtggaggacgaagaacgtcgtcgtcgtgtggcgggtccgcgtaccggtgatgttaacctgagcggcggtggccctcgtggcgcgccgggtggcggtttcgtaccgcgtatggagggtgttcctgagagcccgtttacccgcaccggcgaaggtctggacatccgtggcaatcaaggctttccgtgggttcgtccgagcccgccgcagcagcgcctgccgctgctggaaagcaccccgcagggccagaatttatctaccagcaacccgctgggtttctttccggaccaccaactggacccggcgtttcgtgcgaacaccgcgaatccggattgggacttcaatccgaataaggacacctggccggatgcgaataaagtgggctaa

SEQ ID NO: 198 - DNA, 3282 bp, synthetic construct
  source 1..3282: mol_type = other DNA
atgagccagagcgaaacccgtcgtggccgtcgtggtacccgtgaagaaaccctggaaaagtggatcaccgcgcgtaagaaagcggaagagctggagaaagacctgcgtaagacccgtaaaaccatcaagaaactggaagaggaaaacccgtggctgggcaacattgtgggtatcattcgtaagggcaaagatggtgagggagccccgccggctaagcgtccgcgtaccgaccagatggaagtggatagcggcccgggcaagcgtccgcacaaaagcggtttcaccgacaaggagcgtgaagaccatcgtcgtcgtaaagcgctggaaaacaagaaaaagcaactgagcgcgggtggcaagattttatctaaagaggaagaggaagagttacgtcgtctgaccgacgaggatgaagagcgtaagcgtcgtgttgcgggcccgcgtgtgggtgatgttaacccgagccgtggtggcccgcgtggcgcgccgggtggcggtttcgttccgcagatggcgggtgttccggaaagcccgtttagccgcaccggcgagggtctggatattcgtggcacgcaaggttttccgtgggttagcccgagcccgccgcagcaacgtctgccgctgctggagagcaccccgcagggtggtggcagccgtagcgagagcaagaaaaaccgtggtggccgtgaggaaatcctggaacagtgggttggtgcgcgtaagaaactggaggaactggagcgtgacctgcgtaagatcaagaaaaagattaaaaagctggaggaagagaacccgtggctgggtaacatcaaaggcattctgggtaaaaaggatcgtgagggtgaaggtgctccgccggcgaagcgtgcgcgtgcggaccagatggaagttgatagcggcccgcgtaaacgtccgttccgtggtgagtttaccgacaaggaacgtcgtgatcaccgtcgtcgtaaagcgctggagaacaagcgtaaacaactgagcagcggtggcaagagcctgagcaaagaagaggaagaggaactgcgtaagctgaccgaggaagacgaacgtcgtgaacgtcgtgttgcgggtccgcgtgtgggtggcgttaacccgctggagggtggcacccgtggcgcgccgggtggcggtttcgtgccgagcatgcagggtgttccggagagcccgtttgcgcgtaccggcgaaggtctggatgttcgtggcaatcaaggtttcccgtgggacatcctgtttccggcggacccgccgtttagcccgcagagcagccgtccgcaaggtaccaatttaagtaccagcaacccgctgggtttctttccagaccaccagctggacccggcgttccgcgcgaatagcgcgaatccggactgggacttcaatccgaacaaagacacttggcctgacgcgaacaaagtgggtggccagaacttatctaccagcaacccgctgggtttctttcctgaccaccaactggacccggcgtttcgcgcgaacaccgcgaacccggattgggacttcaacccgaataaagacacttggcccgacgcgaacaaagttggtggatccgctccggttccgtacccggacccgctggaaccgcgtggctcccatcaccatcaccatcactaggaggtaattatgagccgtagcgaaagcaaaaagaaccgtggcggtcgtgaggaagtgctggagcagtgggttaacggccgtaaaaagctggaagagctggagcgtgaactgcgtcgtgcgcgtaaaaagatcaaaaagctggaagacgataatccgtggctgggcaacgtgaagggtatcctgggtaaaaaggacaaagatggtgagggcgctccgccggcgaaacgcgcgcgtaccgaccaaatggagattgatagcggtccgcgtaaacgtccgctgcgtggcggtttcaccgaccgtgagcgtcaggatcaccgtcgtcgtaaggcgctgaaaaacaaaaagaaacaactgagcgcgggcggtaagagcctgagcaaagaggaagaggaagagctgaaacgtctgacccgtgaagacgaagagcgtaagaaagaagagcatggtccgagccgtctgggtgtgaacccgagcgagggcggtccgcgtggcgcgccgggcggtggcttcgttccgagtatgcagggtatcccggagagccgttttacccgcaccggtgaaggtctggacgttcgtggcagccgtggttttccgcaagacattctgttcccgagcgatccgccgttcagcccgcagagcagccgcccgcaaggtggtggtggcagccaaagcgagagcaaaaagaaccgtcgtggcggtcgtgaggacatcctggaaaagtggattaccacccgtcgtaaggcggaggaactggaaaaggacctgcgtaaggcgcgtaaaaccatcaaaaagctggaggatgagaatccgtggctgggtaatatcattggtatcattcgtaagggtaaagatggtgagggggctccgccggctaagcgtccgagaaccgaccagatggagattgatagcggcaccggcaagcgtccgcataagagcggctttaccgataaagagcgtgaagaccaccgtcgtcgtaaagcgctggaaaataaaaagaaacagctgagcagcggcggtaaaaacctgagccgtgaagaggaagaggaattaggtcgtctgaccgtggaggacgaagaacgtcgtcgtcgtgtggcgggtccgcgtaccggtgatgttaacctgagcggcggtggccctcgtggcgcgccgggtggcggtttcgtaccgcgtatggagggtgttcctgagagcccgtttacccgcaccggcgaaggtctggacatccgtggcaatcaaggctttccgtgggttcgtccgagcccgccgcagcagcgcctgccgctgctggaaagcaccccgcagggtaccaacttaagtacttcaaatcctctaggcttcttcccggaccaccaactcgatccggctttccgcgcgaacagcgcgaacccggattgggattttaacccaaataaagatacgtggccggacgccaacaaggtgggcggtcagaatctgtccaccagcaatccgctgggtttttttcctgatcatcagttggacccggcgtttcgtgcgaacaccgcaaacccggattgggacttcaacccgaataaggacacctggcctgacgctaacaaagttggtggctcctggtctcacccgcagttcgaaaaaggctcccaccaccatcatcaccattaa

SEQ ID NO: 199 - DNA, 3000 bp, synthetic construct
  source 1..3000: mol_type = other DNA
atgagccagagcgaaacccgtcgtggccgtcgtggtacccgtgaagaaaccctggaaaagtggatcaccgcgcgtaagaaagcggaagagctggagaaagacctgcgtaagacccgtaaaaccatcaagaaactggaagaggaaaacccgtggctgggcaacattgtgggtatcattcgtaagggcaaagatggtgagggagccccgccggctaagcgtccgcgtaccgaccagatggaagtggatagcggcccgggcaagcgtccgcacaaaagcggtttcaccgacaaggagcgtgaagaccatcgtcgtcgtaaagcgctggaaaacaagaaaaagcaactgagcgcgggtggcaagattttatctaaagaggaagaggaagagttacgtcgtctgaccgacgaggatgaagagcgtaagcgtcgtgttgcgggcccgcgtgtgggtgatgttaacccgagccgtggtggcccgcgtggcgcgccgggtggcggtttcgttccgcagatggcgggtgttccggaaagcccgtttagccgcaccggcgagggtctggatattcgtggcacgcaaggttttccgtgggttagcccgagcccgccgcagcaacgtctgccgctgctggagagcaccccgcagggtggtggcagccgtagcgagagcaagaaaaaccgtggtggccgtgaggaaatcctggaacagtgggttggtgcgcgtaagaaactggaggaactggagcgtgacctgcgtaagatcaagaaaaagattaaaaagctggaggaagagaacccgtggctgggtaacatcaaaggcattctgggtaaaaaggatcgtgagggtgaaggtgctccgccggcgaagcgtgcgcgtgcggaccagatggaagttgatagcggcccgcgtaaacgtccgttccgtggtgagtttaccgacaaggaacgtcgtgatcaccgtcgtcgtaaagcgctggagaacaagcgtaaacaactgagcagcggtggcaagagcctgagcaaagaagaggaagaggaactgcgtaagctgaccgaggaagacgaacgtcgtgaacgtcgtgttgcgggtccgcgtgtgggtggcgttaacccgctggagggtggcacccgtggcgcgccgggtggcggtttcgtgccgagtatgcagggtgttccggagagcccgtttgcgcgtaccggcgaaggtctggatgttcgtggcaatcaaggtttcccgtgggacatcctgtttccggcggacccgccgtttagcccgcagagcagccgtccgcaaggtaccaatttaagcaccagcaacccgctgggtttctttcccgatcatcaactggacccggcgttccgtgcgaattctgctaacccggattgggactttaatcctaataaagatacttggccggacgcgaataaggtgggcggctccgctccggttccgtacccggacccgctggaaccgcgtggctcccatcaccatcaccatcactaggaggtaattatgagccgtagcgaaagcaaaaagaaccgtggcggtcgtgaggaagtgctggagcagtgggttaacggccgtaaaaagctggaagagctggagcgtgaactgcgtcgtgcgcgtaaaaagatcaaaaagctggaagacgataatccgtggctgggcaacgtgaagggtatcctgggtaaaaaggacaaagatggtgagggcgctccgccggcgaaacgcgcgcgtaccgaccaaatggagattgatagcggtccgcgtaaacgtccgctgcgtggcggtttcaccgaccgtgagcgtcaggatcaccgtcgtcgtaaggcgctgaaaaacaaaaagaaacaactgagcgcgggcggtaagagcctgagcaaagaggaagaggaagagctgaaacgtctgacccgtgaagacgaagagcgtaagaaagaagagcatggtccgagccgtctgggtgtgaacccgagcgagggcggtccgcgtggcgcgccgggcggtggcttcgttccgagtatgcagggtatcccggagagccgttttacccgcaccggtgaaggtctggacgttcgtggcagccgtggttttccgcaagacattctgttcccgagcgatccgccgttcagcccgcagagcagccgcccgcaaggtggtggtggcagccaaagcgagagcaaaaagaaccgtcgtggcggtcgtgaggacatcctggaaaagtggattaccacccgtcgtaaggcggaggaactggaaaaggacctgcgtaaggcgcgtaaaaccatcaaaaagctggaggatgagaatccgtggctgggtaatatcattggtatcattcgtaagggtaaagatggtgagggggctccgccggctaagcgtccgagaaccgaccagatggagattgatagcggcaccggcaagcgtccgcataagagcggctttaccgataaagagcgtgaagaccaccgtcgtcgtaaagcgctggaaaataaaaagaaacagctgagcagcggcggtaaaaacctgagccgtgaagaggaagaggaattaggtcgtctgaccgtggaggacgaagaacgtcgtcgtcgtgtggcgggtccgcgtaccggtgatgttaacctgagcggcggtggccctcgtggcgcgccgggtggcggtttcgtaccgcgtatggagggtgttcctgagagcccgtttacccgcaccggcgaaggtctggacatccgtggcaatcaaggctttccgtgggttcgtccgagcccgccgcagcagcgcctgccgctgctggaaagcaccccgcagggccagaatttatctaccagcaacccgctgggtttctttccggaccaccaactggacccggcgtttcgtgcgaacaccgcgaatccggattgggacttcaatccgaataaggacacctggccggatgcgaataaagtgggcggctcctggtctcacccgcagttcgaaaaaggctcccaccaccatcatcaccattaa

SEQ ID NO: 200 - protein, 546 aa, synthetic construct
MSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGGGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGSAPVPYPDPLEPRGSHHHHHH

SEQ ID NO: 201 - protein, 543 aa, synthetic construct
MSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSSRPQGGGGSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLESTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGSWSHPQFEKGSHHHHHH

SEQ ID NO: 202 - protein, 499 aa, synthetic construct
MSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLESTPQGGGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSSRPQGTNLSTSNPLGFFPDHQLDPAFRANSANPDWDFNPNKDTWPDANKVGGSAPVPYPDPLEPRGSHHHHHH

SEQ ID NO: 203 - protein, 496 aa, synthetic construct
MSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSSRPQGGGGSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLESTPQGQNLSTSNPLGFFPDHQLDPAFRANTANPDWDFNPNKDTWPDANKVGGSWSHPQFEKGSHHHHHH

(19) United States
(12) Patent Application Publication
(10) Pub. No.: US 2025/0197452 A1
(43) Pub. Date: Jun. 19, 2025

(54) COMPOSITIONS AND METHODS FOR TREATING AND PREVENTING HEPATITIS B AND D

(71) Applicant: SVF Vaccines AB, Solna (SE)

(72) Inventors: Matti Sällberg, Solna (SE); Lars Frelin, Solna (SE); Nico Mertens, Solna (SE)

(21) Appl. No.: 18/292,059

(22) PCT Filed: Jul. 26, 2022
(86) PCT No.: PCT/US2022/074160

§ 371 Date: Jan. 25, 2024

Related U.S. Application Data
(60) Provisional application No. 63/226,577, filed on Jul. 28, 2021.

(51) Int. Cl. C07K 14/005; C12N 15/11

(52) U.S. Cl. CPC C07K 14/005; C12N 15/11; C12N 2760/10122

(57) ABSTRACT

Disclosed herein are immunogenic compositions or product combinations of engineered hepatitis B and hepatitis D nucleic acids, genes, peptides, or proteins that can be used to elicit an immune response against a hepatitis B and/or hepatitis D infection. Also disclosed are methods of using the immunogenic compositions or product combinations in subjects to generate immune responses against HBV and/or HDV by administering the compositions or combinations, for example, with a nucleic acid prime and polypeptide boost approach.

### CROSS-REFERENCE TO RELATED APPLICATIONS

This application claims the benefit of priority of U.S. Provisional Patent Application 63/226,577, filed Jul. 28, 2021, which is hereby expressly incorporated by reference in its entirety.

### REFERENCE TO SEQUENCE LISTING

The present application is being filed along with a Sequence Listing in electronic format. The Sequence Listing is provided as a file entitled SVF008WOSeqListing.xml, which was created and last modified on Jul. 25, 2022, which is 397,887 bytes in size. The information in the electronic Sequence Listing is hereby expressly incorporated by reference in its entirety.

### FIELD

Aspects of the present disclosure relate generally to immunogenic compositions or product combinations of engineered hepatitis B (HBV) and hepatitis D (HDV) nucleic acids, genes, peptides, or proteins that can be used to elicit an immune response against an HBV and/or HDV infection. This immune response comprises, consists essentially of, or consists of activated immune cells that produce neutralizing antibodies and activated immune cells, such as T cells and B cells, against HBV and/or HDV. The disclosure also relates generally to methods of using the immunogenic compositions or product combinations in subjects to generate immune responses against HBV and/or HDV, for example, by administering the compositions or combinations with a homologous or heterologous nucleic acid and/or polypeptide prime and nucleic acid and/or polypeptide boost approach.

### BACKGROUND

Hepatitis is a disease resulting in swelling and inflammation of the liver. This disorder is commonly caused by viruses, five types of which are currently known (hepatitis A, B, C, D, and E). Hepatitis B infection can be either acute or chronic, with severe chronic infections causing chronic inflammation, fibrosis, cirrhosis, and hepatocellular carcinoma. The hepatitis B virus has a partially double-stranded circular DNA genome that enters the host nucleus and is transcribed by the host RNA polymerase into four viral mRNA molecules. These are used to translate viral proteins such as capsid proteins and surface antigens as well as produce more DNA genomes using a reverse transcriptase. Hepatitis D is a virusoid that relies on hepatitis B coinfection or superinfection to replicate. The circular single-stranded RNA of hepatitis D is amplified using host RNA polymerases, but also contains a single hepatitis D antigen (HDAg) gene. During hepatitis B and D coinfection or superinfection, intact hepatitis D viruses are packaged with an envelope containing hepatitis B surface antigens surrounding the RNA genome coated with HDAg protein. Incorporation of the hepatitis B surface antigens is essential for hepatitis D infectivity, as hepatitis D does not encode its own receptor binding proteins. Coinfection or superinfection with hepatitis D causes more severe complications, with increased risk of liver failure, cirrhosis, and cancer. There is a present need for effective immunogenic compositions and vaccines to establish prophylactic immunity against both hepatitis B and D infections.

### SUMMARY

The present disclosure relates generally to the use of recombinant nucleic acids, DNA, RNA, proteins, polypeptides, or peptides comprising HBV and/or HDV antigens to induce immune responses, antibody production, immune protection, or immunity against HBV or HDV infections. In some embodiments, the recombinant nucleic acids, DNA, RNA, proteins, polypeptides, or peptides comprising HBV and/or HDV antigens are used in a DNA prime/protein boost composition approach. In some embodiments, this DNA prime/protein boost composition approach results in greater immune response, antibody production, immune protection, or immunity against HBV or HDV infections compared to DNA-only, protein-only, or organism-based immunogenic compositions.

Chronic hepatitis B and D virus (HBV/HDV) infections can cause cancer. Current HBV therapy using nucleoside analogues (NAs) is life-long and reduces but does not eliminate the risk of cancer. A hallmark of chronic hepatitis B is a dysfunctional HBV-specific T cell response. In some embodiments is an immunotherapy driven by naïve healthy T cells specific for the HDV antigen (HDAg) to bypass the need for HBV-specific T cells in order to prime PreS1-specific T cells and PreS1 antibodies blocking HBV entry. In some embodiments, combinations of PreS1 and/or HDAg sequences were evaluated for induction of PreS1 antibodies and HBV- and HDV-specific T cells in vitro and in vivo. In some embodiments, neutralization of HBV by PreS1-specific murine and rabbit antibodies was evaluated in cell culture, and rabbit anti-PreS1 were tested for neutralization of HBV in mice repopulated with human hepatocytes. In some embodiments, adoptive transfer of PreS1 antibodies prevented or modulated HBV infection after a subsequent challenge in humanized mice.

In some embodiments, the nucleic acid or polypeptide compositions comprise sequences, genes, or polypeptides of HBV, HDV, PreS1, or HDAg. In some embodiments, the PreS1 is PreS1 A or PreS1 B. In some embodiments, the HDAg is HDAg genotype 1 strain A (1 A), HDAg genotype 1 strain B (1 B), HDAg genotype 2 strain A (2 A), or HDAg genotype 2 strain B (2 B). In some embodiments, the HDAg comprises a C211 mutant. In some embodiments, the HDAg comprises a C211S mutant. In some embodiments, the compositions also comprise an autocatalytic peptide cleavage site. In some embodiments, the autocatalytic peptide cleavage site is a P2A autocatalytic peptide cleavage site. In some embodiments, the PreS1 and HDAg components are grouped together in the compositions. In some embodiments, the PreS1 is downstream or immediately downstream of the HDAg sequence. In some embodiments, the PreS1 and HDAg groups are separated by an autocatalytic peptide cleavage site. In some embodiments, the PreS1 and HDAg groups are separated by a P2A autocatalytic peptide cleavage site.

In some embodiments, the nucleic acid compositions are a plasmid, virus, bacteriophage, cosmid, fosmid, phagemid, bacterial artificial chromosome (BAC), yeast artificial chromosome (YAC), or human artificial chromosome (HAC). In some embodiments, the nucleic acid compositions are circular or linear. In some embodiments, the nucleic acid compositions are produced in a biological system, including but not limited to mammalian cells, human cells, bacteria cells, E. coli, yeast, S. cerevisiae, or other appropriate biological system. In some embodiments, the HBV and/or HDV nucleic acids or genes are found in a cassette that comprises elements needed to transcribe and translate the nucleic acids or genes in a biological system.

In some embodiments, the polypeptide compositions are properly folded or denatured. In some embodiments, the polypeptide compositions are produced in a biological system, including but not limited to mammalian, bacteria, yeast, insect, or cell-free recombinant expression systems. In some embodiments, the polypeptide compositions are produced in mammalian, human, primary, immortalized, cancer, stem, fibroblasts, human embryonic kidney (HEK) 293, Chinese Hamster Ovary (CHO), bacterial, Escherichia coli, yeast, Saccharomyces cerevisiae, Pichia pastoris, insect, Spodoptera frugiperda Sf9, or S. frugiperda Sf21 cells, or in a cell-free system. In some embodiments, the polypeptide compositions are produced in E. coli cells. In some embodiments, the polypeptide compositions are purified using techniques known in the art, including but not limited to extraction, freeze-thawing, homogenization, permeabilization, centrifugation, density gradient centrifugation, ultracentrifugation, precipitation, SDS-PAGE, native PAGE, size exclusion chromatography, liquid chromatography, gas chromatography, hydrophobic interaction chromatography, ion exchange chromatography, anion exchange chromatography, cation exchange chromatography, affinity chromatography, immunoaffinity chromatography, metal binding chromatography, nickel column chromatography, epitope tag purification, or lyophilization. In some embodiments, the polypeptide compositions are purified using metal binding chromatography, also known as immobilized metal affinity chromatography (IMAC).

In some embodiments, the nucleic acid or polypeptide compositions are administered to an animal, including but not limited to humans, mice, rats, rabbits, cats, dogs, horses, cows, pigs, sheep, monkeys, primates, or chickens. In some embodiments, the nucleic acid or polypeptide compositions are administered 1, 2, 3, 4, 5, 6, or 7 days, or 1, 2, 3, 4, 5, 6, 7, 8, 9, or 10 weeks, or 1, 2, 3, 4, 5, 6, 7, 8, 9, 10, 11, 12 months or any time within a range defined by any two of the aforementioned times between each dose. In some embodiments, the nucleic acid compositions are administered before the polypeptide compositions are administered. In some embodiments, the polypeptide compositions are administered before the nucleic acid compositions.

In some embodiments, the nucleic acid or polypeptide compositions are administered in an amount of 1, 10, 100, 1000 ng, or 1, 10, 100, 200, 300, 400, 500, 600, 700, 800, 900, 1000 μg, or 1, 10, 100, or 1000 mg or any amount within a range defined by any two of the aforementioned amounts. In some embodiments, the nucleic acid or polypeptide compositions are administered with excipients. In some embodiments, the nucleic acid or polypeptide compositions are administered with adjuvants. In some embodiments, the nucleic acid compositions are administered with in vivo electroporation.

In some embodiments, the immunogenicity of the nucleic acid or polypeptide compositions are assessed by measuring interferon gamma (IFNγ) producing immune cells using techniques known in the art, including ELISpot, measuring IgG antibody titer specific to HBV, HDV, HBV proteins, HBV nucleic acids, HDV proteins, HDV nucleic acids, PreS1, or HDAg, or measuring the neutralization activity of sera or purified antibodies from immunized animals in an in vitro or in vivo assay.

In some embodiments, the administration of the nucleic acid or polypeptide compositions provide transient, lasting, or permanent protection against an HBV or HDV infection. In some embodiments, the transient, lasting, or permanent protection against an HBV or HDV infection is superior to other immunogenic compositions. In some embodiments, the administration of the nucleic acid or polypeptide compositions is performed in conjunction with an antiviral therapy. In some embodiments, the administration of the nucleic acid or polypeptide compositions to provide transient, lasting, or permanent protection against an HBV or HDV infection is effective in humans. In some embodiments, the nucleic acid or polypeptide compositions are used as vaccines or immunogens for treating, inhibiting, or ameliorating HBV or HDV infection or for providing protection against HBV and/or HDV infection.

Preferred aspects of the present invention related to the following numbered alternatives:
1. A polypeptide comprising at least one hepatitis D antigen (HDAg) polypeptide sequence and at least one PreS1 polypeptide sequence, wherein each of the at least one HDAg polypeptide sequence is a C211S mutant HDAg polypeptide sequence.
2. The polypeptide of alternative 1, wherein the at least one HDAg polypeptide sequence comprises the sequence of SEQ ID NO: 47, SEQ ID NO: 48, SEQ ID NO: 49, SEQ ID NO: 50, or any combination thereof.
3. The polypeptide of alternative 1 or 2, wherein the at least one PreS1 polypeptide sequence comprises the sequence of SEQ ID NO: 11 or SEQ ID NO: 12, or both.
4. The polypeptide of any one of alternatives 1-3, wherein the polypeptide comprises one or more epitope tags.
5. The polypeptide of alternative 4, wherein the one or more epitope tags comprise an E-tag, Myc tag, FLAG tag, Strep2 tag, 6x-histidine tag, or any combination thereof.
6. The polypeptide of any one of alternatives 1-5, wherein the polypeptide comprises a sequence having at least 80%, 85%, 90%, 95%, 99%, or 100% homology or sequence identity to the sequence of any one of SEQ ID NO: 72-95, 140-141, 145-147, 163-177, or 187-195.
7. The polypeptide of any one of alternatives 1-6, wherein the polypeptide is recombinantly expressed.
8. The polypeptide of any one of alternatives 1-7, wherein the polypeptide is recombinantly expressed in a mammalian, bacterial, yeast, insect, or cell-free system.
9. The polypeptide of any one of alternatives 1-8, wherein the polypeptide is recombinantly expressed in a bacterial system, optionally wherein the polypeptide is recombinantly expressed in E. coli.
10. A nucleic acid comprising at least one HDAg nucleic acid sequence and at least one PreS1 nucleic acid sequence, wherein each of the at least one HDAg nucleic acid sequence is a C211S mutant HDAg nucleic acid sequence.
11. The nucleic acid of alternative 10, wherein the at least one HDAg nucleic acid sequence and/or the at least one PreS1 nucleic acid sequence are codon optimized to minimize or reduce the number of repeat sequences.
12. The nucleic acid of alternative 10 or 11, wherein the at least one HDAg nucleic acid sequence comprises the sequence of SEQ ID NO: 43, SEQ ID NO: 44, SEQ ID NO: 45, SEQ ID NO: 46, or any combination thereof.
13. The nucleic acid of any one of alternatives 1-12, wherein the at least one PreS1 nucleic acid sequence comprise the sequence of SEQ ID NO: 51, SEQ ID NO: 52, SEQ ID NO: 53, or any combination thereof.
14. The nucleic acid of any one of alternatives 1-13, wherein the nucleic acid further encodes one or more epitope tags.
15. The nucleic acid of alternative 14, wherein the one or more epitope tags comprise an E-tag, Myc tag, FLAG tag, Strep2 tag, 6x-histidine tag, or any combination thereof.
16. The nucleic acid of any one of alternatives 1-15, wherein the nucleic acid comprises a sequence having at least 80%, 85%, 90%, 95%, 99%, or 100% homology or sequence identity to the sequence of any one of SEQ ID NO: 60-71, 138-139, 142-144, 148-162, or 178-186.
17. The nucleic acid of any one of alternatives 1-16, wherein the nucleic acid is DNA.
18. The nucleic acid of any one of alternatives 1-17, wherein the nucleic acid is provided as a recombinant vector.
19. A nucleic acid operon comprising two or more genes, wherein each of the two or more genes comprise at least one HDAg nucleic acid sequence and at least one PreS1 nucleic acid sequence, and wherein each of the two or more genes comprises a 5′ ribosome binding site that enables translation.
20. The nucleic acid operon of alternative 19, wherein the at least one HDAg nucleic acid is a C211S mutant HDAg nucleic acid sequence.
21. The nucleic acid operon of alternative 19 or 20, wherein the at least one HDAg nucleic acid sequence and/or the at least one PreS1 nucleic acid sequence are codon optimized to reduce the number of repeat sequences.
22. The nucleic acid operon of any one of alternatives 19-21, wherein the at least one HDAg nucleic acid sequence comprises the sequence of SEQ ID NO: 43, SEQ ID NO: 44, SEQ ID NO: 45, SEQ ID NO: 46, or any combination thereof.
23. The nucleic acid operon of any one of alternatives 19-22, wherein the at least one PreS1 nucleic acid sequence comprise the sequence of SEQ ID NO: 51, SEQ ID NO: 52, SEQ ID NO: 53, or any combination thereof.
24. The nucleic acid operon of any one of alternatives 19-23, wherein each of the two or more genes further encode one or more epitope tags.
25. The nucleic acid operon of alternative 24, wherein the one or more epitope tags comprise an E-tag, Myc tag, FLAG tag, Strep2 tag, 6x-histidine tag, or any combination thereof.
26. The nucleic acid operon of any one of alternatives 19-25, wherein each of the two or more genes are configured such that the at least one PreS1 nucleic acid sequence is downstream of the at least one HDAg nucleic acid sequence.
27. The nucleic acid operon of any one of alternatives 19-26, wherein the nucleic acid operon comprises a sequence having at least 80%, 85%, 90%, 95%, 99%, or 100% homology or sequence identity to the sequence of any one of SEQ ID NO: 96-101, or 196-199.
28. The nucleic acid operon of any one of alternatives 19-27, wherein the two or more genes encode two or more polypeptides comprising the sequence of any one of SEQ ID NO: 102-133.
29. A cell comprising the polypeptide of any one of alternatives 1-9, the nucleic acid of any one of alternatives 10-18, or the nucleic acid operon of any one of alternatives 19-28.
30. An immunogenic composition comprising the polypeptide of any one of alternatives 1-9 and a nucleic acid comprising at least one nucleic acid sequence encoding HDAg and at least one nucleic acid sequence encoding PreS1.
31. The immunogenic composition of alternative 30, wherein the at least one nucleic acid sequence encoding HDAg comprises SEQ ID NO: 1-4, or 43-46, or any combination thereof.
32. The immunogenic composition of alternative 30 or 31, wherein the at least one nucleic acid sequence encoding PreS1 comprises SEQ ID NO: 9-10 or 51-53, or any combination thereof.
33. The immunogenic composition of any one of alternatives 30-32, wherein the nucleic acid comprises a sequence having at least 80%, 85%, 90%, 95%, 99%, or 100% homology or sequence identity to SEQ ID NO: 15-24 or 35-36.
34. The immunogenic composition of any one of alternatives 30-33, wherein the nucleic acid comprises a sequence having at least 80%, 85%, 90%, 95%, 99%, or 100% homology or sequence identity to SEQ ID NO: 18.
35. The immunogenic composition of any one of alternatives 30-34, wherein the nucleic acid is DNA, optionally wherein the nucleic acid is provided as a recombinant vector.
36. The immunogenic composition of any one of alternatives 30-35, further comprising an adjuvant, optionally wherein the adjuvant is alum, QS-21, or MF59, or any combination thereof.
37. A method of generating an immune response in a subject comprising administering to the subject the polypeptide of any one of alternatives 1-9, the nucleic acid of any one of alternatives 10-18, the proteins encoded by the nucleic acid operons of any one of alternatives 19-28, or the immunogenic composition of any one of alternatives 30-36.
38. The method of alternative 37, wherein the immunogenic composition is administered in a prime-boost approach, wherein the subject is administered at least one prime dose comprising the nucleic acid of the immunogenic composition, and subsequently administered at least one boost dose comprising the polypeptide of the immunogenic composition.
39. The method of alternative 38, wherein the at least one boost dose is administered at least 1, 2, 3, 4, 5, 6, 7, 8, 9, 10, 11, 12, 24, 36, or 48 days or weeks after the at least one prime dose is administered or within a range of time defined by any two of the aforementioned time points e.g., within 1-48 days or 1-48 weeks.
40. The method of any one of alternatives 37, wherein the administration is provided enterally, orally, intranasally, parenterally, subcutaneously, intramuscularly, intradermally, or intravenously or any combination thereof.
41. The polypeptide of any one of alternatives 1-9, the nucleic acid of any one of alternatives 10-18, the proteins encoded by the nucleic acid operons of any one of alternatives 19-28, or the immunogenic composition of any one of alternatives 30-36 for use as a medicament, e.g., in the treatment, prevention, amelioration, or inhibition of hepatitis B and/or hepatitis D in a subject in need thereof.
42. A polypeptide comprising a sequence having at least 80%, 85%, 90%, 95%, 99%, or 100% homology or sequence identity to the sequence of any one of SEQ ID NO: 136-137, 187-195, or 200-203.
43. A nucleic acid comprising a sequence having at least 80%, 85%, 90%, 95%, 99%, or 100%, homology or sequence identity to the sequence of any one of SEQ ID NO: 134-135, 178-186, or 196-199.
44. An immunogenic composition or product combination comprising:
(a) a nucleic acid comprising at least one nucleic acid sequence encoding hepatitis D antigen (HDAg) and at least one nucleic acid sequence encoding PreS1; and
(b) a polypeptide comprising at least one HDAg polypeptide sequence and at least one PreS1 polypeptide sequence.
45. The immunogenic composition or product combination of alternative 44, wherein the at least one nucleic acid sequence encoding HDAg comprises SEQ ID NO: 1-4 or 43-46, or any combination thereof.
46. The immunogenic composition or product combination of alternative 44 or 45, wherein the at least one nucleic acid sequence encoding PreS1 comprises SEQ ID NO: 9-10 or 51-53, or any combination thereof.
47. The immunogenic composition or product combination of any one of alternatives 44-46, wherein the nucleic acid is configured such that each HDAg nucleic acid sequence is grouped with a PreS1 nucleic acid sequence, and wherein the PreS1 nucleic acid sequence is immediately downstream of the HDAg nucleic acid sequence.
48. The immunogenic composition or product combination of alternative 47, further comprising at least one nucleic acid sequence encoding an autocatalytic peptide cleavage site, wherein the grouped HDAg and PreS1 nucleic acid sequences are separated by the at least one nucleic acid sequence encoding the autocatalytic peptide cleavage site.
49. The immunogenic composition or product combination of alternative 48, wherein the at least one nucleic acid sequence encoding the autocatalytic peptide cleavage site comprises a nucleic acid sequence selected from the group consisting of porcine teschovirus-1 2A (P2A), foot-and-mouth disease virus 2A (F2A), equine rhinitis A virus (ERAV) 2A (E2A) and Thosea asigna virus 2A (T2A) nucleic acid, and wherein each encoded autocatalytic peptide cleavage site may optionally include a GSG (glycine-serine-glycine) motif at its N-terminus.
50. The immunogenic composition or product combination of alternative 48 or 49, wherein the at least one nucleic acid sequence encoding the autocatalytic peptide cleavage site comprises SEQ ID NO: 13.
51. The immunogenic composition or product combination of any one of alternatives 44-50, wherein the nucleic acid is codon optimized for expression in a human.
52. The immunogenic composition or product combination of any one of alternatives 44-51, wherein the nucleic acid comprises a sequence having at least 80%, 85%, 90%, 95%, 99%, or 100% homology or sequence identity to SEQ ID NO: 15-24, 35-36, 60-71, 134-135, or 138-139.
53. The immunogenic composition or product combination of any one of alternatives 44-52, wherein the nucleic acid comprises a sequence having at least 80%, 85%, 90%, 95%, 99%, or 100% homology or sequence identity to SEQ ID NO: 134-135, 138-139, 178-186, or 196-199.
54. The immunogenic composition or product combination of any one of alternatives 44-53, wherein the at least one HDAg polypeptide comprises SEQ ID NO: 5-8 or 47-50, or any combination thereof.
55. The immunogenic composition or product combination of any one of alternatives 44-54, wherein the at least one PreS1 polypeptide sequence comprises SEQ ID NO: 11 or SEQ ID NO: 12, or both.
56. The immunogenic composition or product combination of any one of alternatives 44-55, wherein the at least one PreS1 polypeptide sequence is downstream of the at least one HDAg polypeptide sequence.
57. The immunogenic composition or product combination of any one of alternatives 44-56, wherein the polypeptide comprises a sequence having at least 80%, 85%, 90%, 95%, 99%, or 100% homology or sequence identity to the sequence of SEQ ID NO: 25-34, 37, 72-95, 136-137, or 140-141.
58. The immunogenic composition or product combination of any of alternatives 44-57, wherein the polypeptide comprises a sequence having at least 80%, 85%, 90%, 95%, 99%, or 100% homology or sequence identity to the sequence of SEQ ID NO: 136-137, 140-141, 187-195, or 200-203.
59. The immunogenic composition or product combination of any one of alternatives 44-58, wherein the polypeptide is recombinantly expressed.
60. The immunogenic composition or product combination of alternative 59, wherein the polypeptide is recombinantly expressed in a mammalian, bacterial, yeast, insect, or cell-free system.
61. The immunogenic composition or product combination of any one of alternatives 44-60, further comprising an adjuvant.
62. The immunogenic composition or product combination of alternative 61, wherein the adjuvant is alum, QS-21 or MF59, or any combination thereof.
63. The immunogenic composition or product combination of any one of alternatives 44-62, wherein the nucleic acid comprises DNA.
64. The immunogenic composition or product combination of any one of alternatives 44-63, wherein the nucleic acid is provided in a recombinant vector.
65. A method of generating an immune response in a subject using the immunogenic composition or product combination set forth in any one of alternatives 44-65, comprising:
administering to the subject at least one prime dose comprising the nucleic acid; and
administering to the subject at least one boost dose comprising the polypeptide.
66. The method of alternative 65, wherein the at least one boost dose further comprises an adjuvant.
67. The method of alternative 66, wherein the adjuvant is alum, QS-21, or MF59, or any combination thereof.
68. The method of any one of alternatives 65-67, wherein the at least one boost dose is administered at least 1, 2, 3, 4, 5, 6, 7, 8, 9, 10, 11, 12, 24, 36, or 48 days or weeks after the at least one prime dose is administered or within a range of time defined by any two of the aforementioned time points e.g., within 1-48 days or 1-48 weeks.
69. The method of any one of alternatives 65-68, wherein the administration is provided enterally, orally, intranasally, parenterally, subcutaneously, intramuscularly, intradermally, or intravenously or any combination thereof.
70. The method of any one of alternatives 65-69, wherein the administration is performed in conjunction with an antiviral therapy.
71. The method of alternative 70, wherein the antiviral therapy comprises administration of entecavir, tenofovir, lamivudine, adefovir, telbivudine, emtricitabine, interferon-a, pegylated interferon-a, or interferon alfa-2b, or any combination thereof.

### BRIEF DESCRIPTION OF THE DRAWINGS

In addition to the features described above, additional features and variations will be readily apparent from the following descriptions of the drawings and exemplary embodiments. It is to be understood that these drawings depict typical embodiments and are not intended to be limiting in scope.

FIG. 1A-1B depicts exemplary nucleic acid or polypeptide constructs comprising HBV and/or HDV antigens used herein. Ten constructs are provided, Delta-1 (Δ-1, D1), Delta-2 (Δ-2, D2), Delta-3 (Δ-3, D3), Delta-4 (Δ-4, D4), Delta-5 (Δ-5, D5), Delta-6 (Δ-6, D6), Delta-7 (Δ-7, D7), Delta-8 (Δ-8, D8), Delta-9 (Δ-9, D9), and Delta-10 (Δ-10, D10) (FIG. 1A). Western blot confirms that the ten polypeptide constructs are properly expressed (FIG. 1B). GFP was used as a control for Western blot. The constructs provided herein may be used in either nucleic acid or protein form, or in a nucleic acid prime/protein boost composition approach, where the nucleic acid form of any one or more of these or other constructs is used in conjunction with the protein form of any one or more of these or other constructs.

FIG. 2 depicts additional exemplary nucleic acid or polypeptide constructs comprising HBV and/or HDV antigens used herein. These additional constructs include a fusion of Δ-7 and Δ-8 (Δ-7+Δ-8; Delta-7+Delta-8; D-7+D-8), Δ-7S-8S (Delta-7S-8S; D-7S-8S), and 4-78 (Delta-78; D-78). The constructs provided herein may be used in either nucleic acid or protein form, or in a nucleic acid prime/protein boost composition approach, where the nucleic acid form of any one or more of these or other constructs is used in conjunction with the protein form of any one or more of these or other constructs.

FIG. 3A-3E depict the quantification of interferon gamma (IFNγ) forming spots per 10⁶ cells on an ELISpot assay, which corresponds to T lymphocyte activation, of a purified white blood cell population from sera derived from mice immunized with HBV/HDV DNA compositions in response to exposure to various HBV or HDV antigens. Antigens include purified polypeptides comprising PreS1 A (SEQ ID NO: 11), PreS1 A (SEQ ID NO: 12), HDAg genotype 1 A (SEQ ID NO: 5, “HDAg gtp 1A-pool 1” and “HDAg gtp 1A-pool 2”), HDAg genotype 1 B (SEQ ID NO: 6, “HDAg gtp 1B-pool 3” and “HDAg gtp 1B-pool 4”), HDAg genotype 2 A (SEQ ID NO: 7, “HDAg gtp 2C-pool 5” and “HDAg gtp 2C-pool 6”) and HDAg genotype 2 B (SEQ ID NO: 8, “HDAg gtp 2D-pool 7” and “HDAg gtp 2D-pool 8”). Mice sacrificed 6 weeks after first immunization and pooled splenocytes from each group were stimulated for 48 hours with the HDV peptide pools 1-8 corresponding to genotype 1 (pools 1-4) and genotype 2 (pools 5-8). Pools 1 and 2 of genotype 1 refer to sequence/isolate A while pools 3 and 4 correspond to sequence/isolate B. Similarly, pools 5 and 6 of genotype 2 refer to sequence/isolate C and pools 7 and 8 of genotype 2 refer to sequence/isolate D. Each pool contained 20 or 221 (for pools 1 and 5) 15-mer peptides with 10 aa overlap. Concanavalin A (“ConA”) was used as a positive control, and two ovalbumin peptides (“OVA Th” and “OVA CTL”) and growth medium (“media”) were used as negative controls. Each peptide-stimulated group were run in triplicates and bars show the mean number of IFNγ spot forming cells (SFC) per 106 cells with standard error. A cut-off was set at 100 SFCs/10⁶ splenocytes. Concentrations of the antigens are provided.

FIG. 4A-4C depict the quantification of anti-PreS1 IgG antibody titer in sera derived from mice immunized with HBV/HDV DNA compositions. Constructs Δ-1 to Δ-10 were tested for generation of IgG antibodies against PreS1A and PreS1B consensus sequences in mice (5 mice per group). FIG. 4A-4B cover reactivity against PreS1 amino acids 2-48. FIG. 4C cover cross-reactivity against HBV (sub-) genotypes A1, A2, B, B2, C, D1, E1 and F.

FIG. 5A-5C depict the quantification of interferon gamma (IFNγ) forming spots per 106 cells on an ELISpot assay of a purified white blood cell population from sera derived from C57BL/6 or HLA-A2 transgenic HHD mice immunized with a Δ-4 DNA composition, or naïve C57BL/6 mice in response to exposure to various HBV or HDV antigens or peptides. Antigens include purified polypeptides comprising PreS1 A (SEQ ID NO: 11), PreS1 A (SEQ ID NO: 12), a pool comprising HDAg genotypes 1 A and 1 B (SEQ ID NOS: 5 and 6, “gtp 1-pool 1”, “gtp 1-pool 2”, “gtp 1-pool 3”, “gtp 1-pool 4”), a pool comprising HDAg genotypes 2 A and 2 B (SEQ ID NOs: 7 and 8, “gtp 2-pool B1”, “gtp 2-pool B2”, “gtp 2-pool B3”, “gtp 2-pool B4”), HDAg peptide fragment pools comprising peptides KLEDDNPWL (SEQ ID NO: 40), KLEEENPWL (SEQ ID NO: 41), and FPWDILFPA (SEQ ID NO: 42) (“pep-3-pool”), and individual HDAg peptides KLEDDNPWL (SEQ ID NO: 40), KLEEENPWL (SEQ ID NO: 41), and FPWDILFPA (SEQ ID NO: 42). Concanavalin A (“ConA”) was used as a positive control, and two ovalbumin peptides (“OVA Th” and “OVA CTL”) and growth medium (“media”) were used as negative controls. Concentrations of the antigens are provided.

FIG. 6A-6C depict the quantification of anti-PreS1 IgG titer in New Zealand white rabbits immunized with Δ-3 or Δ-4 DNA compositions. Serum from the rabbits were collected and tested by ELISA against the PreS1A and PreS1B consensus peptides (FIG. 6B). The vaccinated rabbit anti-sera also tested for cross-reactivity to HBV (sub-) genotypes A1, A2, B, B2, C, D1, E1, and F (FIG. 6C). Graph bars show the mean end anti-PreS1 titers for each group determined as the end last serum dilution giving an OD at 405 nm three times higher than the OD of non-immunized sera at the same dilution. The sera was titrated serially with six-fold dilutions starting at 1:60. FIG. 6D shows the percentage of reactivity of D-4 vaccinated rabbit antisera against PreS1 of different HBV (sub-) genotypes. Six weeks old D-4 vaccinated rabbit antisera were tested for reactivity (at OD 405 nm) against HBV (sub-) genotypes D1, F, A1, C, A2, B, B2 and E1 by ELISA. Using individual 20 mer PreS1 peptides with ten aa overlap corresponding to each HBV type of aa 2-21, 12-31, 22-41, and 32-48, neutralizing epitopes mainly localized at aa 22-41 and 32-48 of genotype D1 as indicated by the highest percentage of reactivity, followed by (sub-) types C, E1 and A1 at the same aa region.

FIG. 7A-7C depict the quantification of interferon gamma (IFNγ) forming spots per 106 cells on an ELISpot assay of a purified white blood cell population from sera derived from C57BL/6 mice immunized with a Δ-4 DNA-only, Δ-7 protein-only, or Δ-4 DNA/Δ-8 protein prime/boost composition. Antigens include purified polypeptides comprising PreS1 A (SEQ ID NO: 11), PreS1 A (SEQ ID NO: 12), a pool comprising HDAg genotypes 1 A and 1 B (SEQ ID NOs: 5 and 6, “gtp 1-pool 1”, “gtp 1-pool 2”, “gtp 1-pool 3”, “gtp 1-pool 4”), and a pool comprising HDAg genotypes 2 A and 2 B (SEQ ID NOs: 7 and 8, “gtp 2-pool 5”, “gtp 2-pool 6”, “gtp 2-pool 7”, “gtp 2-pool 8”). Concanavalin A (“ConA”) was used as a positive control, and two ovalbumin peptides (“OVA Th” and “OVA CTL”), DMSO, and growth medium (“media”) were used as negative controls. Concentrations of the antigens are provided.

FIG. 8A-8C depict the quantification of anti-PreS1 IgG titer in C57BL/6 mice immunized with exemplary HBV/HDV DNA-only, protein-only, or DNA prime/protein boost compositions.

FIG. 9 depicts the quantification of anti-PreS1 IgG titer in rabbits immunized with exemplary HBV/HDV DNA-only, protein-only, or DNA prime/protein boost compositions.

FIG. 10A-10B depicts the protective effect against HBV infection at 1, 2, 3, 4, 6 and 8 weeks after first inoculation, as determined based on the HBV titers at each time point. Each line indicates one individual mouse (FIG. 10A). Two negative control mice (grey lines) received non-immunized IgG and three mice (red lines) received D4 PreS1 IgG. One mouse of the PreS1-IgG treated group died at week 4, thus only measurements on week 1, 2 and 3 are available for this mouse. There were no significant differences between the groups with respect to serum levels of alanine transferase, asparagine transferase, alkaline phosphatase, or bilirubin (FIG. 10B).

FIG. 11A-11D depict the assessment of the D-7 and D-8 peptide mixture (10 μg each for administration in mice) with different adjuvants. QS-21, MF59, and alum adjuvants were tested. D-4 DNA compositions administered intramuscularly with electroporation was used as control. FIG. 11A shows the dosing schedule and exemplary end titers with the tested adjuvants. FIG. 11B shows % reactivity in individual mice of each condition assessed by ELISA. The x-axis (“1, 3, 10, 30, 0”) corresponds to ID numbers of individual mice. FIG. 11C shows IFNγ activation of splenocytes by HBV PreS1 and HDV antigen consensus peptides as assessed by ELISpot. FIG. 11D shows end-point PreS1 titers against PreS1A and PreS1B peptides.

FIG. 12A-12D depict a comparison of D-7 and D-8 peptide mixture only, D-7+D-8 fusion peptide only, and D-4 DNA prime and D-7 and D-8 peptide mixture boost compared to D-4 DNA only and naïve controls. FIG. 12A shows IFNγ activation of splenocytes by HBV PreS1 and HDV antigen consensus peptides as assessed by ELISpot. FIG. 12B shows antibody levels against PreS1A assessed 2 weeks after the first round of administration. FIG. 12C shows antibody levels against PreS1A assessed 2 weeks after the second round of administration. FIG. 12D shows antibody levels against PreS1B assessed 2 weeks after the second round of administration. The legends for FIG. 12B-D correspond to ID numbers of individual mice.

FIG. 13 depicts exemplary HBV/HDV immunogenic construct (D-7, D-8, and D-7+D-8 fusion peptide) protein expression in E. coli. SDS-PAGE gels of purified product were stained with Coomassie Brilliant Blue. Lane 1 contains 2 μg of BSA as control, and lane 2 contains 2 μg of the expressed HBV/HDV peptide.

FIG. 14 depicts additional exemplary nucleic acid or polypeptide constructs comprising HBV and/or HDV antigens used herein. Twelve constructs are provided herein, F1-A, F1-B, F2, F3-A, F3-B, F4, F5, F6, F7, F8, F9, and F10. The constructs are each intended to be one contiguous sequence. “GGG” refers to a triple glycine linker (SEQ ID NO: 54). However, it is envisioned that other linkers conventionally known in the art can be substituted. The constructs depicted herein can be used for any of the compositions or methods disclosed herein. The constructs provided herein may be used in either nucleic acid or protein form, or in a nucleic acid prime/protein boost composition approach, where the nucleic acid form of any one or more of these or other constructs is used in conjunction with the protein form of any one or more of these or other constructs.

FIG. 15 depicts additional exemplary nucleic acid constructs comprising HBV and/or HDV antigens used herein to be used in an operon approach. Six constructs are provided herein, O1, O2-A, O2-B, O3, O4, and O5. The constructs are intended to be expressed in a prokaryote, such as E. coli, such that the presence of internal ribosome binding sites (RBS) results in the production of more than one separate protein during translation. The use of a prokaryote such as E. coli enable large scale production, e.g. for use in preparation of an immunogenic composition. “PROM” denotes an appropriate promoter, such as the T7 promoter, and “Term” denotes an appropriate terminator sequence to signal the end of transcription.

FIG. 16 depicts additional exemplary nucleic acid or polypeptide constructs comprising HBV and/or HDV antigens used herein. Three constructs are provided herein, A-7S-8S-L1 (Delta-78-8S-L1; D-7S-8S-L1), Δ-7S-8S-L2 (Delta-78-8S-L2; D-7S-8S-L2), and Δ-7S-8S-L3 (Delta-78-8S-L3; D-7S-8S-L3), which are modifications of Δ-7S-8S with linkers interspersed between the HBV and/or HDV antigens. “GGG” refers to a triple glycine linker (SEQ ID NO: 54). However, it is envisioned that other linkers conventionally known in the art can be substituted. The constructs depicted herein can be used for any of the compositions or methods disclosed herein. The constructs provided herein may be used in either nucleic acid or protein form, or in a nucleic acid prime/protein boost composition approach, where the nucleic acid form of any one or more of these or other constructs is used in conjunction with the protein form of any one or more of these or other constructs.

FIG. 17 depicts additional exemplary nucleic acid or polypeptide constructs comprising HBV and/or HDV antigens used herein. Fifteen constructs are provided herein, Δ-78-L1 (Delta-78-L1; D-78-L1), Δ-78-L2 (Delta-78-L2; D-78-L2), Δ-78-L3 (Delta-78-L3; D-78-L3), Δ-78-L4 (Delta-78-L4; D-78-L4), Δ-78-L5 (Delta-78-L5; D-78-L5), Δ-78-L6 (Delta-78-L6; D-78-L6), Δ-78-L7 (Delta-78-L7; D-78-L7), Δ-78-L8 (Delta-78-L8; D-78-L8), Δ-78-L9 (Delta-78-L9; D-78-L9), Δ-78-L10 (Delta-78-L10; D-78-L10), Δ-78-L11 (Delta-78-L11; D-78-L11), Δ-78-L12 (Delta-78-L12; D-78-L12), Δ-78-L13 (Delta-78-L13; D-78-L13), Δ-78-L14 (Delta-78-L14; D-78-L14), Δ-78-L15 (Delta-78-L15; D-78-L15), which are modifications of Δ-78 with linkers interspersed between the HBV and/or HDV antigens. “GGG” refers to a triple glycine linker (SEQ ID NO: 54). However, it is envisioned that other linkers conventionally known in the art can be substituted. The constructs depicted herein can be used for any of the compositions or methods disclosed herein. The constructs provided herein may be used in either nucleic acid or protein form, or in a nucleic acid prime/protein boost composition approach, where the nucleic acid form of any one or more of these or other constructs is used in conjunction with the protein form of any one or more of these or other constructs.

FIG. 18 depicts a Coomassie Brilliant Blue (CBB) SDS-PAGE gel and corresponding anti-His Western blot, showing that some exemplary constructs disclosed herein (e.g., F2, F5, and F7) exhibit smaller molecular weight degradation products when expressed in E. coli.

FIG. 19 depicts additional exemplary nucleic acid or polypeptide constructs comprising HBV and/or HDV antigens used herein. Five constructs are provided herein, F11, F12, F13A/F13B (which are identical in terms of peptide sequence but are alternative versions of codon optimized nucleic acid sequences), F14, and F15. A common motif exemplified by these constructs is the use of HDAg genotype 2 sequences (e.g., HDAg 2 A or HDAg 2 B) in the 5′ end/N-terminus, and/or not having HDAg genotype 1 sequences (e.g., HDAg 1 A or HDAg 1 B) in the 5′ end/N-terminus. “GGG” refers to a triple glycine linker (SEQ ID NO: 54). However, it is envisioned that other linkers conventionally known in the art can be substituted. The constructs depicted herein can be used for any of the compositions or methods disclosed herein. The constructs provided herein may be used in either nucleic acid or protein form, or in a nucleic acid prime/protein boost composition approach, where the nucleic acid form of any one or more of these or other constructs is used in conjunction with the protein form of any one or more of these or other constructs.

FIG. 20 depicts additional exemplary nucleic acid constructs comprising HBV and/or HDV antigens used herein to be employed in an operon approach. Three constructs are provided herein, O6, O7a, and O7b. The constructs are intended to be expressed in a prokaryote, such as E. coli, such that the presence of internal ribosome binding sites (RBS) results in the production of more than one separate protein during translation. The use of a prokaryote such as E. coli enable large scale production, e.g., for use in preparation of an immunogenic composition. A common motif exemplified by these constructs is the use of HDAg genotype 2 sequences (e.g., HDAg 2 A or HDAg 2 B) as the first gene on the operon construct, and/or not having HDAg genotype 1 sequences (e.g., HDAg 1 A or HDAg 1 B) as the first gene on the operon construct. “PROM” denotes an appropriate promoter, such as the T7 promoter, and “Term” denotes an appropriate terminator sequence to signal the end of transcription.

FIG. 21 depicts the quantified optical densities at 600 nm (OD₆₀₀) of E. coli cultures transformed to express exemplary constructs disclosed herein (F11, F12, F13A, F13B, F14, F15, F2, Δ7-8, O5, O6, F1-A, F1-B, F2, F3-A, F3-B, F4, F5, F6, F7, F8, F9, and F10) at the following conditions: 1) overnight pre-culture, 2) 4 hours of non-induced growth at 25° C. following an initial culture at OD₆₀₀ of approximately 1, and 3) 4 hours of IPTG-induced growth and expression at 25° C. following an initial culture at OD₆₀₀ of approximately 1. “V” denotes an untransformed control.

FIG. 22 depicts CBB stained SDS-PAGE gels of lysates of E. coli induced to express exemplary constructs provided in FIG. 21 showing the protein expression of the constructs. “V” denotes an untransformed control.

FIG. 23 depicts CBB stained SDS-PAGE gels and corresponding anti-His Western blots of lysates of E. coli induced to express exemplary constructs provided in FIG. 21 under reduced and non-reduced conditions.

FIG. 24 depicts an CBB stained SDS-PAGE gel and corresponding Western blots probing for 6×His, E-tag, Myc, FLAG, and Strep2 of E. coli lysates induced to express proteins from the O5 and O6 operon constructs disclosed herein, where these constructs exhibit distinct ordering of HDAg and PreS1 fusions of differing genotypes. In this case, the HDAg 1 A-PreS1A polypeptide is tagged with 6×His and E-tag; the HDAg 2 A-PreS1B polypeptide is tagged with 6×His and Myc tag; the HDAg 1 B-PreS1A polypeptide is tagged with 6×His and FLAG tag; and the HDAg2 B-PreS1B polypeptide is tagged with 6×His and Strep2 tag, permitting detection of these separate polypeptides using the respective tags.

FIG. 25 depicts a CBB stained SDS-PAGE gel of E. coli lysates induced to express the exemplary constructs F12, F13A, F14, and O6, processed using either the BugBuster® or NZY cell lysis reagents.

FIG. 26 depicts CBB stained SDS-PAGE gels, and corresponding quantification of final OD₆₀₀ after induction, of E. coli lysates induced to express the exemplary constructs F12, F13, F14, and O6 after being cultured 1) in Luria-Bertani broth (LB) with an initial OD₆₀₀ of 1, 2) in autoinduction (AI) media with an initial OD₆₀₀ of 0.5, 3) in LB with an initial OD₆₀₀ of 3.5, and 4) in Terrific broth (TB) with an initial OD₆₀₀ of 5.

FIGS. 27A-27B depicts an analysis of a purification run (Run 1) of expressed protein from E. coli induced to express the exemplary construct O3. FIG. 27A depicts a chromatogram of the process and the buffer conditions that were used. FIG. 27B depicts an analytical size exclusion chromatography (AnSEC) plot, CBB stained SDS-PAGE gel, and anti-His Western blot of the purified product.

FIGS. 28A-28B depicts an analysis of a purification run (Run 2) of expressed protein from E. coli induced to express the exemplary construct O3. FIG. 28A depicts a chromatogram of the process and the buffer conditions that were used. FIG. 28B depicts an analytical size exclusion chromatography (AnSEC) plot, CBB stained SDS-PAGE gel, and anti-His Western blot of the purified product.

FIGS. 29A-B depicts an analysis of a purification run (Run 3) of expressed protein from E. coli induced to express the exemplary construct O3. FIG. 29A depicts a chromatogram of the process and the buffer conditions that were used. FIG. 29B depicts an analytical size exclusion chromatography (AnSEC) plot, CBB stained SDS-PAGE gel, and anti-His Western blot of the purified product.

FIG. 30 depicts an analysis of the effects of differing elution buffers for purifying expressed protein from E. coli induced to express the exemplary construct O3. In “Run 3”, a solution of 0.5 M NaCl and 250 mM imidazole was used as a base elution buffer, and iterations of the elution buffer included 1) addition of 0.5 M arginine (Arg), 2) addition of 10% glycerol (Gly), 3) addition of 0.2% sodium dodecyl sulfate (SDS), or 4) diluting the base elution buffer 5 fold. In “Run 4”, a solution of 150 mM NaCl, 250 mM imidazole, and 0.5 M arginine (Arg) was used as a base elution buffer, and iterations of the elution buffer included 1) addition of 0.5 M NaCl, 2) addition of 10% glycerol, or 3) addition of 0.2% SDS.

FIG. 31 depicts an exemplary workflow for testing conditions for purifying expressed protein from E. coli induced to express the exemplary construct O6, and resultant CBB stained SDS-PAGE gels showing the overall protein yield of 4 separate runs. (FT refers to the flow through sample; W1 and W2 refer to the first and second wash, respectively; Elu refers to the elution sample)

FIG. 32 depicts an AnSEC plot of protein purified from E. coli induced to express the exemplary construct O6 following the “Run 3” workflow as shown in FIG. 30, using an elution buffer containing 350 mM imidazole, and a plot comparing the protein yields when using elution conditions of 250 mM or 350 mM imidazole with or without 0.5 mM arginine, quantified 0, 24, 48, or 72 hours after elution.

FIGS. 33A-33B depict an analysis of protein purified from E. coli induced to express the exemplary construct O6 following the “Run 4” workflow as shown in FIG. 31, using an elution buffer containing 350 mM imidazole. FIG. 33A shows an AnSEC plot showing different elution profiles depending on the concentration of arginine. FIG. 33B shows a CBB stained SDS-PAGE gel showing the yield of protein under differing conditions using 250 mM or 350 mM imidazole and with or without arginine, qualified 0, 24, or 48 hours after elution; and an AnSEC profile comparing elution peaks following 48 hours and 72 hours after elution.

FIG. 34 depicts a table quantifying the protein output of the different run workflows depicted in FIG. 31 from E. coli induced to express the exemplary construct O6, in terms of total protein recovered, approximate purity as quantified by a CBB stained gel, and the sum total protein recovered from successive eluates using 250 mM and 350 mM imidazole elution buffers.

FIG. 35 depicts exemplary conditions used for the purification of protein from E. coli induced to express the exemplary construct O6 (methods 1-4), and a CBB stained SDS-PAGE gel and corresponding anti-His Western blot showing the relative yields of purified protein product from these tested methods.

FIGS. 36A-36C depict stability studies of protein eluates according to methods depicted in FIG. 35 after buffer exchange into 1) PBS with 0.5 M arginine, 2) PBS with 0.5 M arginine and 10% glycerol, 3) PBS with 0.5 M arginine and a 10x or 3x dilution, or 4) PBS with 0.5 M arginine and a 10x or 3x dilution, and subsequent addition of 10% glycerol. These samples were tested for stability after storage at 4° C. for 24 or 120 hours, 1-3 freeze/thaws (F/T), or 1 freeze/thaw and subsequent storage at 4° C. for 24 hours. FIG. 36A depicts the stability study of samples purified according to methods 1 and 2. FIG. 36B depicts the stability study of samples purified according to method 3 and a CBB stained SDS-PAGE gel of the corresponding stability tested samples. FIG. 36C depicts the stability study of samples purified according to method 4 and a CBB stained SDS-PAGE gel of the corresponding stability tested samples.

FIG. 37 depicts a CBB stained SDS-PAGE gel of purified protein yield of eluates according to the methods depicted in FIG. 35 (loading 1 or 3 μg per well) and quantification of the yield.

FIG. 38 depicts an analysis of the contents of the insoluble fractions (inclusion bodies; IB) of lysates of E. coli induced to express the exemplary constructs F12, F13A, F14, and O6. CBB stained SDS-PAGE gels and corresponding anti-His Western blots are shown of samples of the insoluble fractions solubilized with either 6 M guanidine or 8 M urea for each exemplary construct. Solubilization over 2 hours compared to overnight using 6 M guanidine was also examined.

FIGS. 39A-39B depicts an analysis of refolding after solubilization of the insoluble fractions of lysates of E. coli induced to express the exemplary constructs F12, F13A, F14, and O6 with 6 M guanidine. FIG. 39A depicts graphs showing the concentration of protein yield recovered after refolding 1 mg/mL or 0.1 mg/mL (diluted 10×) protein samples under differing refolding pH conditions with or without arginine. FIG. 39B depicts the total protein yield recovered after refolding protein samples under differing refolding pH conditions with or without arginine, and a table depicting the different conditions tested.

FIGS. 40A-40C depict an analysis of refolding after solubilization of the insoluble fractions of lysates of E. coli induced to express the exemplary constructs F12, F13A, F14, and O6 with 6 M guanidine. FIG. 40A depicts the conditions tested, which varied in the composition and pH of the refolding buffer, and the original concentration of the sample prior to refolding (0.1 or 1 mg/mL). The different conditions have been annotated as conditions 1-12. FIG. 40B depicts CBB stained SDS-PAGE gels and corresponding anti-His Western blots of refolded F12 and F13A samples after processing according to the shown numbered conditions. FIG. 40C depicts CBB stained SDS-PAGE gels and corresponding anti-His Western blots of refolded F14 and O6 samples after processing according to the shown numbered conditions.

FIGS. 41A-41B depict an analysis of refolding after solubilization of the insoluble fractions of lysates of E. coli induced to express the exemplary construct F12 with variation in refolding incubation times (according to condition 12 shown in FIG. 40A). FIG. 41A depicts the protein yield concentration of samples refolded over 0, 0.5, 1, 1.5, 3, and 18 hours. FIG. 41B depicts a CBB stained SDS-PAGE gel and AnSEC plot of the refolded samples over the tested times.

FIGS. 42A-42B depict an analysis of purifying refolded samples of solubilized inclusion bodies of lysates of E. coli induced to express the exemplary constructs F12 or F13A with IMAC to optimize yield. FIG. 42A depicts the resultant protein yield of F12 samples purified according to method 4 testing a buffer pH of 5 or 7.4. Protein yield of F13A samples purified according to methods 3 and 4 using a buffer pH of 5 was subsequently tested, with the most yield obtained from method 4 with a buffer pH of 5. FIG. 42B depicts a CBB stained SDS-PAGE gel, corresponding anti-His Western blot, and AnSEC plots of F12 inclusion body samples solubilized with 6 M guanidine, refolded, and purified with IMAC according to method 4 using a buffer pH of 5.

FIGS. 43A-43C depict the analysis of a purification run to obtain purified product from inclusion bodies for the candidates F12, F13A, F14 where the insoluble fraction (IB) was refolded prior to loading on an IMAC column. The IB fraction was resuspended at 6 ml/g with 50 mM Tris pH8+6M Guanidine for 2 hours at 400 rpm at 25° C. Half clarified product was diluted 1/10 drop by drop in refolding buffer: PBS pH 8+0.5M Arg before loading on the IMAC column. FIG. 43A depicts a summary table with results for all three constructs, an AnSEC plot, CBB stained SDS-PAGE gel, and anti-His Western blot of the purified product of construct F12. FIG. 43B depicts an AnSEC plot, CBB stained SDS-PAGE gel, and anti-His Western blot of the purified products of F13A. FIG. 43C depicts an AnSEC plot, CBB stained SDS-PAGE gel, and anti-His Western blot of the purified products of F14.

FIGS. 44A-44B depict stability analysis on the protein eluates of FIG. 43A-43C eluted from the IMAC column. Following elution, buffer was exchanged to PBS+0.5M Arg pH 5 and pH 8. These samples were tested for stability after storage at room temperature (RT), 4° C. and −80° C., samples at −80° C. were analyzed after freeze/thaw (F/T) (up to 3 times). FIG. 44A depicts the stability study results of F12 run 7/8. FIG. 44B depicts the stability study results of F13 run 11 and F14 Run 12.

FIG. 45 depicts CBB stained SDS-PAGE gels and WB anti-HIS of E. coli lysates induced to express the exemplary constructs O7a or O7b after being cultured in Luria-Bertani broth (LB) with an initial OD₆₀₀ of 1, or in Terrific broth (TB) with an initial OD₆₀₀ of 1. Both soluble and insoluble fractions were analyzed.

FIG. 46 depicts CBB stained SDS-PAGE gels and WB anti-HIS, anti-Etag, anti-S2tag of the insoluble fraction of E. coli lysates induced to express the exemplary constructs O7a or O7b after being cultured in Luria-Bertani broth (LB) with an initial OD₆₀₀ of 1, or in Terrific broth (TB) with an initial OD₆₀₀ of 1.

FIGS. 47A-47C depict analysis of a purification run of expressed protein from E. coli induced to express the exemplary construct O6. The soluble fraction was recovered and the buffer was exchanged to PBS+arginine before loading the samples into a HiTrap IMAC Sepharose column. Elution was performed according to methods 3 and 4. FIG. 47A depicts the conditions of methods 3 and 4 and an AnSEC plot of the purified product of method 3. FIG. 47B depicts an AnSEC plot of the purified product of method 4, and a table comparing the culture yield, concentration, and endotoxin content between the products of methods 3 and 4. FIG. 47C depicts a CBB stained SDS-PAGE gel of the purified products of methods 3 and 4.

FIGS. 48A-48C depict analysis of a purification run of expressed protein from E. coli induced to express the exemplary construct O6. The insoluble fraction was resuspended at 6 ml/g with 50 mM Tris pH8+6M Guanidine for 2 hours at 400 rpm 25° C. followed by diluting the half clarified product 1/10 by drop-down in refolding buffer PBS pH 8+0.5M arginine before loading the samples into IMAC column. Elution was performed according to methods 3 and 4. FIG. 48A depicts the conditions of methods 3 and 4 and an AnSEC plot of the purified product of method 3. FIG. 48B depicts an AnSEC plot of the purified product of method 4 and a table comparing the culture yield, concentration, and endotoxin content between the products of methods 3 and 4. FIG. 48C depicts a CBB stained SDS-PAGE gel of the purified products of methods 3 and 4.

FIG. 49 depicts the results of endotoxin removal from O6 protein eluate from the soluble fraction using an anion exchange and mixed mode column. Protein loaded (mg), protein recovered (mg), protein recovery (%), and endotoxin levels (EU/mg) were measured.

FIGS. 50A-50B depict a screening of different buffer compositions of NaCl, arginine, and pH to identify which buffer would achieve the best compromise between product loss and conductivity for downstream endotoxin removal via anion exchange or mix mode columns. FIG. 50A depicts results for IMAC purified product from the insoluble fraction where buffer was exchanged to the buffers of conditions 1-9 and where product precipitation was measured by protein recovery (%). FIG. 50B is a graphical representation of the data presented in FIG. 50A.

FIG. 51 depicts an analysis of an endotoxin removal test where purified O6 product from soluble and insoluble fractions underwent buffer exchange into buffer with OM NaCl and 0.25M arginine and was loaded onto anion exchange, mix mode, cation exchange, and hydrophobic interaction chromatography (HIC) columns. Protein recovery (%) was determined from protein loaded (mg) compared to protein recovered (mg) and endotoxin level was measured (EU/mg).

FIGS. 52A-52C depict analysis of an endotoxin removal test where purified O6 product from soluble and insoluble fractions underwent buffer exchange into buffer with OM NaCl and 0.5M arginine with, or without imidazole and was loaded onto an HIC column. FIG. 52A depicts protein recovery (%) determined from protein loaded (mg) compared to protein recovered (mg), endotoxin removal (%) determined from EU/mg initial compared to EU/mg final, and product profile between IMAC purified product before and after endotoxin removal by HIC was compared by CBB stained SDS-PAGE gel for the soluble and insoluble fractions. FIG. 52B depicts an AnSEC plot for the soluble and insoluble fractions following IMAC purification. FIG. 52C depicts an AnSEC plot for the soluble and insoluble fractions following HIC purification.

FIG. 53 depicts the steps of a procedure for purification of the O6 construct from the soluble fraction. 1) 1 L of E. coli induced to express O6 were lysed, and 2) underwent buffer exchange before 3) loading onto an IMAC column, samples were eluted, and 4) underwent another buffer exchange before removal of endotoxins by 5) hydrophobic interaction chromatography, 6) concentration of the sample was performed, and 7) buffer exchange was performed before 8) sterile filtration and aliquoting. Purified O6 product was analyzed by CBB stained SDS-PAGE gel, and anti-His Western blot after storage at 5° C. or-80° C. Protein yield (mg) was determined for steps from IMAC purification to sterile filtration.

FIGS. 54A-54B depict stability tests performed on the purified O6 product of the soluble fraction from FIG. 53. FIG. 54A depicts analysis of the purified O6 product comparing stability of aliquots stored at 5° C. for 120 hours against aliquots stored at −80° C. that underwent three freeze/thaw cycles (F/T), samples were analyzed by ANSEC, CBB stained SDS-PAGE gel, and anti-His Western blot. FIG. 54B depicts a stability study comparing concentrations (mg/mL) of final O6 product following purification (TO), after 24 hours at 5° C., after 120 hours at 5° C., and −80° C. after one, two, or three F/T cycles.

FIGS. 55A-55B depict stability tests performed on the purified O6 product of the soluble fraction from FIG. 53 where an aliquot of frozen O6 purified product was diluted and half of the volume was mixed with the adjuvant QS-21 and the other half was mixed with PBS+0.5M arginine. FIG. 55A depicts AnSEC data comparing samples diluted with PBS and QS-21 freshly diluted (TO), stored for 24 hours at 5° C., or stored for 24 hours at room temperature (RT). FIG. 55B depicts analysis of the samples of FIG. 55A by CBB stained SDS-PAGE gel, and anti-His Western blot, and reports sample concentration estimated by AUC in an AnSEC column.

FIGS. 56A-56C depict purification of O6 purified product from soluble and insoluble fractions. FIG. 56A depicts the AnSEC profile of O6 purified from the soluble fraction. FIG. 56B depicts the AnSEC profile of O6 purified from the insoluble fraction. FIG. 56C depicts the CBB stained SDS-PAGE gel for soluble and insoluble samples after IMAC purification and after HIC.

FIGS. 57A-57B depict a purification procedure for O6 product from the insoluble fraction where, following HIC purification, the sample undergoes a buffer exchange before polishing through a cation exchange column (CatX) and a final buffer exchange. FIG. 57A depicts the AnSEC profiles, total protein and endotoxin levels for IMAC, HIC, CatX flow through and CatX elution samples. FIG. 57B depicts the CBB stained SDS-PAGE gel profile, anti-His Western blot profile for IMAC, HIC, and CatX flow through and CatX elution samples and the ANSEC profile for the CatX flow through sample.

FIG. 58 depicts a purification procedure for O6 product from the insoluble fraction where, following IMAC purification, the sample undergoes a buffer exchange before loading into a cation exchange column (CatX), a two-fold dilution, loading into a HIC column as the final polishing step before a final buffer exchange. FIG. 58 depicts AnSEC profiles, total protein and endotoxin levels for IMAC, CatX, and HIC samples.

FIGS. 59A-59B depict AnSEC analysis of O6 soluble and insoluble fractions and F12 insoluble fraction after elution from a SEC-3000 or SEC-4000 size exclusion chromatography column. FIG. 59A depicts AnSEC analysis of the final O6 soluble product and O6 insoluble product following IMAC purification, O6 insoluble product following CatX, and F12 insoluble after IMAC following elution from a SEC-3000 column. FIG. 59B depicts AnSEC analysis of the final O6 soluble product and O6 insoluble product following IMAC purification, O6 insoluble product following CatX, and F12 insoluble after IMAC following elution from a SEC-4000 column.

FIG. 60 depicts an AnSEC plot, CBB stained SDS-PAGE gel, and anti-His Western blot of the purified product of construct O6 from the soluble fraction collected from 1 L of E. coli culture, captured by IMAC and polished by HIC.

FIG. 61 depicts an AnSEC plot and CBB stained SDS-PAGE gel of the purified product of construct O6 from the insoluble fraction collected from 0.5 L of E. coli culture, captured by IMAC and polished by HIC and CatX.

### DETAILED DESCRIPTION

Despite preventative vaccines and antiviral therapies, chronic hepatitis B virus (HBV) infection currently affects over 250 million people across the globe. One million chronic carriers die every year due to liver related complications caused by HBV, such as liver cirrhosis and eventually hepatocellular carcinoma (HCC). The hepatitis D virus (HDV), an RNA satellite virus to HBV that “steals” the surface antigen from HBV (HBsAg), co-infects 15-25 million of HBV carriers globally and worsens disease progression. Until now, there is no effective functional cure for chronic HBV or HDV infection. The current standard of care therapy for HBV consists of nucleoside analogues (NAs) that inhibit the reverse transcriptase (RT) function of the HBV polymerase. This prevents viral maturation by blocking the synthesis of the partially dsDNA inside the capsid. Thus, NAs only suppress the viral replication during therapy. This is due to the fact that blocking of RT neither affects protein production (including HBsAg) and release, nor synthesis of the covalently closed circular DNA, the main cause for HBV persistence. A life-long NA therapy reduces, but does not eliminate, the risk of HCC. A schedule of at least one-year pegylated interferon (IFN)-alpha is the currently recommended treatment for chronic HDV; however, sustained response rates are rare. Combination treatment with pegylated IFN-alpha and NAs has been shown to have limited efficacy against HDV and HBV.

HBV uses several strategies in order to evade the host immune response. The chronic presence of HBV proteins induces a T cell dysfunction. HBV infected cells overproduce sub-viral HBsAg particles mainly containing small HBsAg (SHBsAg) to block the neutralizing antibody population directed to SHBsAg. This ensures survival of viral particles whose surface are denser with the middle HBsAg (MHBsAg; containing S and PreS2) and large HBsAg (LHBsAg; containing S, PreS2 and PreS1) proteins. Importantly, the PreS1 domain is responsible for binding to the Na⁺-taurocholate co-transporting polypeptide (NTCP) receptor for HBV on hepatocytes. Thus, an obvious way to target infectious HBV particles and prevent the infection of new hepatocytes would be to raise antibodies to the PreS1 domain of the virus.

As disclosed herein, to build an immunotherapy targeting both HBV and HDV infections to induce production of PreS1 antibodies and T cells specific for HBV and HDV, chimeric genes containing PreS1 and the large HDV antigen were generated in different combinations. The advantage of linking PreS1 to HDAg is that HDAg will act as a heterologous T cell epitope carrier in patients that are mono-infected by HBV. Thus, these HDAg-specific T cells support a sustained endogenous production of PreS1 antibodies that block viral entry and bypass the need for HBV-specific T cells. In fact, >90% of HBV carriers are mono-infected with HBV, and in these patients, the heterologous HDAg will prime healthy naïve T cells that support priming of HBV-specific responses. In addition, it is likely that the HDAg-specific T cells and PreS1 antibodies prevent HDV superinfection in these patients. To induce both neutralizing antibodies and T cells, genetic immunization was used, as this strategy has shown to activate immune response to HBV. Overall, this virus entry-blocking strategy complements the maturation inhibitors and the capsid assembly inhibitors, currently under development, in order to achieve sustainable off-therapy responses against HBD and/or HDV infection.

Embodiments provided herein related to immunogenic compositions or product combinations of engineered hepatitis B (HBV) and hepatitis D (HDV) nucleic acids, genes, peptides, or proteins that can be used to elicit an immune response against an HBV or HDV infection. The use of chimeric genes and chimeric proteins comprising HBV and HDV nucleic acids, genes, peptides, or proteins has been characterized, for example, in WO 2017/132332, hereby expressly incorporated by reference in its entirety.

To improve different aspects of immunogenic composition production, manufacturing, and efficacy, several modifications to the antigenic sequences were explored. Both HBV PreS1 and HDV HDAg (large antigen) contain large homologous DNA repeats. This can make the carrier plasmid unstable and prone to homologous recombination. Optimization of codons were performed to minimize the presence of these homologous DNA repeats. In addition, the large HDAg antigen contains an exposed free cysteine in the C-terminal domain, which is prenylated in eukaryotic cells. These cysteines have a high tendency to form disulfide bridges with other proteins, therefore increasing the risk of cross-linked contaminants or potential scrambling of the correct structure of HDAg. Substitution of these cysteines to structurally homologous serine has been explored. Linker sequences may be introduced between HBV and/or HDV sequences, which is thought to help in inducing proper head-to-head interaction of multiple monomers (by the N-terminal L-zipper structure of HDAg). Finally, given that the most abundant proteins in E. coli are around 20-50 kDa, smaller fusions of HBV and HDV antigenic proteins were explored.

Additionally, a new operon approach for production of the immunogenic compositions was explored. With this approach, multiple smaller constructs can be expressed efficiently in E. coli for subsequent isolation and purification. The simultaneous expression of multiple peptides containing HDAg and HBV PreS1 sequences can lead to the natural formation oligomeric complexes.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

### Terms

Unless defined otherwise, all technical and scientific terms used herein have the same meaning as is commonly understood by one of ordinary skill in the art. All patents, applications, published applications and other publications referenced herein are expressly incorporated by reference in their entireties unless stated otherwise. In the event that there are a plurality of definitions for a term herein, those in this section prevail unless stated otherwise.

The articles “a” and “an” are used herein to refer to one or to more than one (for example, at least one) of the grammatical object of the article. By way of example, “an element” means one element or more than one element.

The terms “about” or “around” as used herein refer to a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The practice of the present disclosure will employ, unless indicated specifically to the contrary, conventional methods of molecular biology and recombinant DNA techniques within the skill of the art, many of which are described below for the purpose of illustration. Such techniques are explained fully in the literature. See, e.g., Sambrook, el al, Molecular Cloning: A Laboratory Manual (3rd Edition, 2000); DNA Cloning: A Practical Approach, vol. 1 & II (D. Glover, ed.); Oligonucleotide Synthesis (N. Gait, ed., 1984); Oligonucleotide Synthesis: Methods and Applications (P. Herdewijn, ed., 2004); Nucleic Acid Hybridization (B. Hames & S. Higgins, eds., 1985); Nucleic Acid Hybridization: Modern Applications (Buzdin and Lukyanov, eds., 2009); Transcription and Translation (B. Hames & S. Higgins, eds., 1984); Animal Cell Culture (R. Freshney, ed., 1986); Freshney, R. I. (2005) Culture of Animal Cells, a Manual of Basic Technique, 5th Ed. Hoboken NJ, John Wiley & Sons; B. Perbal, A Practical Guide to Molecular Cloning (3rd Edition 2010); Farrell, R., RNA Methodologies: A Laboratory Guide for Isolation and Characterization (3rd Edition 2005).

The term “purity” of any given substance, compound, or material as used herein refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between. Purity may be affected by unwanted impurities, including but not limited to side products, isomers, enantiomers, degradation products, solvent, carrier, vehicle, or contaminants, or any combination thereof. Purity can be measured technologies including but not limited to chromatography, liquid chromatography, gas chromatography, spectroscopy, UV-visible spectrometry, infrared spectrometry, mass spectrometry, nuclear magnetic resonance, gravimetry, or titration, or any combination thereof.

The terms “function” and “functional” as used herein refer to a biological, enzymatic, or therapeutic function.

The phrases “effective amount” or “effective dose” as used herein refers to an amount sufficient to achieve the desired result and accordingly will depend on the ingredient and its desired result. Nonetheless, once the desired effect is known, determining the effective amount is within the skill of a person skilled in the art.

In general, the “error bars” provided in the figures represent the standard error of the mean value.

“Formulation” and “composition” as used interchangeably herein are equivalent terms referring to a composition of matter for administration to a subject.

The term “isolated” as used herein refers to material that is substantially or essentially free from components that normally accompany it in its native state. For example, an “isolated cell,” as used herein, includes a cell that has been purified from the milieu or organisms in its naturally occurring state, a cell that has been removed from a subject or from a culture, for example, it is not significantly associated with in vivo or in vitro substances.

The term “subject” as used herein has its ordinary meaning as understood in light of the specification and refers to an animal that is the object of treatment, inhibition, or amelioration, observation or experiment. “Animal” has its ordinary meaning as understood in light of the specification and includes cold- and warm-blooded vertebrates and/or invertebrates such as fish, shellfish, or reptiles and, in particular, mammals. “Mammal” has its ordinary meaning as understood in light of the specification, and includes but is not limited to mice, rats, rabbits, guinea pigs, dogs, cats, sheep, goats, cows, horses, primates, such as humans, monkeys, chimpanzees, or apes. In some embodiments, the subject is human.

Some embodiments disclosed herein relate to selecting a subject or patient in need. In some embodiments, a patient is selected who is in need of a treatment, inhibition, or amelioration of a viral infection, such as HBV and/or HDV infection, which may be chronic, or protection from a viral infection, such as HBV and/or HDV infection. In some embodiments, a patient is selected who has previously been treated for a viral infection, such as HBV and/or HDV infection, which may be chronic. In some embodiments, a patient is selected who has previously been treated for being at risk of a viral infection, such as HBV and/or HDV infection. In some embodiments, a patient is selected who has developed a recurrence of a viral infection, such as HBV and/or HDV infection, which may be chronic. In some embodiments, a patient is selected who has developed resistance to therapies for a viral infection, such as HBV and/or HDV infection, which may be chronic. In some embodiments, a patient is selected who may have any combination of the aforementioned selection criteria. In some embodiments, the viral infection for which the patient is selected is a chronic viral infection. The basis for selection can be made by clinical evaluation or diagnostic test showing the presence of a viral infection or a sequela thereof.

The terms “treating,” “treatment,” “therapeutic,” or “therapy” as used herein has its ordinary meaning as understood in light of the specification, and do not necessarily mean total cure or abolition of the disease or condition. The term “treating” or “treatment” as used herein (and as well understood in the art) also means an approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delaying or slowing of disease progression, amelioration or palliation of the disease state, diminishment of the reoccurrence of disease, and remission, whether partial or total and whether detectable or undetectable. “Treating” and “treatment” as used herein also include prophylactic treatment. Treatment methods comprise administering to a subject a therapeutically effective amount of an active agent. The administering step may consist of a single administration or may comprise a series of administrations. The compositions are administered to the subject in an amount and for a duration sufficient to treat the patient. The length of the treatment period depends on a variety of factors, such as the severity of the condition, the age and genetic profile of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required.

The term “inhibit” as used herein has its ordinary meaning as understood in light of the specification, and may refer to the reduction or prevention of a viral infection. The reduction can be by 10%, 20%, 30%, 40%, 50%, 60%, 70%, 80%, 90%, or 100%, or an amount that is within a range defined by any two of the aforementioned values. As used herein, the term “delay” has its ordinary meaning as understood in light of the specification, and refers to a slowing, postponement, or deferment of an event, such as a viral infection, to a time which is later than would otherwise be expected. The delay can be a delay of 0%, 10%, 20%, 30%, 40%, 50%, 60%, 70%, 80%, 90%, 100%, or an amount within a range defined by any two of the aforementioned values. The terms inhibit and delay may not necessarily indicate a 100% inhibition or delay. A partial inhibition or delay may be realized.

The term “immunogenic composition” as used herein refers to a substance or mixture of substances, including but not limited to antigens, epitopes, nucleic acids, peptides, polypeptides, proteins, polysaccharides, lipids, haptens, toxoids, inactivated organisms, or attenuated organisms, or any combination thereof, intended to elicit an immune response when administered to a host. The immune response includes both an innate and adaptive immune response, the latter of which establishes a lasting immunological memory through cells such as memory T cells and memory B cells. The antibodies created during the initial immune response to the immunogenic composition can be produced in subsequent challenges of the same antigens, epitopes, nucleic acids, peptides, polypeptides, proteins, polysaccharides, lipids, haptens, toxoids, inactivated organisms, or attenuated organisms, or a live organism or pathogen that exhibits the antigens, epitopes, nucleic acids, peptides, polypeptides, proteins, polysaccharides, lipids, haptens, or toxoids or any combination thereof. In this manner, the immunogenic composition may serve as a vaccine against a specific pathogen. Immunogenic compositions may also include one or more adjuvants to stimulate the immune response and increase the efficacy of protective immunity.

The term “product combination” as used herein refers to set of two or more individual compounds, substances, materials, or compositions that can be used together for a unified function. In some embodiments, a product combination comprises at least one nucleic acid composition and at least one polypeptide composition that are used together to elicit an immune response when administered to a host, optionally to a greater degree than would be elicited if only one composition type were to be administered.

The terms “nucleic acid” or “nucleic acid molecule” as used herein refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate. The term “nucleic acid molecule” also includes so-called “peptide nucleic acids,” which comprise naturally-occurring or modified nucleic acid bases attached to a polyamide backbone. Nucleic acids can be either single stranded or double stranded. “Oligonucleotide” can be used interchangeable with nucleic acid and can refer to either double stranded or single stranded DNA or RNA. A nucleic acid or nucleic acids can be contained in a nucleic acid vector or nucleic acid construct (e.g. plasmid, virus, bacteriophage, cosmid, fosmid, phagemid, bacterial artificial chromosome (BAC), yeast artificial chromosome (YAC), or human artificial chromosome (HAC)) that can be used for amplification and/or expression of the nucleic acid or nucleic acids in various biological systems. Typically, the vector or construct will also contain elements including but not limited to promoters, enhancers, terminators, inducers, ribosome binding sites, translation initiation sites, start codons, stop codons, polyadenylation signals, origins of replication, cloning sites, multiple cloning sites, restriction enzyme sites, epitopes, reporter genes, selection markers, antibiotic selection markers, targeting sequences, peptide purification tags, or accessory genes, or any combination thereof.

A nucleic acid or nucleic acid molecule can comprise one or more sequences encoding different peptides, polypeptides, or proteins. These one or more sequences can be joined in the same nucleic acid or nucleic acid molecule adjacently, or with extra nucleic acids in between, e.g. linkers, repeats or restriction enzyme sites, or any other sequence that is 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a nucleic acid as used herein refers to a sequence being after the 3′-end of a previous sequence, on the strand containing the encoding sequence (sense strand) if the nucleic acid is double stranded. The term “upstream” on a nucleic acid as used herein refers to a sequence being before the 5′-end of a subsequent sequence, on the strand containing the encoding sequence (sense strand) if the nucleic acid is double stranded. The term “grouped” on a nucleic acid as used herein refers to two or more sequences that occur in proximity either directly or with extra nucleic acids in between, e.g. linkers, repeats, or restriction enzyme sites, or any other sequence that is 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths, but generally not with a sequence in between that encodes for a functioning or catalytic polypeptide, protein, or protein domain.

The term “codon optimized” regarding a nucleic acid as used herein refers to the substitution of codons of the nucleic acid, for example, to enhance or maximize translation in a host of a particular species without changing the polypeptide sequence based on species-specific codon usage biases and relative availability of each aminoacyl-tRNA in the target cell cytoplasm. Codon optimization and techniques to perform such optimization is known in the art. Programs containing algorithms for codon optimization are known to those skilled in the art. Additionally, synthetic codon optimized sequences can be obtained commercially. Those skilled in the art will appreciate that gene expression levels are dependent on many factors, such as promoter sequences and regulatory elements. As noted for most bacteria, small subsets of codons are recognized by tRNA species leading to translational selection, which can be an important limit on protein expression. In this aspect, many synthetic genes can be designed to increase their protein expression level. As used herein, codon optimization has also been used to prepare HBV PreS1 and HDV HDAg antigen sequences that are non-homologous or partly non-homologous. These viral proteins contain repeat sequences, which may cause plasmid instability, and codon optimization was used to minimize recombination and/or other undesired effects resulting from the repeat sequences.

The nucleic acids described herein comprise nucleobases. Primary, canonical, natural, or unmodified bases are adenine, cytosine, guanine, thymine, and uracil. Other nucleobases include but are not limited to purines, pyrimidines, modified nucleobases, 5-methylcytosine, pseudouridine, dihydrouridine, inosine, 7-methylguanosine, hypoxanthine, xanthine, 5,6-dihydrouracil, 5-hydroxymethylcytosine, 5-bromouracil, isoguanine, isocytosine, aminoallyl bases, dye-labeled bases, fluorescent bases, or biotin-labeled bases.

The terms “peptide”, “polypeptide”, and “protein” as used herein refers to macromolecules comprised of amino acids linked by peptide bonds. The numerous functions of peptides, polypeptides, and proteins are known in the art, and include but are not limited to enzymes, structure, transport, defense, hormones, or signaling. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths.

In some embodiments, the nucleic acid or peptide sequences presented herein and used in the examples are functional in various biological systems including but not limited to humans, mice, rabbits, E. coli, yeast, and mammalian cells. In other embodiments, nucleic acid or peptide sequences sharing 0%, 10%, 20%, 30%, 40%, 50%, 60%, 70%, 80%, 90%, 91%, 92%, 93%, 94%, 95%, 96%, 97%, 98%, 99%, or 100% similarity, or any percentage within a range defined by any two of the aforementioned percentages similarity to the nucleic acid or peptide sequences presented herein and used in the examples can also be used with no effect on the function of the sequences in biological systems. As used herein, the term “similarity” refers to a nucleic acid or peptide sequence having the same overall order of nucleotide or amino acids, respectively, as a template nucleic acid or peptide sequence with specific changes such as substitutions, deletions, repetitions, or insertions within the sequence. In some embodiments, two nucleic acid sequences sharing as low as 0%, 10%, 20%, 30%, 40%, 50%, 60%, 70%, 80%, 90%, 91%, 92%, 93%, 94%, 95%, 96%, 97%, 98%, or 99% similarity can encode for the same polypeptide by comprising different codons that encode for the same amino acid during translation.

The term “recombinantly expressed” as used herein refers to the production of proteins in optimized or adapted biological systems. These systems provide advantages over protein expression in a natural host, including but not limited to high expression (overexpression), ease of purification, ease of transformation, inducibility, low cost, or stability of the protein. In some embodiments, proteins are expressed in mammalian, bacteria, yeast, insect, or cell-free recombinant expression systems. Each system has its own advantages or disadvantages. For example, bacterial expression systems are highly optimized for overexpression, but may cause misfolding or aggregation of the produced protein, yeast systems are useful when post-translational modifications are necessary, and insect and mammalian systems are useful for proper RNA splicing that occurs in higher-order organisms. In some embodiments, Δ-7, Δ-8, and other recombinant polypeptides are produced and purified from mammalian, human, primary, immortalized, cancer, stem, fibroblasts, human embryonic kidney (HEK) 293, Chinese Hamster Ovary (CHO), bacterial, Escherichia coli, yeast, Saccharomyces cerevisiae, Pichia pastoris, insect, Spodoptera frugiperda Sf9, or S. frugiperda Sf21 cells, or in a cell-free system. In some embodiments, expression genes, vectors, or constructs are delivered to the recombinant expression systems in the form of plasmids, bacteriophages, viruses, adeno-associated viruses (AAVs), baculovirus, cosmids, fosmids, phagemids, BACs, YACs, or HACs. For more discussion on recombinant expression systems, see Gomes et al. “An Overview of Heterologous Expression Host Systems for the Production of Recombinant Proteins” ((2016) Adv. Anim. Vet. Sci. 4(7):346-356), hereby expressly incorporated by reference in its entirety.

The term “HDAg” as used herein refers the hepatitis D antigen gene or protein. A small (24 kDa) and large (27 kDa, 213 amino acids excluding the start methionine) isoform exist for HDAg and are translated from the same open reading frame on the HDV genome. Deamination of the adenosine in a UAG stop codon at codon 196 of the coding sequence allows for translation to continue and produce the large isoform. Unless expressly stated otherwise, the embodiments described herein comprise the large isoform of HDAg. In some embodiments, the HDAg sequences comprise at least one of four different HDAg strain sequences: “HDAg genotype 1 A”, “HDAg genotype 1 B”, “HDAg genotype 2 A”, or “HDAg genotype 2 B”. In some embodiments, the nucleic acid sequence encoding at least one HDAg polypeptide comprises the nucleic acid sequence of HDAg genotype 1 A (SEQ ID NO: 1), HDAg genotype 1 B (SEQ ID NO: 2), HDAg genotype 2 A (SEQ ID NO: 3), or HDAg genotype 2 B (SEQ ID NO: 4). In some embodiments, the polypeptide comprising at least one HDAg polypeptide comprises the polypeptide sequence of HDAg genotype 1 A (SEQ ID NO: 5), HDAg genotype 1 B (SEQ ID NO: 6), HDAg genotype 2 A (SEQ ID NO: 7), or HDAg genotype 2 B (SEQ ID NO: 8).

The term “PreS1” as used herein refers to a segment of the N-terminal domain on the large surface antigen of HBV (HBsAg). A 47 amino acid long PreS1 segment of the 108-119 amino acid long N-terminal domain of the large HBsAg is effective in eliciting an immune response and generating high titer of anti-PreS1/anti-HBV antibodies in mammalian models. In some embodiments, the PreS1 sequences comprise at least one of two different PreS1 consensus sequences: “PreS1 A” and/or “PreS1 B”. In some embodiments, the nucleic acid sequence encoding at least one PreS1 polypeptide comprises the nucleic acid sequence of PreS1 A (SEQ ID NO: 9) or PreS1 B (SEQ ID NO: 10). In some embodiments, the polypeptide comprising at least one PreS1 polypeptide comprises the polypeptide sequence of PreS1 A (SEQ ID NO: 11) or PreS1 B (SEQ ID NO: 12).

In some embodiments, the PreS1 A and PreS1 B consensus sequences of HBV are obtained or derived from sequence similarity of PreS1 in known genotypes of HBV. There are ten known or prevalent HBV genotypes (genotypes A, B, C, D, E, F, G, H, I, and J) exhibiting up to or about 8% nucleotide differences in genomic sequence. Of these, there are additional sub-genotypes exhibiting up to or about 4%-8% nucleotide differences in genomic sequence. Sub-genotypes of HBV include but are not limited to A1, A2, A3, A4, A5, A6, A7, B2, B3, B4, B5, B6, B7, B9, C1, C2, C3, C4, C5, C6, C7, C8, C9, C10, D1, D2, D3, D4, D5, D6, D7, F1, F2, F2a, F3, or F4. For more discussion on HBV genotypes, see Sunbul “Hepatitis B virus genotypes: Global distribution and clinical importance” ((2014) World. J. Gastroenterology. 20(18):5427-5434, hereby expressly incorporated by reference in its entirety.

The terms “autocatalytic peptide cleavage site” or “2A peptide” as used herein refer to a peptide sequence that undergo cleavage of a peptide bond between two constituent amino acids, resulting in separation of the two proteins that flank the sequence. The cleavage is believed to be a result of a ribosomal “skipping” of the peptide bond formation between the C-terminal proline and glycine in the 2A peptide sequence. Four autocatalytic peptide cleavage site sequences identified to date have seen substantial use in biomedical research: foot-and-mouth disease virus 2A (F2A); equine rhinitis A virus (ERAV) 2A (E2A); porcine teschovirus-1 2A (P2A), and Thosea asigna virus 2A (T2A). In some embodiments, the P2A autocatalytic peptide cleavage site nucleic acid (SEQ ID NO: 13) and polypeptide (SEQ ID NO: 14) sequences are used.

The term “HBeAg” as used herein refers to an HBV antigen protein found between the nucleocapsid core and lipid envelope of the virus. HBeAg produced in a host is secreted into the blood serum and is a good marker for an active HBV infection. Quantification of in vitro HBeAg secretion in a cell culture model can be used to assess effect of biological or pharmaceutical compounds or compositions on HBV infectivity.

The term “excipient” has its ordinary meaning as understood in light of the specification, and refers to other substances, compounds, or materials found in an immunogenic composition or vaccine. Excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), citric acid, salts, sodium chloride, sodium bicarbonate, sodium phosphate, sodium borate, sodium citrate, potassium chloride, potassium phosphate, magnesium sulfate sugars, dextrose, fructose, mannose, lactose, galactose, sucrose, sorbitol, cellulose, serum, amino acids, polysorbate 20, polysorbate 80, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. Some excipients may be in residual amounts or contaminants from the process of manufacturing the immunogenic composition or vaccine, including but not limited to serum, albumin, ovalbumin, antibiotics, inactivating agents, formaldehyde, glutaraldehyde, B-propiolactone, gelatin, cell debris, nucleic acids, peptides, amino acids, or growth medium components or any combination thereof. The amount of the excipient may be found in an immunogenic composition or vaccine at a percentage of 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

The term “adjuvant” as used herein refers to a substance, compound, or material that stimulates the immune response and increase the efficacy of protective immunity and is administered in conjunction with the immunogenic antigen, epitope, or composition. Adjuvants serve to improve immune responses by enabling a continual release of antigen, up-regulation of cytokines and chemokines, cellular recruitment at the site of administration, increased antigen uptake and presentation in antigen presenting cells, or activation of antigen presenting cells and inflammasomes. Commonly used adjuvants include but are not limited to alum, aluminum salts, aluminum sulfate, aluminum hydroxide, aluminum phosphate, calcium phosphate hydroxide, potassium aluminum sulfate, oils, mineral oil, paraffin oil, oil-in-water emulsions, detergents, MF59®, squalene, AS03, α-tocopherol, polysorbate 80, AS04, monophosphoryl lipid A, virosomes, nucleic acids, polyinosinic: polycytidylic acid, saponins, QS-21, proteins, flagellin, cytokines, chemokines, IL-1, IL-2, IL-12, IL-15, IL-21, imidazoquinolines, CpG oligonucleotides, lipids, phospholipids, dioleoyl phosphatidylcholine (DOPC), trehalose dimycolate, peptidoglycans, bacterial extracts, lipopolysaccharides, or Freund's Adjuvant, or any combination thereof.

The terms “prime” and “boost” as used herein related to separate immunogenic compositions used in a heterologous prime-boost immunization approach. Immunizations or vaccines commonly require more than one administration of an immunogenic composition to induce a successful immunity against a target pathogen in a host. Compared to this homologous approach where the same composition is provided for all administrations, a heterologous prime-boost administration may be more effective in establishing robust immunity with greater antibody levels and improved clearing or resistance against some pathogens such as HBV or HDV. In a heterologous prime-boost administration, at least one prime dose comprising one type of immunogenic composition is first provided. After the at least one prime dose is provided, at least one boost dose comprising another type of immunogenic composition is then provided. Administration of the at least one boost dose is performed at least 1, 2, 3, 4, 5, 6, 7, 8, 9, 10, 11, 12, 24, 36, or 48 days or weeks after the at least one prime dose is administered or within a range of time defined by any two of the aforementioned time points e.g., within 1-48 days or 1-48 weeks. In some embodiments, the prime dose comprises a nucleic acid (e.g., DNA or RNA) that encodes for one or more antigens or epitopes, and the boost dose comprises a polypeptide that comprises one or more antigens or epitopes. In the host, the nucleic acid prime is translated in vivo to elicit an immune reaction and causes a greater response against the subsequent polypeptide boost. In some embodiments, the nucleic acid prime comprises sequences that encodes for at least one HDAg polypeptide, at least one PreS1 peptide, and at least one autocatalytic peptide cleavage site. In some embodiments, the polypeptide boost comprises at least one HDAg polypeptide and at least one PreS1 polypeptide.

In some embodiments, administration of the nucleic acid prime and polypeptide boost comprising HBV and HDV components in an experimental organism results in greater anti-HDAg, anti-PreS1, anti-HBV, or anti-HDV antibody titer at a ratio of 1, 2, 3, 4, 5, 6, 7, 8, 9, 10, 50, 100, 150, 200, 300, 400, 500, 600, 700, 800, 900, 1000, 5000, 10000, 100000, or 1000000 or any ratio within a range defined by any two of the aforementioned ratios compared to nucleic acid-only or polypeptide-only immunized, or unimmunized control organisms, quantified by techniques known in the art such as ELISA. In some embodiments, administration of the nucleic acid prime and polypeptide boost comprising HBV and HDV components in an experimental organism results in serum that neutralizes HBV or HDV infectivity in vitro more effectively and reduces the incidence of infection to a ratio of 0.00001, 0.00005, 0.0001, 0.0005, 0.001, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1.0 or any ratio within a range defined by any two of the aforementioned ratios compared to sera from nucleic acid-only or polypeptide-only immunized, or unimmunized control organisms. In some embodiments, administration of the nucleic acid prime and polypeptide boost comprising HBV and HDV components in an experimental organism results in a greater number of interferon gamma (IFNγ)-positive cells (e.g. T cells) at a ratio of 1, 2, 3, 4, 5, 6, 7, 8, 9, 10, 20, 30, 40, 50, 60, 70, 80, 90, 100, 110, 120, 130, 140, 150, 200, 250, 300, 350, 400, 450, 500, 550, 600, 650, 700, 750, 800, 850, 900, 950, 1000, 5000, or 10000, or any ratio within a range defined by any two of the aforementioned ratios compared to nucleic acid-only or polypeptide-only immunized, or unimmunized control organisms.

In some embodiments, the immunogenic compositions or product combinations are administered with an adjuvant. In some embodiments, the immunogenic compositions or product combinations are administered enterally, orally, intranasally, parenterally, subcutaneously, intramuscularly, intradermally, or intravenously or any combination thereof. In some embodiments, the immunogenic compositions or product combinations are administered in conjunction with an antiviral therapy compound known to have an effect against HBV or HDV, including but not limited to entecavir, tenofovir, lamivudine, adefovir, telbivudine, emtricitabine, interferon-a, pegylated interferon-a, or interferon alfa-2b, or any combination thereof.

The terms “in vivo electroporation”, “electroporation”, and “EP” as used herein refers to the delivery of genes, nucleic acids, DNA, RNA, proteins, or vectors into cells of living tissues or organisms using electrical currents using techniques known in the art. Electroporation can be used as an alternative to other methods of gene transfer such as viruses (transduction), lipofection, gene gun (biolistics), microinjection, vesicle fusion, or chemical transformation. Electroporation limits the risk of immunogenicity and detrimental integration or mutagenesis of the cell genome. DNA vectors such as plasmids are able to access the cell nucleus, enabling transcription and translation of constituent genes. In some embodiments, the genes, nucleic acids, DNA, RNA, proteins, or vectors are added to the target tissue or organism by subcutaneous, intramuscular, or intradermal injection. An electroporator then delivers short electrical pulses via electrodes placed within or proximal to the injected sample. As used herein, the term “im/EP” refers to in vivo electroporation of a sample delivered intramuscularly (“im”).

The term “uPA^{+/+}-SCID” as used herein refers to an immunodeficient mouse model used for studying liver diseases including hepatitis viral infections. These mice are homozygous for Prkde^{scid}, causing deficiencies in functional T and B lymphocytes. Overexpression of urokinase-type plasminogen activator (uPA) also causes severe liver cytotoxicity and hepatic insufficiency during development. Subsequent transplantation and engraftment of human liver tissue to these mice results in a model ideal for studying hepatic illnesses of humans. For more discussion on uPA^{+/+}-SCID mice, see Meuleman et al. “The human liver-uPA-SCID mouse: A model for the evaluation of antiviral compounds against HBV and HCV” ((2008) Antiviral Research 80(3):231-238), hereby expressly incorporated by reference in its entirety.

The term “% w/w” or “% wt/wt” as used herein has its ordinary meaning as understood in light of the specification and refers to a percentage expressed in terms of the weight of the ingredient or agent over the total weight of the composition multiplied by 100. The term “% v/v” or “% vol/vol” as used herein has its ordinary meaning as understood in the light of the specification and refers to a percentage expressed in terms of the liquid volume of the compound, substance, ingredient, or agent over the total liquid volume of the composition multiplied by 100.

The invention is generally disclosed herein using affirmative language to describe the numerous embodiments. The invention also includes embodiments in which subject matter is excluded, in full or in part, such as substances or materials, method steps and conditions, protocols, or procedures.

### Immunogenic Compositions and Product Combinations

Disclosed herein are immunogenic compositions or product combinations. In some embodiments, these immunogenic compositions or product combinations are configured to induce an immunogenic response against a particular antigen. In some embodiments, the immunogenic compositions or product combinations comprise (a) a nucleic acid comprising at least one nucleic acid sequence encoding hepatitis D antigen (HDAg) and at least one nucleic acid sequence encoding PreS1; and (b) a polypeptide comprising at least one HDAg polypeptide sequence and at least one PreS1 polypeptide sequence. In some embodiments, the at least one nucleic acid sequence encoding HDAg comprises SEQ ID NO: 1, SEQ ID NO: 2, SEQ ID NO: 3 or SEQ ID NO: 4, or any combination thereof. In some embodiments, the at least one nucleic acid sequence encoding PreS1 comprises SEQ ID NO: 9 or SEQ ID NO: 10 or both. In some embodiments, the nucleic acid is configured such that each HDAg nucleic acid sequence is grouped with a PreS1 nucleic acid sequence, and wherein the PreS1 nucleic acid sequence is immediately downstream of the HDAg nucleic acid sequence. In some embodiments, the immunogenic compositions or product combinations further comprise at least one nucleic acid sequence encoding an autocatalytic peptide cleavage site, wherein the grouped HDAg and PreS1 nucleic acid sequences are separated by the at least one nucleic acid sequence encoding the autocatalytic peptide cleavage site. In some embodiments, the at least one nucleic acid sequence encoding the autocatalytic peptide cleavage site comprises a nucleic acid sequence selected from the group consisting of porcine teschovirus-1 2A (P2A), foot-and-mouth disease virus 2A (F2A), equine rhinitis A virus (ERAV) 2A (E2A) and Thosea asigna virus 2A (T2A) nucleic acid, and wherein each encoded autocatalytic peptide cleavage site may optionally include a GSG (glycine-serine-glycine) motif at its N-terminus. In some embodiments, the at least one nucleic acid sequence encoding the autocatalytic peptide cleavage site comprises SEQ ID NO: 13. In some embodiments, the nucleic acid is codon optimized for expression in a human. In some embodiments, the nucleic acid comprises a sequence having at least 80%, 85%, 90%, 95%, 99%, or 100% homology or sequence identity to SEQ ID NO: 15-24 or 35-36. In some embodiments, the nucleic acid comprises a sequence having at least 80%, 85%, 90%, 95%, 99%, or 100% homology or sequence identity to SEQ ID NO: 18, or SEQ ID NO: 35-36. In some embodiments, the at least one HDAg polypeptide comprises SEQ ID NO: 5, SEQ ID NO: 6, SEQ ID NO: 7 or SEQ ID NO: 8 or any combination thereof. In some embodiments, the at least one PreS1 polypeptide sequence comprises SEQ ID NO: 11 or SEQ ID NO: 12 or both. In some embodiments, the at least one PreS1 polypeptide sequence is downstream of the at least one HDAg polypeptide sequence. In some embodiments, the polypeptide comprises a sequence having at least 80%, 85%, 90%, 95%, 99%, or 100% homology or sequence identity to the sequence of SEQ ID NO: 25-34 or 37. In some embodiments, the polypeptide comprises a sequence having at least 80%, 85%, 90%, 95%, 99%, or 100% homology or sequence identity to the sequence of SEQ ID NO: 29, 31, 32, or 37. In some embodiments, the polypeptide is recombinantly expressed. In some embodiments, the polypeptide is recombinantly expressed in a mammalian, bacterial, yeast, insect, or cell-free system. In some embodiments, the immunogenic compositions or product combinations further comprise an adjuvant. In some embodiments, the adjuvant is alum, QS-21 or MF59, or any combination thereof. In some embodiments, the nucleic acid comprises DNA. In some embodiments, the nucleic acid is provided in a recombinant vector.

Disclosed herein are immunogenic compositions or product combinations. In some embodiments, these immunogenic compositions or product combinations are configured to induce an immunogenic response against a particular antigen. In some embodiments, the immunogenic compositions or product combinations comprise (a) a nucleic acid comprising at least one nucleic acid sequence encoding hepatitis D antigen (HDAg) and at least one nucleic acid sequence encoding PreS1; and (b) a polypeptide comprising at least one HDAg polypeptide sequence and at least one PreS1 polypeptide sequence. In some embodiments, the at least one nucleic acid sequence encoding HDAg comprises SEQ ID NO: 1-4 or 43-46, or any combination thereof. In some embodiments, the at least one nucleic acid sequence encoding PreS1 comprises SEQ ID NO: 9-10 or 51-53, or any combination thereof. In some embodiments, the nucleic acid is configured such that each HDAg nucleic acid sequence is grouped with a PreS1 nucleic acid sequence, and wherein the PreS1 nucleic acid sequence is immediately downstream of the HDAg nucleic acid sequence. In some embodiments, the immunogenic compositions or product combinations further comprise at least one nucleic acid sequence encoding an autocatalytic peptide cleavage site, wherein the grouped HDAg and PreS1 nucleic acid sequences are separated by the at least one nucleic acid sequence encoding the autocatalytic peptide cleavage site. In some embodiments, the at least one nucleic acid sequence encoding the autocatalytic peptide cleavage site comprises a nucleic acid sequence selected from the group consisting of porcine teschovirus-1 2A (P2A), foot-and-mouth disease virus 2A (F2A), equine rhinitis A virus (ERAV) 2A (E2A) and Thosea asigna virus 2A (T2A) nucleic acid, and wherein each encoded autocatalytic peptide cleavage site may optionally include a GSG (glycine-serine-glycine) motif at its N-terminus. In some embodiments, the at least one nucleic acid sequence encoding the autocatalytic peptide cleavage site comprises SEQ ID NO: 13. In some embodiments, the nucleic acid is codon optimized for expression in a human. In some embodiments, the nucleic acid comprises a sequence having at least 80%, 85%, 90%, 95%, 99%, or 100% homology or sequence identity to SEQ ID NO: 15-24, 35-36, 60-71, 134-135, 138-139, 142-144, or 148-162. In some embodiments, the nucleic acid comprises a sequence having at least 80%, 85%, 90%, 95%, 99%, or 100% homology or sequence identity to SEQ ID NO: 134-135, 138-139, 142-144, or 148-162. In some embodiments, the at least one HDAg polypeptide comprises SEQ ID NO: 5-8 or 47-50 or any combination thereof. In some embodiments, the at least one PreS1 polypeptide sequence comprises SEQ ID NO: 11 or SEQ ID NO: 12 or both. In some embodiments, the at least one PreS1 polypeptide sequence is downstream of the at least one HDAg polypeptide sequence. In some embodiments, the polypeptide comprises a sequence having at least 80%, 85%, 90%, 95%, 99%, or 100% homology or sequence identity to the sequence of SEQ ID NO: 25-34, 37, 72-95, 136-137, 140-141, 145-147, or 162-177. In some embodiments, the polypeptide comprises a sequence having at least 80%, 85%, 90%, 95%, 99%, or 100% homology or sequence identity to the sequence of SEQ ID NO: 136-137, 140-141, 145-147, or 162-177. In some embodiments, the polypeptide is recombinantly expressed. In some embodiments, the polypeptide is recombinantly expressed in a mammalian, bacterial, yeast, insect, or cell-free system. In some embodiments, the immunogenic compositions or product combinations further comprise an adjuvant. In some embodiments, the adjuvant is alum, QS-21 or MF59, or any combination thereof. In some embodiments, the nucleic acid comprises DNA. In some embodiments, the nucleic acid is provided in a recombinant vector.

Additionally disclosed herein are polypeptides comprising at least one HDAg polypeptide sequence and at least one HBV PreS1 polypeptide sequence. In some embodiments, each of the at least one HDAg polypeptide sequence is a C211 mutant HDAg polypeptide sequence. In some embodiments, each of the at least one HDAg polypeptide sequence is a C211S mutant HDAg polypeptide sequence. C211 is a cysteine appearing at the C-terminus of the HDAg sequence that may cause issues with unintended protein cross-linking and/or post-translational modifications. Accordingly, C211 in embodiments of HDAg sequences provided herein have been mutated to a serine. It is envisioned that other amino acids other than serine can be substituted according to conventional knowledge of amino acid compatibility known in the art. In some embodiments, the at least one HDAg polypeptide sequence comprises the sequence of SEQ ID NO, 47, SEQ ID NO: 48, SEQ ID NO: 49, SEQ ID NO: 50, or any combination thereof. These sequences correspond to C211S mutants of the different HDAg genotype consensus sequences disclosed herein. In some embodiments, the at least one PreS1 polypeptide sequence comprises the sequence of SEQ ID NO: 11 or SEQ ID NO: 12. In some embodiments, the polypeptide comprises one or more epitope tags. In some embodiments, the one or more epitope tags comprise an E-tag, Myc tag, FLAG tag, Strep2 tag, 6×-histidine tag, or any combination thereof. In some embodiments, the E-tag may comprise the sequence of SEQ ID NO: 55, the Myc tag may comprise the sequence of SEQ ID NO: 56, the FLAG tag may comprise the sequence of SEQ ID NO: 57, the Strep2 tag may comprise the sequence of SEQ ID NO: 58, and the 6×-histidine tag may comprise the sequence of SEQ ID NO: 59. However, any other epitope tag generally known in the art can be used. In some embodiments, the polypeptide further comprises one or more linkers. For example, the linker may be a “GGG” linker (SEQ ID NO: 54). However, any other linker as conventionally known in the art can be used. This linker may be placed in between any two components of the polypeptide. For example, a linker may be placed between two HDAg sequences, between two PreS1 sequences, or between an HDAg and PreS1 sequence. In some embodiments, the polypeptide comprises a sequence having at least 80%, 85%, 90%, 95%, 99%, or 100% homology or sequence identity to the sequence of any one of SEQ ID NO: 72-95, 140-141, 145-147, 163-177. In some embodiments, the polypeptide comprises a sequence having at least 80%, 85%, 90%, 95%, 99%, or 100% homology or sequence identity to the sequence of any one of SEQ ID NO: 72-95. These sequences correspond to the fusion constructs provided in Table 5. In some embodiments, the polypeptide is recombinantly expressed. In some embodiments, the polypeptide is recombinantly expressed in a mammalian, bacterial, yeast, insect, or cell-free system. In some embodiments, the polypeptide is recombinantly expressed in a bacterial system, optionally wherein the polypeptide is recombinantly expressed in E. coli.

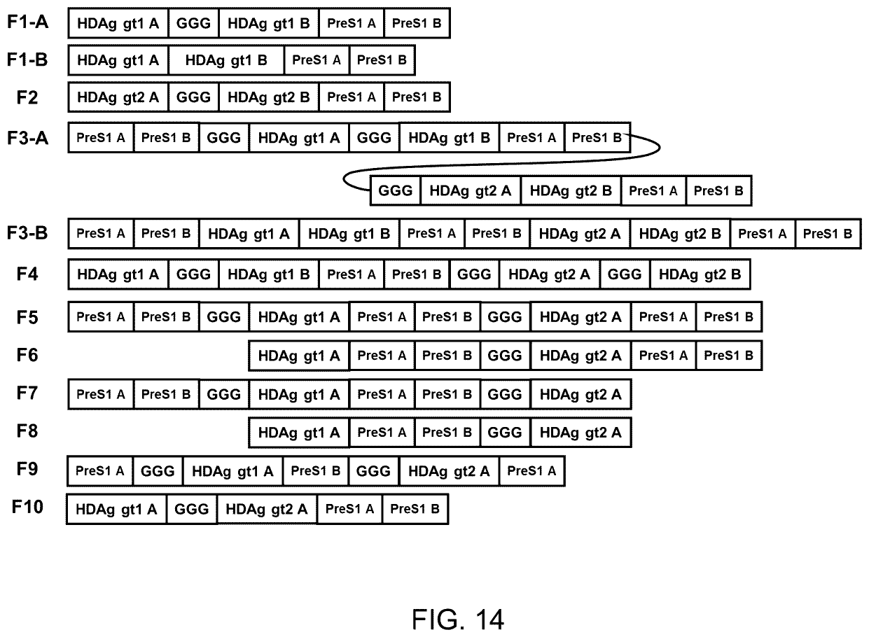

Also disclosed herein are nucleic acids comprising at least one HDAg nucleic acid sequence and at least one PreS1 nucleic acid sequence. In some embodiments, each of the at least one HDAg nucleic acid sequence is a C211 mutant HDAg nucleic acid sequence. In some embodiments, each of the at least one HDAg nucleic acid sequence is a C211S mutant HDAg nucleic acid sequence. In some embodiments, the at least one HDAg nucleic acid sequence and/or the at least one PreS1 nucleic acid sequence are codon optimized to minimize or reduce the number of repeat sequences. In some embodiments, the at least one HDAg nucleic acid sequence comprises the sequence of SEQ ID NO: 43, SEQ ID NO: 44, SEQ ID NO: 45, SEQ ID NO: 46, or any combination thereof. These sequences correspond to C211S mutants of the different HDAg genotype consensus sequences disclosed herein. In some embodiments, the at least one PreS1 nucleic acid sequence comprise the sequence of SEQ ID NO: 51, SEQ ID NO: 52, SEQ ID NO: 53, or any combination thereof. The sequences provided in SEQ ID NO: 51-53 have been codon optimized to reduce repeat sequences that may cause unwanted recombination or plasmid instability. In some embodiments, the nucleic acid further encodes one or more epitope tags. In some embodiments, the one or more epitope tags comprise an E-tag, Myc tag, FLAG tag, Strep2 tag, 6×-histidine tag, or any combination thereof. However, any other epitope tag generally known in the art can be used. In some embodiments, the nucleic acid further comprises sequences encoding for one or more linkers. For example, the sequences may encode for a “GGG” linker (SEQ ID NO: 54). However, any other linker as conventionally known in the art can be used. This sequence encoding for a linker may be placed in between any two components of the nucleic acid encoding any one or more of the polypeptides disclosed herein. For example, the nucleic acid can encode a linker that is placed between two HDAg sequences, between two PreS1 sequences, or between an HDAg and PreS1 sequence in any one or more of the polypeptides described herein. In some embodiments, the nucleic acid comprises a sequence having at least 80%, 85%, 90%, 95%, 99%, or 100% homology or sequence identity to the sequence of any one of SEQ ID NO: 60-71, 138-139, 142-144, 148-162. In some embodiments, the nucleic acid comprises a sequence having at least 80%, 85%, 90%, 95%, 99%, or 100% homology or sequence identity to the sequence of any one of SEQ ID NO: 60-71. These sequences correspond to exemplary nucleic acids encoding for fusion constructs provided in Table 5 and FIG. 14. In some embodiments, the nucleic acid is DNA. In some embodiments, the nucleic acid is provided as a recombinant vector.

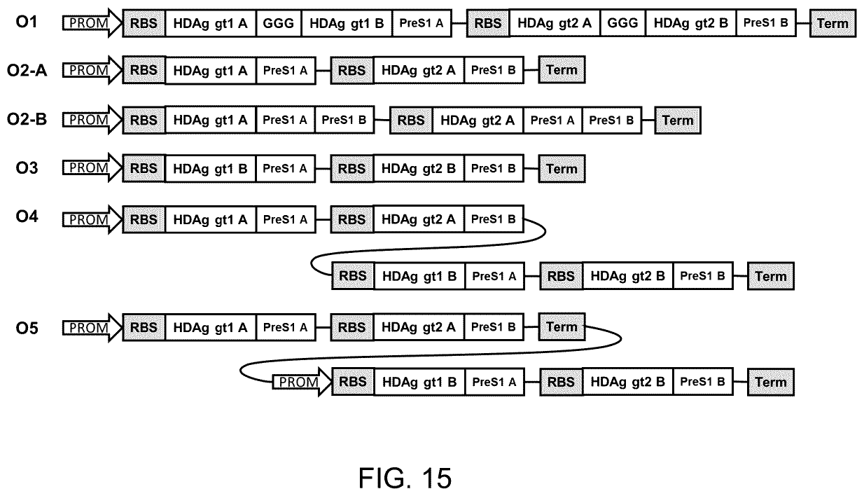

Also disclosed herein are nucleic acid operons (e.g., for expression in a prokaryote, including but not limited to E. coli), comprising two or more genes. In some embodiments, each of the two or more genes comprise at least one HDAg nucleic acid sequence and at least one PreS1 nucleic acid sequence. In some embodiments, each of the two or more genes comprises a 5′ ribosome binding site that enables translation. In some embodiments, the at least one HDAg nucleic acid is a C211 mutant HDDAg nucleic acid sequence. In some embodiments, the at least one HDAg nucleic acid is a C211S mutant HDAg nucleic acid sequence. In some embodiments, the at least one HDAg nucleic acid sequence and/or the at least one PreS1 nucleic acid sequence are codon optimized to reduce the number of repeat sequences. In some embodiments, the at least one HDAg nucleic acid sequence comprises the sequence of SEQ ID NO: 43, SEQ ID NO: 44, SEQ ID NO: 45, SEQ ID NO: 46, or any combination thereof. These sequences correspond to C211S mutants of the different HDAg genotype consensus sequences disclosed herein. In some embodiments, the at least one PreS1 nucleic acid sequence comprise the sequence of SEQ ID NO: 51, SEQ ID NO: 52, SEQ ID NO: 53, or any combination thereof. In some embodiments, each of the two or more genes further encode one or more epitope tags. In some embodiments, the one or more epitope tags comprise an E-tag, Myc tag, FLAG tag, Strep2 tag, 6×-histidine tag, or any combination thereof. However, any other epitope tag generally known in the art can be used. In some embodiments, each of the two or more genes are configured such that the at least one PreS1 nucleic acid sequence is downstream of the at least one HDAg nucleic acid sequence. In some embodiments, the nucleic acid operon further comprises sequences encoding for one or more linkers. For example, the sequences may encode for a “GGG” linker (SEQ ID NO: 54). However, any other linker as conventionally known in the art can be used. This linker may be placed in between any two components of any of the polypeptides described herein. For example, a linker may be placed between two HDAg sequences, between two PreS1 sequences, or between an HDAg and PreS1 sequence of any of the polypeptides described herein. In some embodiments, the nucleic acid operon comprises a sequence having at least 80%, 85%, 90%, 95%, 99%, or 100% homology or sequence identity to the sequence of any one of SEQ ID NO: 96-101. These sequences correspond to the nucleic acid operon constructs depicted in Table 6 and FIG. 15. In some embodiments, the two or more genes encode two or more polypeptides comprising the sequence of any one of SEQ ID NO: 102-133. These two or more polypeptides may or may not comprise epitope tags, which may be used for testing but are not necessarily needed for the final immunogenic compositions.

Also provided herein are polypeptides comprising a sequence having at least 80%, 85%, 90%, 95%, 99%, or 100% homology or sequence identity to the sequence of any one of SEQ ID NO: 136-137.

Also provided herein are nucleic acids comprising a sequence having at least 80%, 85%, 90%, 95%, 99%, or 100%, homology or sequence identity to the sequence of any one of SEQ ID NO: 134-135.

Also disclosed herein are cells comprising any of the polypeptides, nucleic acids, or nucleic acid operons disclosed herein. In some embodiments, the cells are eukaryotes, such as human or animal cells. In some embodiments, the cells are prokaryotes, such as E. coli.

Provided herein are immunogenic compositions comprising any of the polypeptides disclosed herein and a nucleic acid comprising at least one nucleic acid sequence encoding HDAg and at least one nucleic acid sequence encoding PreS1. In some embodiments, the at least one nucleic acid sequence encoding HDAg comprises SEQ ID NO: 1-4, or 43-46, or any combination thereof. In some embodiments, the at least one nucleic acid sequence encoding PreS1 comprises SEQ ID NO: 9-10 or 51-53, or any combination thereof. In some embodiments, the nucleic acid comprises a sequence having at least 80%, 85%, 90%, 95%, 99%, or 100% homology or sequence identity to SEQ ID NO: 15-24 or 35-36. In some embodiments, the nucleic acid comprises a sequence having at least 80%, 85%, 90%, 95%, 99%, or 100% homology or sequence identity to SEQ ID NO: 18. In some embodiments, the nucleic acid is DNA, optionally wherein the nucleic acid is provided as a recombinant vector. In some embodiments, the immunogenic compositions further comprise an adjuvant, optionally wherein the adjuvant is alum, QS-21, or MF59, or any combination thereof.

### Methods of Use

Disclosed herein are methods of generating an immune response in a subject using an immunogenic composition or product combination. In some embodiments, the immunogenic composition or product combination is any one of the immunogenic compositions or product combinations disclosed herein. In some embodiments, the methods comprise administering to the subject at least one prime dose comprising the nucleic acid; and administering to the subject at least one boost dose comprising the polypeptide. In some embodiments, the at least one boost dose further comprises an adjuvant. In some embodiments, the adjuvant is alum, QS-21, or MF59, or any combination thereof. In some embodiments, the at least one boost dose is administered at least 1, 2, 3, 4, 5, 6, 7, 8, 9, 10, 11, 12, 24, 36, or 48 days or weeks after the at least one prime dose is administered or within a range of time defined by any two of the aforementioned time points e.g., within 1-48 days or 1-48 weeks. In some embodiments, the administration is provided enterally, orally, intranasally, parenterally, subcutaneously, intramuscularly, intradermally, or intravenously or any combination thereof. In some embodiments, the administration is performed in conjunction with an antiviral therapy. In some embodiments, the antiviral therapy comprises administration of entecavir, tenofovir, lamivudine, adefovir, telbivudine, emtricitabine, interferon-a, pegylated interferon-a, or interferon alfa-2b, or any combination thereof.

Also disclosed herein are immunogenic compositions or product combinations for use in the treatment, amelioration, or inhibition of hepatitis B and/or hepatitis D infection or protection therefrom. In some embodiments, the immunogenic compositions or product combinations are any one of the immunogenic compositions or product combinations disclosed herein. In some embodiments, the immunogenic compositions or product combinations comprise (a) a nucleic acid comprising at least one nucleic acid sequence encoding hepatitis D antigen (HDAg), and at least one nucleic acid sequence encoding PreS1; and (b) a polypeptide comprising at least one HDAg polypeptide sequence and at least one PreS1 polypeptide sequence. In some embodiments, the at least one nucleic acid sequence encoding HDAg comprises SEQ ID NO: 1, SEQ ID NO: 2, SEQ ID NO: 3 or SEQ ID NO: 4. In some embodiments, the at least one nucleic acid sequence encoding PreS1 comprises SEQ ID NO: 9 or SEQ ID NO: 10 or both. In some embodiments, the nucleic acid is configured such that each HDAg nucleic acid sequence is grouped with a PreS1 nucleic acid sequence, and wherein the PreS1 nucleic acid sequence is immediately downstream of the HDAg nucleic acid sequence. In some embodiments, the immunogenic compositions or product combinations further comprise at least one nucleic acid sequence encoding an autocatalytic peptide cleavage site, wherein the grouped HDAg and PreS1 nucleic acid sequences are separated by the at least one nucleic acid sequence encoding the autocatalytic peptide cleavage site. In some embodiments, the at least one nucleic acid sequence encoding the autocatalytic peptide cleavage site comprises a nucleic acid sequence selected from the group consisting of porcine teschovirus-1 2A (P2A), foot-and-mouth disease virus 2A (F2A), equine rhinitis A virus (ERAV) 2A (E2A) and Thosea asigna virus 2A (T2A) nucleic acid, and wherein each encoded autocatalytic peptide cleavage site may optionally include a GSG (glycine-serine-glycine) motif at its N-terminus. In some embodiments, the at least one nucleic acid sequence encoding the autocatalytic peptide cleavage site comprises SEQ ID NO: 13. In some embodiments, the nucleic acid is codon optimized for expression in a human. In some embodiments, the nucleic acid comprises a sequence having at least 80%, 85%, 90%, 95%, 99%, or 100% homology or sequence identity to SEQ ID NO: 15-24 or 35-36. In some embodiments, the nucleic acid comprises a sequence having at least 80%, 85%, 90%, 95%, 99%, or 100% homology or sequence identity to SEQ ID NO: 18, or SEQ ID NO: 35-36. In some embodiments, the at least one HDAg polypeptide comprises SEQ ID NO: 5, SEQ ID NO: 6, SEQ ID NO: 7 or SEQ ID NO: 8 or any combination thereof. In some embodiments, the at least one PreS1 polypeptide sequence comprises SEQ ID NO: 11 or SEQ ID NO: 12 or both. In some embodiments, the at least one PreS1 polypeptide sequence is downstream of the at least one HDAg polypeptide sequence. In some embodiments, the polypeptide comprises a sequence having at least 80%, 85%, 90%, 95%, 99%, or 100% homology or sequence identity to the sequence of SEQ ID NO: 25-34 or 37. In some embodiments, the polypeptide comprises a sequence having at least 80%, 85%, 90%, 95%, 99%, or 100% homology or sequence identity to the sequence of SEQ ID NO: 29, 31, 32, or 37. In some embodiments, the polypeptide is recombinantly expressed. In some embodiments, the polypeptide is recombinantly expressed in a mammalian, bacterial, yeast, insect, or cell-free system. In some embodiments, the immunogenic compositions or product combinations further comprise an adjuvant. In some embodiments, the adjuvant is alum, QS-21, or MF59, or any combination thereof. In some embodiments, the nucleic acid comprises DNA. In some embodiments, the nucleic acid is provided in a recombinant vector.

Also disclosed herein are immunogenic compositions or product combinations for use in the treatment, amelioration, or inhibition of hepatitis B and/or hepatitis D infection or protection therefrom. In some embodiments, the immunogenic compositions or product combinations are any one of the immunogenic compositions or product combinations disclosed herein. In some embodiments, the immunogenic compositions or product combinations comprise (a) a nucleic acid comprising at least one nucleic acid sequence encoding hepatitis D antigen (HDAg), and at least one nucleic acid sequence encoding PreS1; and (b) a polypeptide comprising at least one HDAg polypeptide sequence and at least one PreS1 polypeptide sequence. In some embodiments, the at least one nucleic acid sequence encoding HDAg comprises SEQ ID NO: 1-4 or 43-46. In some embodiments, the at least one nucleic acid sequence encoding PreS1 comprises SEQ ID NO: 9-10 or 51-53, or any combination thereof. In some embodiments, the nucleic acid is configured such that each HDAg nucleic acid sequence is grouped with a PreS1 nucleic acid sequence, and wherein the PreS1 nucleic acid sequence is immediately downstream of the HDAg nucleic acid sequence. In some embodiments, the immunogenic compositions or product combinations further comprise at least one nucleic acid sequence encoding an autocatalytic peptide cleavage site, wherein the grouped HDAg and PreS1 nucleic acid sequences are separated by the at least one nucleic acid sequence encoding the autocatalytic peptide cleavage site. In some embodiments, the at least one nucleic acid sequence encoding the autocatalytic peptide cleavage site comprises a nucleic acid sequence selected from the group consisting of porcine teschovirus-1 2A (P2A), foot-and-mouth disease virus 2A (F2A), equine rhinitis A virus (ERAV) 2A (E2A) and Thosea asigna virus 2A (T2A) nucleic acid, and wherein each encoded autocatalytic peptide cleavage site may optionally include a GSG (glycine-serine-glycine) motif at its N-terminus. In some embodiments, the at least one nucleic acid sequence encoding the autocatalytic peptide cleavage site comprises SEQ ID NO: 13. In some embodiments, the nucleic acid is codon optimized for expression in a human. In some embodiments, the nucleic acid comprises a sequence having at least 80%, 85%, 90%, 95%, 99%, or 100% homology or sequence identity to SEQ ID NO: 15-24, 35-36, 60-71, 134-135, or 138-139. In some embodiments, the nucleic acid comprises a sequence having at least 80%, 85%, 90%, 95%, 99%, or 100% homology or sequence identity to SEQ ID NO: 134-135. In some embodiments, the at least one HDAg polypeptide comprises SEQ ID NO: 5-8 or 47-50, or any combination thereof. In some embodiments, the at least one PreS1 polypeptide sequence comprises SEQ ID NO: 11 or SEQ ID NO: 12 or both. In some embodiments, the at least one PreS1 polypeptide sequence is downstream of the at least one HDAg polypeptide sequence. In some embodiments, the polypeptide comprises a sequence having at least 80%, 85%, 90%, 95%, 99%, or 100% homology or sequence identity to the sequence of SEQ ID NO: 25-34, 37, 72-95, 136-137, or 140-141. In some embodiments, the polypeptide comprises a sequence having at least 80%, 85%, 90%, 95%, 99%, or 100% homology or sequence identity to the sequence of SEQ ID NO: 136-137 or 140-141. In some embodiments, the polypeptide is recombinantly expressed. In some embodiments, the polypeptide is recombinantly expressed in a mammalian, bacterial, yeast, insect, or cell-free system. In some embodiments, the immunogenic compositions or product combinations further comprise an adjuvant. In some embodiments, the adjuvant is alum, QS-21, or MF59, or any combination thereof. In some embodiments, the nucleic acid comprises DNA. In some embodiments, the nucleic acid is provided in a recombinant vector.

Also provided herein are methods of generating an immune response in a subject. The methods comprise administering to the subject any one of the polypeptides, nucleic acids, proteins encoded by any one of the nucleic acid operons, or immunogenic compositions disclosed herein. In some embodiments, the immunogenic composition is administered in a prime-boost approach, wherein the subject is administered at least one prime dose comprising the nucleic acid of the immunogenic composition, and subsequently administered at least one boost dose comprising the polypeptide of the immunogenic composition. In some embodiments, the at least one boost dose is administered at least 1, 2, 3, 4, 5, 6, 7, 8, 9, 10, 11, 12, 24, 36, or 48 days or weeks after the at least one prime dose is administered or within a range of time defined by any two of the aforementioned time points e.g., within 1-48 days or 1-48 weeks. In some embodiments, the administration is provided enterally, orally, intranasally, parenterally, subcutaneously, intramuscularly, intradermally, or intravenously or any combination thereof.

Also disclosed herein are the polypeptides, nucleic acids, proteins encoded by the nucleic acid operons, or immunogenic compositions disclosed herein for use as a medicament, e.g., in the treatment, prevention, ameliorate, or inhibition of hepatitis B and/or hepatitis D in a subject in need thereof.

### EXAMPLES

Some aspects of the embodiments discussed above are disclosed in further detail in the following examples, which are not in any way intended to limit the scope of the present disclosure. Those in the art will appreciate that many other embodiments also fall within the scope of the invention, as it is described herein above and in the claims.

### Example 1: Methodology

### Animals

Female C57BL/6 (H-2b) mice were obtained from Charles River Laboratories. Human leukocyte antigen A2 (HLA-A2) transgenic HHD mice were bred inhouse. All mice were 8-10 weeks old at the start of the experiments and maintained under standard conditions. uPA^{+/+}-SCID mice with humanized liver were produced and maintained. New Zealand White rabbits were purchased from commercial vendors.

### DNA Plasmids

Plasmids encoding for genotypes 1 and 2 of the L-HDAg and the PreS1 domain (aa 2-48) of the HBsAg, were used in this study as fusion constructs, optionally cleaved by a P2A, consisting of different combinations of HDAg/PreS1 sequences. The HDAg sequences of genotypes 1 and 2 obtained from four different clinical isolates; US-2 and CB, and Jul. 18, 1983 and TW2476, respectively. All genes were cloned into the pVAX1 backbone (Invitrogen, Carlsbad, CA) using restriction sites EcoR I and HindIII. Plasmids were grown in TOP10 E. coli cells (Life Technologies, Carlsbad, CA) and purified for in vivo injections using Qiagen Endofree DNA purification kit (Qiagen GmbH) following manufacturer's instructions. The correct gene size was confirmed by restriction enzyme digests using EcoRI and HindIII (Fast Digest, Thermo Fisher Scientific).

### Western Blot

Western Blot was essentially performed as generally known in the art. Hela cells were transfected with each pVAX1 D1-D10 DNA plasmids and pVAX1 with the reporter gene GFP as control, using Lipofectamine® 3000 Transfection Reagent (Thermo Fisher Scientific). For protein detection, serum from the D4 vaccinated rabbit diluted 1:1000 (primary antibody) and goat anti-Rabbit Immunoglobulins HRP 0.25 g/L (DAKO) diluted 1:4000 (secondary antibody) used. For Chemiluminescence detection, Pierce TM ECL Plus Western Blotting Substrate was used and images were collected with Gel Doc XR+ System (Biorad).

### Peptides

A total of 168 HDAg 15-mer peptides with 10 aa overlap were purchased from Sigma-Aldrich (St. Louis, MO). The 168 peptides were divided in 8 pools each containing 20 or 21 peptides. Four pools correspond to genotype 1 (pool 1₁₋₂₁, pool 2₂₂₋₄₂, pool 3₄₃₋₆₃ and pool 4₆₄₋₈₄) and four pools correspond to genotype 2 (pool 1₁₋₂₁, pool 2₂₂₋₄₂, pool 3₄₃₋₆₃ and pool 4₆₄₋₈₄) for sequences A, B, C and D with each sequence referring to each clinical isolate.

Two consensus sequences of the PreS1 HBsAg (PreS1A and PreS1B) consisting of 47 aa and 20-mer PreS1 peptides with 10 aa overlap for HBV (sub-) genotypes A1, A2, B, B2, C, D1, E1 and F were purchased from Sigma-Aldrich (St. Louis, MO). All peptides have passed QC (Sigma-Aldrich PEPscreen® Directory) and have purity >70%. OVA 257-264 CTL (SIINFEKL (SEQ ID NO:) and OVA 323-339 Th (ISQAVHAAHAEINEAGR (SEQ ID NO: 39)) ovalbumin peptides were used as negative peptide controls while Concanavalin A (ConA) purchased from Sigma Aldrich (St. Louis, MO) was used as positive control at final concentration of 0.5 μg/μL.

### Immunization Protocols for Evaluating Immunogenicity of HBV/HDV Plasmids in Mice and Rabbits

To evaluate the immunogenicity of the constructs in vivo, mice and rabbits were immunized essentially as described boosted at monthly intervals and sacrificed two weeks later for spleens and blood collection. In brief, female C57BL/6 mice (five per group) were immunized intramuscularly (i.m.) in the tibialis cranialis anterior (TA) muscle with 50 μg plasmid DNA in a volume of 50 μL in sterile PBS by regular needle (27G) injection followed by in vivo electroporation (EP) using the Cliniporator2 device (IGEA, Carpi, Italy). During in vivo electroporation, a 1 ms 600 V/cm pulse followed by a 400 ms 60 V/cm pulse pattern was used to facilitate better uptake of the DNA. Prior to vaccine injections, mice were given analgesic and kept under isoflurane anesthesia during the vaccinations. For studies in rabbits, two New Zealand White rabbits per group were immunized with 300 μg D3 and D4 DNA vaccines. Vaccines were administered by i.m. injection in 300 μL sterile PBS to the right TA muscle followed by in vivo EP.

### Detection of IFNγ Producing T Cells by Enzyme-Linked Immunospot Assay (ELISpot)

Two weeks after last vaccination, splenocytes from each immunized group of mice pooled (five mice/group) and tested for their ability to induce HBV/HDV-specific T cells based on IFN-γ secretion after peptide stimulation for 48h as known in the art using a commercially available ELISpot assay (Mabtech, Nacka Strand, Sweden).

### Antibody Detection by Enzyme-Linked Immunosobent Assay (ELISA)

Detection of mouse and rabbit IgG against PreS1 consensus and overlapping 20 mer-peptides (10 μg/mL) was performed using protocols known in the art. Antibody titers determined as endpoint serum dilutions at which the OD value at 405 nm is at least twice the OD of the negative control (non-immunized or control animal serum) at the same dilution.

HBV Neutralization Assay in Human-Liver uPA-SCID Mouse Model

HepG2-NTCP-A3 is a selected cell clone derived from the HepG2 cell expressing human NTCP as described previously. It was cultivated in DMEM medium supplemented with 10% fetal calf serum, 2 mM 1-glutamine, 50 U/mL penicillin, and 50 μg/mL streptomycin. During and after inoculation, 2.5% DMSO was added to the medium to enhance HBV infection and replication. HBV virus stock used for infection was prepared from HepAD38 cells by PEG precipitation as described. Cell culture medium between day 3-6 post infection were collected and diluted 1:5 with PBS for ELISA analysis of HBeAg quantification using commercial antibodies.

### Statistical Analysis

Data was analyzed using GraphPad Prism V.5, V.8, and V.9 software and Microsoft Excel V.16.13.1, and V.16.62.

### Example 2: Exemplary HBV and HDV Immunogenic Constructs

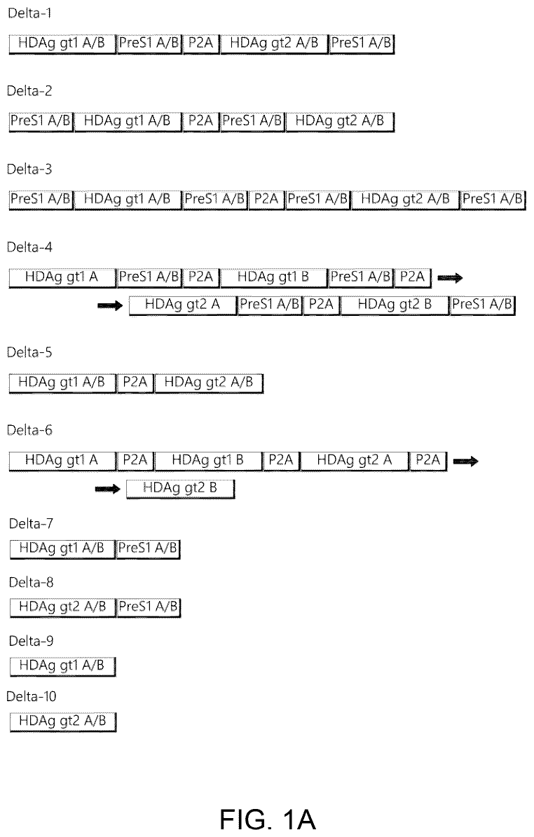

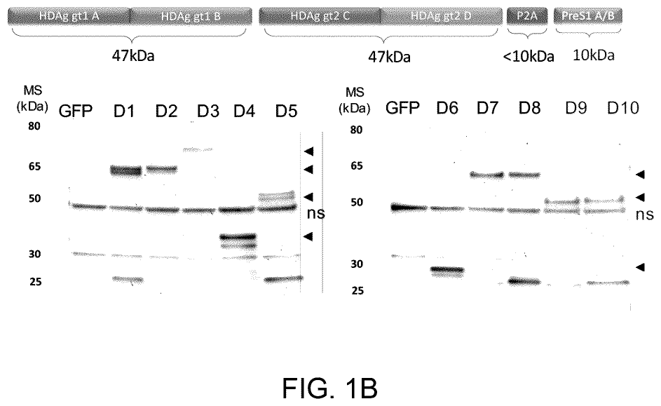

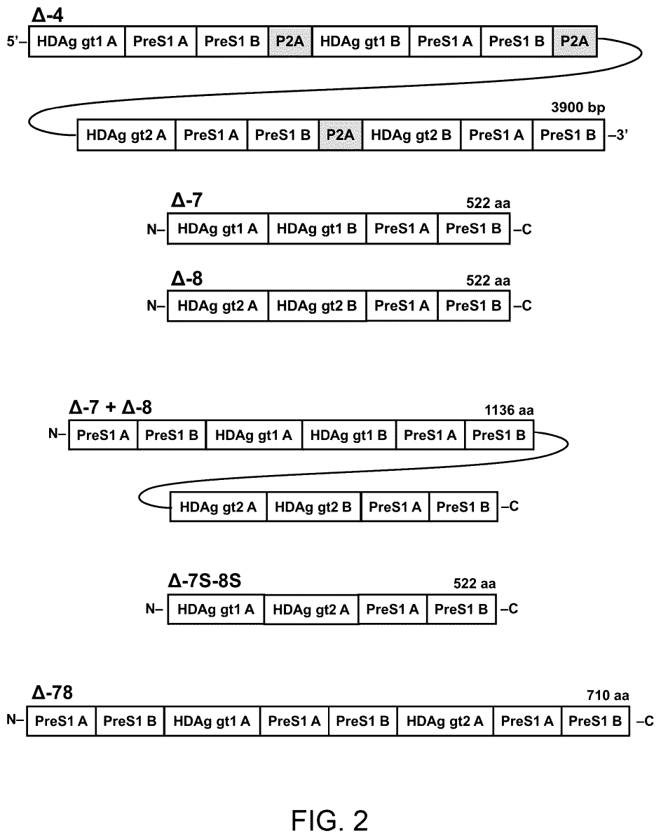

The use of recombinant HBV and HDV polypeptide constructs have been shown to be effective in eliciting antibody formation and immune protection against the two hepatitis viruses, for example, in WO 2017/132332, hereby expressly incorporated by reference in its entirety. These recombinant polypeptide constructs were assembled by combining HDAg chosen from four distinct HDV genotypes (HDAg genotype 1 A, HDAg genotype 1 B, HDAg genotype 2 A, and HDAg genotype 2 B), PreS1 chosen from two genotype consensus sequences (PreS1 A and PreS1B), and, optionally, one or more P2A autocatalytic peptide cleavage sites. Schematics for thirteen exemplary recombinant constructs are shown in FIG. 1A and FIG. 2, and corresponding SEQ ID NOs for DNA and polypeptide sequences, if applicable, are provided in Table 1. A Western blot confirms that polypeptides properly express from Δ-1 to Δ-10 recombinant constructs (FIG. 1B).

TABLE 1
SEQ ID NOs for the HBV/HDV immunogenic constructs

| | DNA Sequence | |
|---|---|---|
| | (human codon | |
| | optimized sequence | Polypeptide |
| Name | unless indicated) | Sequence |
| Δ-1 (Delta-1; D1; D-1) | SEQ ID NO: 15 | SEQ ID NO: 25 |
| Δ-2 (Delta-2; D2; D-2) | SEQ ID NO: 16 | SEQ ID NO: 26 |
| Δ-3 (Delta-3; D3; D-3) | SEQ ID NO: 17 | SEQ ID NO: 27 |
| Δ-4 (Delta-4; D4; D-4) | SEQ ID NO: 18 | SEQ ID NO: 28 |
| Δ-5 (Delta-5; D5; D-5) | SEQ ID NO: 19 | SEQ ID NO: 29 |
| Δ-6 (Delta-6; D6; D-6) | SEQ ID NO: 20 | SEQ ID NO: 30 |
| Δ-7 (Delta-7; D7; D-7) | SEQ ID NO: 21 | SEQ ID NO: 31 |
| Δ-8 (Delta-8; D8; D-8) | SEQ ID NO: 22 | SEQ ID NO: 32 |
| Δ-9 (Delta-9; D9; D-9) | SEQ ID NO: 23 | SEQ ID NO: 33 |
| Δ-10 (Delta-10; D10; D-10) | SEQ ID NO: 24 | SEQ ID NO: 34 |
| Δ-7 + Δ-8 fusion (Delta- | SEQ ID NO: 36 | SEQ ID NO: 37 |
| 7 + Delta-8; D7 + D8; | (Wild type sequence: | |
| D-7 + D-8) | SEQ ID NO: 35) | |
| Δ-7S-8S (Delta-7S-8S; D- | Wild type sequence: | SEQ ID NO: 136 |
| 7S-8S) | SEQ ID NO: 134 | |
| Δ-78 (Delta-78; D-78) | Wild type sequence: | SEQ ID NO: 137 |
| | SEQ ID NO: 135 | |

### Example 3: HBV/HDV DNA Compositions Induce Immunogenic Response in Mice

While immunogenic compositions and vaccines have traditionally been either whole organisms or antigenic proteins, it has been recently shown that in vivo administration of DNA to living tissue and the subsequent transcription and translation of antigenic proteins are also highly effective in triggering an immune response. These DNA immunogenic compositions are being explored as potential vaccine candidates against various diseases.

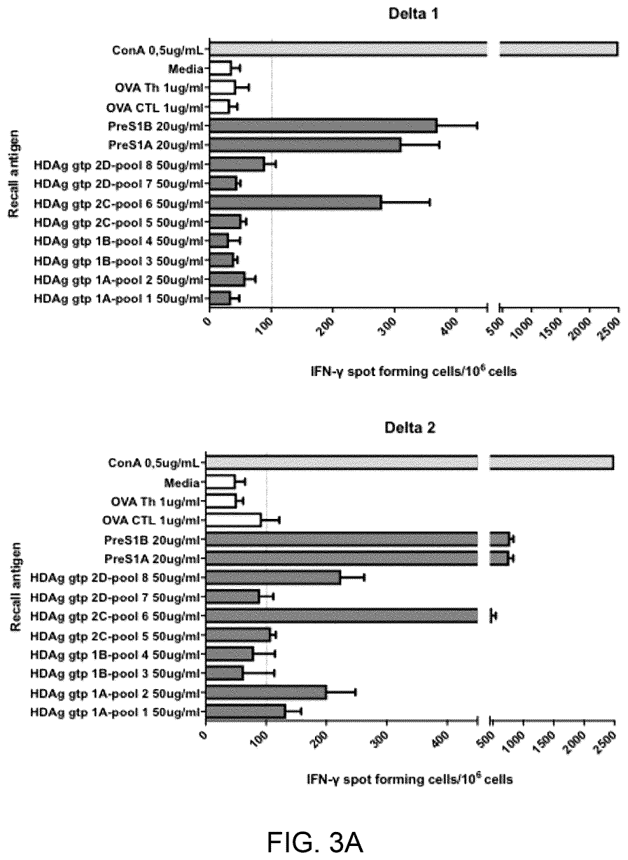

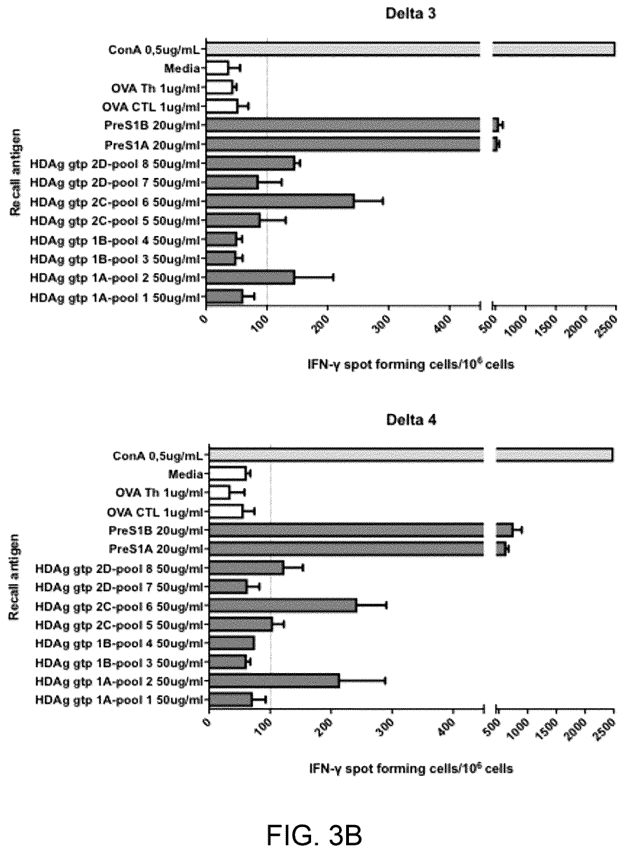

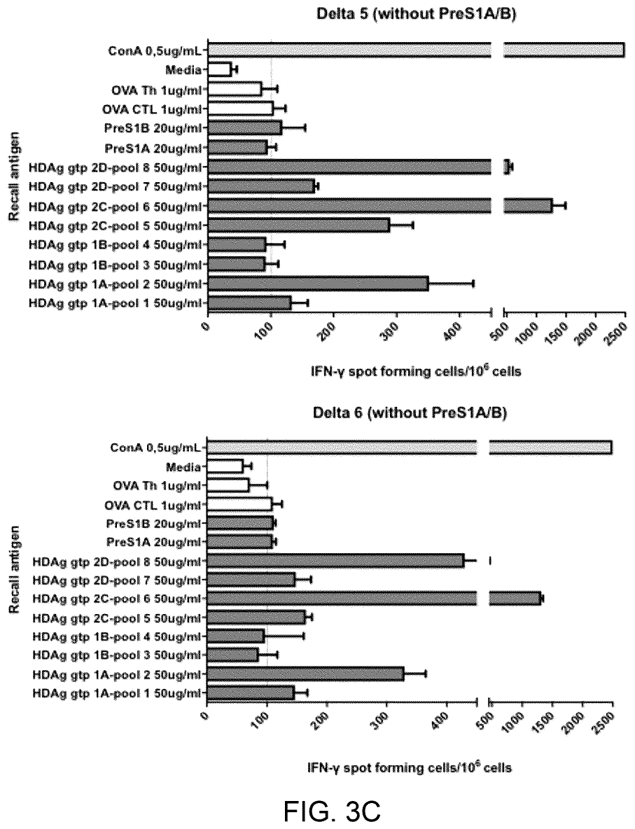

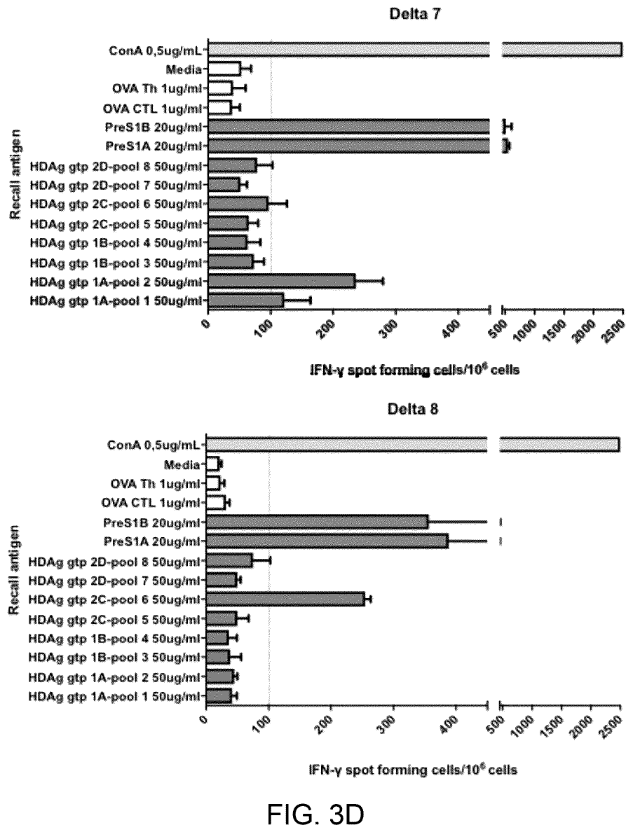

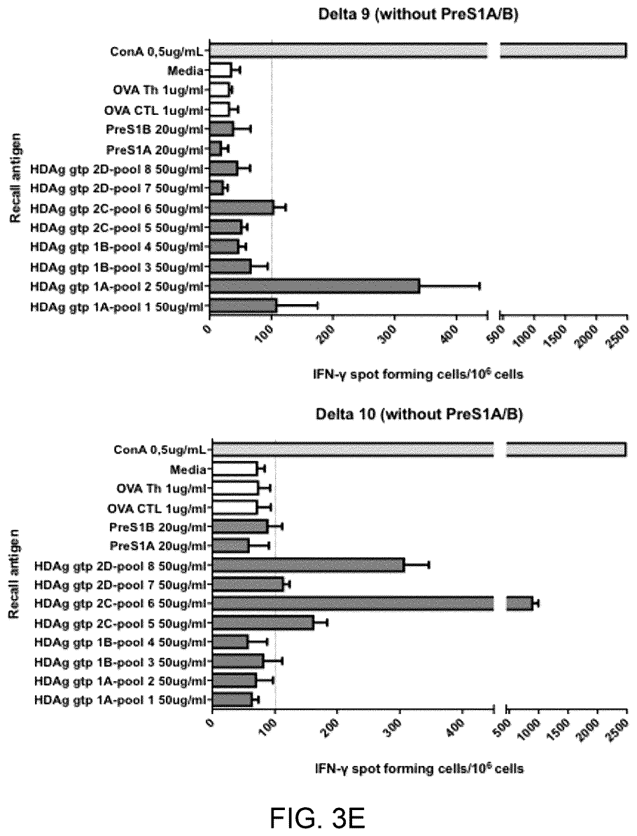

Following 2 weeks after the second administration of the DNA construct compositions, immunity of the mice against HBV and HDV antigens were assessed. White blood cells were purified from mouse whole blood samples and incubated with purified polypeptide antigens, including PreS1 A, PreS1 B, HDAg genotypes 1 A, 1 B, 2 A, and 2 B. Cells were also incubated with Concanavalin A (“ConA”) as a positive control, and two ovalbumin peptides (“OVA Th” and “OVA CTL”) as negative controls. The population frequency of interferon gamma (IFNγ) producing cells in response to antigen exposure was assessed by enzyme-linked immunospot assay (ELISpot). Briefly, white blood cells were incubated with antigen in wells coated with IFNγ antibodies. The cells were then removed, and biotinylated IFNγ antibodies, alkaline phosphatase-crosslinked streptavidin, and alkaline phosphatase substrate colorimetric reagents were added to the wells in succession with thorough washing in between. The plate was then allowed to dry and the remaining colored spots that correspond to IFNγ-secreting cells were counted by microscopy. The quantitative total of IFNγ spot forming cells per 106 total cells in response to the various peptide antigens for each of the mice are shown in FIG. 3A (Δ-1 and Δ-2), 3B (Δ-3 and Δ-4), 3C (Δ-5 and Δ-6), 3D (Δ-7 and Δ-8), and 3E (Δ-9 and Δ-10).

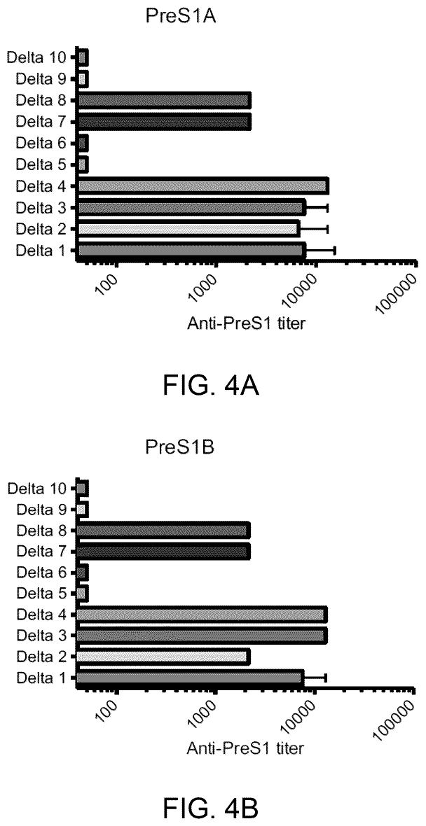

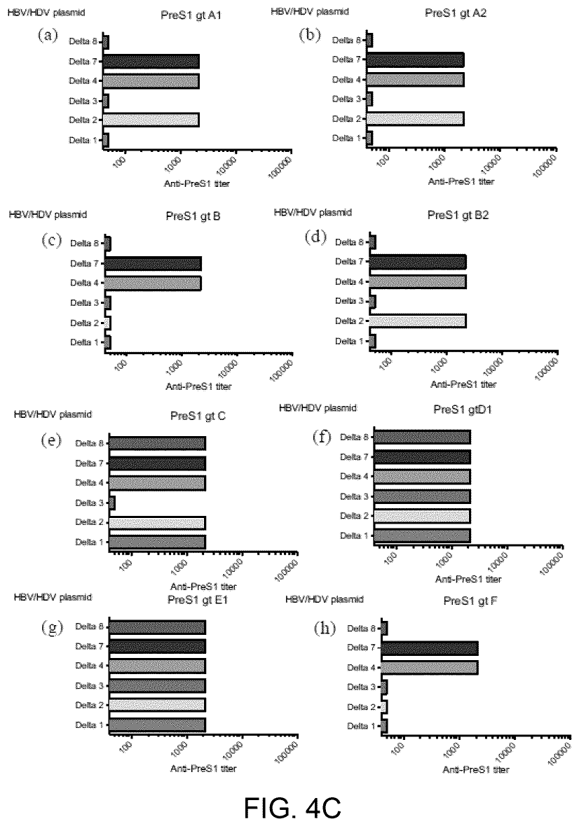

Anti-sera were tested for reactivity against PreS1A and PreS1B consensus peptides (aa 2-48) and for cross-reactivity against HBV (sub-) types A1, A2, B, B2, C, D1, E1 and F using pools of 20-mer PreS1-peptides. Immunogenic compositions comprising Δ-1, Δ-2, Δ-3, Δ-4, Δ-7, and Δ-8 resulted in robust immunogenicity against both HBV PreS1 antigens (FIG. 4A-4B). Δ-3 and Δ-4 induced antibody titers >10⁴ in mice, followed by constructs 4-1, 4-2, Δ-7 and Δ-8. Importantly, anti-sera from Δ-4 and Δ-7 immunized mice effectively cross-reacted between all tested HBV types (FIG. 4C). The immune response to HDAg peptides were more variable, likely due to differences in genotypic sequences, but typically greater than the ovalbumin controls. Notably, in Δ-3 and Δ-4 treated groups, a slight reduction in HDV T cell responses is observed when i.e., compared to constructs that contain only HDAg (Δ-5, Δ-6, Δ-9, Δ-10) which might be attributed to epitope recognition competition with the simultaneous priming of PreS1-specific T cells. Overall, this shows that active immunization is able to induce functional T cells to PreS1 and HDAg antigens and suggests that a broadly functional immunotherapy should contain both HDV genotypes 1 and 2 to ensure induction of specific T cells.

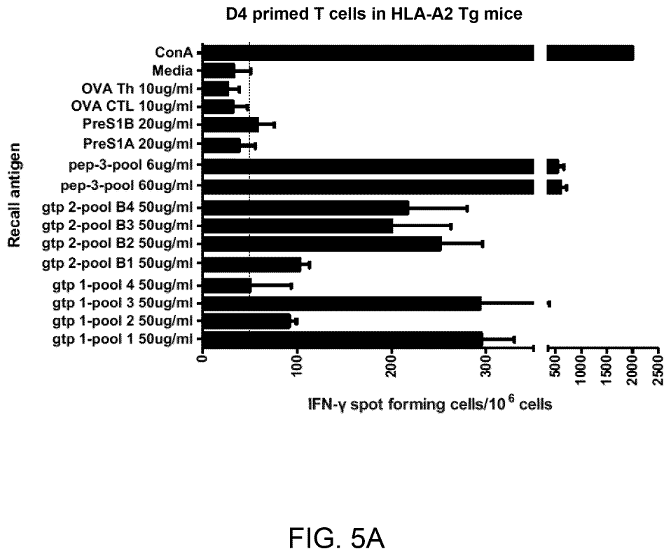

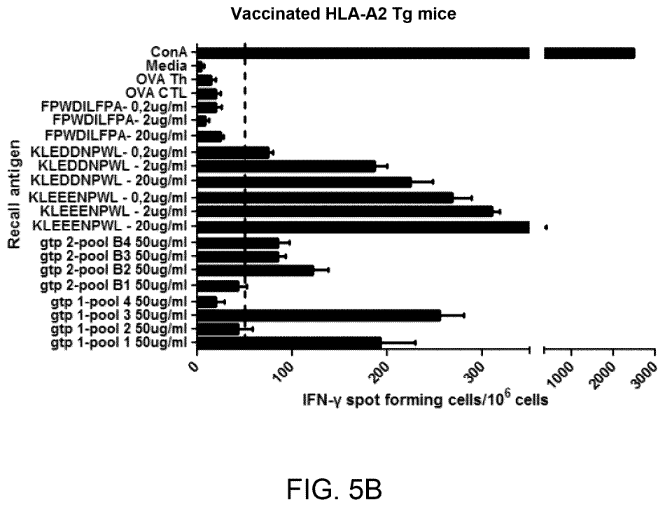

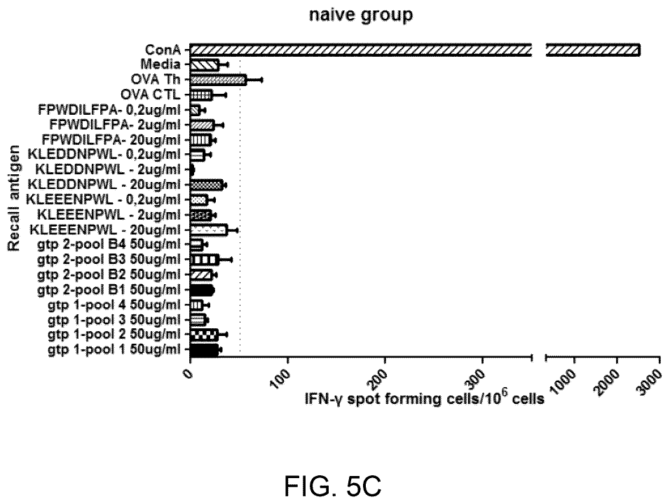

Similar experiments were performed with HLA-A2 restricted T cells purified from HLA-A2 transgenic HHD mice. IFNγ ELISpot of normal C57BL/6 (FIG. 5A) and HLA-A2 HHD (FIG. 5B) mice electroporated with a composition comprising Δ-4, along with naïve HLA-A2 HHD mice as control (FIG. 5C), confirms immunogenicity in the transgenic mice, suggesting efficacy of the DNA compositions for treating humans.

### Example 4: HBV/HDV DNA Compositions Induce Immunogenic Response in Rabbits

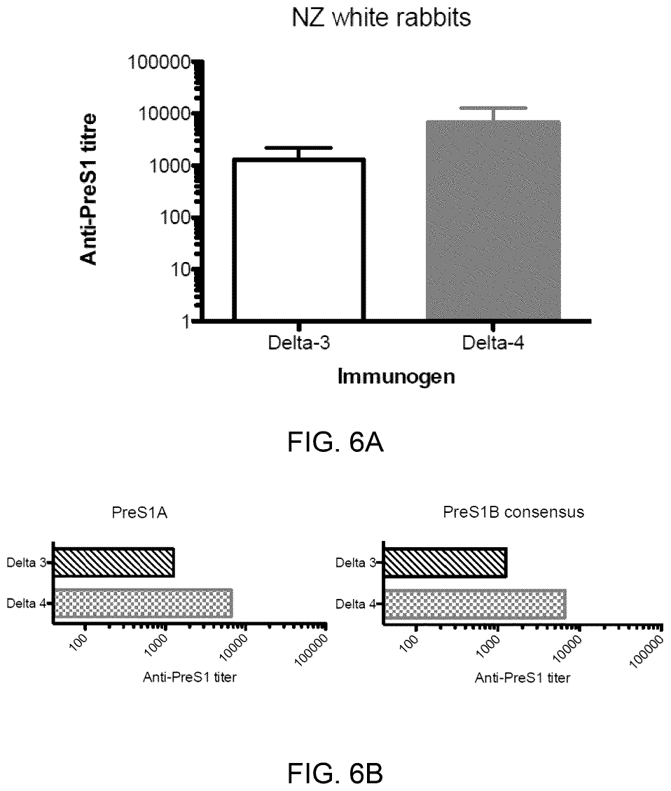

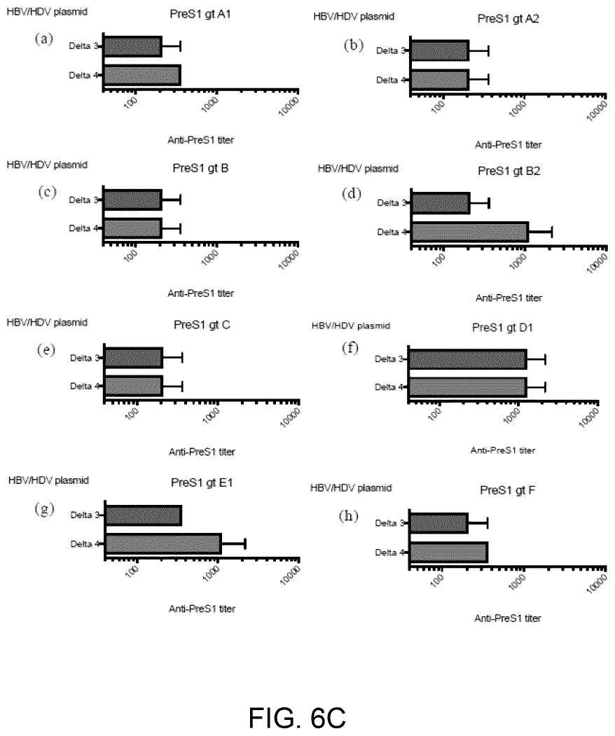

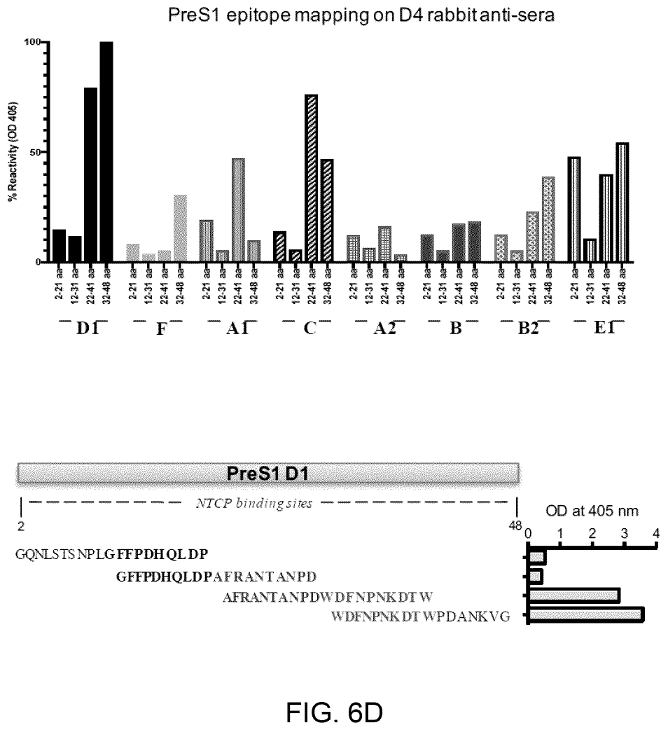

Corresponding experiments described in Example 3 were also performed in rabbits (Oryctolagus cuniculus). New Zealand white rabbits were injected intramuscularly with a saline solution containing 900 μg of DNA compositions comprising either Δ-3 or Δ-4 and subjected to electroporation. Doses were administered at 0 and 4 weeks. After immunization, anti-PreS1 antibody titers in the rabbit sera were observed for both DNA compositions comprising Δ-3 or Δ-4, with Δ-4 being more effective (>10³) (FIG. 6A-6B). Cross-reactivity of the rabbit antisera against HBV (sub-) types A1, A2, B, B2, C, D1, E1 and F using pools of 20-mer PreS1 peptides was also tested (FIG. 6C). The fine specificity of the rabbit D4 anti-sera was determined using individually 20-mer PreS1 peptides of HBV types A1, A2, B, B2, C, D1, E1 and F (FIG. 6D). This mapped the epitopes to PreS1 located to region 22-48 aa of genotype D1, as indicated by the higher reactivity, followed by lower reactivity to genotypes C, E1 and A1. This overlaps with the NTCP binding site and partly with previously identified epitopes recognized by neutralizing antibodies.

Table 2 summarizes the immunity effects of the ten DNA immunogenic compositions. DNA compositions comprising Δ-4 resulted in the greatest titer of anti-PreS1/anti-HBV antibodies in both mice and rabbits and are used in the prime/boost immunizations of the subsequent Examples. Δ-4 also shows the broadest reactivity to the different HBV genotypes. “n.d” denotes low or undetectable levels of antibody activity. “n/a” denotes that the experiment was not performed.

TABLE 2
HBV/HDV DNA vaccine screen (50 μg DNA im/EP)

| | HDV | Anti-PreS1 | Genotype cross- | Anti-PreS1 |
|---|---|---|---|---|
| | genotype | titer | reactivity | titer in |
| Candidate | (Gt) | in mice | in mice | rabbits |
| Δ-1 | Gt 1 and 2 | 1:2160 | C, D, E | n/a |
| Δ-2 | Gt 1 and 2 | 1:2160 | A1, A2, | <1:60 |
| | | | B2, C, | |
| | | | D, E | |
| Δ-3 | Gt 1 and 2 | 1:2160- | D, E | 1:360 |
| | | 1:12960 | | |
| Δ-4 | Gt 1 and 2 | 1:12960 | A-F | 1:2560 |
| Δ-5 | Gt 1 and 2 | n.d. | n.d. | n/a |
| Δ-6 | Gt 1 and 2 | n.d. | n.d. | n/a |
| Δ-7 | Gt 1 | 1:2160 | A-E | n/a |
| Δ-8 | Gt 2 | 1:2160 | C, D, E | n/a |
| Δ-9 | Gt 1 | n.d. | n.d. | n/a |
| Δ-10 | Gt 2 | n.d. | n.d. | n/a |

### Example 5: DNA Prime/Protein Boost Approach with HBV/HDV Constructs Improves Immunogenic Response in Mice

DNA compositions comprising Δ-4 (SEQ ID NO: 18) and polypeptide compositions comprising Δ-7 (SEQ ID NO: 31) or 4-8 (SEQ ID NO: 32) were used for a DNA prime/protein boost immunization approach to build adaptive immunity and induce antibody production against HBV and/or HDV in vivo (FIG. 2).

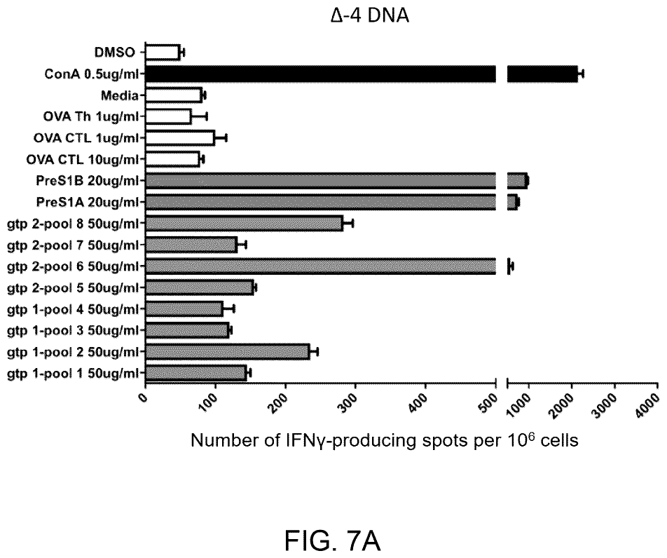

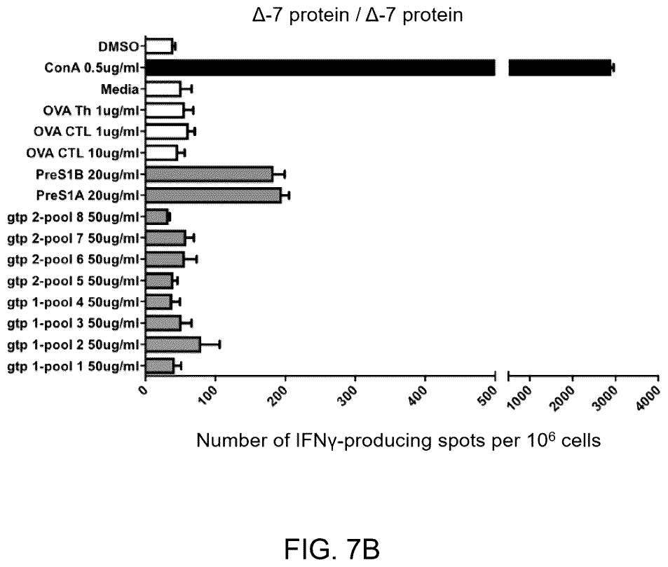

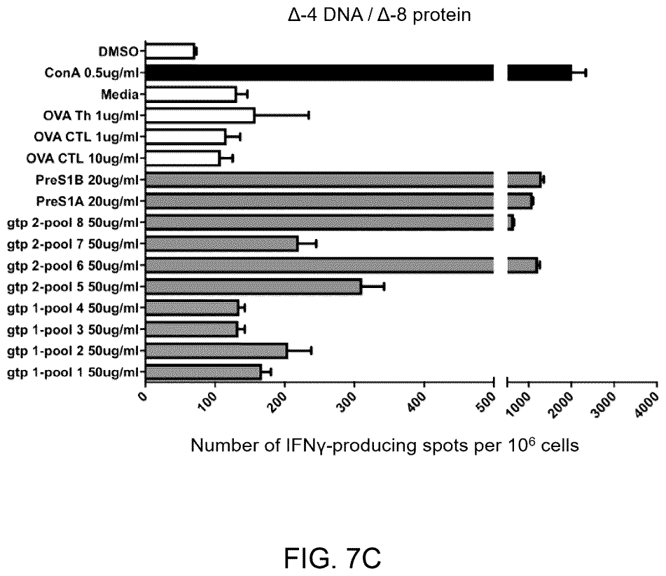

C57BL/6 mice were immunized with (1) a DNA composition comprising Δ-4 (3 sequential doses of 50 μg DNA), (2) a polypeptide composition comprising Δ-7 (3 sequential does of 20 μg protein with alum adjuvant), or (3) a DNA composition comprising Δ-4 followed by a polypeptide composition comprising Δ-8 (2 doses of 50 μg DNA then 2 doses of 20 μg protein with alum). After administration of the compounds, purified white blood cells were tested for IFNγ production in response to HBV and HDV antigens by ELISpot (as described in Examples 1 and 2). Mice treated with (1) exhibited a commensurate response to hepatitis antigens (FIG. 7A) observed in Example 3 and FIG. 3B, but mice treated with the DNA prime/protein boost compositions of (3) resulted in a comparatively stronger immune cell response overall (FIG. 7C). As Δ-8 includes sequences for the HDAg genotype 2 polypeptides, the assayed immune response is particularly improved against these antigens (FIG. 7C, gtp 2-pool 5, 6, 7 and 8). Conversely, the protein-only approach of (2) using Δ-7 polypeptides fails to both elicit an equally effective immune response for both HBV and HDV antigens (FIG. 7B). This demonstrates that this DNA prime/protein boost approach may be effective at inducing a robust immunogenic response greater than traditional protein or organism-based compositions for certain pathogens, including HBV and HDV.

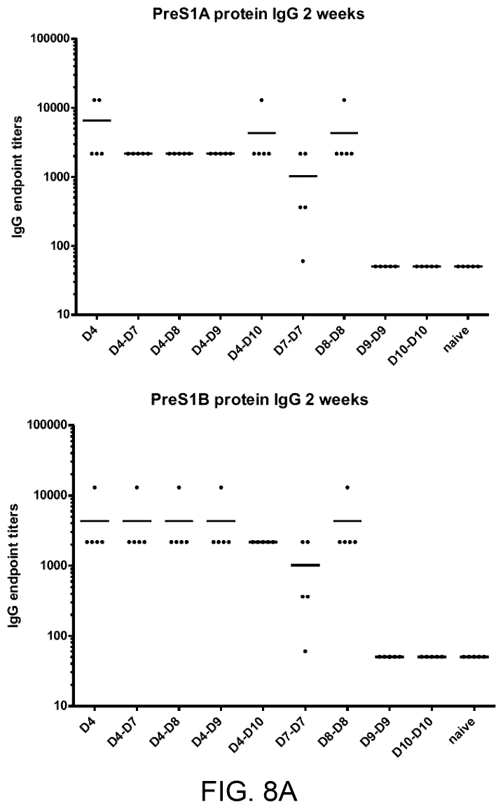

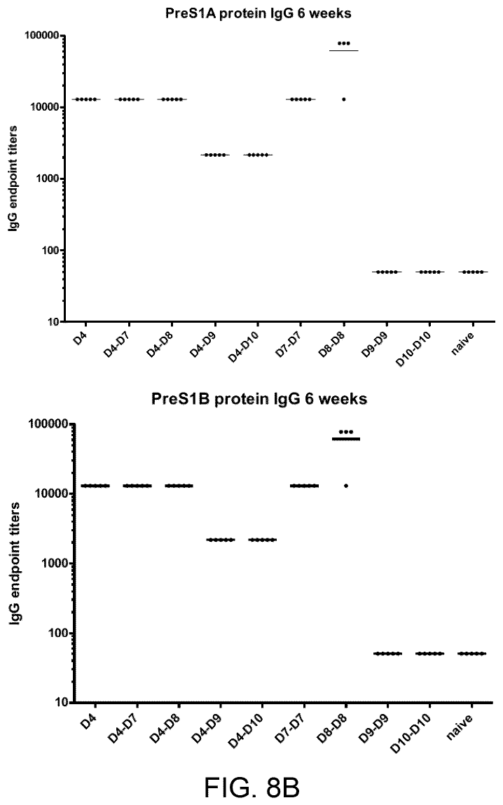

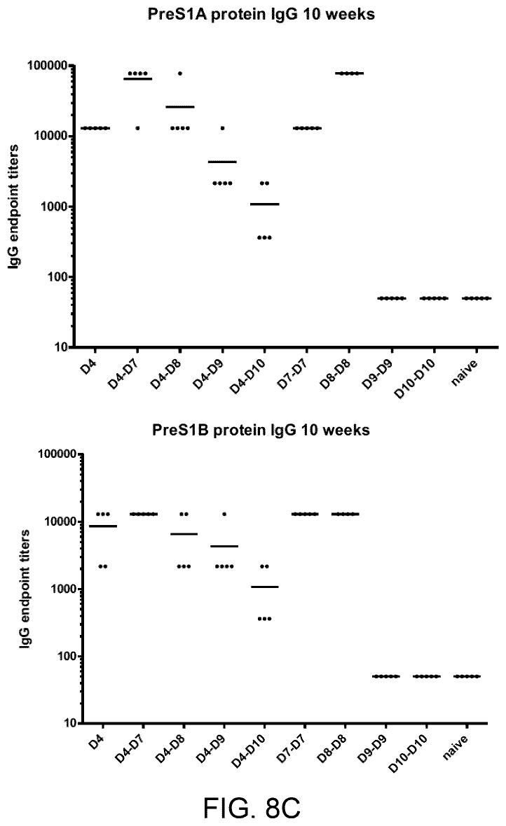

Other DNA prime/protein boost combinations were also assessed in mice. Anti-PreS1 IgG titers in mice were measured after immunization with (1) a DNA-only composition comprising Δ-4 (“D4”), (2) protein-only compositions comprising Δ-7 (“D7-D7”), Δ-8 (“D8-D8”), Δ-9 (“D9-D9”), or Δ-10 (“D10-D10”), or (3) DNA-protein compositions comprising Δ-4 DNA with Δ-7 (“D4-D7”), Δ-8 (“D4-D8”), Δ-9 (“D4-D9”), or Δ-10 (“D4-D10”) protein. Compositions were administered three times at weeks 0, 4, and 8, with either 50 μg DNA im/EP or 20 μg protein with alum administered for each dose. For DNA-protein compositions (3), 50 μg DNA im/EP was administered for the first dose at week 0, and 20 μg protein with alum was administered for the second and third doses at weeks 4 and 8. Anti-PreS1 IgG titers in sera were assessed after 2 weeks (FIG. 8A), 6 weeks (FIG. 8B), and 10 weeks (FIG. 8C) after the first dosage (i.e., 2 weeks after each dosage). DNA prime/protein boost composition D4-D7 results in superior anti-PreS1 titers after the completion of the dose administration schedule.

### Example 6: DNA Prime/Protein Boost Approach with HBV/HDV Constructs Improves Immunogenic Response in Rabbits

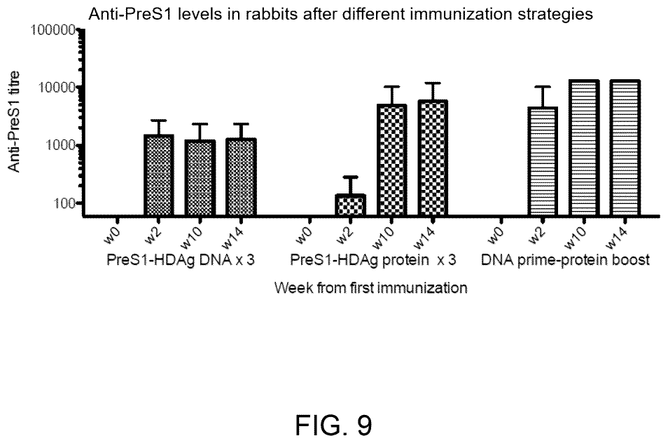

New Zealand white rabbits were immunized with (1) a DNA-only composition comprising Δ-4, (2) a protein-only composition comprising Δ-4, or (3) a DNA prime/protein boost composition comprising Δ-4 DNA and 4-4 protein. Compositions were administered four times as weeks 0, 4, 8, and 12, with either 900 μg DNA im/EP or 300 μg protein with alum administered for each dose. For DNA-protein compositions (3), 900 μg DNA im/EP was administered for the first dose at week 0, and 300 μg protein with alum was administered for the second, third, and fourth doses at weeks 4, 8, and 12. Anti-PreS1 IgG titers in sera were assessed at weeks 0, 2, 10, and 14 (i.e., 2 weeks after each dosage) (FIG. 9). Not only does the DNA prime/protein boost composition (3) result in greater overall titers compared to DNA-only (1) and protein-only (2) compositions, but also induces robust antibody production more rapidly, by week 2, relative to the protein-only composition.

### Example 7: Adoptive Transfer of Sera or Purified IgG from Immunized Animals Protects Humanized Mice Against HBV and HDV Challenges

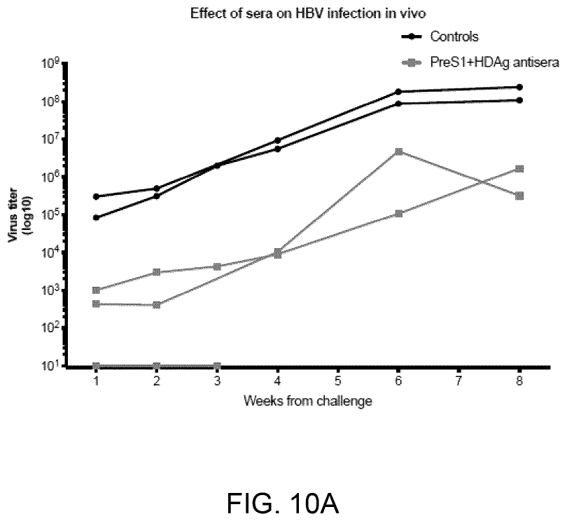

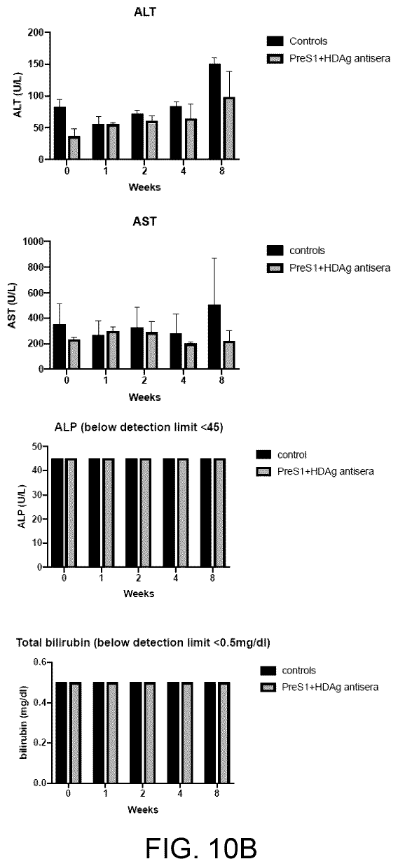

The ability of D4 induced antibodies to neutralize HBV infection in vivo was determined using human-liver chimeric uPA^{+/+}-SCID mouse model as described. Total IgG was purified from D4-immunized and non-immunized rabbits and were injected in uPA^{+/+}-SCID mice repopulated with human hepatocytes three days prior to HBV challenge. The D4-induced PreS1 IgG antibodies protected, or significantly delayed peak viremia in all challenged mice (FIG. 10A). Out of three challenged mice, one was protected (weeks 1 to 3) whereas the other two developed serum levels of HBV <10⁴ IU/ml up to a monthly screening and remained lower compared to the controls up to 8 weeks follow up. The control mice treated with IgG from a naïve rabbit all reached serum HBV DNA levels exceeding 108 IU/ml. There were no significant differences between the groups with respect to serum levels of alanine transferase, asparagine transferase, alkaline phosphatase, or bilirubin (FIG. 10B). In conclusion, passive immunization with D4-specific PreS1 IgG antibodies given as a single dose was able to prevent, or significantly delayed HBV infection in vivo in mice repopulated with human hepatocytes (Table 3). Importantly, the inoculum contains high levels of sub-viral particles SHBsAg showing that these antibodies indeed escape being blocked by SHBsAg. The PreS1 antibodies present at inoculation and during the first weeks clearly block infection, or the first rounds of infection, and limit the number of infected hepatocytes. This limits the viral spread and delay development of peak viremia.

TABLE 3
Adoptive transfer from DNA/protein
vaccinated animals protects against HBV/HDV

| | | | | Absence | | High |
|---|---|---|---|---|---|---|
| | Transfer | | Number | of | Low level | level |
| Infection | source | Treatment | of mice | infection | infection | infection |
| HBV | Purified | Control | 3 | 0 | 0 | 3 |
| | rabbit IgG | | | | | |
| HBV | Purified | D4 DNA | 3 | 1 | 2 | 0 |
| | rabbit IgG | | | | | |
| HBV/HDV | Whole | Control | 3 | 0 | 0 | 3 |
| | mouse | | | | | |
| | sera | | | | | |
| HBV/HDV | Whole | D7 Protein/ | 2 | 2 | 0 | 0 |
| | mouse | alum | | | | |
| | sera | | | | | |
| HBV/HDV | Whole | D4 DNA + | 3 | 3 | 0 | 0 |
| | mouse | D7/alum | | | | |
| | sera | | | | | |

### Example 8: Challenge of HBV/HDV Peptide Constructs with Different Adjuvants

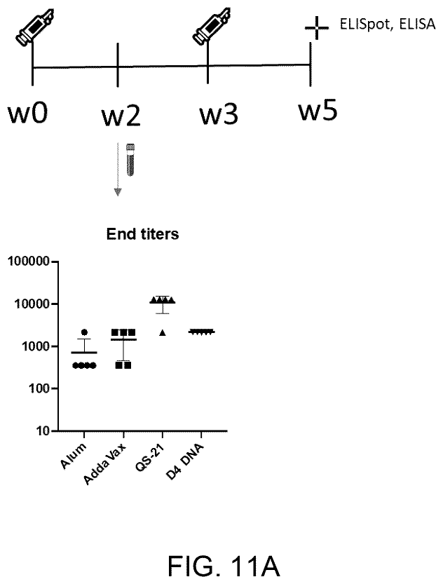

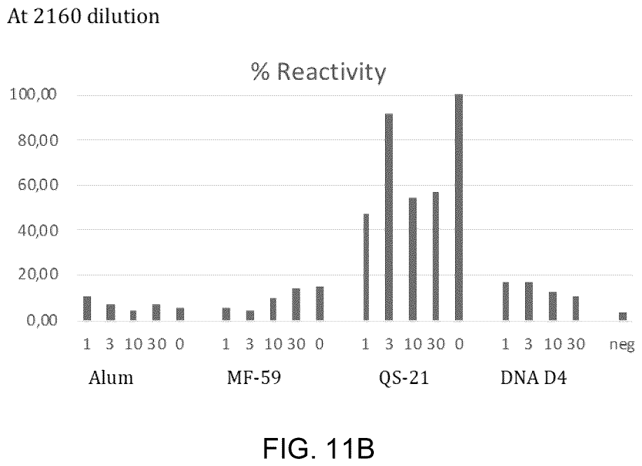

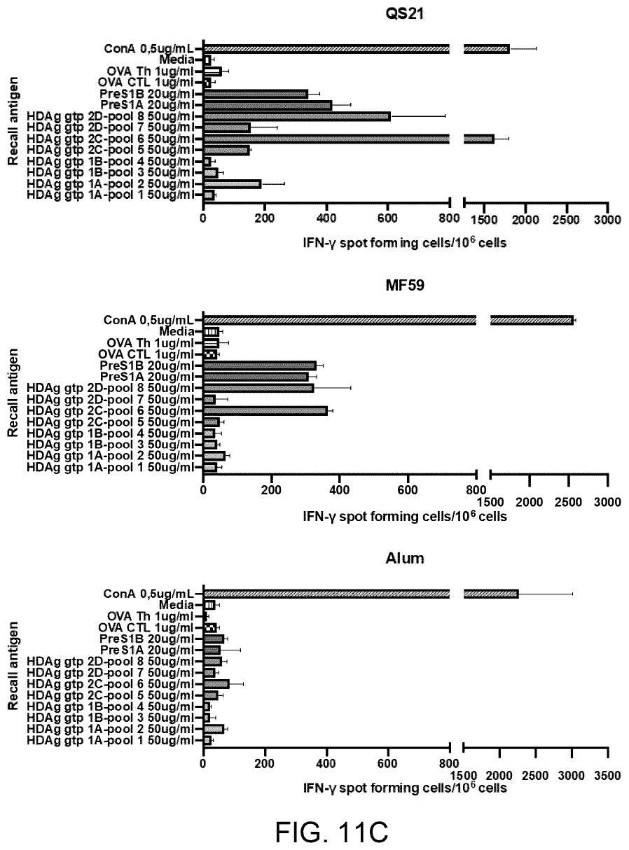

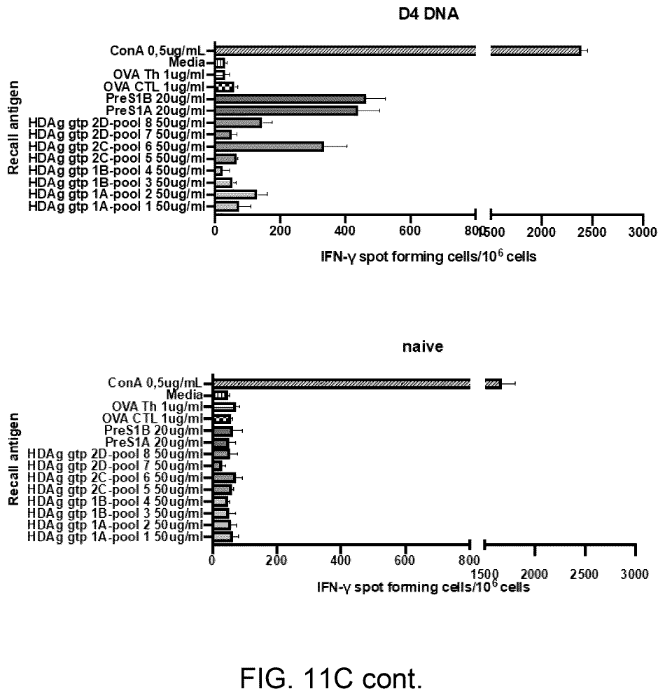

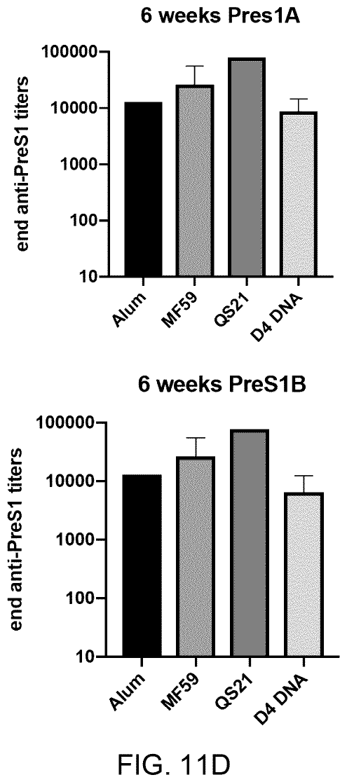

Mixtures of D-7 and D-8 peptides were assessed using different adjuvants. C57BL/6J mice were administered with 2 rounds of a 20 μg mixture of D-7 and D-8 peptides (10 μg each of D-7 and D-8) administered at week 0 and week 3 (FIG. 11A). Peripheral blood samples were taken at week 2 (between the two rounds) to determine end titers for HBV and HDV reactivity by ELISA (FIG. 11A-11B), and splenocytes were isolated for HBV and HDV reactivity by ELISpot (FIG. 11C-11D) at week 5. Peptide compositions were administered subcutaneously with QS-21, MF59, and alum adjuvants. Naïve mice and mice administered D-4 DNA plasmid by intramuscular electroporation were used as controls. IFNγ ELISpot was performed as described above using pools of HDAg peptides, PreS1A and PreS1B peptides, with OVA peptides and Concanavalin A as controls (FIG. 11C-11D). Compositions administered with QS-21 adjuvant exhibited elevated HDAg reactivity compared to the other adjuvants. 5 mice per group were tested.

### Example 9: Comparison of Exemplary HBV/HDV DNA and/or Peptide Constructs

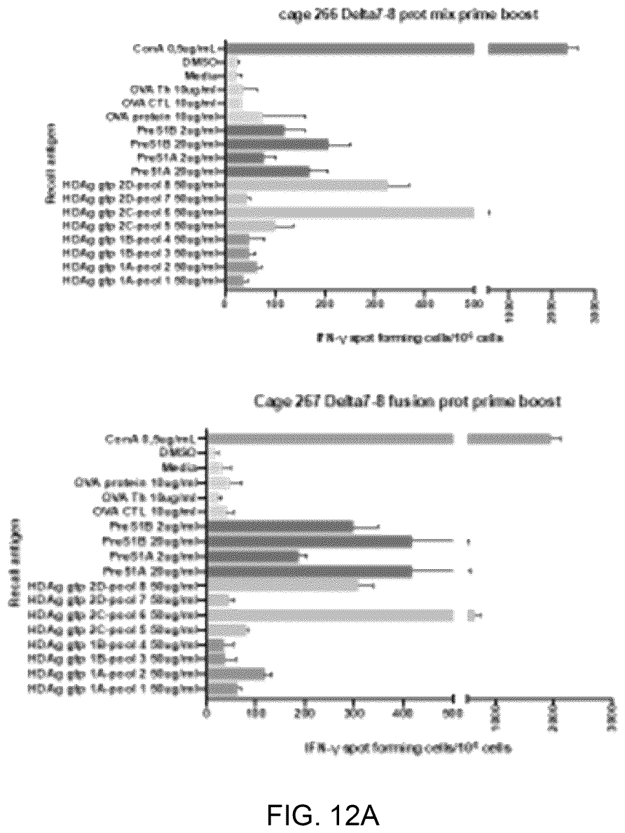

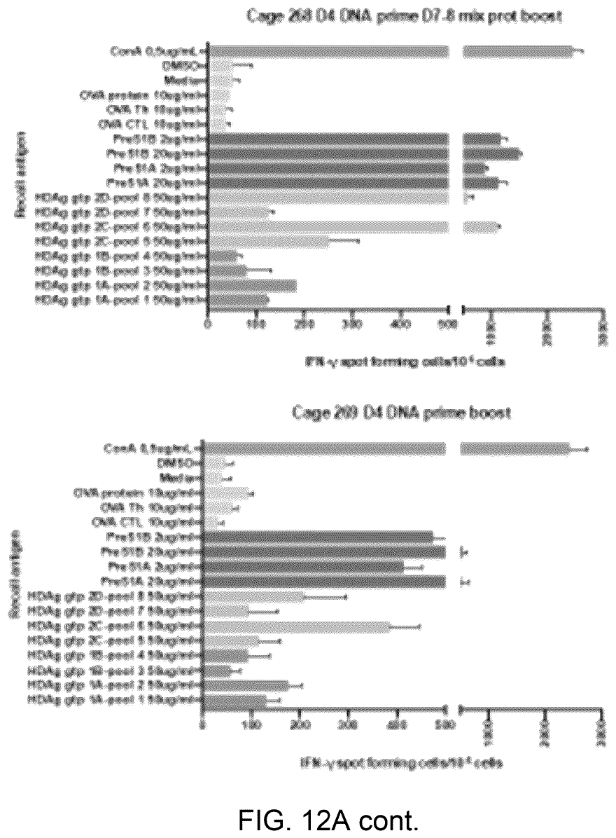

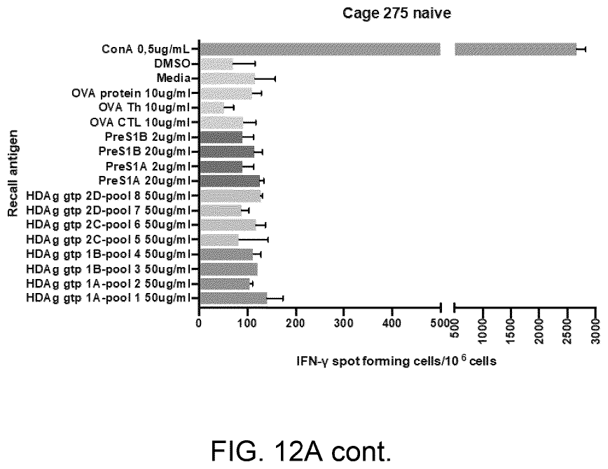

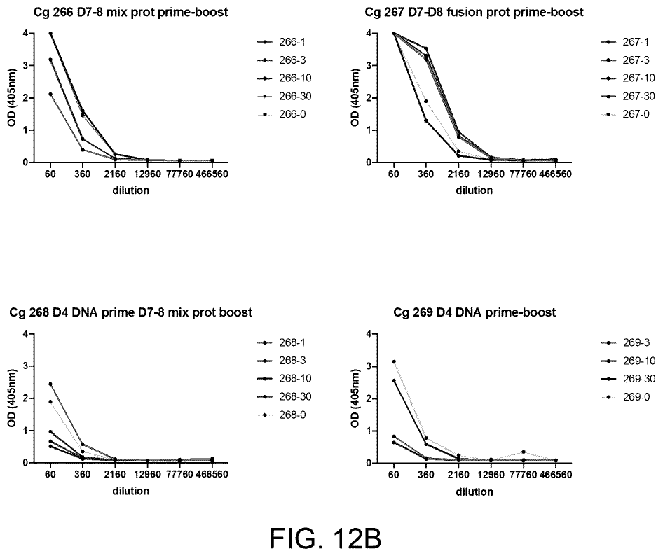

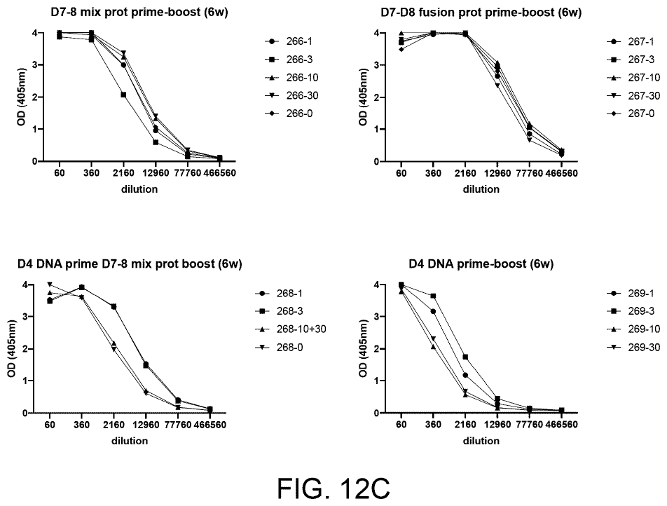

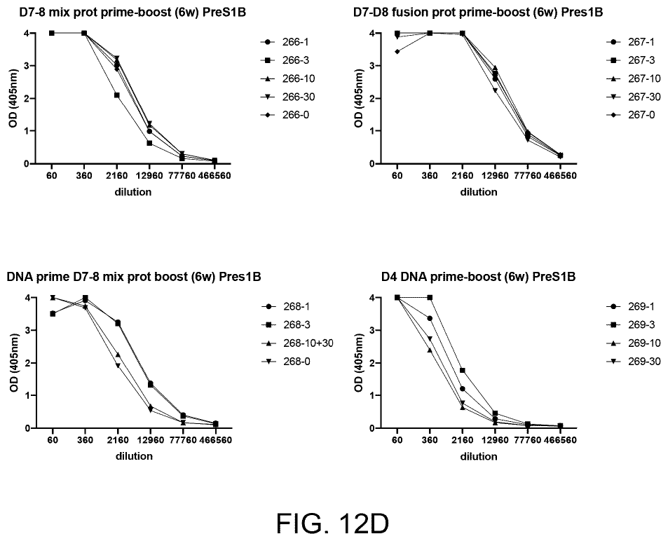

A comparison of immunogenicity for 1) a mixture of D-7 and D-8 peptide only, 2) D-7+D-8 fusion peptide only, 3) D-4 DNA prime and a mixture of D-7 and D-8 peptide boost were tested, with D-4 DNA only and naïve conditions as controls. Mice were administered with either 20 μg of the D-7+D-8 fusion protein or 10 μg each of D-7 and D-8 peptide in the mixed conditions with QS-21 adjuvant, subcutaneously at the tail base in a volume of 100 μL. 2 rounds of administration was performed at weeks 0 and 4. D-4 DNA control was administered at 50 μg intramuscular in 50 μL PBS with electroporation. At week 6 (following two rounds of administration), T cell response to PreS1 and HDV antigen genotypes 1 and 2 were determined by IFNγ ELISpot (FIG. 12A). In addition, at weeks 2 (following one round of administration) and 6 weeks (following two rounds of administration), antibody levels to PreS1A (FIG. 12B-12C) and PreS1B (FIG. 12D) consensus peptides were assessed. The greatest HBV and HDV reactivity are observed in the DNA prime, peptide boost condition.

### Example 10: DNA or Protein Prime with DNA or Protein Boost Immunization Against HBV and/or HDV in Human Clinical Trials

The following example describes embodiments of using an immunogenic composition or product combination, optionally comprised of a nucleic acid component and a polypeptide component, used to treat or prevent viral infections caused by viruses such as HBV and HDV.

The DNA prime/protein boost compositions disclosed herein are administered to human patients enterally, orally, intranasally, parenterally, subcutaneously, intramuscularly, intradermally, or intravenously. These human patients may be currently infected with HBV and/or HDV, previously infected with HBV and/or HDV, at risk of being infected with HBV and/or HDV, or uninfected with HBV and/or HDV.

The DNA prime doses are administered first, at an amount of 1, 10, 100, 1000 ng, or 1, 10, 50, 100, 200, 300, 400, 500, 600, 700, 800, 900, 1000 μg, or 1, 10, 100, 200, 300, 400, 500, 600, 700, 800, 900, 1000 mg, or any amount within a range defined by any two of the aforementioned amounts, or any other amount appropriate for optimal efficacy in humans. After the first DNA prime dose, 1, 2, 3, 4, or 5 additional DNA prime doses can be administered 1, 2, 3, 4, 5, 6, 7, 8, 9, 10, 11, 12, 24, 36, or 48 days or weeks or any time within a range defined by any two of the aforementioned times after administration of the previous DNA prime dose, e.g., within 1-48 days or 1-48 weeks. The protein boost doses are administered following the DNA prime doses, at an amount of 1, 10, 100, 1000 ng, or 1, 10, 50, 100, 200, 300, 400, 500, 600, 700, 800, 900, 1000 μg, or 1, 10, 100, 200, 300, 400, 500, 600, 700, 800, 900, 1000 mg, or any amount within a range defined by any two of the aforementioned amounts, or any other amount appropriate for optimal efficacy in humans. The first protein boost dose is administered 1, 2, 3, 4, 5, 6, 7, 8, 9, 10, 11, 12, 24, 36, or 48 days or weeks or any time within a range defined by any two of the aforementioned times after administration of the final DNA prime dose. After the first protein boost dose, 1, 2, 3, 4, or 5 additional protein boost doses can be administered 1, 2, 3, 4, 5, 6, 7, 8, 9, 10, 11, 12, 24, 36, or 48 days or weeks or any time within a range defined by any two of the aforementioned times after administration of the previous protein boost dose.

Patients will be monitored for successful response against HBV and/or HDV, for example, production of anti-HBV, anti-HDV, anti-PreS1, or anti-HDAg antibodies in sera, rapid activation of T cells and other immune cells when exposed to HBV and/or HDV antigens, and protection against future HBV and/or HDV infections.

In patients currently infected, previously infected, or at risk for infection with HBV and/or HDV, administration of the DNA prime/protein boost compositions may be performed in conjunction with antiviral therapy. Potential antiviral therapy therapeutics that have been shown to be effective against HBV or HDV include but are not limited to entecavir, tenofovir, lamivudine, adefovir, telbivudine, emtricitabine, interferon-a, pegylated interferon-a, or interferon alfa-2b, or any combination thereof. Patients will be monitored for side effects such as dizziness, nausea, diarrhea, depression, insomnia, headaches, itching, rashes, fevers, or other known side effects of the provided antiviral therapeutics.

### Example 11: Expression of HBV/HDV Fusion Constructs in E. coli

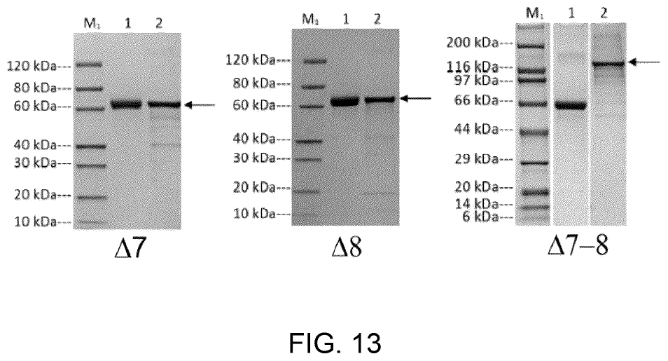

Constructs D-7, D-8, and D-7+D-8 fusion peptide were expressed in E. coli using T7 expression. Proteins were expressed as insoluble inclusion bodies. Expression was tested at 15° C. for 16 hours or at 37° C. for 4 hours with 0.5 M IPTG induction. Six refolding buffers were tried (listed below) of which the ones containing 1 M or 0.5 M of L-arginine at pH 8.0 were best in refolding the expressed protein. All fusion products isolated showed some degradation, particularly for the D-7+D-8 fusion peptide (FIG. 13). This is consistent with literature data, which describe degradation of the large and small HDAg antigen when produced in E. coli. Furthermore, it has been reported that the large HDAg antigen expresses as a soluble protein in E. coli. Fusion with the PreS1 A/B sequences and more than one extra copy of the large HDAg was suspected to change its solubility and/or its ability to fold natively. It is noted that the HDAg antigens form oligomers through N-terminal L-zipper structures. Indeed, the soluble produced small HDAg antigen (24 kDa) is found in complexes ranging from 160-350 kDa, indicating complexes with 10 or more subunits.

The six refolding buffers tested were as follows:

Buffer T1: 50 mM Tris pH 8.0, 150 mM NaCl, 10% glycerol.

Buffer T2: 50 mM Tris pH 8.0, 500 mM NaCl, 10% glycerol.

Buffer T3: 50 mM Tris pH 8.0, 150 mM NaCl, 10% glycerol, 0.5 M L-arginine

Buffer T4: 50 mM Tris pH 8.0, 150 mM NaCl, 10% glycerol, 1 M L-arginine.

Buffer T5: 50 mM Tris pH 8.0, 150 mM NaCl, 10% glycerol, 0.2% sodium dodecyl sulfate (SDS).

Buffer T6: 50 mM Tris pH 8.0, 150 mM NaCl, 10% glycerol, 0.2% sodium lauryl sarcosine (SKL).

### Example 12: Additional Exemplary HBV/HDV Immunogenic Fusion Constructs

Alternative exemplary HBV/HDV constructs were prepared using different combinations of HBV PreS1 (A and B consensus sequences) and HDV HDAg large antigen, where the HDAg sequences have been mutated to substitute a free C-terminal cysteine to serine (C211S). In addition, nucleic acid sequences were codon optimized to reduce instances of homologous repeat sequences, which may cause plasmid instability. Table 4 depicts the nucleic acid and accompanying translated peptide sequences for the HDAg large antigen cysteine mutant (HDAg-C211S) and HBV PreS1 A and B consensus sequences. The codon optimized PreS1 sequence translate to the same peptide sequences (SEQ ID NOs: 11-12, respectively) as disclosed herein (i.e., only the nucleic acid sequence has changed).

TABLE 4
Modified HBV and HDV immunogenic component sequences

| Component | Nucleic acid sequence | Peptide sequence |
|---|---|---|
| HDAg genotype 1 A C211S | SEQ ID NO: 43 | SEQ ID NO: 47 |
| HDAg genotype 1 B C211S | SEQ ID NO: 44 | SEQ ID NO: 48 |
| HDAg genotype 2 A C211S | SEQ ID NO: 45 | SEQ ID NO: 49 |
| HDAg genotype 2 B C211S | SEQ ID NO: 46 | SEQ ID NO: 50 |
| PreS1 A (Alternative 1) | SEQ ID NO: 51 | SEQ ID NO: 11 |
| PreS1 A (Alternative 2) | SEQ ID NO: 52 | SEQ ID NO: 11 |
| PreS1 B | SEQ ID NO: 53 | SEQ ID NO: 12 |

FIG. 14 depicts a schematic for the additional exemplary fusion constructs, which comprise different combinations and ordering of the HBV and HDV sequences. Optionally, the viral components are separated by a linker (e.g., “GGG” linker (SEQ ID NO: 54), although other linkers as conventionally understood in the art can be used). Table 5 correlates named constructs with their nucleic acid and peptide sequences. The sequences provided here include the HDAg-C211S cysteine mutant, but other mutants and wild type sequences may also be used. Peptide sequence variants without epitope tags, or with C-terminal 6×-histidine tags are provided. The nucleic acid sequences correspond to peptide sequences with the 6×-histidine tag.

TABLE 5
Additional exemplary fusion construct sequences

| | Nucleic acid | Peptide | Peptide sequence |
|---|---|---|---|
| | sequence (with | sequence (with | (without C- |
| Construct | 6x-His tag) | 6x-His tag) | terminal tag) |
| F1-A | SEQ ID NO: 60 | SEQ ID NO: 72 | SEQ ID NO: 84 |
| F1-B | SEQ ID NO: 61 | SEQ ID NO: 73 | SEQ ID NO: 85 |
| F2 | SEQ ID NO: 62 | SEQ ID NO: 74 | SEQ ID NO: 86 |
| F3-A | SEQ ID NO: 63 | SEQ ID NO: 75 | SEQ ID NO: 87 |
| F3-B | SEQ ID NO: 64 | SEQ ID NO: 76 | SEQ ID NO: 88 |
| F4 | SEQ ID NO: 65 | SEQ ID NO: 77 | SEQ ID NO: 89 |
| F5 | SEQ ID NO: 66 | SEQ ID NO: 78 | SEQ ID NO: 90 |
| F6 | SEQ ID NO: 67 | SEQ ID NO: 79 | SEQ ID NO: 91 |
| F7 | SEQ ID NO: 68 | SEQ ID NO: 80 | SEQ ID NO: 92 |
| F8 | SEQ ID NO: 69 | SEQ ID NO: 81 | SEQ ID NO: 93 |
| F9 | SEQ ID NO: 70 | SEQ ID NO: 82 | SEQ ID NO: 94 |
| F10 | SEQ ID NO: 71 | SEQ ID NO: 83 | SEQ ID NO: 95 |
| Δ-7S-8S C211S | SEQ ID NO: 138 | n/a | SEQ ID NO: 140 |
| Δ-78 C211S | SEQ ID NO: 139 | n/a | SEQ ID NO: 141 |
| Δ-7S-8S-L1 | SEQ ID NO: 142 | n/a | SEQ ID NO: 145 |
| Δ-7S-8S-L2 | SEQ ID NO: 143 | n/a | SEQ ID NO: 146 |
| Δ-7S-8S-L3 | SEQ ID NO: 144 | n/a | SEQ ID NO: 147 |
| Δ-78-L1 | SEQ ID NO: 148 | n/a | SEQ ID NO: 163 |
| Δ-78-L2 | SEQ ID NO: 149 | n/a | SEQ ID NO: 164 |
| Δ-78-L3 | SEQ ID NO: 150 | n/a | SEQ ID NO: 165 |
| Δ-78-L4 | SEQ ID NO: 151 | n/a | SEQ ID NO: 166 |
| Δ-78-L5 | SEQ ID NO: 152 | n/a | SEQ ID NO: 167 |
| Δ-78-L6 | SEQ ID NO: 153 | n/a | SEQ ID NO: 168 |
| Δ-78-L7 | SEQ ID NO: 154 | n/a | SEQ ID NO: 169 |
| Δ-78-L8 | SEQ ID NO: 155 | n/a | SEQ ID NO: 170 |
| Δ-78-L9 | SEQ ID NO: 156 | n/a | SEQ ID NO: 171 |
| Δ-78-L10 | SEQ ID NO: 157 | n/a | SEQ ID NO: 172 |
| Δ-78-L11 | SEQ ID NO: 158 | n/a | SEQ ID NO: 173 |
| Δ-78-L12 | SEQ ID NO: 159 | n/a | SEQ ID NO: 174 |
| Δ-78-L13 | SEQ ID NO: 160 | n/a | SEQ ID NO: 175 |
| Δ-78-L14 | SEQ ID NO: 161 | n/a | SEQ ID NO: 176 |
| Δ-78-L15 | SEQ ID NO: 162 | n/a | SEQ ID NO: 177 |

### Example 13: Production of HBV/HDV Immunogenic Constructs Using an Operon Approach

As an alternative approach, the expression of different fusions organized in a single operon will be examined. As a prokaryote, E. coli have genes organized in operons, and translation is dependent on a good translation initiation region containing a ribosome binding site (RBS). This operon approach is based on the fact that the HDAg protein oligomerizes in complexes with 10 or more subunits. Upon expression and/or refolding, the protein constructs are expected to form oligomers containing a certain quantity of each fusion. This can serve as the basis of a therapeutic formulation once the final protein mix is characterized, and the different subunits are quantified. Although different subtypes of the product gene (different variations of HDAg and PreS1 fusions) are formed, this allows them to be made in a single batch.

A few variations will be tested to find the optimal configuration of this approach. FIG. 15 depicts a schematic for exemplary fusion constructs to be used in the operon approach. Construct 01 contains the D-7 and D-8 genes. Constructs 02-A, 02-B, and 03 contain one copy of each of the HDAg genotype 1 and genotype 2 subtypes in a two gene operon. O2-A contain a non-tandem PreS1 A or PreS1 B in respective fusions, and 02-B contain the tandem PreS1 A/B sequences. Construct 04 contains all 4 subtypes in one operon. As a general rule, genes that are more 5′ in the operon are more highly expressed, meaning that translation efficiency decreases towards the 3′ end. Construct O5 is a variation of construct 04 where two transcripts are used, each containing two fusion proteins. Optionally, the viral components can also be separated by a linker.

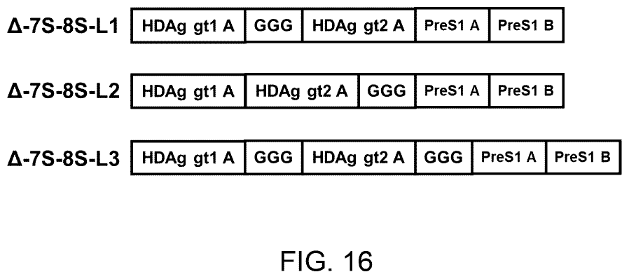

Additional exemplary nucleic acid or polypeptide constructs comprising HBV and/or HDV antigens used herein are provided in FIG. 16. Three constructs are provided herein, Δ-7S-8S-L1 (Delta-78-8S-L1; D-7S-8S-L1), Δ-7S-8S-L2 (Delta-78-8S-L2; D-7S-8S-L2), and Δ-7S-8S-L3 (Delta-78-8S-L3; D-7S-8S-L3), which are modifications of Δ-7S-8S with linkers interspersed between the HBV and/or HDV antigens. “GGG” refers to a triple glycine linker (SEQ ID NO: 54).

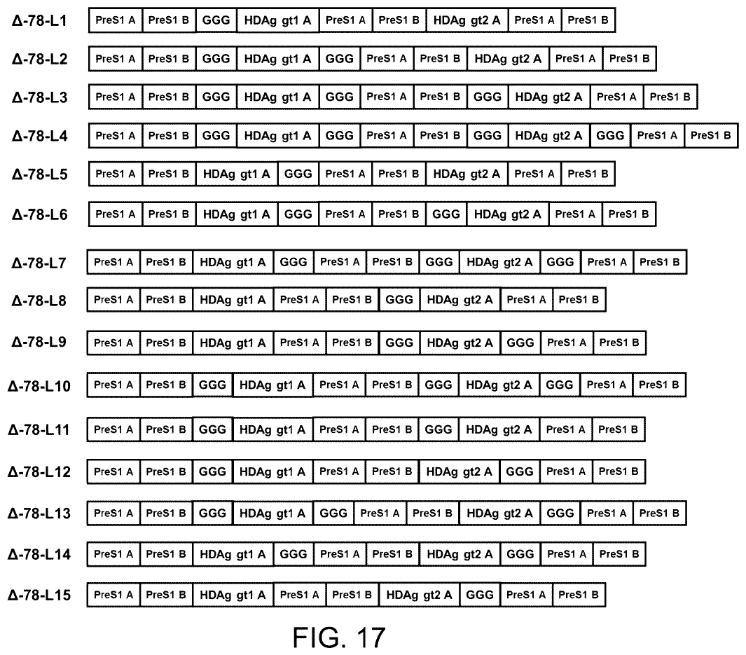

Additional exemplary nucleic acid or polypeptide constructs comprising HBV and/or HDV antigens used herein are provided in FIG. 17. Fifteen constructs are provided herein, Δ-78-L1 (Delta-78-L1; D-78-L1), Δ-78-L2 (Delta-78-L2; D-78-L2), Δ-78-L3 (Delta-78-L3; D-78-L3), Δ-78-L4 (Delta-78-L4; D-78-L4), Δ-78-L5 (Delta-78-L5; D-78-L5), Δ-78-L6 (Delta-78-L6; D-78-L6), Δ-78-L7 (Delta-78-L7; D-78-L7), Δ-78-L8 (Delta-78-L8; D-78-L8), Δ-78-L9 (Delta-78-L9; D-78-L9), Δ-78-L10 (Delta-78-L10; D-78-L10), Δ-78-L11 (Delta-78-L11; D-78-L11), Δ-78-L12 (Delta-78-L12; D-78-L12), Δ-78-L13 (Delta-78-L13; D-78-L13), Δ-78-L14 (Delta-78-L14; D-78-L14), Δ-78-L15 (Delta-78-L15; D-78-L15), which are modifications of Δ-78 with linkers interspersed between the HBV and/or HDV antigens. “GGG” refers to a triple glycine linker (SEQ ID NO: 54).

Table 6 correlates named constructs with their nucleic acid and resultant translated peptide sequences. The sequences provided here include the HDAg-C211S cysteine mutant, but other mutants and wild type sequences may also be used. Peptide sequence variants without epitope tags, or with C-terminal epitope tags are provided. The nucleic acid sequences correspond to peptide sequences with the C-terminal epitope tags. The epitope tags can be selected from E-tag, Myc, FLAG, Strep2, or 6×His tags. However, other epitope tags known in the art can be used in these constructs. The presence of the epitope tags enable ease of detection and/or purification during testing and are not necessarily to be used in the final immunogenic composition.

TABLE 6
Additional exemplary fusion constructs for use in an operon approach

| | Nucleic acid operon | Peptide sequence | Peptide sequence |
|---|---|---|---|
| | sequence (with C- | (with C- | (without C- |
| Construct | terminal tags) | terminal tags) | terminal tags) |
| O1 | SEQ ID NO: 96 | 1A1BS-E-tag-6xHis | 1A1BS (SEQ ID NO: 118) |
| | | (SEQ ID NO: 102) | 2A2BS (SEQ ID NO: 119) |
| | | 2A2BS-Myc-6xHis | |
| | | (SEQ ID NO: 103) | |
| O2-A | SEQ ID NO: 97 | 1AS-E-tag-6xHis | 1AS (SEQ ID NO: 120) |
| | | (SEQ ID NO: 104) | 2AS (SEQ ID NO: 121) |
| | | 2AS-Myc-6xHis | |
| | | (SEQ ID NO: 105) | |
| O2-B | SEQ ID NO: 98 | 1ASS-E-tag-6xHis | 1ASS (SEQ ID NO: 122) |
| | | (SEQ ID NO: 106) | 2ASS (SEQ ID NO: 123) |
| | | 2ASS-Myc-6xHis | |
| | | (SEQ ID NO: 107) | |
| O3 | SEQ ID NO: 99 | 1BS-FLAG-6xHis | 1BS (SEQ ID NO: 124) |
| | | (SEQ ID NO: 108) | 2BS (SEQ ID NO: 125) |
| | | 2BS-FLAG-6xHis | |
| | | (SEQ ID NO: 109) | |
| O4 | SEQ ID NO: 100 | 1AS-E-tag-6xHis | 1AS (SEQ ID NO: 126) |
| | | (SEQ ID NO: 110) | 1BS (SEQ ID NO: 127) |
| | | 1BS-FLAG-6xHis | 2AS (SEQ ID NO: 128) |
| | | (SEQ ID NO: 111) | 2BS (SEQ ID NO: 129) |
| | | 2AS-Myc-6xHis | |
| | | (SEQ ID NO: 112) | |
| | | 2BS-Strep2-6xHis | |
| | | (SEQ ID NO: 113) | |
| O5 | SEQ ID NO: 101 | 1AS-E-tag-6xHis | 1AS (SEQ ID NO: 130) |
| | | (SEQ ID NO: 114) | 1BS (SEQ ID NO: 131) |
| | | 1BS-FLAG-6xHis | 2AS (SEQ ID NO: 132) |
| | | (SEQ ID NO: 115) | 2BS (SEQ ID NO: 133) |
| | | 2AS-Myc-6xHis | |
| | | (SEQ ID NO: 116) | |
| | | 2BS-Strep2-6xHis | |
| | | (SEQ ID NO: 117) | |

### Example 14: Additional Exemplary HBV/HDV Immunogenic Fusion Constructs

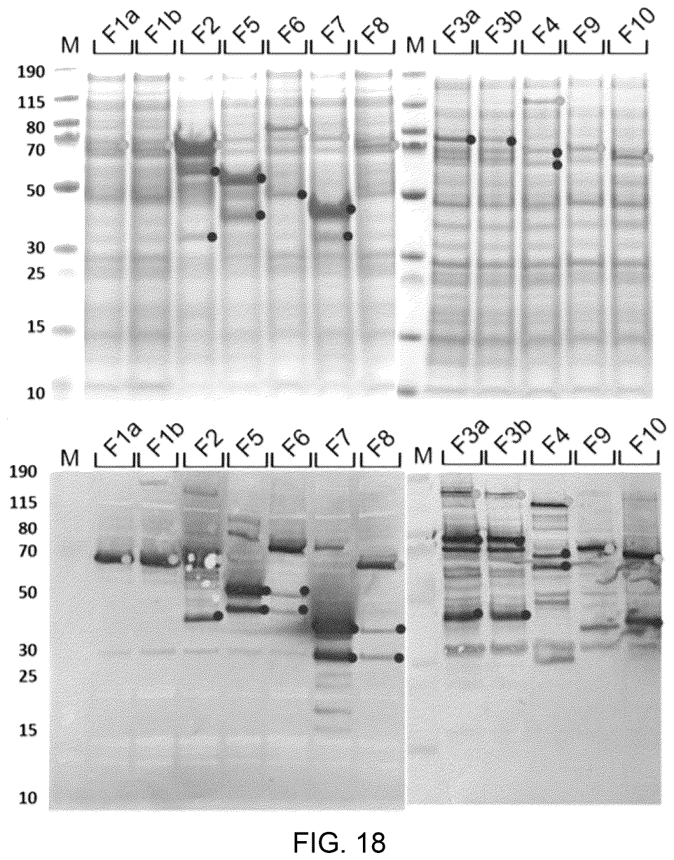

Upon expression of constructs disclosed herein, it was observed by SDS-PAGE and anti-His Western blot that some of the expressed polypeptides, such as F5 and F7, resulted in significant degradation products (FIG. 18). It was hypothesized that this degradation occurred in E. coli due to the presence of an N-terminal PreS1A/B or internal PreS1A/B, which is found in some construct disclosed herein, including F5 and F7.

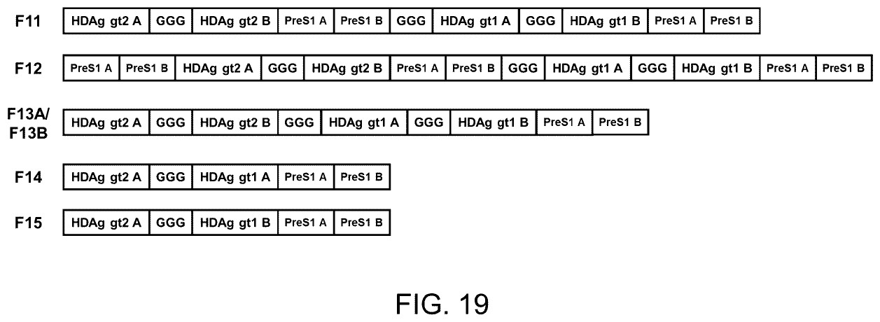

Accordingly, additional alternative exemplary HBV/HDV constructs were prepared so as to reduce unwanted degradation products by using different combinations and ordering of the HBV PreS1 (A and B consensus sequences) and HDV HDAg large antigen sequences. The HDAg sequences may be mutated to substitute a free C-terminal cysteine to serine (C211S). In addition, nucleic acid sequences were codon optimized. FIG. 19 depicts a schematic for these additional constructs F11, F12, F13A, F13B, F14, and F15 and Table 7 depicts their corresponding sequences.

F11 (as relative to construct 47-8) omits the N-terminal PreS1A and PreS1B to avoid inducing degradation and exhibits an inverted order of HDAg genotype 1 A/B and HDAg genotype 2 A/B (whereby the construct starts with HDAg genotype 2 A/B sequences). The latter change was done to obtain a better translation initiation region (TIR). This construct was optionally codon optimized using the conventionally available GenScript algorithm, which does not require the avoidance of DNA direct repeats.

F12 is based on the F11 construct but restores the N-terminal PreS1A and PreS1B sequences for use as a control.

F13A and F13B are based on the F11 construct but removes the internal PreS1A and PreS1B sequences. F13A and F13B result in the same polypeptide sequence when expressed but correspond to different nucleic acid sequences that were codon optimized using the GenScript and JCAT algorithm, respectively.

F14 is based on F10 but switching the order of HDAg genotype 2A and genotype 1A sequences, whereby the construct starts with the HDAg genotype 2A sequence.

F15 is based on F14 but swapping out the HDAg genotype 1A sequence for an HDAg genotype 1B sequence.

TABLE 7
Additional exemplary fusion construct sequences

| | Nucleic acid | Peptide | Peptide sequence |
|---|---|---|---|
| | sequence (with 6x- | sequence (with | (without C- |
| Construct | His tag) | 6x-His tag) | terminal tag) |
| F11 | SEQ ID NO: 178 | SEQ ID NO: 187 | n/a |
| F12 | SEQ ID NO: 179 | SEQ ID NO: 188 | SEQ ID NO: 193 |
| F13A | SEQ ID NO: 181 | SEQ ID NO: 189 | SEQ ID NO: 194 |
| F13B | SEQ ID NO: 182 | SEQ ID NO: 190 | SEQ ID NO: 194 |
| F14 | SEQ ID NO: 184 | SEQ ID NO: 191 | SEQ ID NO: 195 |
| F15 | SEQ ID NO: 186 | SEQ ID NO: 192 | n/a |

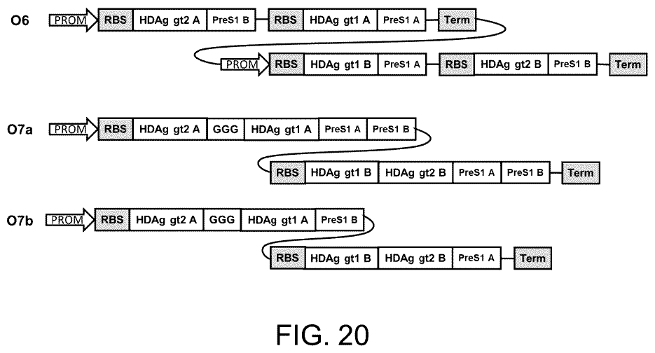

Similarly, additional operon-based constructs O6 and O7 were constructed for use in the operon approach described in Example 13. FIG. 20 depicts the construction of these constructs for the operon approach, and Table 8 depicts their corresponding sequences.

O6 differs from O5 by swapping the HDAg genotype 1A-PreS1A and HDAg genotype 2A-PreS1B sequences, whereby the HDAg genotype 2A-PreS1B sequence appears at the N-terminus.

The N-terminus of O7a comprises the HDAg genotypes 2A and 1A connected by a GGG linker and followed by Pres1A and Pres1B sequences. The Pres1A and Pres1B sequences are followed by HDAg genotypes 1B and 2B followed by Pres1A and Pres1B sequences.

O7b differs from the O7a construct by lacking the two Pres1B sequences.

TABLE 8
Additional exemplary fusion constructs for use in an operon approach

| | Nucleic acid operon | Peptide sequence | Peptide sequence |
|---|---|---|---|
| | sequence (with C- | (with C- | (without C- |
| Construct | terminal tags) | terminal tags) | terminal tags) |
| O6 | SEQ ID NO: 196 | 1AS-E-tag-6xHis | 1AS (SEQ ID NO: 130) |
| | | (SEQ ID NO: 114) | 1BS (SEQ ID NO: 131) |
| | | 1BS-FLAG-6xHis | 2AS (SEQ ID NO: 132) |
| | | (SEQ ID NO: 115) | 2BS (SEQ ID NO: 133) |
| | | 2AS-Myc-6xHis | |
| | | (SEQ ID NO: 116) | |
| | | 2BS-Strep2-6xHis | |
| | | (SEQ ID NO: 117) | |
| O7a | SEQ ID NO: 198 | 2A1AS1ab-EH | n/a |
| | | (SEQ ID NO: 200) | |
| | | 1B2BS1ab-SH | |
| | | (SEQ ID NO: 201) | |
| O7b | SEQ ID NO: 199 | O7b-2A1AS1a-EH | n/a |
| | | (SEQ ID NO: 202) | |
| | | O7b-1B2BS1b-SH | |
| | | (SEQ ID NO: 203) | |

### Example 15: Optimization of Large-Scale Construct Expression and Purification

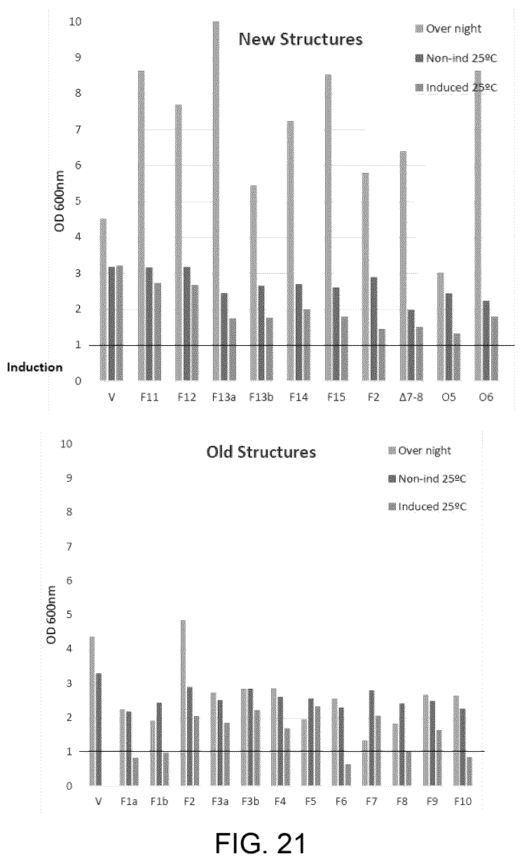

Cultures of E. coli were transformed with lac inducible vectors with the F11, F12, F13A, F13B, F14, F15, F2, Δ7-8, O5, O6, F1-A, F1-B, F2, F3-A, F3-B, F4, F5, F6, F7, F8, F9, and F10 constructs disclosed herein. The cells were pre-cultured at 30° C. overnight and, the next day, a culture was taken from the pre-cultures and grown until an OD₆₀₀ of approximately 1. Protein expression was induced with 0.5 mM IPTG for 4 hours at 25° C. At the same time, separate cultures were grown under similar conditions without IPTG for a non-induced control. FIG. 21 depicts the quantified OD₆₀₀ of the pre-cultures, induced cultures, and non-induced controls for each of the tested constructs. No growth inhibition was observed in the overnight cultures, and each exhibited a similar growth profile. After induction, the cultures exhibit slower growth as expected.

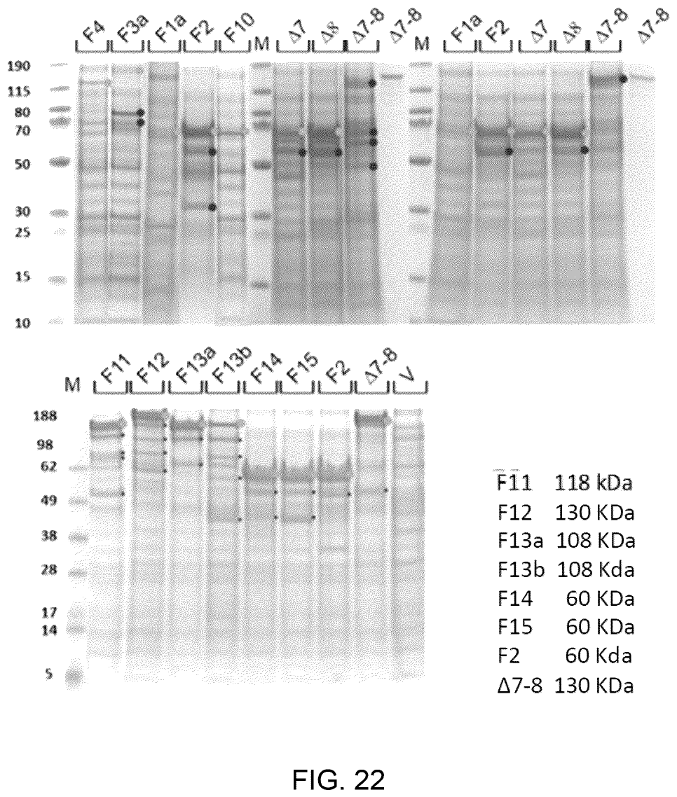

After 4 hours of induction, the cells were pelleted, lysed using BugBuster® Protein Extraction Reagent (Millipore), and run on SDS-PAGE gels, which were then stained with Coomassie Brilliant Blue (CBB) (FIG. 22). All of the tested constructs gave clearly identifiable bands. The constructs described in Example 14, addressing the potential formation of degradation products due to the PreS1A/PreS1B sequence, exhibited less degradation as compared to other constructs (e.g., F1, F3, F4). F14 and F15 were observed to express similarly to F2, where these constructs start with HDAg genotype 2A. Expression of both the full length 47-8 and F12 construct and A7-8 and F12 degradation products were observed. Higher expression was observed for F13A, which was codon optimized using the GenScript algorithm, compared to F13B, which was codon optimized by the JCAT algorithm.

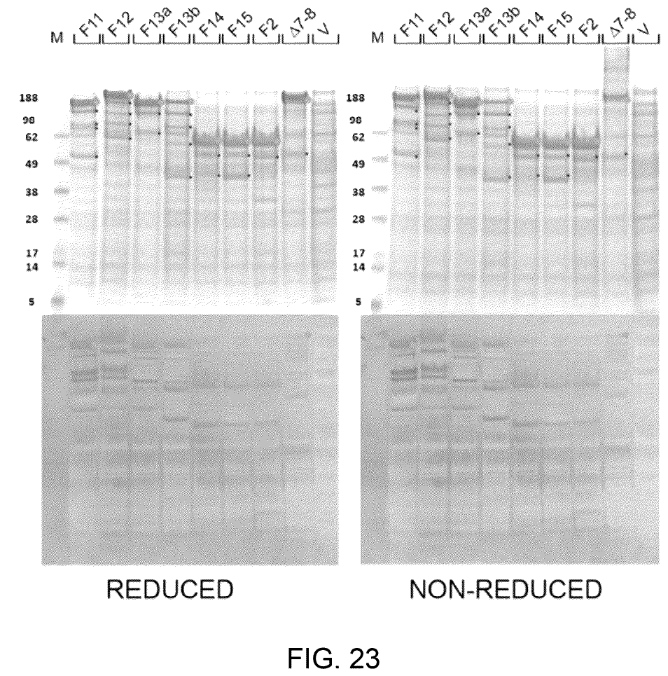

The same samples were compared by running on SDS-PAGE gels under reduced and non-reduced conditions and stained with either CBB or probed with anti-His on a Western blot (FIG. 23). All of the tested constructs gave clearly identifiable bands by CBB and anti-His staining. Both full length 47-8 and its degradation products shifted to higher molecular weight complexes under non-reduced conditions, while this was not observed for other constructs. This was suspected to be due to the presence of the exposed C211 cysteine in the A7-8 construct, which has been removed in the other constructs.

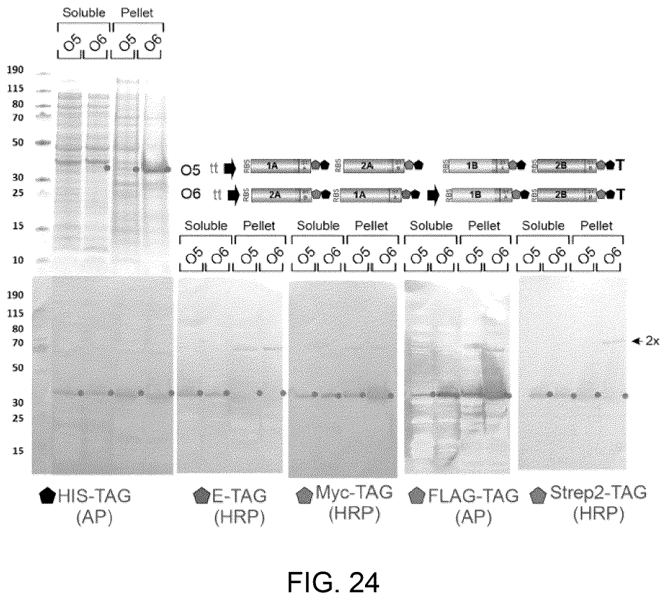

The operon-based constructs O5 and O6 were induced in E. coli and expression was probed by CBB staining and Western blot, using the variants disclosed herein where each gene of the operon is tagged with distinct epitope tags (FIG. 24). Expression of all of the genes as expected in the operons was confirmed by Western blot using respective anti-tag antibodies, and an improvement of expression of O6 over O5 was observed, indicating that the presence of the N-terminal HDAg genotype 2A sequence enhances expression. Interestingly, overexpression of the O6 construct in the insoluble fraction was observed.

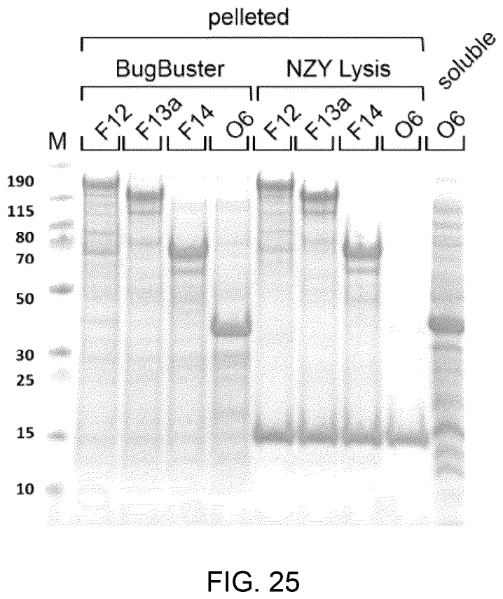

It was investigated whether the use of different lysis buffers could improve yield. E. coli cells transformed with the F12, F13A, F14, and O6 constructs were grown and induced with 0.5 mM IPTG for 4 hours at 25° C. These cells were lysed using the BugBuster® Protein Extraction Reagent and NZY Bacterial Cell Lysis Buffer (NZYTech) and the insoluble fractions of the lysates were evaluated by SDS-PAGE (FIG. 25). Similar levels of inclusion bodies were observed for both conditions. However, for O6, the NZY lysis buffer resulted in extraction of the protein contents from the insoluble to the soluble fractions.

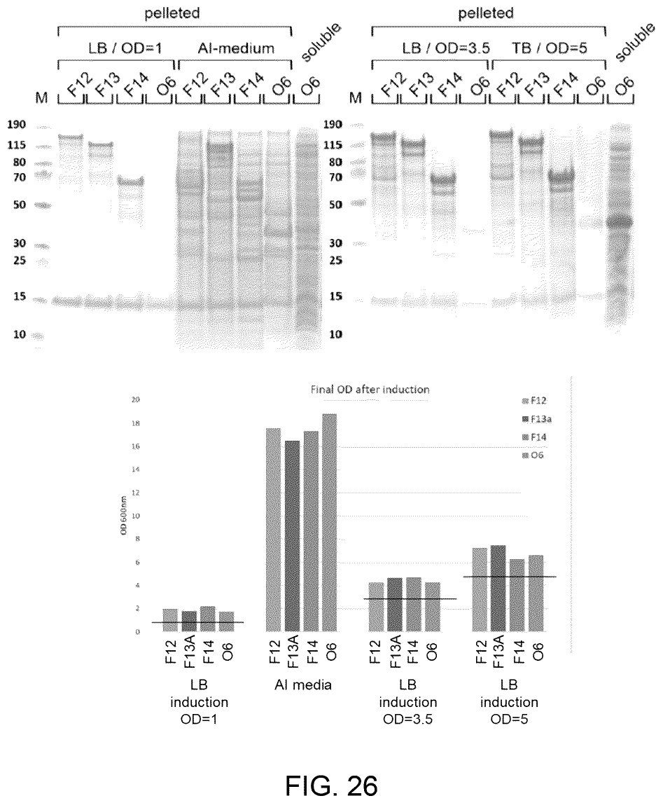

The induction conditions were also examined. Expression was tested by growing the bacterial cells in 1) autoinduction (AI) media inoculated at an OD₆₀₀ of 0.5 and cultured for 28 hours at 25° C., 2) standard LB broth but induced at an OD₆₀₀ of 3.5, 3) Terrific broth (TB) induced at an OD₆₀₀ of 5, and 4) control LB broth induced at an OD₆₀₀ of 1 (FIG. 26). The culture grown in AI media lead to product degradation and precipitation, which was suspected to be due to the prolonged incubation. Induction at higher ODs in both LB and TB allowed for an increase in volumetric productivity without compromising product stability. The condition using TB improved expression compared to control by at least 2 times as quantified by CBB band intensity.

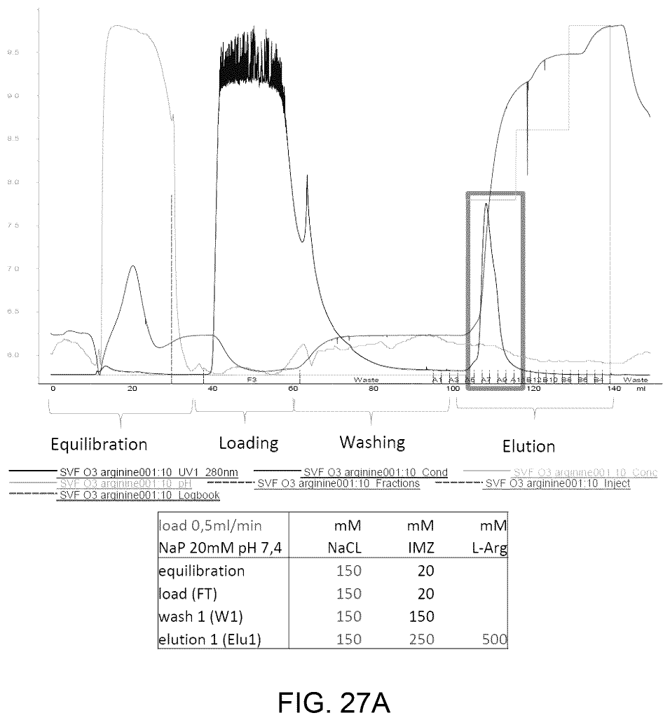

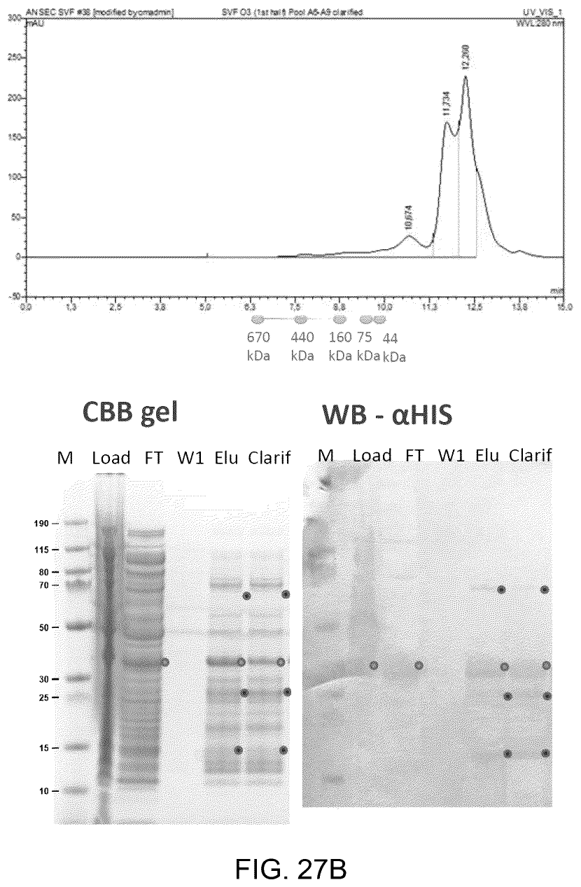

IMAC column based purification was utilized to isolate exemplary construct 03 expressed in E. coli. In the purification run (Run 1), an E. coli pellet from a 500 mL culture was lysed with lysis buffer supplemented with DNAse 25U/ml BB and 1 KU rLysozyme (C). The soluble fraction of the lysate was recovered, and the buffer was exchanged to PBS. The lysate was loaded into a HiTrap IMAC Sepharose 1 ml column (Cytiva) where the column was subjected to equilibration, loading, washing, and elution steps (FIG. 27A). The eluate was evaluated via analytical size exclusion chromatography (AnSEC), CBB staining of a SDS-PAGE gel, and anti-His Western blotting of the purified product (FIG. 27B). The 03 eluate was found to be composed of low molecular weight (MW) species (<44 kDA) by AnSEC and have a purity of 18% by CBB. These results indicated that the product was not fully captured and a high amount of host cell proteins were present in the eluate. The results further indicated that the product precipitated immediately after elution.

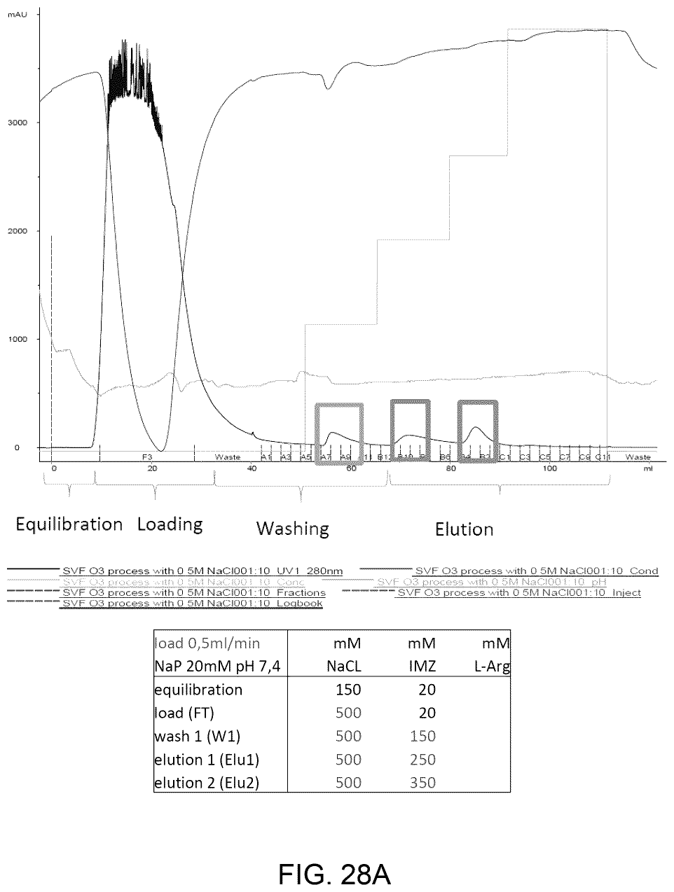

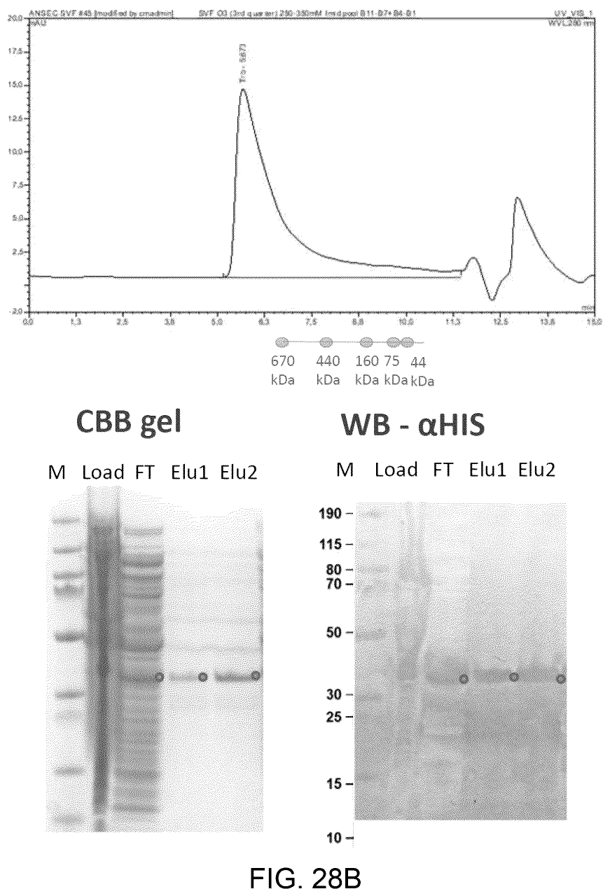

IMAC column based purification was repeated under alternative conditions to isolate exemplary construct 03 expressed in E. coli. In Run 2, the lysis conditions were similar to Run 1, but a smaller 250 mL bacterial culture was used. The buffer conditions were changed from Run 1, 500 mM NaCl was used in the loading, wash 1 and elution steps, instead of 150 mM NaCl as used in Run 1. Additionally, Run 2 featured a second elution step where elution was performed in imidazole (IMZ) without L-arginine (FIG. 28A). AnSEC data showed that the eluate was composed of high MW species (>670 kDa) and purity was observed to be 45% by CBB (FIG. 28B). Similarly, to Run 1, the 03 product was not fully captured, and the eluted fraction was used in a stability screen study.

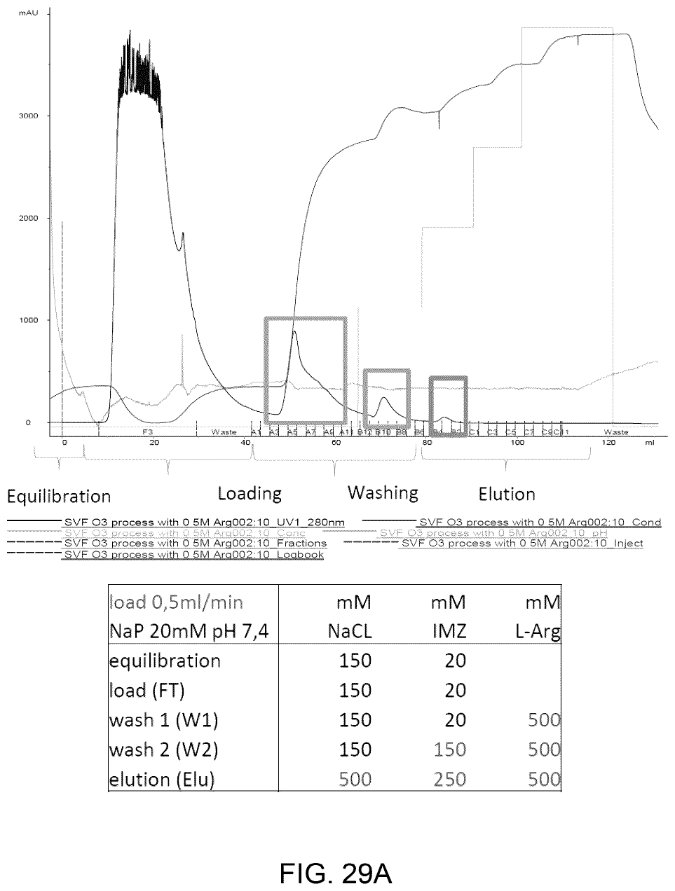

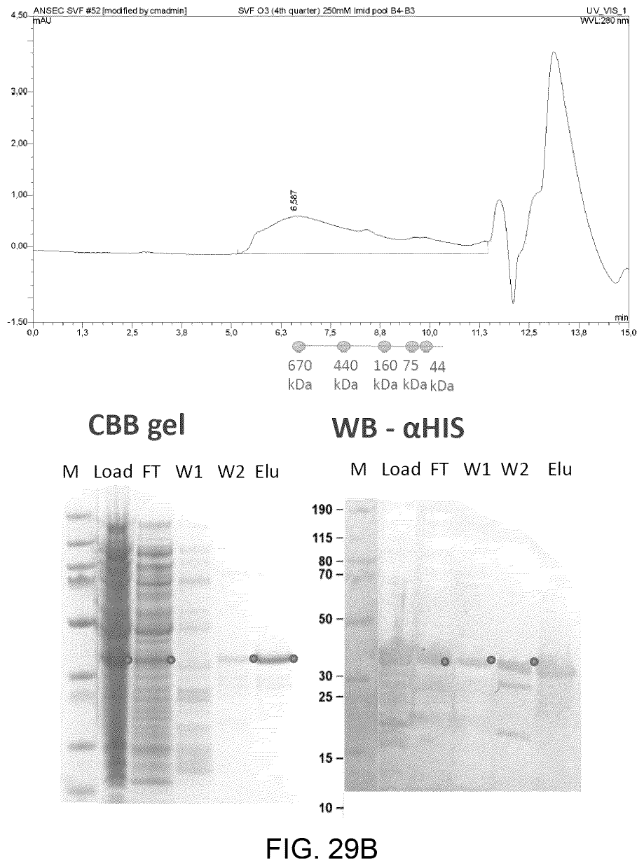

IMAC column based purification was repeated to isolate exemplary construct 03 expressed in E. coli in Run 3 under a new set of experimental conditions. The lysis conditions in Run 3 were the same as those performed above in Run 2. Compared to Run 2, Run 3 included two wash steps, a single elution step, and 500 mM L-arginine was used in both wash steps and the elution step (FIG. 29A). The eluate was determined to be a heterogenous mix of MWs by AnSEC and was found to possess about 78% purity by CBB (FIG. 29B). It was determined that similarly to Run1 and Run 2, the product was not fully captured. Additionally, the recovery was 30× lower than in Run 2 where a higher concentration of NaCl was used, the eluted fraction was further used for stability analysis.

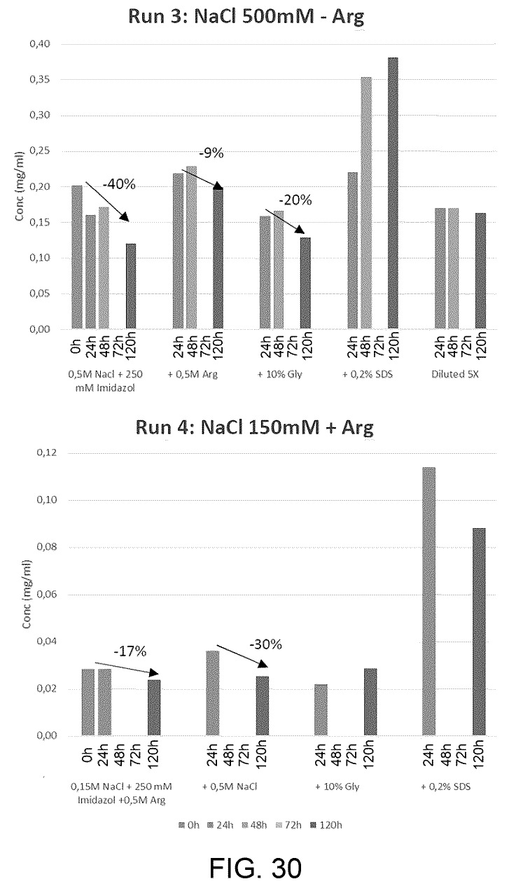

Next, an analysis of the effect of increasing residence time in the IMAC column was performed to evaluate 03 product capture from E. coli induced to express the exemplary construct. In Run 3, a solution of 0.5 M NaCl and 250 mM imidazole was used as a base elution buffer, and iterations of the elution buffer included 1) addition of 0.5 M arginine (Arg), 2) addition of 10% glycerol (Gly), 3) addition of 0.2% sodium dodecyl sulfate (SDS), or 4) diluting the base elution buffer 5 fold (FIG. 30). In Run 4, a solution of 150 mM NaCl, 250 mM imidazole, and 0.5 M arginine (Arg) was used as a base elution buffer, and iterations of the elution buffer included 1) addition of 0.5 M NaCl, 2) addition of 10% glycerol, or 3) addition of 0.2% SDS (FIG. 30). In both Runs 3 and 4, product was evaluated after 0, 48, 72, and 120 hours residence time in the IMAC column and product concentration was determined by AnSEC. The stability screen study showed that the 03 product was not completely captured by the IMAC column. Increasing residence time did not improve capture, as 40% of protein was lost from the column after 120 hours. Furthermore, the introduction of arginine into the IMAC column improved purity, but lowered recovery. The addition of 0.5M arginine to the column appeared to improve product stability and decrease protein degradation. However, the addition of glycerol provided no major improvement, and the addition of NaCl to reduce degradation was not beneficial if the protein was eluted with arginine.

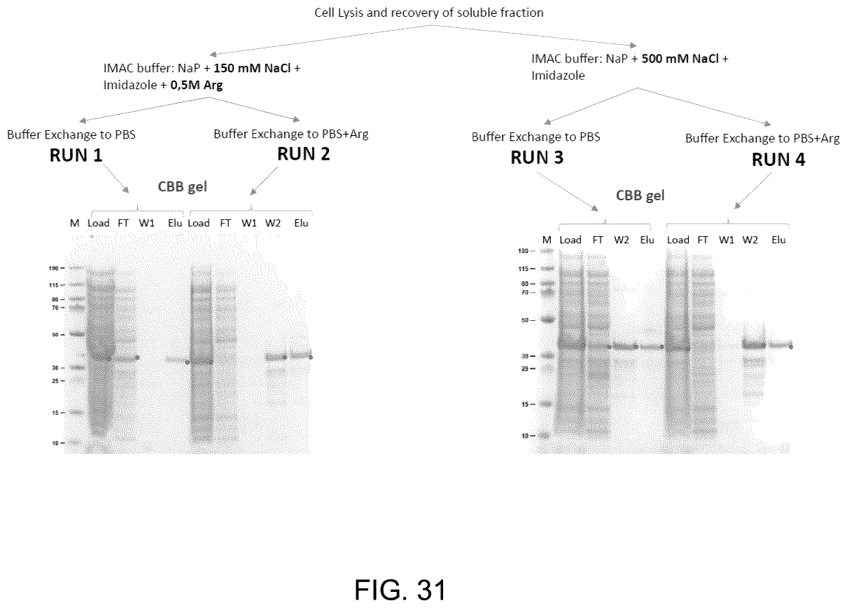

The workflow for the testing conditions for purifying expressed protein from E. coli induced to express the exemplary construct O6 is depicted in FIG. 31. Four separate experimental runs were performed, Runs 1 and 2 used an IMAC buffer comprised of NaP plus 150 mM NaCl, plus imidazole and 0.5M arginine. Runs 3 and 4 used an IMAC buffer comprised of NaP plus 500 mM NaCl, plus imidazole and no arginine. A buffer exchange step was performed to change the buffer to PBS, and Runs 2 and 4 changed to a buffer of PBS plus arginine. The flow through, wash 1, wash 2, and elution fractions were evaluated on a CBB gel (FIG. 31). It was observed that the presence of arginine improved sample binding to the column, and the CBB gel showed more product recovered in Run 4. Furthermore, the product was eluting with less host cell protein in buffer with 350 mM imidazole compared to buffer with 250 mM imidazole (92% to 66%).

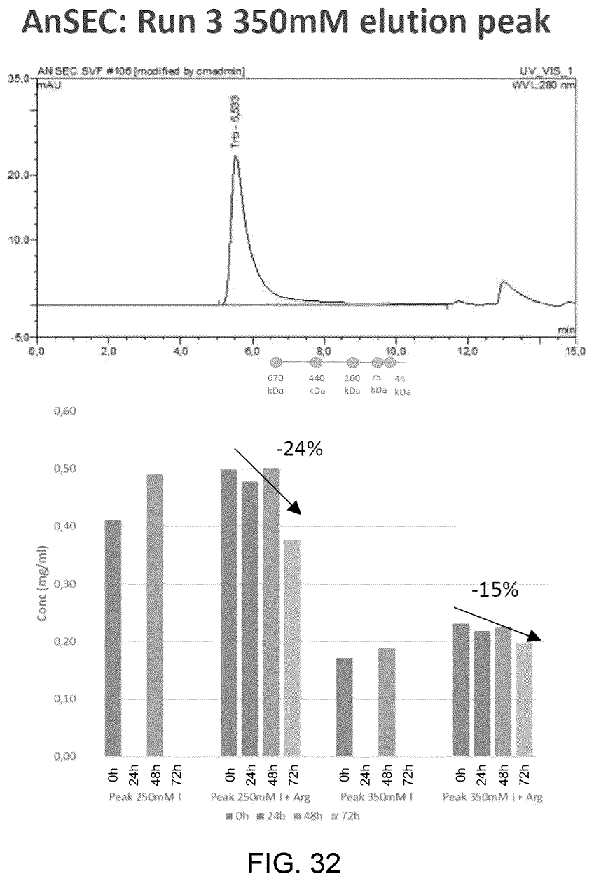

Next, a stability study at 4° C. was carried out to evaluate purification the exemplary construct O6 from E. coli following the Run 3 workflow as shown in FIG. 30. The study was carried out using an elution buffer containing 350 mM imidazole, and protein yields were compared between elution conditions of 250 mM or 350 mM imidazole with or without 0.5 mM arginine, quantified 0, 24, 48, or 72 hours after elution (FIG. 32). It was observed that different AnSEC profiles were established depending on the presence of arginine. Furthermore, adding arginine to the eluted peak reduced degradation, especially when fewer low MWs proteins were present.

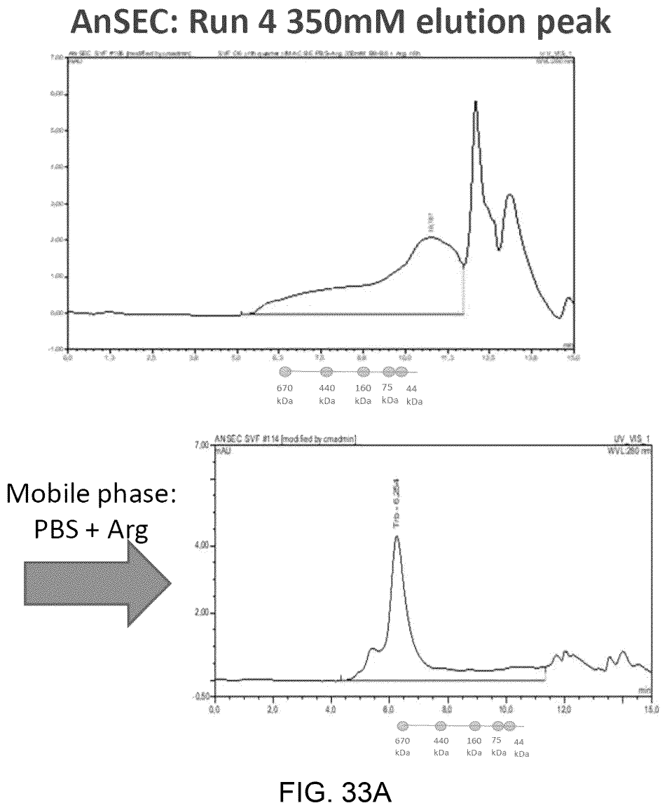

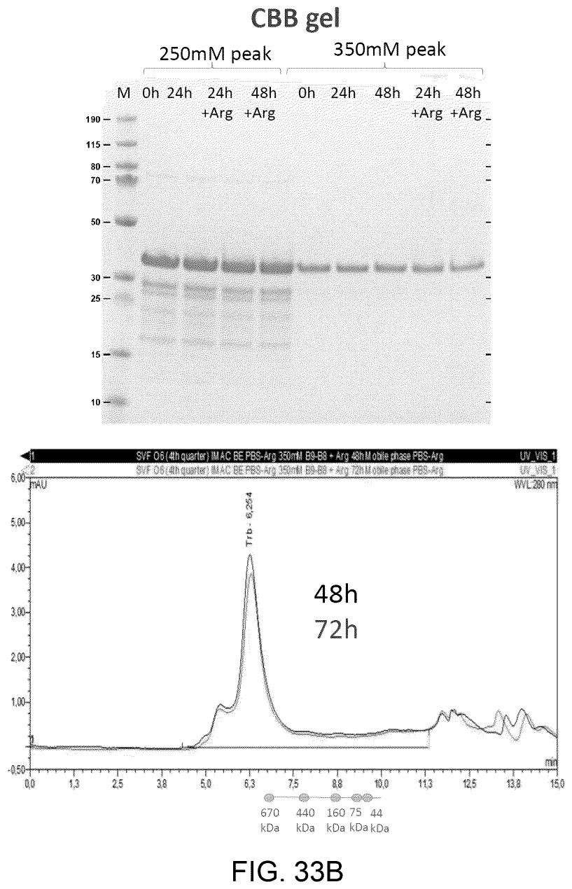

A similar study was performed analyzing protein purified from E. coli induced to express the exemplary construct O6 following the Run 4 workflow as shown in FIG. 31 using an elution buffer containing 350 mM imidazole. FIG. 33A shows AnSEC plots comparing the elution profiles with and without arginine. Additionally, the yield of protein under differing conditions using 250 mM or 350 mM imidazole and with or without arginine, was qualified 0, 24, or 48 hours after elution (FIG. 33B); Also, elution peaks were compared following 48 hours and 72 hours after elution using AnSEC analysis (FIG. 33B). After 48 h at 4° C. no degradation or aggregation was observed by CBB staining SDS-PAGE. Furthermore, the 72 hour AnSEC profile of the eluted sample was similar to 48 hours sample.

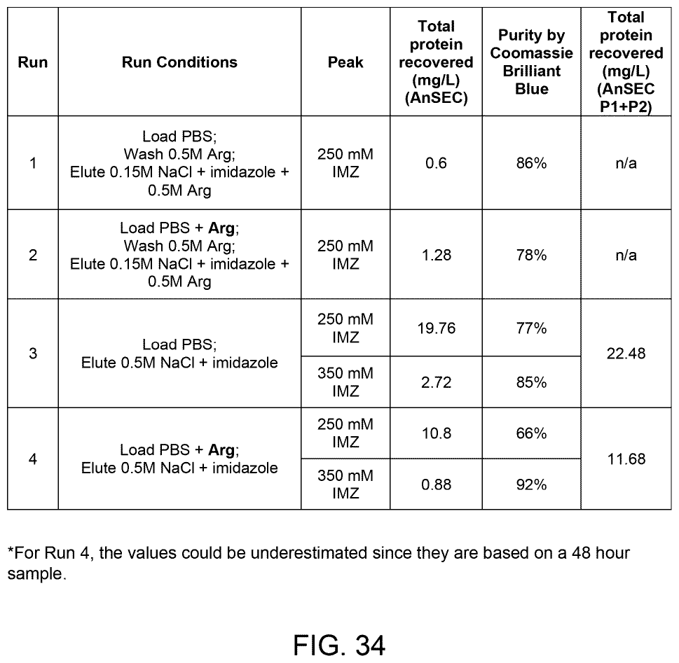

Protein output of the different run workflows depicted in FIG. 31 from E. coli induced to express the exemplary construct O6 Nis depicted in FIG. 34. Protein output is quantified in terms of total protein recovered, approximate purity as quantified by a CBB stained gel, and the sum total protein recovered from successive eluates using 250 mM and 350 mM imidazole elution buffers. Elution of IMAC with 500 mM NaCl showed better yields when compared with elution with 150 mM NaCl plus arginine.

Overall, it was observed that during loading, 0.5M arginine allows less host-cell protein binding and more HDV-Ag adsorption. Additionally, when using a 0.5 arginine buffer, more host-cell protein is removed during was steps. Furthermore, 0.5M arginine was found to influence stability by keeping the HDV-Ag from precipitation during longer time storage, and 0.5M arginine was needed during analytic columns to prevent sticking to column.

### Example 16: Further Optimization of Capture Conditions

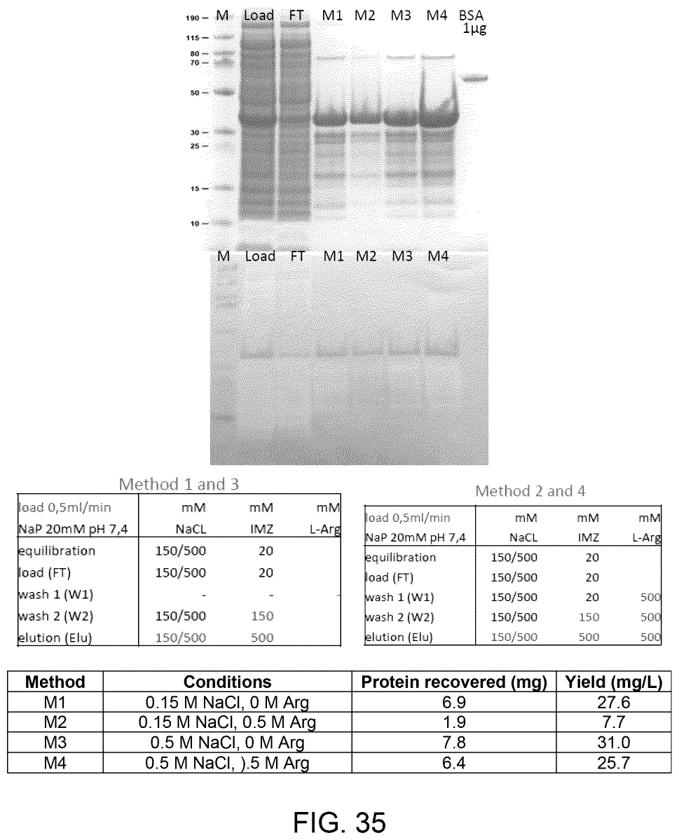

Exemplary conditions used to purify protein from E. coli induced to express the exemplary construct O6 (Methods 1-4) are depicted in FIG. 35. CBB stained SDS-PAGE gel and corresponding anti-His Western blot show the relative yields of purified protein product from these tested methods. The product was not found to be fully captured. Recoveries improved with higher NaCl concentration and lack of arginine in the running buffers. As evidenced by highest final protein yield in Method 3 at 31 mg/mL, and the lowest protein yield in Method 2 at 7.7 mg/L. Furthermore, these methods demonstrated low molecular weight proteins remaining in the eluate.

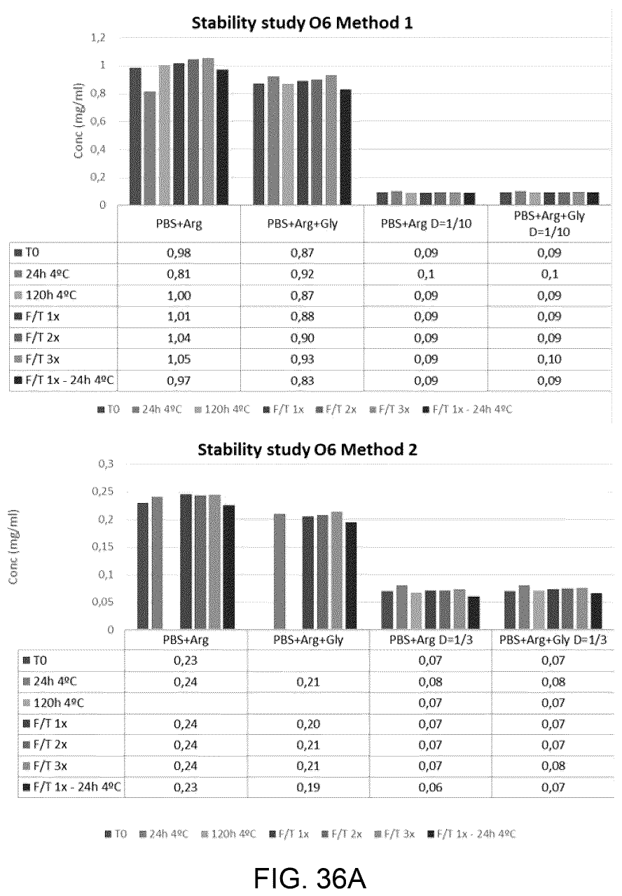

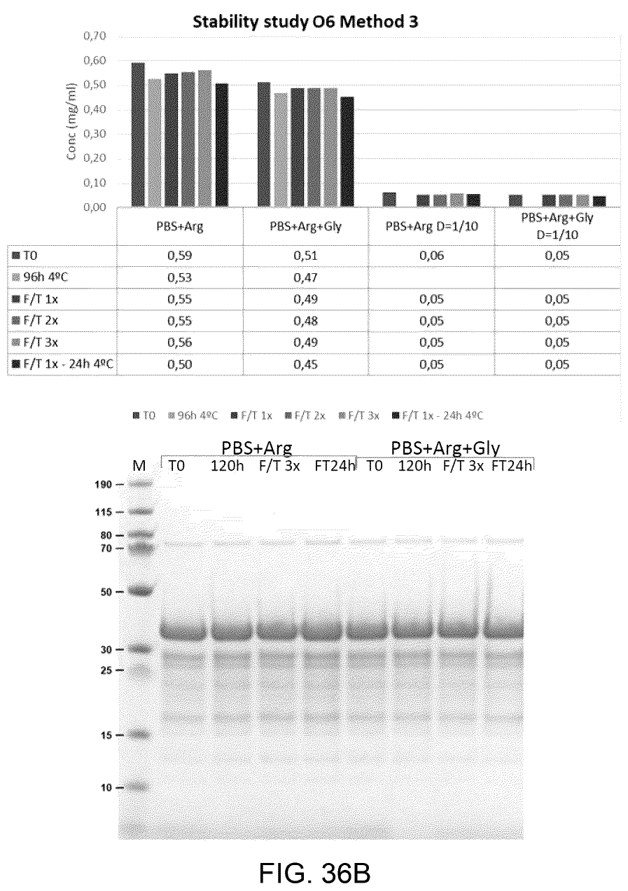

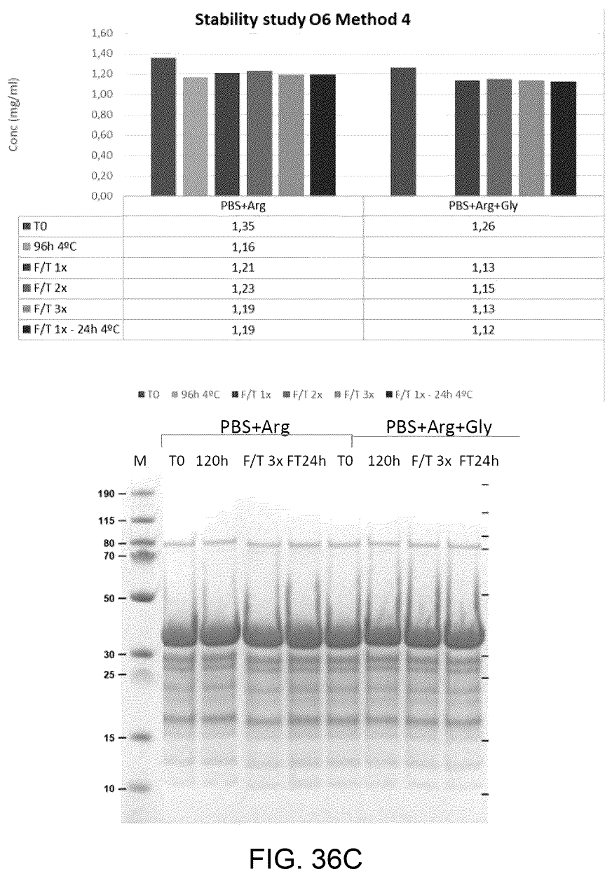

Stability studies were conducted on protein eluates according to methods depicted in FIG. 35 after buffer exchange into 1) PBS with 0.5 M arginine, 2) PBS with 0.5 M arginine and 10% glycerol, 3) PBS with 0.5 M arginine and a 10× or 3× dilution, or 4) PBS with 0.5 M arginine and a 10× or 3× dilution, and subsequent addition of 10% glycerol (FIG. 36A-36C). These samples were tested for stability after storage at 4° C. for 24 or 120 hours, 1-3 freeze/thaws (F/T), or 1 freeze/thaw and subsequent storage at 4° C. for 24 hours. Protein concentration in mg/mL of samples purified according to methods 1 and 2 is depicted in FIG. 36A. The results of samples purified according to Method 3 and a CBB stained SDS-PAGE gel of the corresponding stability tested samples are depicted in FIG. 36B. The results for samples purified according to method 4 and a CBB stained SDS-PAGE gel of the corresponding stability tested samples are depicted in FIG. 36C. No variation in protein concentration was detected up to 120 hours 4° C. and with 3 cycles of freeze/thaw. 10% product loss was detected 24 hours after 1 freeze/thaw cycle. On the SDS PAGE gel, no visible product alteration by CBB stained independently of the buffer and concentration used was observed.

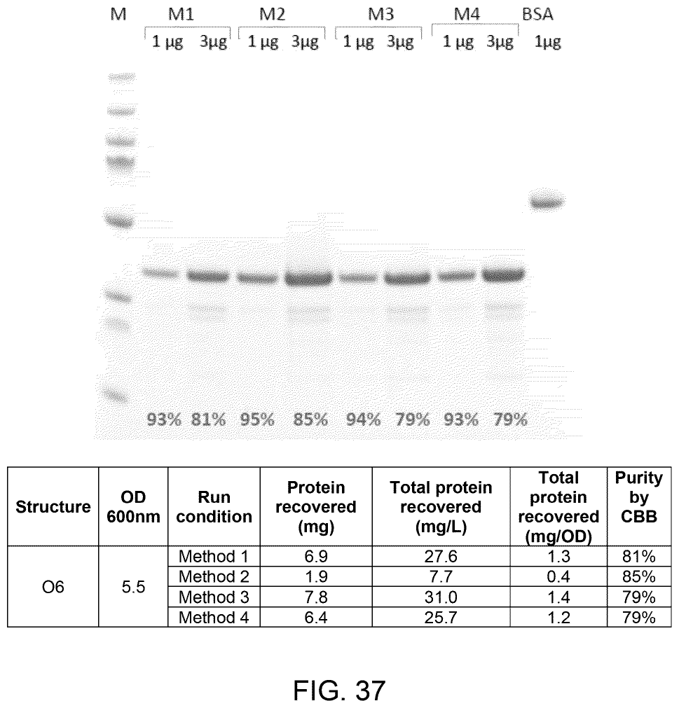

The purified protein yield of eluates collected according to the methods depicted in FIG. 35 (loading 1 or 3 μg per well) was analyzed with a CBB stained SDS-PAGE gel and the yield was quantified (FIG. 37). Method 3 achieved the highest protein recovery with 31.0 mg/L and a purity of 79% by CBB. Method 2 achieved the lowest protein recovery at 7.7 mg/L and a purity of 85% by CBB. These results were consistent with what was previously seen in FIG. 35.

### Example 17 Solubilization of IB (Insoluble Fraction)

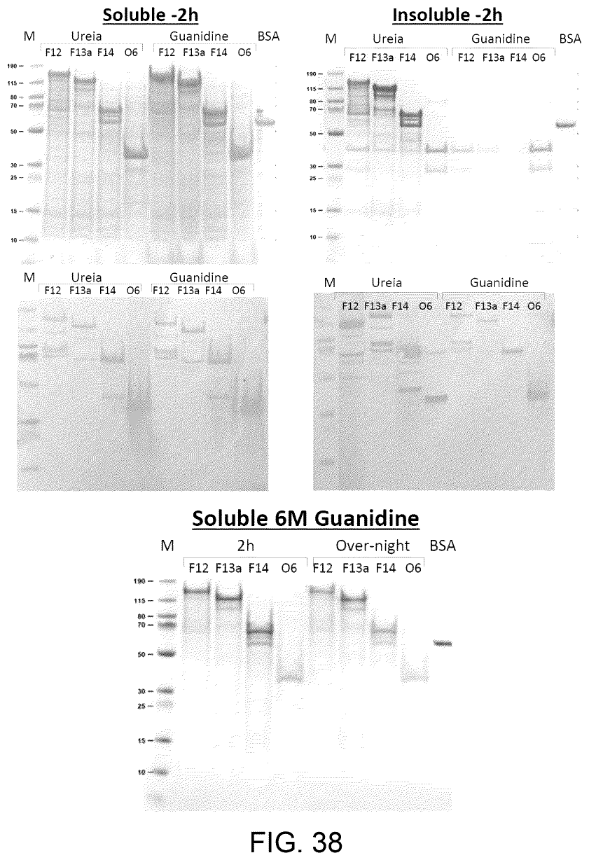

Next, the possibility of obtaining purified product from E. coli inclusion bodies for the candidates F12, F13A, F14 and O6 was evaluated. An E. coli pellet from 500 ml culture was lysed with Lysis Buffer comprising DNAse 25U/ml BB and 1 KU rLysozyme. The insoluble fraction (IB) was recovered and resuspended at 6 ml/g of IB with: 50 mM Tris pH8+6M Guanidine, 50 mM Tris pH8+8M urea, and incubated at 400 rpm 25° C. overnight. Sample was collected after 2h of incubation and clarified by centrifugation 40 min 11.500×g 4° C. The pellet was resuspended in solubilization buffer for further analysis. An analysis of the contents of the insoluble fractions (inclusion bodies; IB) was performed for lysates of E. coli induced to express the exemplary constructs F12, F13A, F14, and O6. CBB stained SDS-PAGE gels and corresponding anti-His Western blots are shown of samples of the insoluble fractions solubilized with either 6 M guanidine or 8 M urea for each exemplary construct (FIG. 38). Solubilization over 2 hours compared to overnight using 6 M guanidine was also examined. More product was found to be solubilized with 6M Guanidine after 2 hours. Furthermore, after 2 hours, the urea was not able to solubilize all the IBs. Incubation overnight was not found to increase proportion of product recovered after solubilization.

### Example 18 Refolding Buffer Screening

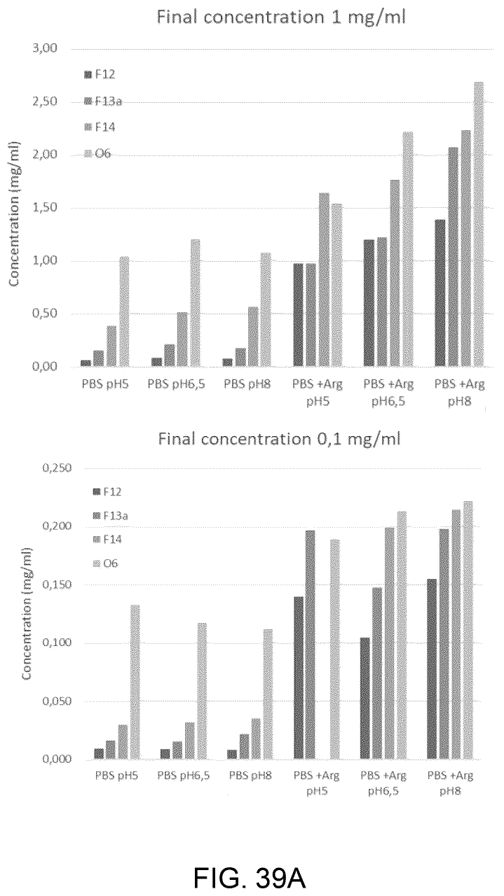

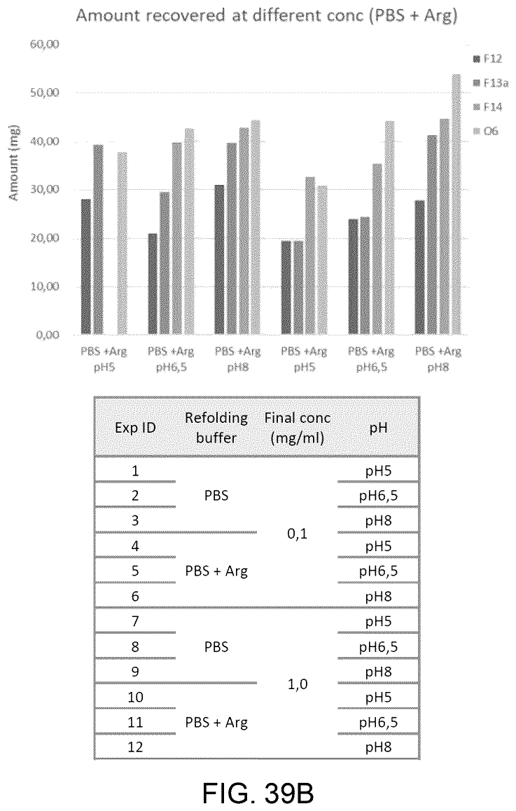

Analysis of refolding after solubilization was conducted for the insoluble fractions of lysates of E. coli induced to express the exemplary constructs F12, F13A, F14, and O6 with 6 M guanidine. Bacterial pellets from 500 ml culture were lysed with Lysis Buffer+DNAse 25U/ml BB+1 KU rLysozyme. The insoluble fraction (IB) was recovered via resuspension at 6 ml/g of IB with 50 mM Tris pH8+6M Guanidine for 2 h at 400 rpm 25° C. Clarification was performed by centrifugation 40 min 11.500×g 4° C. Following clarification, samples were diluted 1/10 by drop by drop in refolding buffer. Half of the material was prediluted by 1/10 in solubilisation buffer to refold at low concentration. Incubation was performed with agitation over-night at 4° C. followed by clarification by centrifugation 40 min 11.500×g 4° C. Concentration of protein yield recovered from 1 mg/mL or 0.1 mg/mL (diluted 10×) protein samples under differing refolding pH conditions with or without arginine was recorded from AnSEC data (FIG. 39A). The amount recovered in mg was also recorded for PBS+Arg samples (FIG. 39B). The different refolding conditions tested, which have been annotated as conditions 1-12, are depicted in the table of FIG. 39B. The protein product in buffer without arginine precipitated out of solution. However, less precipitation was detected at pH8. Additionally, no influence of protein concentration on refolding was identified.

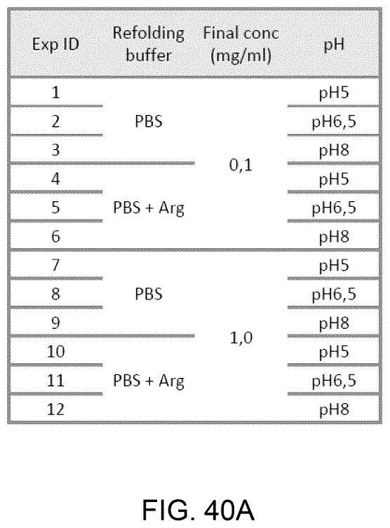

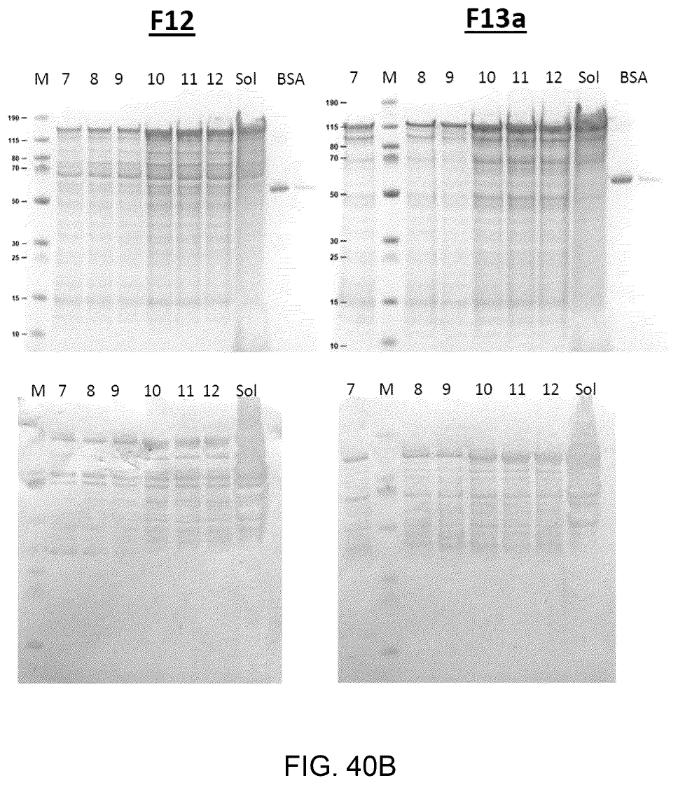

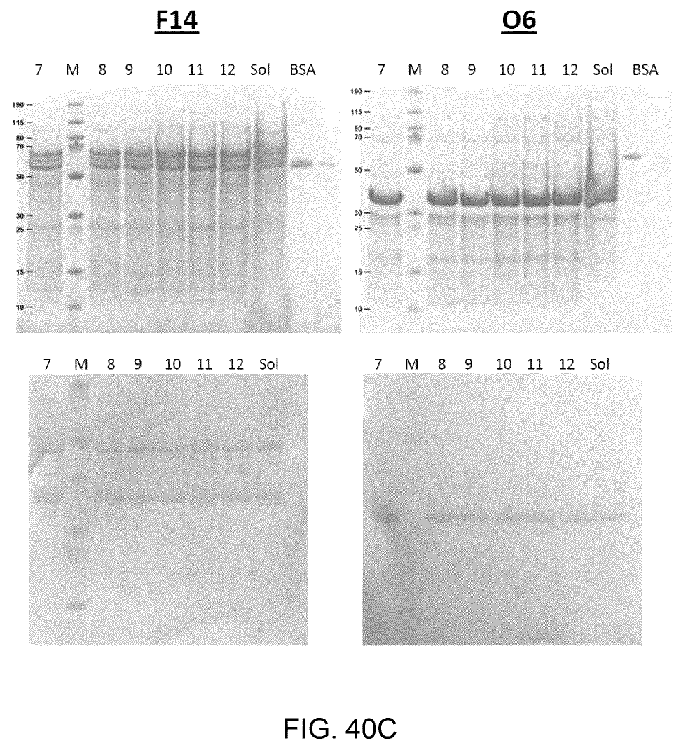

Refolded F12, F13A, F14 and O6 samples processed according to the conditions of Exp ID 1-12 (FIG. 40A) were also analyzed by CBB stained SDS-PAGE gels and anti-His western blot analysis (FIG. 40B, 40C). The conditions of EXP ID 12, involving refolding at 1 mg/ml with PBS pH8+0.5M arginine, were selected for further analysis.

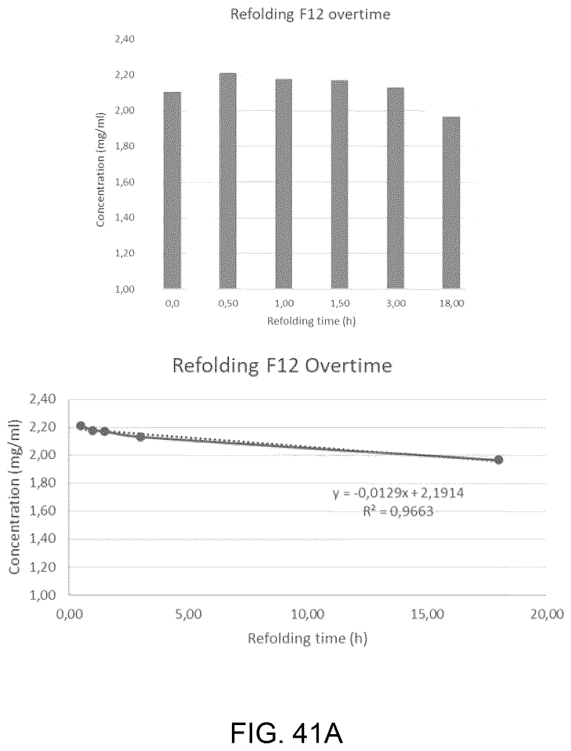

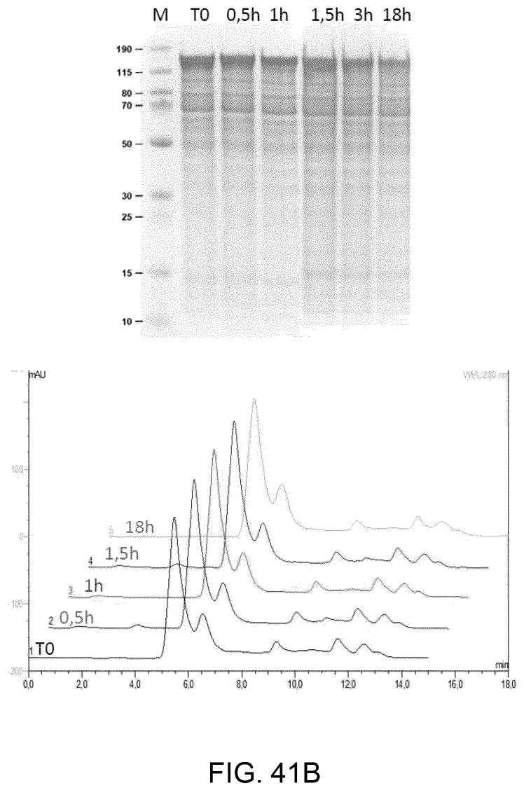

Further analysis of refolding conditions, according to EXP ID 12 shown in FIG. 40A, was performed for the exemplary construct F12 in order to establish the optimal sample incubation time. Bacterial pellets from 500 ml culture with Lysis Buffer+DNAse 25U/ml BB+1 KU rLysozyme. The insoluble fraction (IB) was recovered by resuspension at 6 ml/g of IB with 50 mM Tris pH8+6M Guanidine for 2 h at 400 rpm 25° C. Clarification by centrifugation was performed for 40 min 11.500×g 4° C. Samples were diluted 1/10 by drop-down in refolding buffer: PBS pH8+0.5M arginine. Incubation was performed with agitation over-night at 4° C. Sampling was performed at Oh; 0,5h; 1h; 1,5h; 3h; 18h followed by clarification by centrifugation 40 min 11.500×g 4° C. Samples were analyzed by CBB stained SDS-PAGE gel and AnSEC, and the protein yield (mg/mL) of samples refolded over 0, 0.5, 1, 1.5, 3, and 18 hours was recorded (FIG. 41A, 41B). Maximum concentration was detected after 0.5 hours incubation in refolding buffer. After 0.5 hours, product concentration decrease is linear up to 18 hours with a loss of approximately 2% per hour. No difference in profile was detected by CBB staining or AnSEC. From this data a refolding time of 0.5 hours was selected for further experiments.

### Example 19 IMAC Method Optimization

Purification of the exemplary constructs F12 and F13a from solubilized inclusion bodies of lysates of E. coli was further optimized by the testing of multiple methods and buffer conditions during IMAC capture. The bacterial pellet from 500 ml culture was lysed with Lysis Buffer+DNAse 25U/ml BB+1 KU rLysozyme. The insoluble fraction (IB) was resuspended at g/mL of IB with 50 mM Tris pH8+6M Guanidine for 2 hours at 400 rpm 25° C. Clarification was performed by centrifugation 40 min 11.500×g 4° C. Samples were diluted 1/10 by drop-down in refolding buffer of PBS pH8+0.5M Arg. Incubation was performed with agitation 0.5h at 4° C. followed by clarification by centrifugation 40 min 11.500×g 4° C. pH was adjusted to 5.0 and followed by clarification by centrifugation 10 min 11.500×g 4° C. before application to a HiTrap IMAC Sepharose 1 ml column (Cytiva).

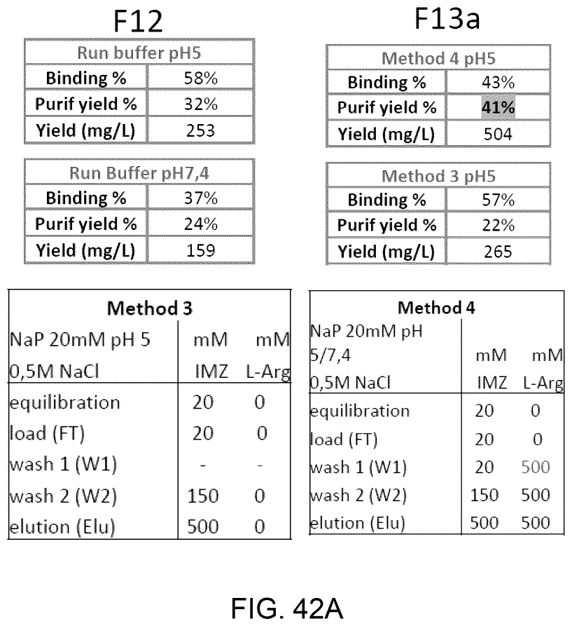

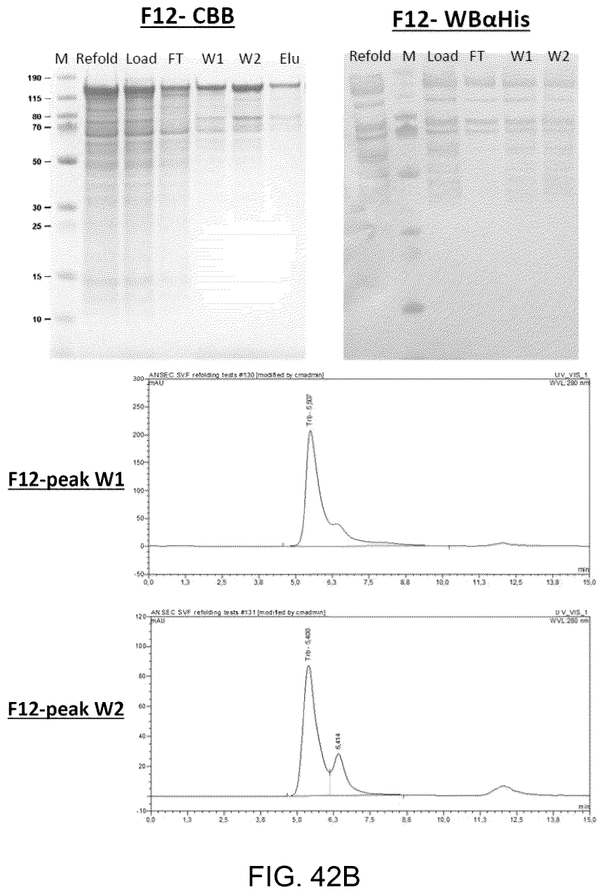

IMAC capture of F12 was tested with Method 4 where binding %, purification yield %, and yield (mg/mL) were compared between a running buffer with a pH of 5 and a running buffer with a pH of 7.4. Binding %, purification yield %, and yield (mg/mL) were all increased in the condition with a pH of 5 over the conditions with a pH of 7.4 (FIG. 42A). IMAC capture of F13a was tested with Methods 3, which included 500 mM arginine in wash 1, wash 2 and elution steps, and Method 4 which did not include arginine. While binding % and yield (mg/mL) were higher in Method 3, F13a purification yield % was higher in Method 4 (FIG. 42A). F12 inclusion body samples solubilized with 6 M guanidine, refolded, and purified with IMAC according to method 4 using a buffer pH of 5 were analyzed by CBB stained SDS-PAGE gel, corresponding anti-His Western blot, and AnSEC plots (FIG. 42B). A peak of approximately 600 KDa was obtained. However, the product was not fully captured, and a various number of product degradation products were detected by SDS-PAGE and anti-His.

### Example 20 Purification F12, F13a, F14 from Inclusion Bodies

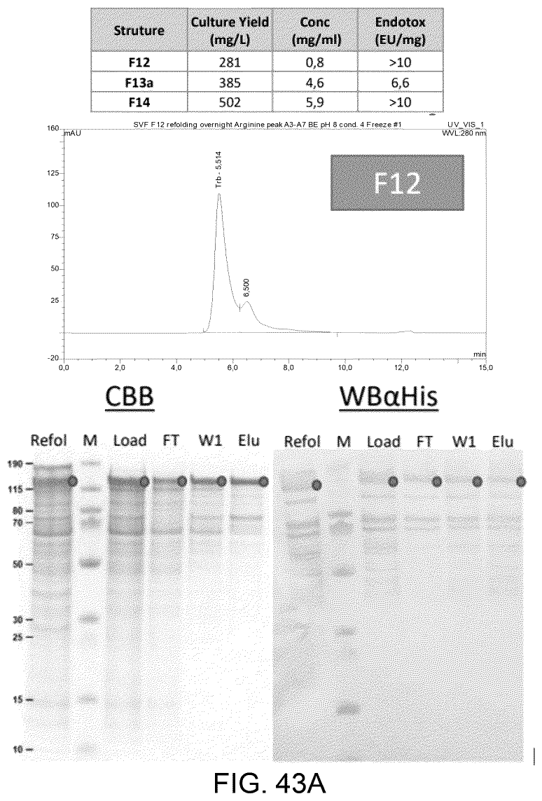

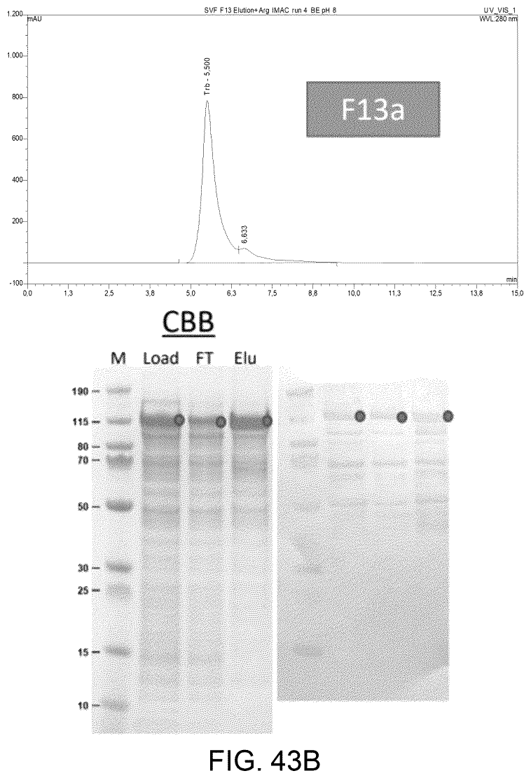

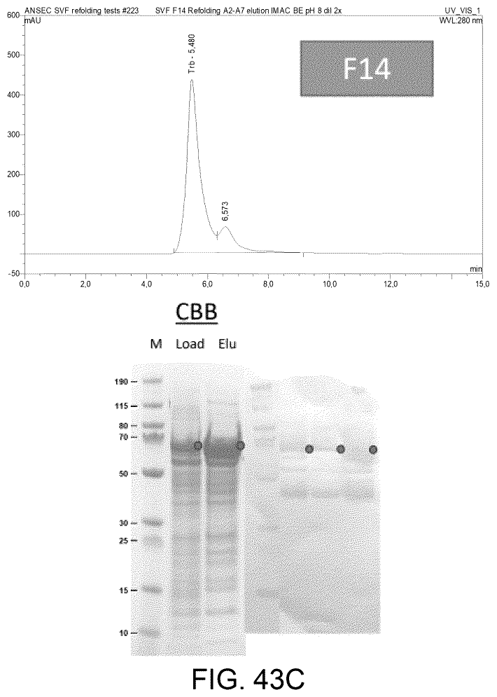

Next, a purification run to obtain purified F12, F13A, F14 product from inclusion bodies was performed, where the insoluble fraction (IB) was refolded prior to loading on the IMAC column. The bacterial pellet from 500 ml culture was lysed with Lysis Buffer+DNAse 25U/ml BB+1 KU rLysozyme. The insoluble fraction (IB) was recovered and resuspended at 6 ml/g with 50 mM Tris pH8+6M Guanidine for 2 hours at 400 rpm at 25° C. Clarification by centrifugation was performed for 40 minutes 11,500×g at 4° C. The half clarified product was diluted 1/10 drop by drop in refolding buffer: PBS pH 8+0.5M Arg and incubated with agitation 0.5 hours at 4° C. Incubation was followed by clarification by centrifugation 10 minutes 11,500×g 4° C. and adjustment of pH to 5.0 with HCl. The sample was then loaded into a 5 ml IMAC column. AnSEC plots, CBB stained SDS-PAGE gels, and anti-His Western blots of the purified products for F12, F13a, and F14 are shown in FIG. 43A, FIG. 43B, and FIG. 43C respectively. Not all product was captured and concentration was found to be especially low for F12 (FIG. 43A). Additionally, endotoxin levels as measured by limulus amebocyte lysate (LAL) testing were found to be high in F12 and F14 samples, with over 10 EU/mg detected (FIG. 43A).

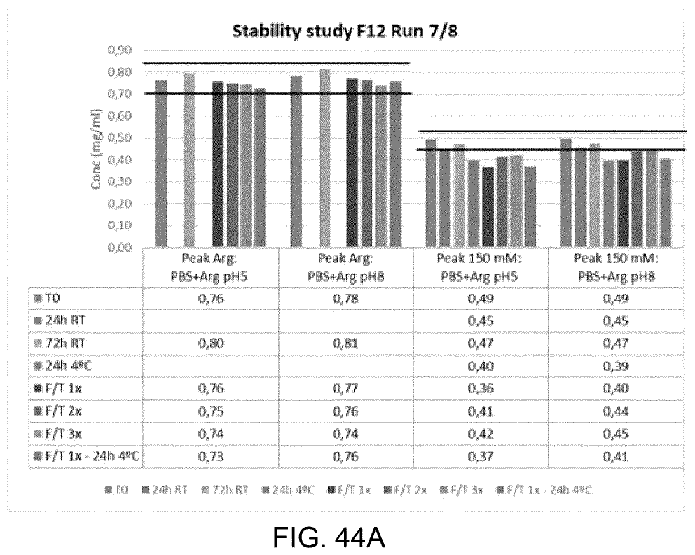

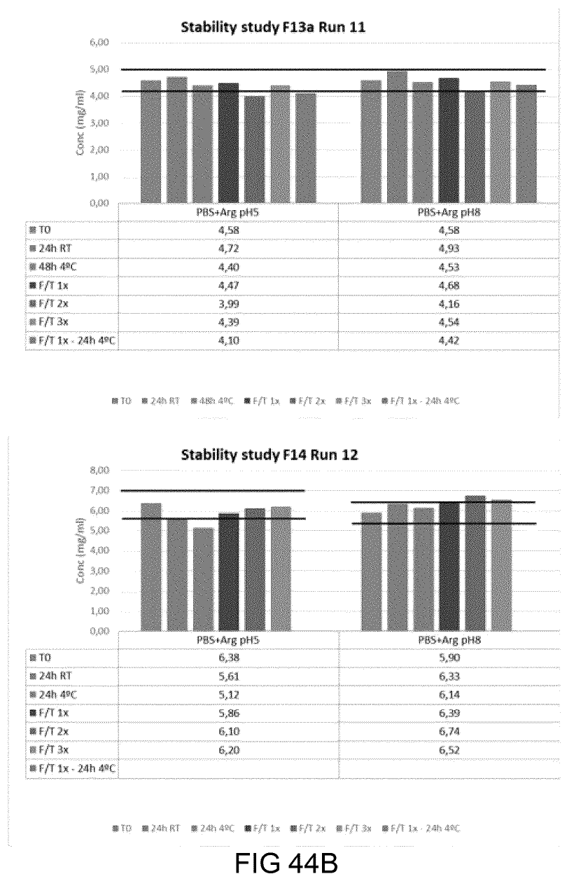

Stability analysis was performed on the protein eluates from FIG. 43A-C following elution from the IMAC column. Eluted material from the IMAC column underwent buffer exchange with PD-10 columns (Cytiva) to PBS+0.5M Arg pH 5 and pH 8. Aliquots were stored at RT, 4° C. and −80° C. for 24 or 72 hours. Samples at −80° C. were analyzed after Freeze/thaw (F/T) (up to 3 times). Samples were analyzed at different time points by AnSEC and concentration was estimated. As shown in the stability study results for F12 run 7/8 (FIG. 44A), and the stability study results got F13 run 11 and F14 Run 12 (FIG. 44B), no variation in protein concentration up to 72 hours 4° C. and with 3 cycles of F/T was observed.

### Example 21 Generation and Purification of O7 Product

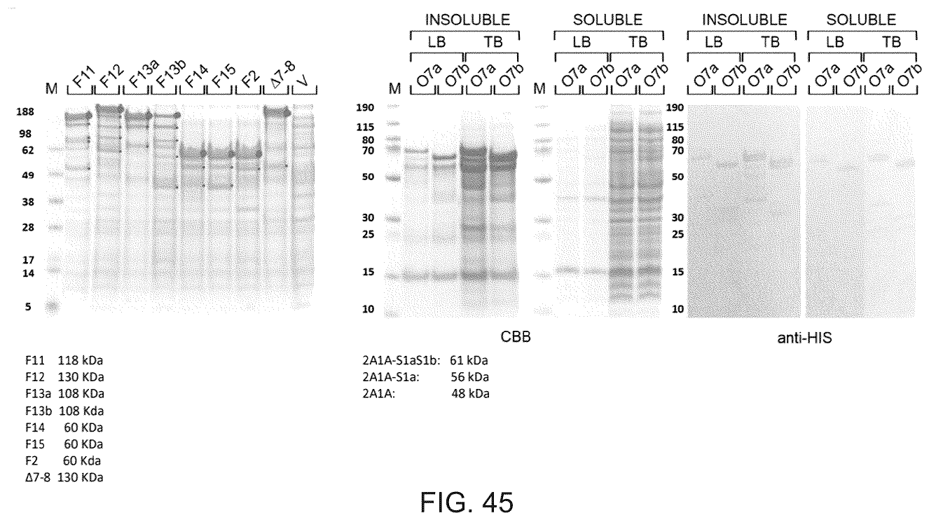

Having observed that translational coupling was successful in all operon constructs, the next objective was to test an operon structure based on dimeric structures. The newly developed dimeric operons O7a and O7b were transformed into BL21 Star (DE3) E. coli and grown in small scale flask cultures in LB and TB. Pre-culture was conducted at 30° C. Induction and temperature shift were performed at OD₆₀₀˜1. Induction took place over 4 hours at 25° C. with IPTG 0.5 mM. Protein was collected and compared through CBB stained SDS-PAGE gels, and anti-His Western blots from soluble and insoluble fractions (IB) for O7a and O7b constructs from cultures from LB and TB (FIG. 45). CBB staining data for F11-F15, F2, 47-8, and the empty vector (V) were included in FIG. 45 for comparison. Both O7a and O7b were expressed at a high level in inclusion bodies. Culturing in TB gave better results as compared to LB, with stronger CBB bands at the appropriate sizes observed in samples from TB cultures. The major degradation product corresponded to dimer (2A/1A and 1B/2B) without Sla/b, and this band did not react with anti-HIS.

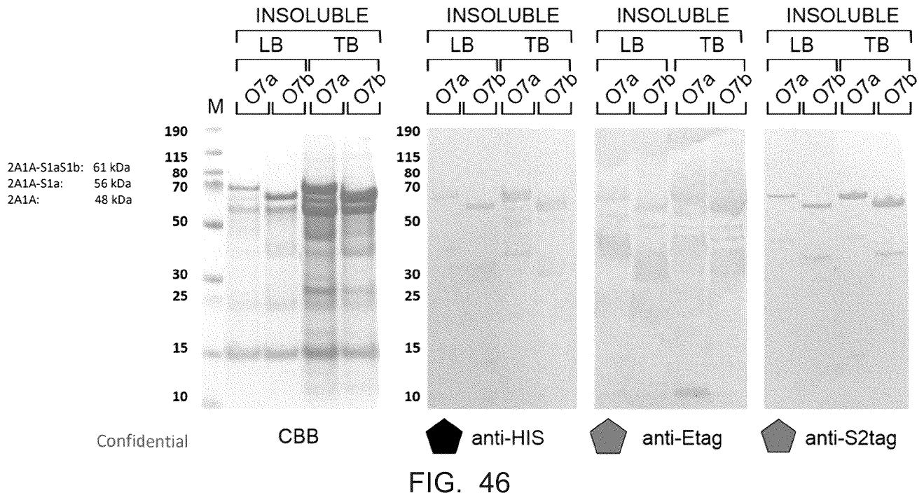

Additionally, the protein products from the insoluble fractions of O7a and O7b in FIG. 45 from LB and TB cultures were analyzed by anti-Etag and anti-S2tag western blots (FIG. 46). Both the 2A/1A (E-tag) and 1B/2B (S2-tag) fusions were found to be expressed in the O7a and O7b products.

### Example 22 Further Optimization of O6 Purification

Previous purification strategies of O6 achieved recovery yields of 20-30 mg/L of culture by IMAC purification using 0.5M NaCl (method 3 or 4). Furthermore, addition of 0.5M Arg increased stability up to 120 hours at 4° C. and with 3 cycles of Freeze/thaw. The next objective was to perform O6 purification under endotoxin free conditions, determine the best chromatography technique for endotoxin removal and assess stability of the purified protein product with and without adjuvant.

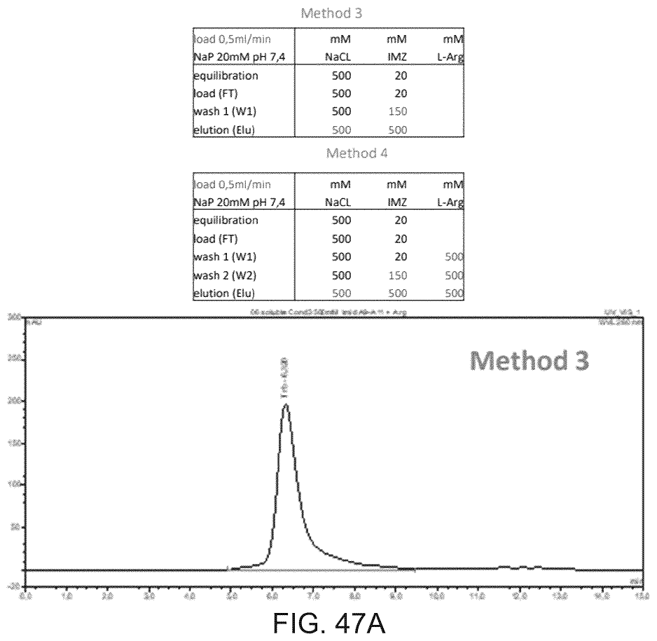

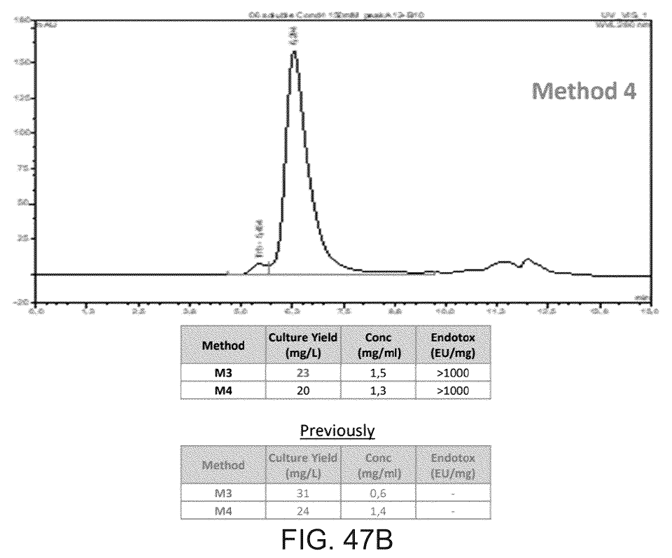

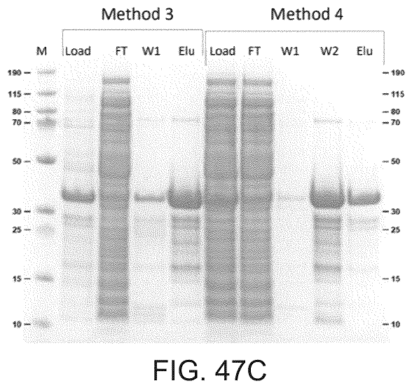

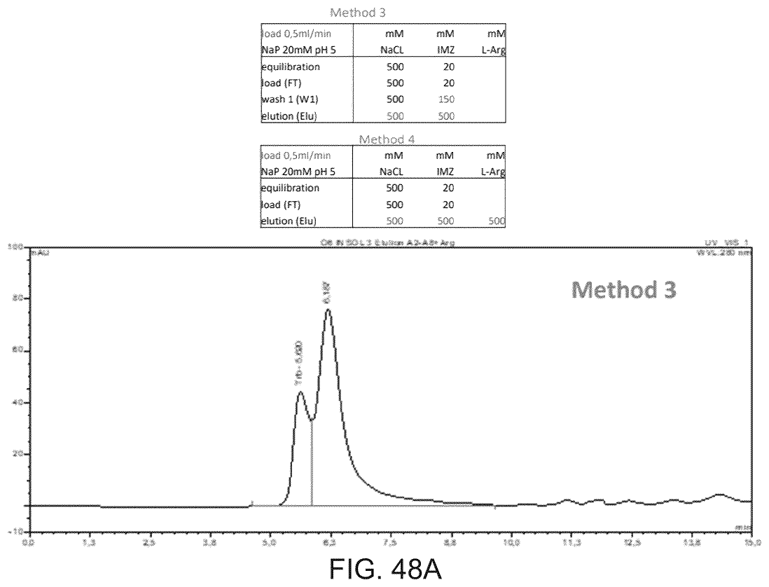

First, a purification run was performed on protein from the soluble fraction of E. coli induced to express the exemplary construct O6. The bacterial pellet from 500 ml culture was lysed with Lysis Buffer+DNAse 25U/ml BB+1 KU rLysozyme (C) and the soluble fraction was recovered. Buffer was exchanged to PBS+Arg (Load) and the sample was loaded into a HiTrap IMAC Sepharose 1 ml column (Cytiva) The soluble fraction was recovered and the buffer was exchanged to PBS+arginine before loading the samples into a HiTrap IMAC Sepharose column. Elution was performed according to methods 3 and 4, where method 3 included a single wash step, while method 4 included 2 wash steps and incorporated arginine in both wash steps and the elution buffer (FIG. 47A). Flow through (FT), wash (W1, W2), and eluate (Elu) were analyzed by AnSEC (FIG. 47A, 47B) and CBB stained SDS-PAGE gel (FIG. 47C). The final yield for method 3 and AnSEC profile were improved over those from method 4 (consistent with previous data) (FIG. 47B). However, low molecular weight proteins remained in the eluate (estimated purity >80% by densitometry). Additionally, a high level of endotoxins was found in the final product of both methods, greater than 1000EU/mg (FIG. 47A).

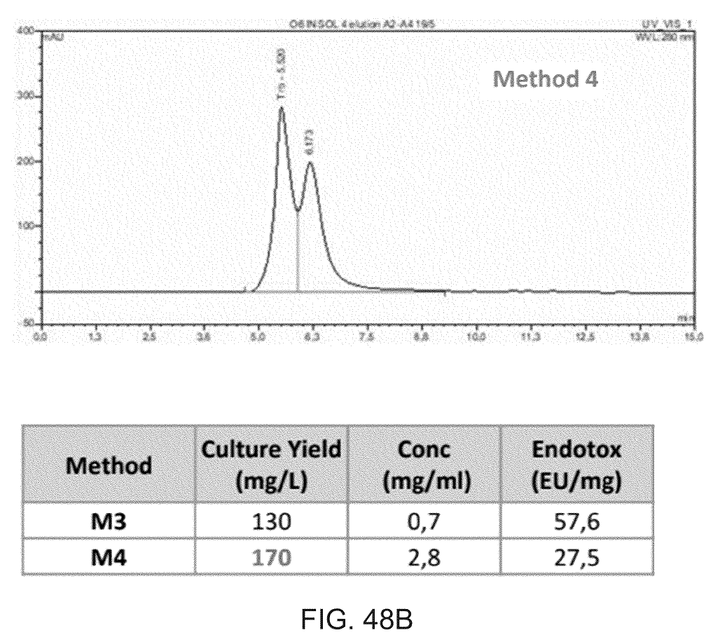

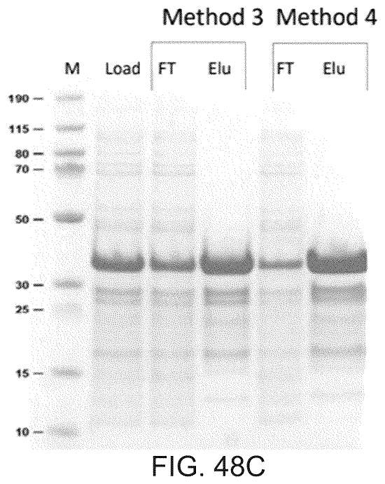

Purification of the O6 insoluble fraction was performed following purification of the soluble fraction in FIG. 47A-47C. The insoluble fraction (IB) was recovered from 500 ml culture and resuspended at 6 ml/g of IB with 50 mM Tris pH8+6M Guanidine and incubated for 2 hours at 400 rpm at 25° C. Next, samples were clarified by centrifugation for 40 minutes at 11,500×g 4° C. The half-clarified product was diluted 1/10 drop by drop in refolding buffer: PBS pH8+0.5M Arg. Incubation was performed with agitation for 0.5 hour at 4° C. Clarification by centrifugation was performed for 10 minutes at 11,500×g 4° C. before loading the refolded sample onto a 5 ml IMAC column. The clarified insoluble product was purified according to IMAC methods 3 or 4, as shown in FIG. 48A. AnSEC data for samples processed according to methods 3 or 4 is shown in FIG. 48A and FIG. 48B respectively, and the CBB stained SDS-PAGE gel is shown in FIG. 48C. Different AnSEC profiles were observed depending on whether the IMAC method applied included arginine (FIGS. 48A, 48B). Method 4 achieved superior production yields compared to method 3, 170 as compared to 130 mg/L (FIG. 48B). Endotoxins in the final product were above 10 EU/mg.

### Example 23 Removal of Endotoxins from O6 Product

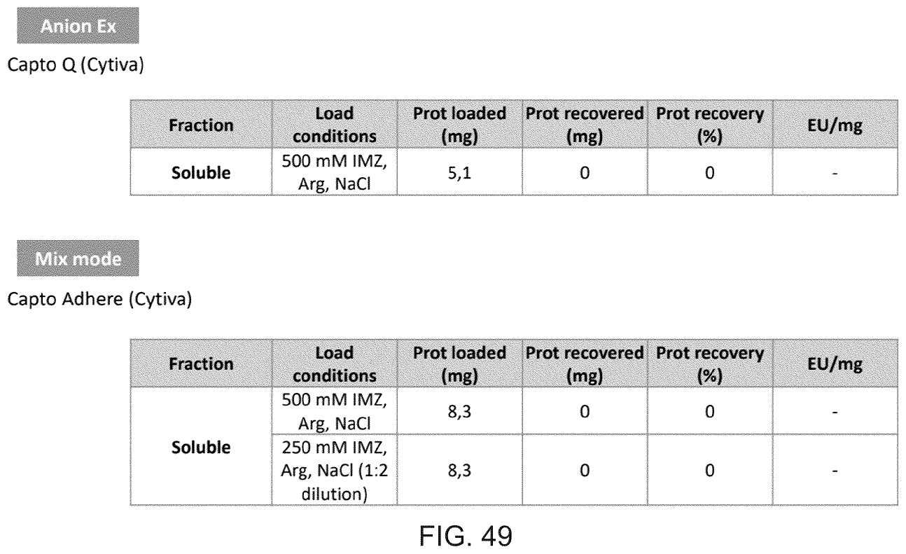

Purification tests of both soluble and insoluble fractions of O6 protein resulted in the generation of product with high levels of endotoxins. The next objective was to determine the best way to remove the endotoxins from the protein product without compromising protein yield. Considering that endotoxins are negatively charged and hydrophobic, and that O6, having a pI of 9.6 at pH 7.4, is theoretically positively charged, endotoxin removal with anion exchange and mixed mode columns was evaluated. It was hypothesized that with anion exchange and mix mode columns, endotoxins would be retained in the column, and the product would be recovered in flow-through mode. However, it was observed that IMAC purified O6 from the soluble fraction that had been stored at 4° C. for <72 hours failed to bind to either the anion exchange (Capto Q (Cytiva)) or mix mode columns (Capto Adhere (Cytiva)) (FIG. 49). No protein was recovered, regardless of whether the buffer was 500 mM IMZ, Arg, NaCl or a 1:2 dilution of the buffer. Therefore, it was necessary to screen different buffer compositions to determine if altering conductivity could prevent all of the O6 product from binding to the resins of the columns.

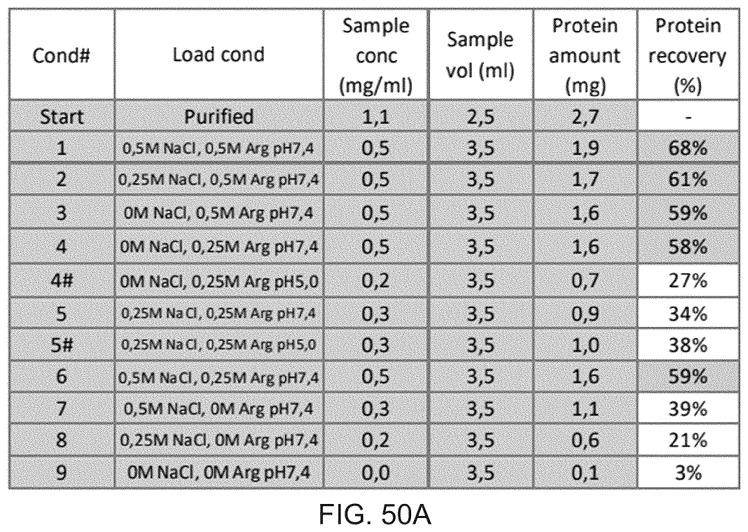

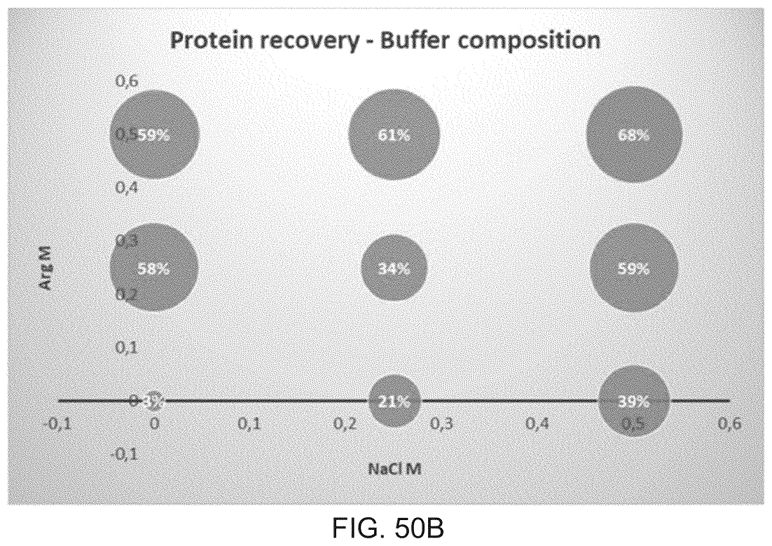

A buffer screening was performed using nine different buffer conditions where NaCl varied from OM-0.5M, Arg varied from OM-0.5M, and pH varied from 5.0 to 7.4 (FIG. 50A). Purified Material from the insoluble fraction following IMAC purification was stored at 4° C. for less than 72 hours. Buffer exchange to one of the nine conditions in FIG. 50A was performed in PD-10 columns (Cytiva). Samples were stored for 4 hours at RT, clarified by centrifugation, and analyzed by AnSEC and quantified by AUC. Protein recovery (%) was recorded for each condition (FIG. 50A) and was graphically represented in comparison to NaCl and Arg concentration (FIG. 50B). Buffer exchange to remove IMZ present in the start condition allowed recovery of 68% of product after 4 hours. However, removing arginine resulted in significant losses compared to removing NaCl. All the product was found to precipitate without the presence of Arg or NaCl. The best compromise between product loss and conductivity was achieved at OM NaCl and 0.25M Arg.

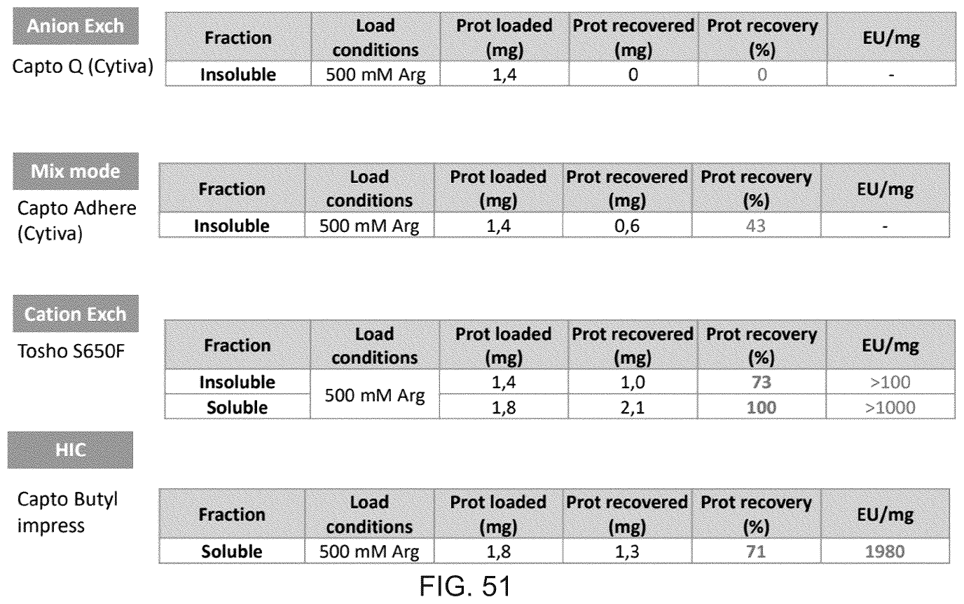

Having identified a suitable buffer for downstream endotoxin removal, O6 samples from soluble fraction were purified via IMAC column and stored at 4° C. for less than 72 hours were exchanged to OM NaCl and 0.25M Arg buffer (conductivity ˜20 mS) and loaded onto ion exchange and mix mode columns. Samples from the insoluble fraction were loaded into anion exchange (CaptoQ Cytiva), mix mode (Capto Adhere Cytiva), cation exchange columns (Tosho S650F) (FIG. 51). Samples from soluble fractions were loaded onto cation exchange and hydrophobic interaction columns (HIC) (Capto Butyl impress). All samples were loaded with 500 mM Arg, 1.4 mg of protein were loaded from samples from insoluble fraction and 1.8 mg of protein were loaded from samples from the soluble fraction. O6 protein was still binding to the anion exchange column and partially to the mix mode column with protein recovery percentages of 0% and 43% respectively (FIG. 51). The majority of the product bound to the cation exchange column, as expected, and was recovered, with 73% recovered in insoluble sample and 100% recovered in soluble sample (FIG. 51). However, no endotoxins were removed by the cation exchange column with levels of >100 EU/ml detected for the insoluble sample and >1000 EU/ml detected for the soluble sample. On the HIC column, 71% of the product was recovered and level of endotoxins decreased (reduced to 172EU/ml from a sample with a starting amount of >1000 EU/ml) (FIG. 51). As the HIC column was capable of achieving both high recovery and endotoxin removal, further optimization was performed using the HIC column.

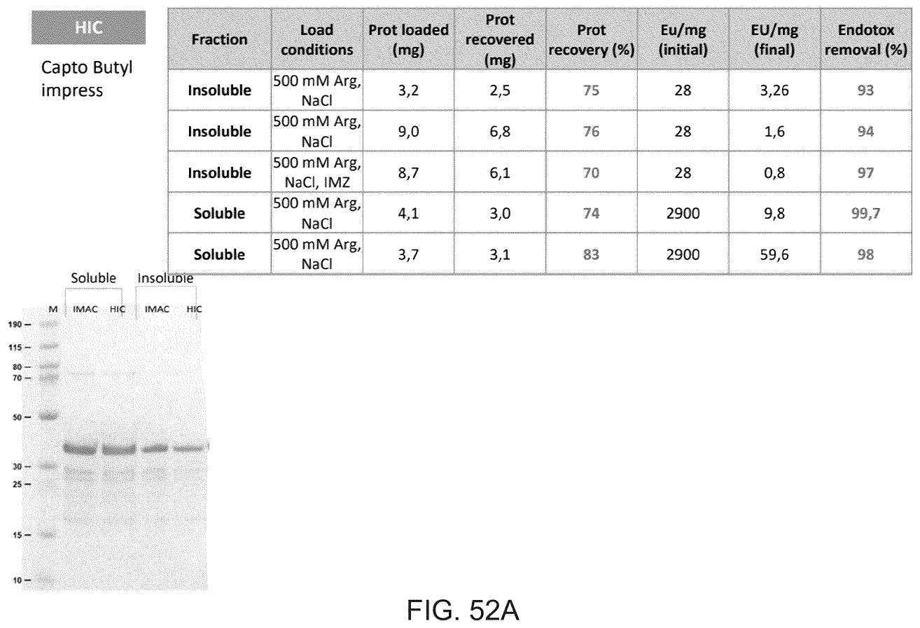

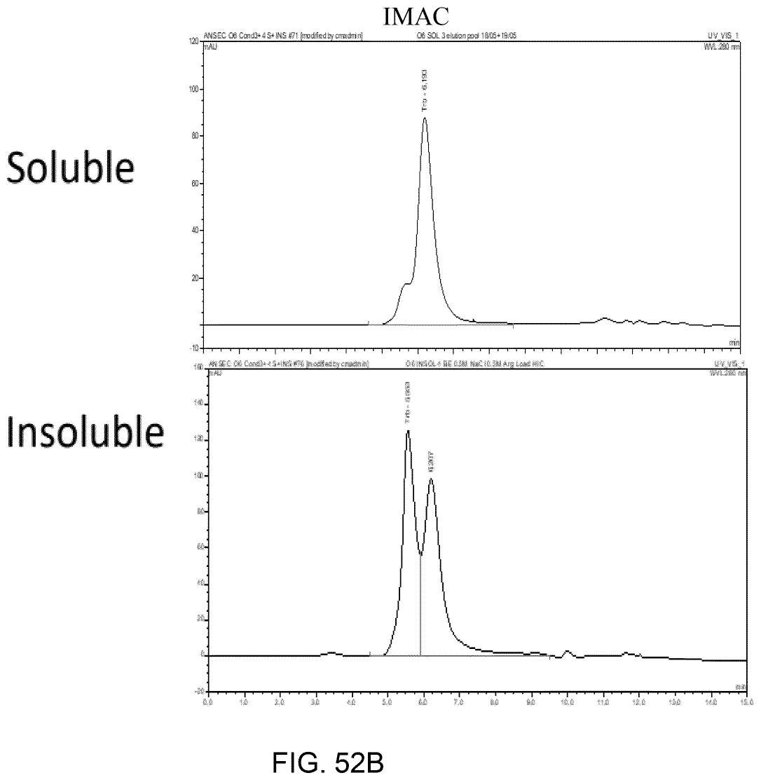

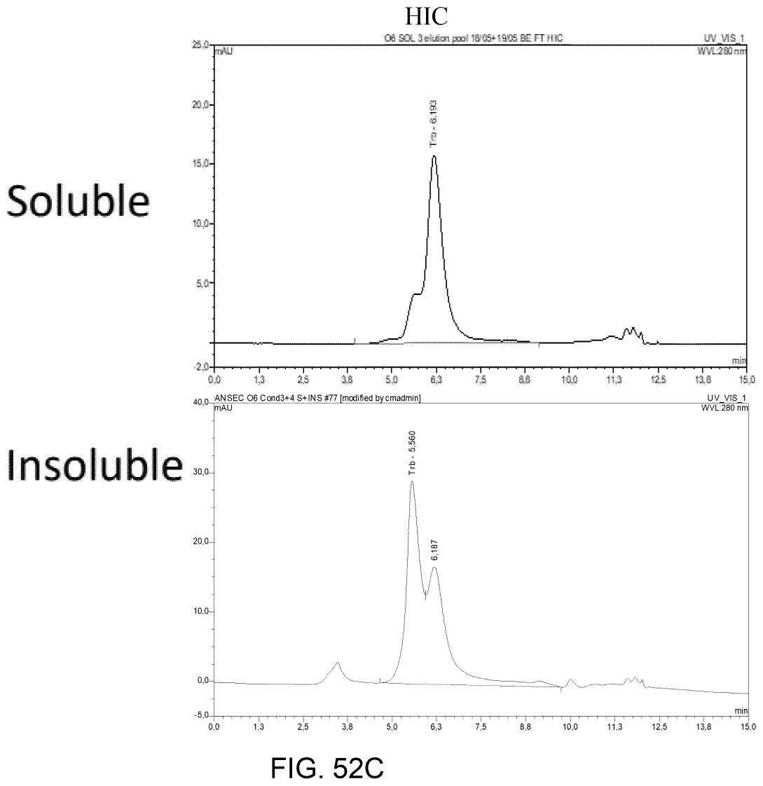

Material from soluble and insoluble samples purified by IMAC and stored at 4° C. for less than 72 hours underwent buffer exchange or pH adjustment before loading into a HIC column. Buffer was exchanged to 500 mM Arg, NaCl, with or without imidazole. Protein recovery and endotoxin removal were determined by comparing initial and final values after HIC. Protein recovery above 74% was achieved when sample was loaded without imidazole, regardless of which fraction was used (FIG. 52A). Endotoxins from insoluble fraction were reduced from 28EU/mg to 1.6EU/mg, while endotoxins from soluble fraction were reduced from 2900EU/mg to 9.8-59.6EU/mg, representing a removal above 98% (FIG. 52A). Due to the incomplete removal of endotoxins from the soluble fraction, it was determined that a second run in HIC may be required if endotoxin value is above 10EU/mg after a single run is performed. Product profile was determined to be maintained before and after HIC by SDS-PAGE (FIG. 52A) and AnSEC (FIG. 52B, 52C).

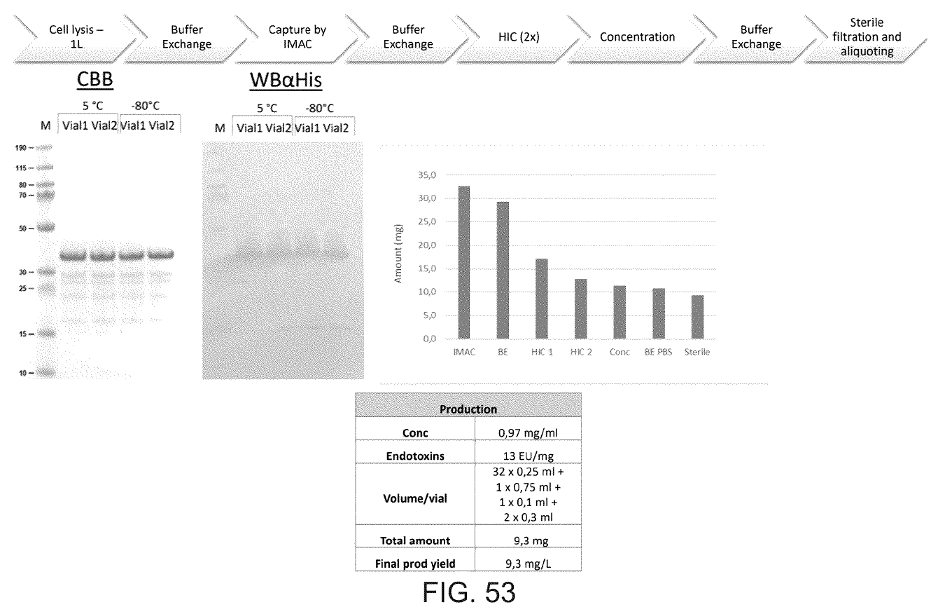

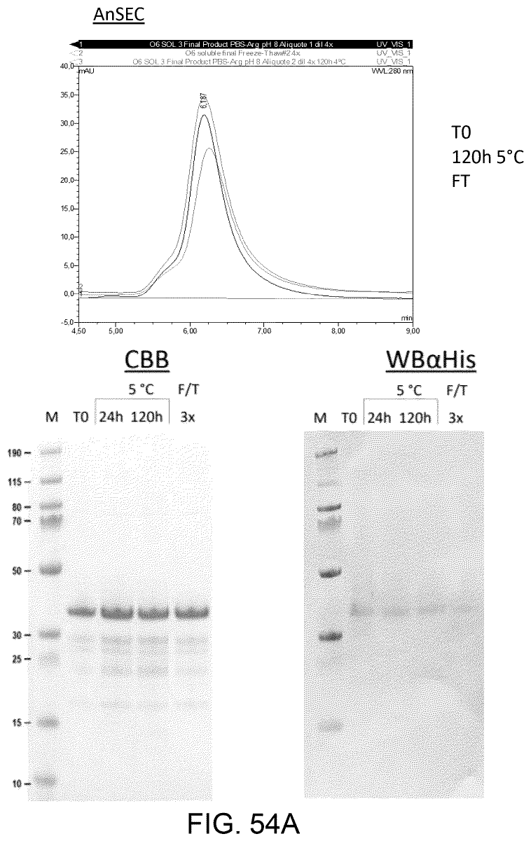

A schematic for a purification process for O6 from the soluble fraction was tested in FIG. 53A-53C. The steps of the purification process are: 1) lysis of 1 L of E. coli culture. 2) Buffer exchange to PBS+0.5M Arg with pH 7.4. 3) Capture by IMAC and elution with 0.5M NaCl+0.5M IMZ. 4) Buffer exchange to PBS+0.5M Arg+0.5M NaCl pH 7.4. 5) Two runs through HIC and elution with PBS+0.5M NaCL+0.5M Arg. 6) Concentration of the sample. 7) Buffer exchange to PBS+0.5M Arg+pH 8.0. 8) Sterile filtration and aliquoting. Aliquots of final product were stored at 5° C. and −80° C. before analysis by CBB stained SDS-PAGE gels, and anti-His Western blots, where sample profile appeared similar between the storage conditions (FIG. 53). Additionally, protein amount (mg) was recorded throughout the process with the final amount of purified protein measuring 9.3 mg (FIG. 53A).

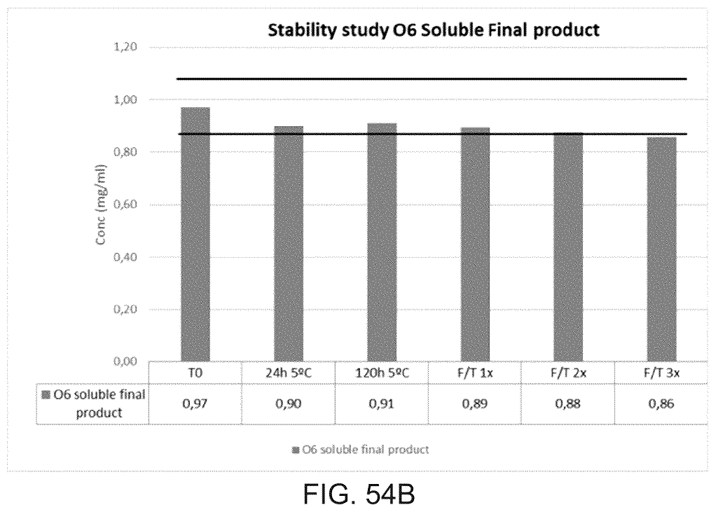

A stability study was conducted on samples purified in FIG. 53A-53C. Freshly isolated samples (TO) were compared to samples stored at 5° C. for 120 hours and samples stored at −80° C. that underwent freeze/thaw up to three times. AnSEC data, CBB stained SDS-PAGE gels, and anti-His Western blots showed similar profiles regardless of storage conditions (FIG. 54A). Sample concentrations estimated by AUC in an AnSEC plot were less than 10% below the initial concentration estimated (FIG. 54B). Stability was confirmed at 5° C. up to 120 hours and up to 3 cycles of freeze/thaw.

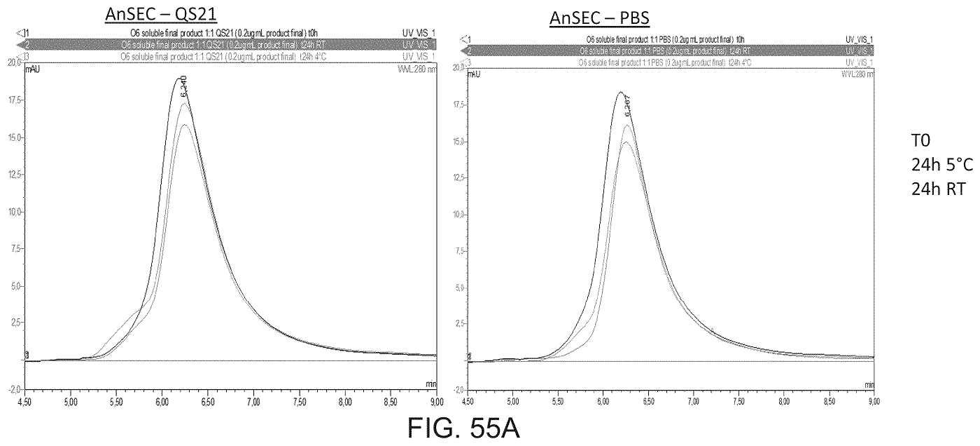

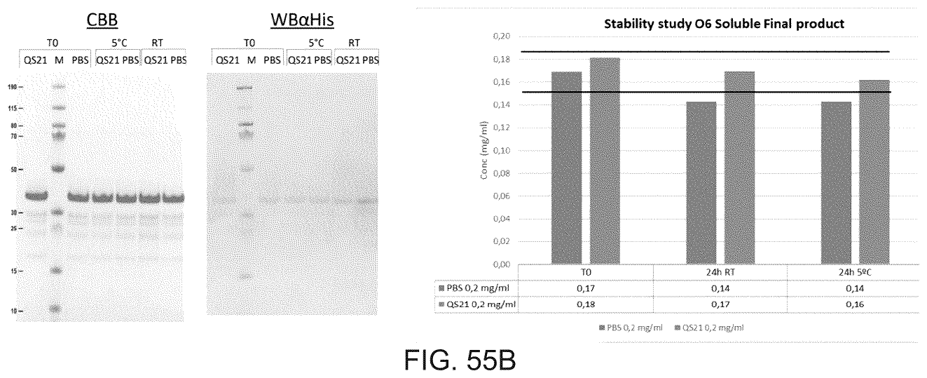

Next, stability was evaluated after the addition of the adjuvant QS-21. Aliquots of final product were defrosted and diluted to ˜ 0.4 mg/ml. Half of the product was mixed 1:1 with QS-21 (final concentration at 0.2 mg/ml). The half was diluted 1:1 with PBS+0.5M Arg. Samples were stored 24 hours at RT and 5° C. Samples were analyzed by AnSEC (FIG. 55A), CBB stained SDS-PAGE gels (FIG. 55B), and anti-His Western blots (FIG. 55B) where similar profiles were observed between samples with and without QS-21. Protein concentration was estimated using & and the AUC. Samples stored 24 hours at RT and 5° C. were within 10% of initial concentration with QS-21, and the addition of QS-21 improved stability of protein after 24 hours at RT and 5° C. (FIG. 55B).

### Example 24 Polishing of O6 Insoluble Sample for Removal of High MW Peak

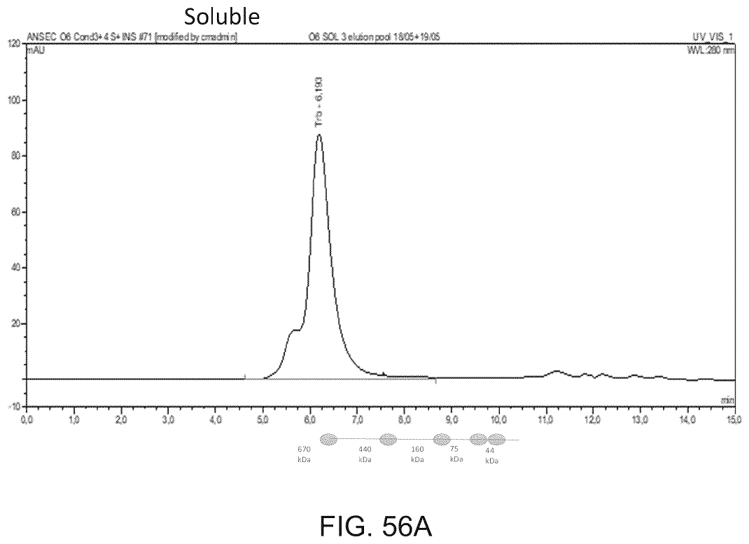

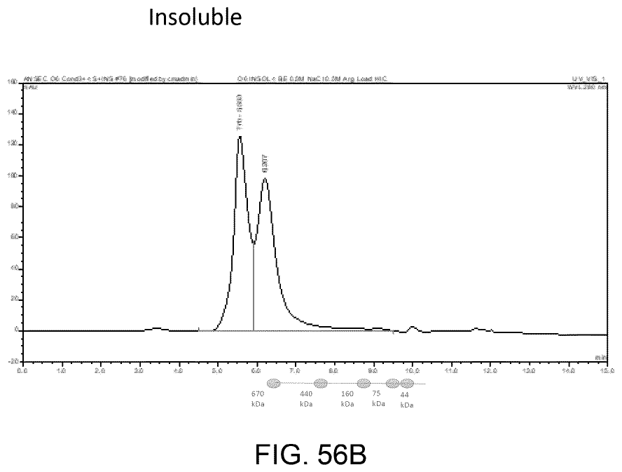

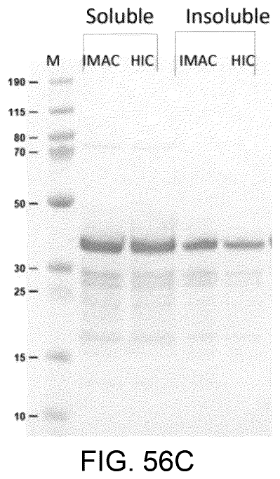

Different AnSEC profiles were observed for O6 samples from soluble (FIG. 56A) versus insoluble (FIG. 56B) samples, where a high molecular weight peak was present in the insoluble samples but not the soluble samples. However, soluble and insoluble fractions had similar CBB stained SDS-PAGE profiles (FIG. 56C). It was next of interest to perform additional polishing of the product to eliminate the high molecular weight (MW) peak present in samples from inclusion bodies.

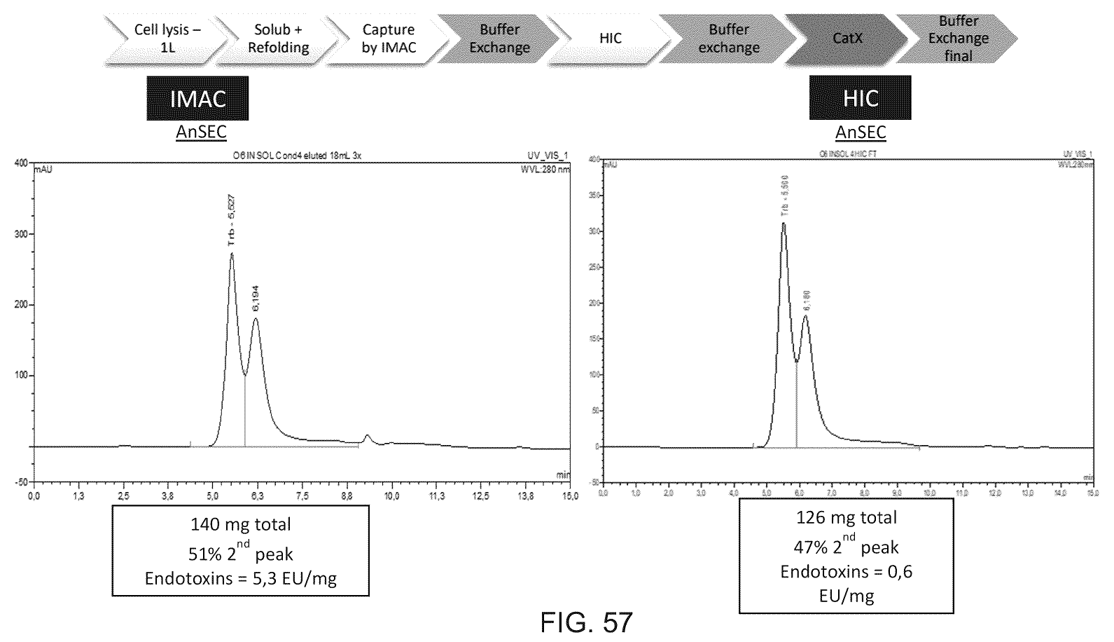

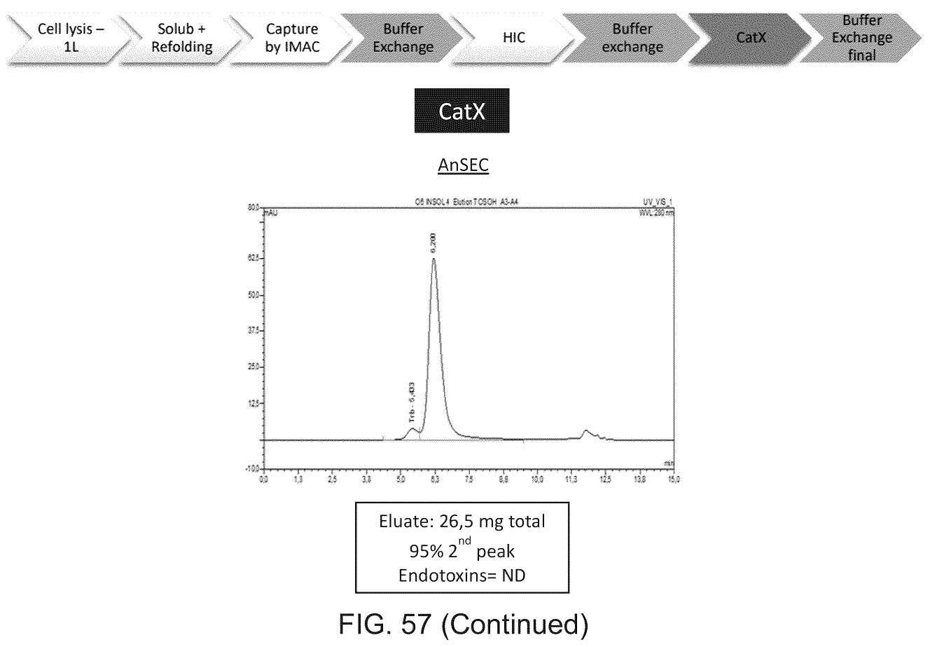

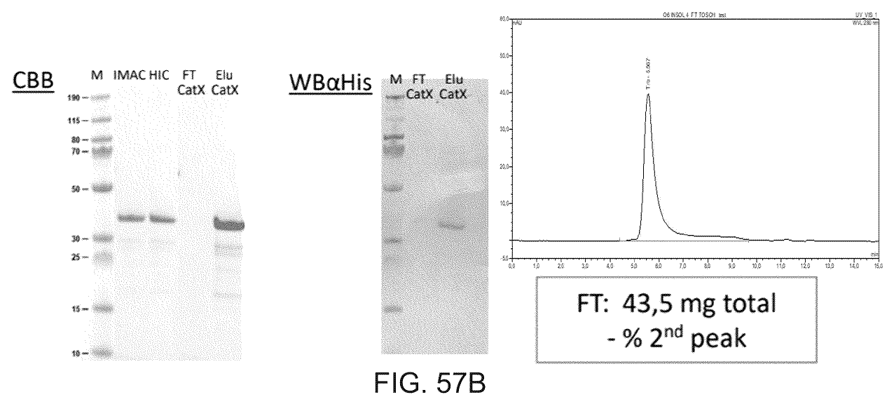

A schematic for a polishing process for O6 from the insoluble fraction performed in FIG. 57A-57C is shown in FIG. 57A. The steps of the polishing process were: 1) Bacterial pellet from 500 ml culture was lysed with Lysis Buffer+DNAse 25U/ml BB+1 KU rLysozyme. 2) Insoluble fraction (IB) was recovered and resuspended at 6 ml/g of IB with 50 mM Tris pH8+6M Guanidine for 2 hours at 400 rpm 25° C. Product was refolded for 0.5 hours at 4° C. in PBS pH8+0.5M Arg. 3) Refolded product was loaded onto a 2× 5 ml IMAC column. 4) Buffer was exchanged to 0.5M Arg 0.5M NaCl 0.02M Phosphate. 5) Samples were loaded onto a Capto Butyl impress (HIC) and flow-through was captured. 6) Buffer exchange was performed. 7) Samples were loaded onto a Tosho S650F (CatX) column and eluted with 0.5M Arg, 0.02M Phos+1M NaCl. FIG. 57A shows AnSEC data, mg protein, recovery yield, and endotoxin levels for IMAC, HIC, and CatX eluate, CatX flowthrough data is shown in FIG. 57B. HIC removed 99% of endotoxins with a protein recovery of 86%. The first peak observed by AnSEC did not bind to the cation exchange column. Eluate was mainly 2nd peak (RT 6.2 min) comprising about 95% of total area. CBB stained SDS-PAGE and anti-His Western blots did not show protein for CatX flow-through, only the eluate (FIG. 57B). A Final yield of 53 mg/L of purified product was achieved.

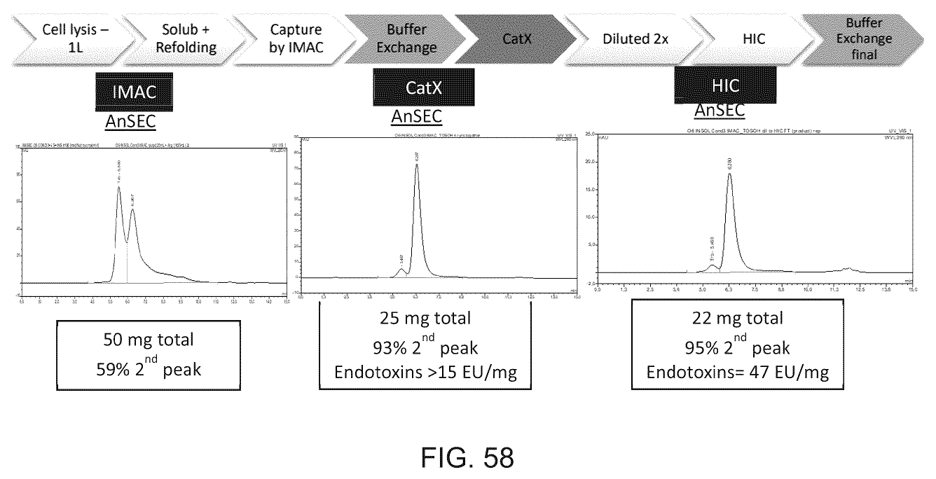

An alternative polishing process for O6 from the insoluble fraction was evaluated in FIG. 58. In this process, HIC is the final polishing step, thus reducing the number of necessary buffer changes where an average of 10% of the product is lost with each buffer change. AnSEC data, total protein, % recovery, and endotoxin levels for IMAC, CatX, and HIC samples are shown in FIG. 58. Using this strategy, recovery was 44% of IMAC capture material (19% was achieved when using CatX as final polishing step). However, endotoxins were relatively high when HIC was used following CatX (47EU/mg), whereas, endotoxins were undetectable when CatX followed HIC.

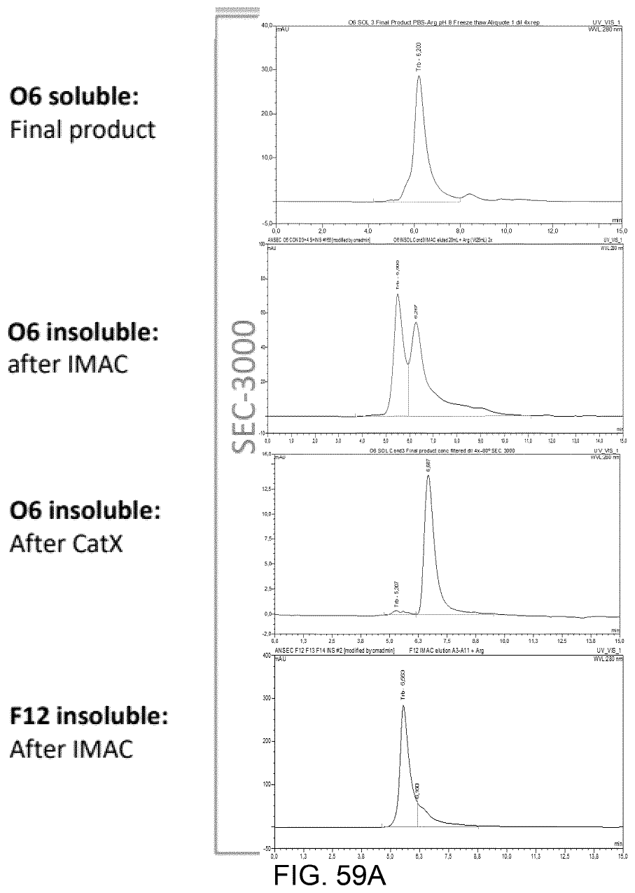

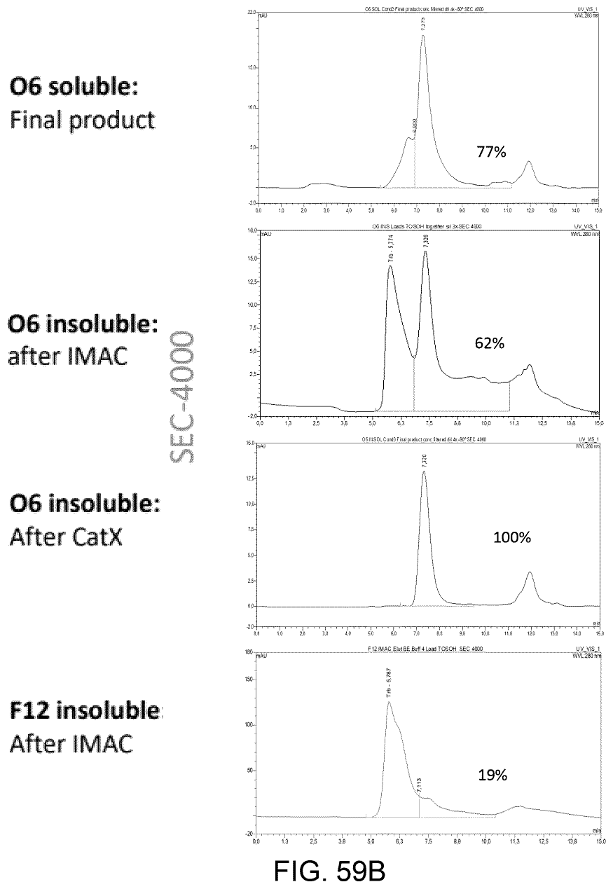

O6 soluble and insoluble samples and F12 insoluble samples were loaded into SEC-3000 (FIG. 59A) or SEC-4000 (FIG. 59B) columns to better resolve the high molecular weight peaks appearing in the AnSEC data. In the O6 soluble final product AnSEC plot, a high MW peak was clearly detected in the sample loaded into the SEC-4000 (FIG. B) representing less than 13% of total protein. After CatX, the first peak in the O6 insoluble sample obtained after IMAC with RT of 5.8 min was completely removed. In the F12 samples, the main peak detected on SEC-3000 appeared to be 2 different forms according to SEC-4000 (FIG. 59A, 59B). In both columns the peak corresponding to the POI (RT 7.1 min) corresponded to 19% of total AUC. By using a SEC-4000 column it was possible to better resolve the high MW peaks.

### Example 23 Evaluation of Purified Products

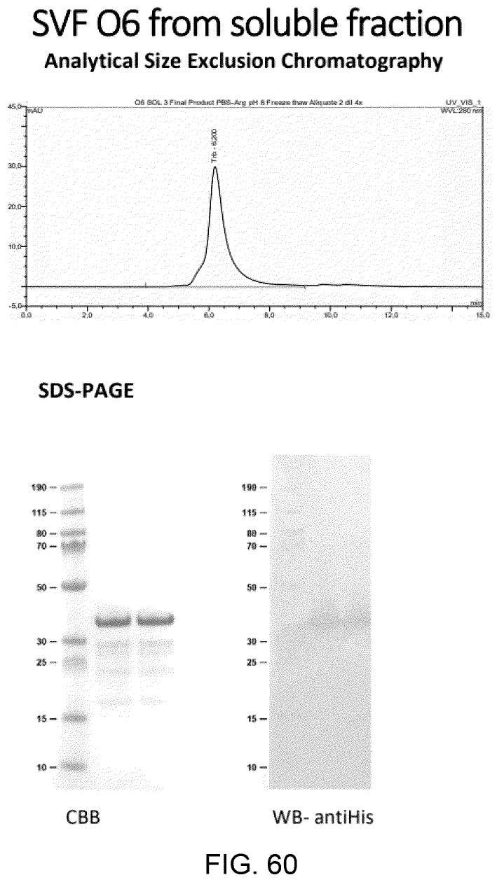

Purified sample of O6 from the soluble fraction was analyzed in FIG. 60. Following gene design and vector construction, 1000 ml of transformed E. coli culture was induced with IPTG. Cells were lysed and the soluble fraction was recovered. Buffer exchange was performed. Sample was captured by IMAC. Buffer exchange was performed a second time. Sample was polished by HIC. Concentration of the sample was performed. Buffer exchange was performed a third time and the sample was filter sterilized and aliquoted. A concentration of 0.97 mg/ml was achieved with an endotoxin level of 13 EU/mg as determined by AnSEC. Additionally, the product had a size of 32 kDa on a reducing SDS-PAGE gel and was positive for HIS tag via western blotting (FIG. 60).

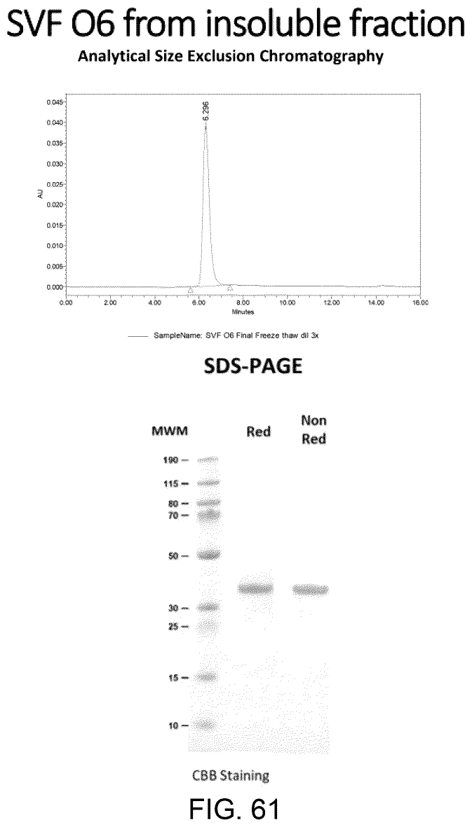

Purified sample of O6 from the insoluble fraction was analyzed in FIG. 61. Following gene design and vector construction, 500 ml transformed E. coli culture was induced with IPTG. Cells were lysed and the insoluble fraction was recovered. The insoluble fraction underwent solubilization, refolding and clarification before capture by IMAC. IMAC capture was followed by buffer exchange and polishing by CatX and HIC. Concentration of the sample was performed prior to a second buffer exchange. Sample was filter sterilized and aliquoted. A concentration of 1.14 mg/ml was achieved with an endotoxin level of 14 EU/mg as determined by AnSEC. Additionally, the product had a size of 32 kDa on a reducing SDS-PAGE gel (FIG. 61).

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

Lengthy table referenced here
US20250197452A1-20250619-T00001
Please refer to the end of the specification for access instructions.

### REFERENCES

Ahlén G, Söderholm J, Tjelle T, Kjeken R, Frelin L, Höglund U, et al. In vivo electroporation enhances the immunogenicity of hepatitis C virus nonstructural 3/4A DNA by increased local DNA uptake, protein expression, inflammation, and infiltration of CD3+ T cells. J Immunol 2007; 179:4741-53.
Bian Y, Zhang Z, Sun Z, Zhao J, Zhu D, Wang Y, et al. Vaccines Targeting PreS1 Domain Overcome Immune Tolerance in HBV Carrier Mice HHS Public Access. 2017; 66:1067-1082.
Blank A, Eidam A, Haag M, Hohmann N, Burhenne J, Schwab M, et al. The NTCP-inhibitor Myrcludex B: Effects on Bile Acid Disposition and Tenofovir Pharmacokinetics. Clin Pharmacol Ther 2018; 103:341-348.
Bogomolov P, Alexandrov A, Voronkova N, Macievich M, Kokina K, Petrachenkova M, et al. Treatment of chronic hepatitis D with the entry inhibitor myrcludex B: First results of a phase Ib/IIa study. J Hepatol 2016; 65:490-498.
Brass A, Frelin L, Milich D R, Sällberg M, Ahlen G. Functional Aspects of Intrahepatic Hepatitis B Virus-specific T Cells Induced by Therapeutic DNA Vaccination. Mol Ther 2015; 23:578-590.
Chen A, Ahlen G, Brenndorfer E D, Brass A, Holmstrom F, Chen M, et al. Heterologous T Cells Can Help Restore Function in Dysfunctional Hepatitis C Virus Nonstructural 3/4A-Specific T Cells during Therapeutic Vaccination. J Immunol 2011; 186:5107-5118.
Chen H-Y, Shen D-T, Ji D-Z, Han P-C, Zhang W-M, Ma J-F, et al. Prevalence and burden of hepatitis D virus infection in the global population: a systematic review and meta-analysis. Gut 2018; gutjnl-2018-316601.
Chen M, Sällberg M, Thung S N, Hughes J, Jones J, Milich D R. Nondeletional T-cell receptor transgenic mice: model for the CD4 (+) T-cell repertoire in chronic hepatitis B virus infection. J Virol 2000; 74:7587-99.
Chen M, Sallberg M, Hughes J, Jones J, Guidotti L G, Chisari F V., et al. Immune Tolerance Split between Hepatitis B Virus Precore and Core Proteins. J Virol 2005; 79:3016-3027.
Chen M, Jagya N, Bansal R, Frelin L, Sällberg M. Prospects and progress of DNA vaccines for treating hepatitis B. Expert Rev Vaccines 2016; 15:629-640.
Chen M T, Billaud J-N, Sallberg M, Guidotti L G, Chisari F V., Jones J, et al. A function of the hepatitis B virus precore protein is to regulate the immune response to the core antigen. Proc Natl Acad Sci 2004; 101:14913-14918.
Chen, P. J., Chang, F. L., Wang, C. J., Lin, C. J., Sung, S. Y., & Chen, D. S. (1992). Functional study of hepatitis delta virus large antigen in packaging and replication inhibition: role of the amino-terminal leucine zipper. J Virol, 66(5), 2853-2859. doi:10.1128/JVI.66.5.2853-2859.1992
Donkers J M, Zehnder B, van Westen G J P, Kwakkenbos M J, IJzerman A P, Oude Elferink R P J, et al. Reduced hepatitis B and D viral entry using clinically applied drugs as novel inhibitors of the bile acid transporter NTCP. Sci Rep 2017; 7:15307.
Dryden K A, Wieland S F, Whitten-Bauer C, Gerin J L, Chisari F V., Yeager M. Native Hepatitis B Virions and Capsids Visualized by Electron Cryomicroscopy. Mol Cell 2006; 22:843-850.
Feld J J, Terrault N A, Lin H S, Belle S H, Chung R T, Tsai N, et al. Entecavir and peginterferon alfa-2a in adults with HB eAg-positive immune tolerant chronic hepatitis B virus infection. Hepatology 2018; hep.30417.
Gripon P, Le Seyec J, Rumin S, Guguen-Guillouzo C. Myristylation of the Hepatitis B Virus Large Surface Protein Is Essential for Viral Infectivity. Virology 1995; 213:292-299.
Heidrich B, Yurdaydin C, Kabaçam G, Ratsch B A, Zachou K, Bremer B, et al. Late HDV RNA relapse after peginterferon alpha-based therapy of chronic hepatitis delta. Hepatology 2014; 60:87-97.
Hong H J, Ryu C J, Hur H, Kim S, Oh H K, Oh M S, et al. In vivo neutralization of hepatitis B virus infection by an anti-preS1 humanized antibody in chimpanzees. Virology 2004; 318:134-141.
Jayan, G. C., & Casey, J. L. (2002). Inhibition of hepatitis delta virus RNA editing by short inhibitory RNAmediatedknockdown of ADAR1 but not ADAR2 expression. J Virol, 76(23), 12399-12404.doi:10.1128/jvi.76.23.12399-12404.2002
Kosinska A D, Zhang E, Johrden L, Liu J, Seiz P L, Zhang X, et al. Combination of DNA Prime-Adenovirus Boost Immunization with Entecavir Elicits Sustained Control of Chronic Hepatitis B in the Woodchuck Model. PLOS Pathog 2013; 9: e1003391.
Lee, C. Z., Chen, P. J., & Chen, D. S. (1995). Large hepatitis delta antigen in packaging and replication inhibition: role of the carboxyl-terminal 19 amino acids and amino-terminal sequences. J Virol, 69(9), 5332-5336. doi:10.1128/JVI.69.9.5332-5336.1995
Levander S, Holmström F, Frelin L, Ahlén G, Rupp D, Long G, et al. Immune-mediated effects targeting hepatitis C virus in a syngeneic replicon cell transplantation mouse model. Gut 2018; 67:1525-1535.
Liang T J, Block T M, McMahon B J, Ghany M G, Urban S, Guo J-T, et al. Present and future therapies of hepatitis B: From discovery to cure. Hepatology 2015; 62:1893-1908.
Liu J, Li T, Zhang L, Xu A. The Role of Hepatitis B Surface Antigen in Nucleos(t)ide Analogues Cessation among Asian Chronic Hepatitis B Patients: A Systematic Review. Hepatology Published Online First: 18 Dec. 2018. doi:10.1002/hep.30474
Lok ASF, McMahon B J, Brown R S, Wong J B, Ahmed A T, Farah W, et al. Antiviral therapy for chronic hepatitis B viral infection in adults: A systematic review and meta-analysis. Hepatology 2016; 63:284-306.
Lu, X. X., Yi, Y., Su, Q. D., & Bi, S. L. (2016). Expression and Purification of Recombinant Hepatitis Delta Virus (HDV) Antigen for Use in a Diagnostic ELISA for HDV Infection Using the High-Density Fermentation Strategy in Escherichia coli. Biomed Environ Sci, 29(6), 417-423. doi:10.3967/bes2016.054
Mancini-Bourgine M, Fontaine H, Scott-Algara D, Pol S, Bréchot C, Michel M-L. Induction or expansion of T-cell responses by a hepatitis B DNA vaccine administered to chronic HBV carriers. Hepatology 2004; 40:874-882.
Maravelia, P., Frelin, L., Ni, Y., Caro Perez, N., Ahlen, G., Jagya, N., . . . Sallberg, M. (2021). Blocking Entry of Hepatitis B and D Viruses to Hepatocytes as a Novel Immunotherapy for Treating Chronic Infections. J Infect Dis, 223(1), 128-138. doi:10.1093/infdis/jiaa036
Mason W S, Gill U S, Litwin S, Zhou Y, Peri S, Pop O, et al. HBV DNA Integration and Clonal Hepatocyte Expansion in Chronic Hepatitis B Patients Considered Immune Tolerant. Gastroenterology 2016; 151:986-998.e4.
Meuleman P, Vanlandschoot P, Leroux-Roels G. A simple and rapid method to determine the zygosity of uPA-transgenic SCID mice. doi:10.1016/S0006-291X(03)01388-3
Meuleman P, Libbrecht L, De Vos R, de Hemptinne B, Gevaert K, Vandekerckhove J, et al. Morphological and biochemical characterization of a human liver in a uPA-SCID mouse chimera. Hepatology 2005; 41:847-856.
Meuleman P, Lerouxroels G. The human liver-uPA-SCID mouse: A model for the evaluation of antiviral compounds against HBV and HCV. Antiviral Res 2008; 80:231-238.
Milich D R. The Concept of Immune Tolerance in Chronic Hepatitis B Virus Infection Is Alive and Well. Gastroenterology 2016; 151:801-804.
Mitra B, Thapa R J, Guo H, Block T M. Host functions used by hepatitis B virus to complete its life cycle: Implications for developing host-targeting agents to treat chronic hepatitis B. Antiviral Res 2018; 158:185-198.
Nassal M. HBV cccDNA: viral persistence reservoir and key obstacle for a cure of chronic hepatitis B. Gut 2015; 64:1972-1984.
Neurath A R, Kent S B, Strick N, Parker K. Identification and chemical synthesis of a host cell receptor binding site on hepatitis B virus. Cell 1986; 46:429-36.
Ni Y, Sonnabend J, Seitz S, Urban S. The Pre-S2 Domain of the Hepatitis B Virus Is Dispensable for Infectivity but Serves a Spacer Function for L-Protein-Connected Virus Assembly. J Virol 2010; 84:3879-3888.
Ni Y, Lempp F A, Mehrle S, Nkongolo S, Kaufman C, Fälth M, et al. Hepatitis B and D Viruses Exploit Sodium Taurocholate Co-transporting Polypeptide for Species-Specific Entry into Hepatocytes. Gastroenterology 2014; 146:1070-1083.e6.
Ni Y, Urban S. Hepatitis B Virus Infection of HepaRG Cells, HepaRG-hNTCP Cells, and Primary Human Hepatocytes. In: Methods in molecular biology (Clifton, N.J.).; 2017. pp. 15-25.
Papatheodoridis G V., Manolakopoulos S, Touloumi G, Vourli G, Raptopoulou-Gigi M, Vafiadis-Zoumbouli I, et al. Virological suppression does not prevent the development of hepatocellular carcinoma in HBeAg-negative chronic hepatitis B patients with cirrhosis receiving oral antiviral(s) starting with lamivudine monotherapy: results of the nationwide HEPNET. Greece cohort study. Gut 2011; 60:1109-1116.
Passioura T, Watashi K, Fukano K, Sureau C, Suga H, Correspondence T W. De Novo Macrocyclic Peptide Inhibitors of Hepatitis B Virus Cellular Entry. Cell Chem Biol 2018; 25:906-915.
Razavi-Shearer D, Gamkrelidze I, Nguyen M H, Chen D-S, Van Damme P, Abbas Z, et al. Global prevalence, treatment, and prevention of hepatitis B virus infection in 2016: a modelling study. Lancet Gastroenterol Hepatol 2018; 3:383-403.
Revill P A, Chisari F V, Block J M, Dandri M, Gehring A J, Guo H, et al. A global scientific strategy to cure hepatitis B. Lancet Gastroenterol Hepatol Published Online First: 10 Apr. 2019. doi:10.1016/S2468-1253(19)30119-0
Rydell G E, Prakash K, Norder H, Lindh M. Hepatitis B surface antigen on subviral particles reduces the neutralizing effect of anti-HBs antibodies on hepatitis B viral particles in vitro. Virology 2017; 509:67-70.
Sheu, G. T., & Lai, M. M. (2000). Recombinant hepatitis delta antigen from E. coli promotes hepatitis delta virus RNA replication only from the genomic strand but not the antigenomic strand. Virology, 278(2), 578-586. doi:10.1006/viro.2000.0680
Short J M, Chen S, Roseman A M, Butler PJG, Crowther R A. Structure of Hepatitis B Surface Antigen from Subviral Tubes Determined by Electron Cryomicroscopy. J Mol Biol 2009; 390:135-141.
Suslov A, Boldanova T, Wang X, Wieland S, Heim M H. Hepatitis B Virus Does Not Interfere With Innate Immune Responses in the Human Liver. Gastroenterology 2018; 154:1778-1790.
Trépo C, Chan HLY, Lok A. Hepatitis B virus infection. Lancet 2014; 384:2053-2063.
Wedemeyer H, Yurdaydin C, Dalekos G N, Erhardt A, çakaloğlu Y, Değertekin H, et al. Peginterferon plus Adefovir versus Either Drug Alone for Hepatitis Delta. N Engl J Med 2011; 364:322-331.
WHO|Global hepatitis report, 2017. WHO 2018.
Wi J, Jeong M S, Hong H J. Construction and characterization of an anti-hepatitis B virus preS1 humanized antibody that binds to the essential receptor binding site. J Microbiol Biotechnol 2017; 27:1336-1344.
Yalcin K, Danis R, Degertekin H, Alp M N, Tekes S, Budak T. The lack of effect of therapeutic vaccination with a pre-S2/S HBV vaccine in the immune tolerant phase of chronic HBV infection. J Clin Gastroenterol 2003; 37:330-5.
Yuen M F, Gane E J, Kim D J, Weilert F, Chan HLY, Lalezari J, et al. Antiviral Activity, Safety, and Pharmacokinetics of Capsid Assembly Modulator NVR 3-778 in Patients with Chronic HBV Infection. Gastroenterology Published Online First: 6 Jan. 2019. doi:10.1053/J.GASTRO.2018.12.023
Zhao H-J, Han Q-J, Wang G, Lin A, Xu D-Q, Wang Y-Q, et al. I: C-based rHBVvac therapeutic vaccine eliminates HBV via generatioPolyn of HBV-specific CD8+effector memory T cells. Gut 2019; gutjnl-2017-315588.
Zoulim F, Mason W S. Reasons to consider earlier treatment of chronic HBV infections. Gut 2012; 61:333-336.

LENGTHY TABLES
The patent application contains a lengthy table section. A copy of the table is available in electronic form from the USPTO web site (https://seqdata.uspto.gov/?pageRequest=docDetail&DocID=US20250197452A1). An electronic copy of the table will also be available from the USPTO upon request and payment of the fee set forth in 37 CFR 1.19(b)(3).

1. A polypeptide comprising at least one hepatitis D antigen (HDAg) polypeptide sequence and at least one PreS1 polypeptide sequence, wherein each of the at least one HDAg polypeptide sequence is a C211S mutant HDAg polypeptide sequence.

2-56. (canceled)

57. The polypeptide of claim 1, wherein the at least one HDAg polypeptide sequence comprises the sequence of HDAg genotype 1 A C211S (SEQ ID NO: 47), HDAg genotype 1 B C211S (SEQ ID NO: 48), HDAg genotype 2 A C211S (SEQ ID NO: 49), HDAg genotype 2 B C211S (SEQ ID NO: 50), or any combination thereof.

58. The polypeptide of claim 1, wherein the at least one PreS1 polypeptide sequence comprises the sequence of SEQ ID NO: 11 or SEQ ID NO: 12, or both.

59. The polypeptide of claim 1, wherein the polypeptide comprises one or more epitope tags.

60. The polypeptide of claim 1, wherein the polypeptide comprises a sequence having at least 80%, 85%, 90%, 95%, 99%, or 100% homology or sequence identity to the sequence of any one of SEQ ID NO: 72-95, 140-141, 145-147, 163-177, or 187-195.

61. The polypeptide of claim 1, wherein the polypeptide is recombinantly expressed.

62. The polypeptide of claim 1, wherein the polypeptide is recombinantly expressed in a mammalian, bacterial, yeast, insect, or cell-free system.

63. The polypeptide of claim 62, wherein the polypeptide is recombinantly expressed in E. coli.

64. A nucleic acid operon comprising two or more genes, wherein each of the two or more genes comprise at least one HDAg nucleic acid sequence and at least one PreS1 nucleic acid sequence, and wherein each of the two or more genes comprises a 5′ ribosome binding site, wherein the at least one HDAg nucleic acid sequence encodes a polypeptide comprising a C211 mutation.

65. The nucleic acid operon of claim 64, wherein the at least one HDAg nucleic acid sequence and/or the at least one PreS1 nucleic acid sequence are codon optimized to reduce the number of repeat sequences.

66. The nucleic acid operon of claim 64, wherein the at least one HDAg nucleic acid sequence comprises the sequence of HDAg genotype 1 A C211S (SEQ ID NO: 43), HDAg genotype 1 B C211S (SEQ ID NO: 44), HDAg genotype 2 A C211S (SEQ ID NO: 45), HDAg genotype 2 B C211S (SEQ ID NO: 46), or any combination thereof.

67. The nucleic acid operon of claim 64, wherein the at least one PreS1 nucleic acid sequence comprise the sequence of SEQ ID NO: 51, SEQ ID NO: 52, SEQ ID NO: 53, or any combination thereof.

68. The nucleic acid operon of claim 64, wherein each of the two or more genes further encode one or more epitope tags.

69. The nucleic acid operon of claim 64, wherein the nucleic acid operon comprises a sequence having at least 80%, 85%, 90%, 95%, 99%, or 100% homology or sequence identity to the sequence of any one of SEQ ID NO: 96-101 or 196-199.

70. The nucleic acid operon of claim 64, wherein the two or more genes encode two or more polypeptides comprising the sequence of any one of SEQ ID NO: 102-133 or 200-203.

71. The nucleic acid operon of claim 70, wherein each of the two or more genes do not encode one or more epitope tags.

72. A product comprising the polypeptide of claim 1, and a nucleic acid comprising at least one nucleic acid sequence encoding HDAg and at least one nucleic acid sequence encoding PreS1, wherein each of the at least one HDAg nucleic acid sequence encodes a polypeptide comprising a C211 mutation.

73. The product of claim 72, wherein the at least one HDAg nucleic acid sequence or the at least one PreS1 nucleic acid sequence are codon optimized to minimize or reduce the number of repeat sequences.

74. The product of claim 72, wherein the nucleic acid further encodes one or more epitope tags.

75. The product of claim 72, wherein the nucleic acid comprises a sequence having at least 80%, 85%, 90%, 95%, 99%, or 100% homology or sequence identity to the sequence of any one of SEQ ID NO: 60-71, 138-139, 142-144, 148-162, or 178-186.

76. The product of claim 72, wherein the nucleic acid is DNA.

77. The product of claim 72, wherein the nucleic acid is RNA.

78. The polypeptide of claim 1, wherein at least one of the HDAg polypeptide sequences in the polypeptide comprises a C211S mutation.
